(19) United States
(12) Patent Application Publication
(10) Pub. No.: US 2007/0185013 A1
(43) Pub. Date: Aug. 9, 2007

(54) Use of chk1 inhibitors to control cell proliferation

(75) Inventor: Darcey Clark, Woodinville, WA (US)

Correspondence Address: MARSHALL, GERSTEIN & BORUN LLP, 233 S. WACKER DRIVE, SUITE 6300, SEARS TOWER, CHICAGO, IL, 60606, US

(73) Assignee: ICOS CORPORATION, Bothell, WA (US)

(21) Appl. No.: 10/572,543

(22) PCT Filed: Sep. 17, 2004
(86) PCT No.: PCT/US04/30806

§ 371 Date: Nov. 28, 2006

Related U.S. Application Data
(60) Provisional application No. 60/503,925, filed on Sep. 17, 2003.

(51) Int. Cl. A61K 38/14; A61K 31/7076; A61K 31/7072; A61K 31/7048; A61K 31/513; A61K 31/66; A61K 31/525; A61K 31/522; A61K 31/53; A61K 31/15; A61K 31/13; A61K 31/19

(52) U.S. Cl. 514/8; 514/241; 514/109; 514/672; 514/251; 514/49; 514/269; 514/706; 514/263.31; 514/47; 514/283; 514/27; 514/575; 514/664; 600/1

(57) ABSTRACT

The present invention relates to improved methods for inhibiting aberrant cell proliferation involving the scheduling of administration of Chk1 activators (e.g., chemotherapeutic agents) and Chk1 inhibitors. At least one Chk1 activator is administered at a dose and for a time sufficient to induce substantial synchronization of cell cycle arrest in proliferating cells. Upon achieving substantial phase synchronization, at least one Chk1 inhibitor is administered to abrogate the cell cycle arrest and induce therapeutic cell death. The invention is useful with any Chk1 activator and any Chk1 inhibitor, and finds application in treating or preventing cancerous and non-cancerous aberrant cell proliferation.

The present invention relates to methods for inhibiting aberrant cell proliferation involving the chemotherapeutic agents and Chk1 inhibitors.

### BACKGROUND

An important goal in healthcare is to develop and make available safer and more effective drugs and drug combinations for the treatment of aberrantly proliferating cells, such as for treatment of cancer. Most anti-proliferation therapies (including chemotherapy and radiation) act by disrupting vital processes such as DNA metabolism, DNA synthesis, DNA transcription, and microtubule spindle function, or by perturbing chromosomal structural integrity by introducing DNA lesions. These; processes affect both normal and aberrantly proliferating (e.g., tumor) cells, however. As the maintenance of DNA integrity is essential to cell viability in normal cells, anticancer drugs have the lowest therapeutic index (i.e., the highest proportion of damage to normal cells as well as tumor cells) of any drug class.

Recent work has focused on ways to increase the therapeutic index of cancer and other anti-cell proliferation therapeutics. In this regard, cellular mechanisms, known as cell cycle checkpoints, have received attention. Individual cells create an exact copy of their chromosomes and then segregate each copy into two cells by a process called mitosis. Cells have sensing mechanisms, called cell R cycle checkpoints, to maintain the order of these steps and to insure that each step is executed with high fidelity. [Hartwell et al., Science, 246:629-634 (1989); Weinert et al., Genes and Development, 8:652 (1994).]

When cells detect DNA damage induced by a chemotherapeutic agent or by radiation, cell cycle checkpoints arrest the cell cycle, allowing time for the cells to repair the DNA damage, often to a point sufficient to continue proliferation and prevent cell death. For instance, the chemotherapeutic gemcitabine, a nucleoside analog, is incorporated into synthesizing DNA causing improper synthesis and inducing cell cycle arrest. If the cells could not overcome this cell cycle arrest, the cells would die. Some cancers appear to have generated a mechanism of overcoming this cell cycle arrest, however. These resistant tumor cells simply accumulate in S phase while the chemotherapeutic agent is administered, and as soon as the drug is removed, repair the DNA damage and progress through the remainder of the cell cycle (Shi et al., Cancer Res. 61:1065-1072. 2001). The inhibition of DNA damage checkpoints is therefore expected to sensitize aberrantly proliferating cells to DNA damaging agents. Such sensitization is in turn expected to increase the therapeutic index of such chemotherapeutic agents or radiation. Thus, Keegan et al., (PCT/US02/06452, the contents of which are incorporated herein by reference), have disclosed certain small molecule compounds that selectively inhibit Chk1 kinase and their use in inhibiting Chk1.

The cell cycle is structurally and functionally conserved in its basic process and mode of regulation across all eukaryotic species. The mitotic (somatic) cell cycle consists of four phases, the G1 (gap) phase, the S (synthesis) phase, the G2 (gap) phase, and the M (mitosis) phase. The G1, S, and G2 phases are collectively referred to as interphase of the cell cycle. During the G1 phase, biosynthetic activities of the cell progress at a high rate. The S phase begins when DNA synthesis starts and ends when the DNA content of the nucleus of the cell has been replicated and two identical sets of chromosomes are formed. The cell then enters the G2 phase which continues until mitosis starts. In mitosis, the chromosomes pair and separate and two new nuclei form, and cytokinesis occurs in which the cell itself splits into two daughter cells each receiving one nucleus containing one of the two sets of chromosomes. Cytokinesis terminates the M phase and marks the beginning of interphase of the next cell cycle. The sequence in which the events in the cell cycle proceed is tightly regulated such that the initiation of one cell cycle event is dependent on the completion of the prior cell cycle event. This allows fidelity in the duplication and segregation of genetic material from one generation of somatic cells to the next.

It has been reported that cell cycle checkpoints comprise at least three distinct classes of polypeptides which act sequentially in response to cell cycle signals or defects in chromosomal mechanisms (Carr, A. M., Science, 271:314-315 (1996). The first class is a family of proteins which detect or sense DNA damage or abnormalities in the cell cycle. These sensors include Atm and Atr. The second class of polypeptides amplify and transmit the signal detected by the detector and is exemplified by Rad53 [Alen et al. Genes Dev. 8:2416-2488 (1994)] and Chk1. A third class of polypeptides includes cell cycle effectors such as p53 that mediate a cellular response, for example, arrest of mitosis and apoptosis.

Much of the current understanding of the function of cell cycle checkpoints has been derived from the study of tumor-derived cell lines. In many cases, tumor cells have lost key cell cycle check-points (Hartwell et al., Science 266: 1821-28, 1994). It has been reported that a key step in the evolution of cells to a neoplastic state is the acquisition of mutations that inactivate cell cycle checkpoint pathways, such as those involving p53 (Weinberg, R. A. Cell 81:323-330, 1995; Levine, A. J. Cell 88: 3234-331, 1997). Loss of these cell cycle-checkpoints results in the replication of tumor cells despite DNA damage.

Noncancerous tissue, which has intact cell cycle checkpoints; typically is insulated from temporary disruption of a single checkpoint pathway. Tumor cells, however, have defects in pathways controlling cell cycle progression such that the perturbation of additional checkpoints renders them particularly sensitive to DNA damaging agents. For example, tumor cells that contain mutant p53 are defective both in the G1 DNA damage checkpoint and in the ability to maintain the G2 DNA damage checkpoint (Bunz et al., Science, 282:1497-501, 1998). Checkpoint inhibitors that target initiation of the G2 checkpoint or the S phase checkpoint are expected to further cripple the ability of these tumor cells to repair DNA damage and, therefore, are candidates to enhance the therapeutic index of both radiation and systemic chemotherapy (Gesner, T., Abstract at SRI Conference: Protein Phosphorylation and Drug Discovery World-Summit. March 2003.)

In the presence of DNA damage or any block to DNA replication, the checkpoint proteins Atm and Atr initiate a signal transduction pathway leading to cell, cycle arrest. Atm has been shown to play a role in a DNA damage check-point in response to ionizing radiation (IR). Atr is stimulated by agents that cause double strand DNA breaks, single strand DNA breaks, and agents that block DNA from radiation.

Chk1 is a protein kinase that lies downstream from Atm and/or Atr in the DNA damage checkpoint signal transduction pathway. (Sanchez et al., Science, 277:1497-1501, 1997; U.S. Pat. No. 6,218,109) In mammalian cells, Chk1 is phosphorylated in response to agents that cause DNA damage including ionizing radiation (IR), ultraviolet (UV) light, and hydroxyurea (Sanchez et al., supra; Lui et al., Genes Dev., 14:1448-1459, 2000). The phosphorylation and activation of Chk1 in mammalian cells is dependent on Atm (Chen et al., Oncogene, 18:249-256, 1999) and Atr (Lui et al., supra). Furthermore, Chk1 has been shown to phosphorylate both wee1 (O'Connell et al., EMBO J., 16:545-554, 1997) and Pds1 (Sanchez et al., Science, 286:1166-1171, 1999) gene products known to be important in cell cycle control.

These studies demonstrate that mammalian Chk1 plays a role in the Atm-dependent DNA damage checkpoint leading to arrest at S phase. A role for Chk1 in the S phase mammalian cells has recently been elucidated (Feijoo et al., J. Cell Biol., 154:913-923, 2001; Zhao et al., PNAS USA, 99:14795-800, 2002; Xiao et al.; J Biol Chem., 278(24):21767-21773, 2003; Sorensen et al., Cancer Cell, 3(3):247-58, 2003) highlighting the role of Chk1 in monitoring the integrity of DNA synthesis. Chk1 invokes an S-phase arrest by phosphorylatinig Cdc25A, which regulates cyclinA/cdk2 (Xiao et al., supra and Sorensen et al., supra). Chk1 also invokes a G2 arrest by phosphorylating and inactivating Cdc25C, the dual specificity phosphatase that normally dephosphorylates cyclin-B/cdc2 (also known as Cdk1) as cells progress, into mitosis (Fernery et al., Science, 277: 1495-7, 1997; Sanchez et al., supra; Matsuoka et al., Science. 282:1893-1897, 1998; and Blasina et al., Curr. Biol., 9:1-10, 1999). In both cases, regulation of Cdk activity induces a cell cycle arrest to prevent cells from entering mitosis in the presence of DNA damage or unreplicated DNA.

Additional classes of cell cycle checkpoint inhibitors inhibit the cell cycle at either the G1 or G2/M phase. UCN-01, or 7-hydroxystaurosporine, a derivative of staurosporine, was originally isolated as a non-specific kinase inhibitor, and was found to have its primary effect on protein kinase C, but has recently been found to inhibit the activity of Chk1 and abrogate the G2 cell cycle checkpoint (Shi et. al., supra). Thus, UCN-01 is a non-selective Chk1 inhibitor. As a result, UCN-01 is toxic to cells at high doses. At low doses, it non-specifically inhibits many cellular kinases and also inhibits the G1 checkpoint (Tenzer and Pruschy, Curr. Med. Chem. Anti-Cancer Agents, 3:35-46, 2003).

UCN-01 has been used in conjunction with chemotherapeutic therapies, such as irradiation, and with the anti-cancer agent camptothecin (Tenzer and Pruschy, supra), and gemcitabine (Shi et al., supra) with limited success. In addition, UCN-01 has also been used to potentiate the effects of temozolomide (TMZ) induced DNA mismatch repair (MMR) in glioblastoma cells (Hirose et al., Cancer Res., 61:5843-5849, 2001). In the clinic, UCN-01 is not as effective a chemotherapeutic as once was hoped, perhaps due to a failure in treatment scheduling and a lack of identification of particular key molecular targets (Grant and Roberts, Drug Resistance Updates, 6:15-26, 2003). Thus, Mack et al. report cell cycle-dependent potentiation of cisplatin by UCN-01 in cultured non-small-cell lung carcinoma cell line, but do not identify with specificity the key cell cycle checkpoint(s) targeted by UCN-01. (Mack et al., Cancer Chemother Pharmacol., 51(4):337-348, 2003).

Several other strategies exist for sensitizing tumor cells to treatment with cell cycle affecting chemotherapeutics. For example, administration of 2-aminopurine abrogates multiple cell cycle checkpoint mechanisms, such as mimosine-induced G1 arrest or hydroxyurea-induced S phase arrest, allowing the cell to progress into and through mitosis (Andreassen et al., Proc Natl Acad Sci USA., 86:2272-2276, 1992). Caffeine, a methylxanthine, has also been used to enhance cytotoxicity of DNA-damaging agents, such as cisplatin and ionizing radiation, by mediating progression through the G2 checkpoint and thereby inducing cell death. (Bracey et al., Clin Cancer Res., 3:1371-1381, 1997). However, the dose of caffeine used to accomplish the cell cycle abrogation exceeds clinically acceptable levels and is not a viable therapeutic option. Additionally, antisense nucleotides to Chk1 kinase have been used to increase sensitivity to the topoisomerase inhibitor BNP1350 (Yin et al., Biochem. Biophys. Res. Commun., 295:435-44, 2002), but demonstrate the problems typically associated with antisense treatment and gene therapy.

Thus, treatments that modulate the underlying molecular mechanisms of cell cycle progression and resistance to DNA damage were expected to potentiate tumor cell killing and enhance the therapeutic index of existing therapies. Inhibition of additional DNA damage checkpoints by Chk1 inhibitors was expected to potentiate such treatments by selectively sensitizing abnormally proliferating cells to DNA damaging agents. However, the degree of selective sensitization or potentiation obtained was not as effective as hoped in these methods.

Consequently, there is a need in the art to develop a therapeutic regimen that more specifically targets particular cell cycle checkpoints in aberrantly dividing cells, thus providing better, faster and safer therapies to patients with proliferative diseases. The present invention addresses this need.

### BRIEF DESCRIPTION OF THE DRAWINGS

FIG. 1 describes the effects of Chk1 inhibitor on HeLa cells, FIG. 1A depicts the effects of ionizing radiation and Chk1 inhibitor on CyclinB/cdc2 kinase activity and induction of mitosis. Activity is shown as a percent relative to nocodazole (noc)-treated cells. FIG. 1B depicts Chk1 inhibitor effects on HeLa cell cycle progression as shown by mitotic index experiments. Activity is based on CyclinB/cdc2 kinase activity.

FIG. 2 describes the effects of Chk1 inhibitor on HT29 colon carcinoma cells. FIG. 2A depicts the percent of cells in S phase after treatment with camptothecin and Chk1 inhibitor. FIG. 2B depicts the effects of camptothecin and Chk1 inhibitor on HT29 cells as shown by mitotic index experiments. FIG. 2C depicts the percent of HT29 cells in mitosis after treatment with either Ara-C, aphidicolin or fludarabine and Chk1 inhibitor.

FIG. 3 is a Western blot showing the phosphorylation state of Chk1 after gemcitabine treatment of HT29 cells.

FIG. 4 is a Western blot showing the phosphorylation state of serine 296 of Chk1 after treatment of HT29 cells with gemcitabine alone or gemcitabine plus Chk1 inhibitor.

### SUMMARY OF THE INVENTION

The present invention provides a method for controlling aberrant cell proliferation. The method comprises contacting a cell population comprising aberrantly proliferating cells with at least one Chk1 activator in an amount and for a time sufficient to substantially synchronize cell cycle arrest among the aberrantly proliferating cells. Upon achieving substantial synchronization of cell cycle arrest in said population, the cell population is contacted with at least one Chk1 inhibitor in an amount and for a time sufficient to substantially abrogate the cell cycle arrest.

In one embodiment the present invention provides a method for sensitizing a population of aberrantly proliferating cells to the effects of at least one Chk1 activator. In another embodiment, the present invention provides a method for increasing the therapeutic index of at least one Chk1 activator in the treatment of at least one disease, condition, or disorder associated with, mediated by, or caused by aberrant cell proliferation.

The present invention also comprises articles of manufacture. Such articles comprise at least one Chk1 inhibitor, optionally together with a pharmaceutical carrier or diluent, and at least one label describing a method of use of the Chk1 inhibitor according to the invention. Such articles of manufacture may also optionally comprise at least one Chk1 activator.

The present invention also calls for use of a composition comprising at least one Chk1 inhibitor in the manufacture of a medicament for the inhibition or prevention of aberrant cell proliferation, or for the treatment or prophylaxis of a disease, condition, or disorder in a subject characterized or mediated by aberrant cell proliferation.

“Aberrant cell proliferation” means cell proliferation that deviates from the normal, proper, or expected course. For example, aberrant cell proliferation may include inappropriate proliferation of cells whose DNA or other cellular components have become damaged or defective. Aberrant cell proliferation may include cell proliferation whose characteristics are associated with a disease, condition, or disorder caused by, mediated by, or resulting in inappropriately high levels of cell division, inappropriately low levels of apoptosis, or both. Such diseases, conditions, or disorders may be characterized, for example, by single or multiple local abnormal proliferations of cells, groups of cells or tissue(s), whether cancerous or non-cancerous, benign or malignant, described more fully below.

“Controlling” aberrant cell proliferation encompasses inhibiting and preventing aberrant cell proliferation in either an in vivo or ex vivo contexts as described herein.

“Inhibiting aberrant cell proliferation” means to slow or stop the rate at which aberrantly proliferating cells proliferate. This may result either from a decreased rate of replication, an increased rate of cell death, or both. Cell death may occur by any mechanism, including apoptosis and mitotic catastrophe. Use of the present invention may result in partial or complete regression of aberrantly proliferating cells, i.e., the partial or complete disappearance of such cells from the cell population. Thus, for example, when the population of aberrantly proliferating cells are tumor cells, the method of the invention may be used to slow the rate of tumor growth, decrease the size or number of tumors, or to induce partial or complete tumor regression.

“Preventing aberrant cell proliferation” means that the present invention may be used prophylactically to prevent or inhibit aberrant cell proliferation before it occurs, or to prevent or inhibit the recurrence thereof. Thus, in all embodiments; the invention may be used in vivo or ex vivo where no aberrant cell proliferation has been identified or where no aberrant cell proliferation is ongoing, but where aberrant cell proliferation is suspected or expected, respectively. Moreover, the invention may also be used in all its embodiments wherever aberrant cell proliferation has been previously treated to prevent or inhibit recurrence of the same. In these and related embodiments, the “cell population comprising aberrantly proliferating cells” may refer to any cell population where no aberrant cell proliferation has been identified or is ongoing, but where aberrant cell proliferation is suspected or expected, respectively, and/or any cell population previously treated for aberrant cell proliferation to prevent or inhibit recurrence of the same.

“Chk1 activator” means any agent, whether now known or after discovered, whether naturally occurring or man-made, having an ability to activate Chk1 kinase sufficient to induce a cell cycle arrest. An agent may be identified as a Chk1 activator for purposes of this invention by methods known in the art. In one non-limiting method, the phosphorylation state of Chk1 is measured as an indication of Chk1 activation. For example, the phosphorylation of Chk1 serines 317 and 345 have been shown to correlate with Chk1 activation after treatment with agents known to activate Chk1, as described in Example 12 hereinbelow. Chk1 activators include those capable of arresting the cell cycle at a specific phase of the cell cycle, which phase may be referred to herein as the “target phase” for that activator. Target phases include any of the cell cycle phases except mitosis. Thus, in certain embodiments, the Chk1 activator will induce cell cycle arrest at the G1 phase. In certain other embodiments, the Chk1 activator will induce cell cycle arrest at the S phase. In certain other embodiments, the Chk1 activator will induce cell cycle arrest at the G2 phase.

Any chemotherapeutic agent, known or after-discovered, capable of functioning as a Chk1 activator may be used in the present invention. Any radiotherapeutic agent, known or after-discovered, capable of functioning as a Chk1 activator may be used in the present invention. The selection of a suitable Chk1 activator is within the level of skill of the ordinarily skilled artisan. Factors used in the selection will depend, for example, upon the condition being treated, the cell type of aberrantly proliferating cells targeted, whether such cells are to be exposed to the Chk1 activator in vivo or ex vivo, the recipient's health, and other factors which are known to those of ordinary skill in the art. Available Chk1 activators may be adapted for use in the control of any aberrantly proliferating cell type or the conditions listed herein. For example, when the method is used to treat non-cancerous aberrantly proliferating cells, lower levels will typically be used than when treating cancerous aberrantly proliferating cells. For example, levels of radiation, e.g., ultraviolet (UV) radiation, and/or low levels of suitable chemotherapeutic agents (e.g., methotrexate) may be used in the control of aberrantly proliferating cells according to the invention.

Examples of chemotherapeutic agents capable of serving as Chk1 activators include, but are not limited to

Alkylating agents, such as nitrogen mustards. (e.g., mechlorethamine, cyclophosphamide, ifosfamide, melphalan, and chlorambucil); nitrosoureas (e.g., carmustine (BCNU), lomustine (CCNU), and semustine (methyl-CCNU)); ethylenimines and methyl-melamines (e.g., triethylenemelamine (TEM), triethylene thiophosphoramide (thiotepa), and hexamethylmelamine (HMM, altretamine)); alkyl sulfonates (e.g., buslfan); and triazines (e.g., dacabazine (DTIC));

Antimetabolites, such as folic acid analogs (e.g., methotrexate, trimetrexate, and pemetrexed (multi-targeted antifolate)); pyrimidine analogs (such as 5-fluorouracil (5-FU), fluorodeoxyaridine, gemcitabine, cytosine arabinoside (AraC, cytarabine), 5-azacytidine, and 2,2′-difluorodeoxycytidine); and purine analogs (e.g., 6-mercaptopurine, 6-thioguanine, azathioprine, 2′-deoxycoformycin (pentostatin), erythrohydroxynonyladenine (EHNA), fludarabine phosphate, 2-chlorodeoxyadenosine (cladribine, 2-CdA));

Type I topoisomerase inhibitors such as camptothecin (CPT), topotecan, and irinotecan;

Certain natural products, such as epipodophylotoxins (e.g., etoposide and teniposide); and vinca alkaloids (e.g., vinblastine, vincristine, and vinorelbine);

Anti-tumor antibiotics such as actinomycin D, doxorubicin, and bleomycin;

Certain radiosensitizers such as 5-bromodeozyuridine, 5-iododeoxyuridine, and bromodeoxycytidine;

Platinum coordination complexes such as cisplatin, carboplatin, and oxaliplatin;

Substituted ureas, such as hydroxyurea; and

Methylhydrazine derivatives such as N-methylhydrazine (MIH) and procarbazine.

Examples of radiotherapeutic Chk1 activators include, but are not limited to, ionizing radiation, such as x-ray radiation, ultraviolet light and mixtures thereof.

At least one Chk1 activator is used in the method of the invention. If more than one Chk1 activator is used, the Chk1 activators may be co-administered or administered at separate times as determined by those of ordinary skill in the art.

Chk1 activators may be used alone or in combination with other chemotherapeutic or radiotherapeutic agents that may or may not function as Chk1 activators. Radiotherapeutic agents may be used in conjunction with radiosensitizers and/or photosensitizers, as are known in the art. Any of the foregoing agents may be used in conjunction with other active and inactive agents, such as those capable of reducing side effects. Combination treatments are well known in the art or may readily be determined by those of ordinary skill in the art. Non-limiting examples of chemotherapeutic agents, radiotherapeutic agents and other active and ancillary agents are shown in Table 1.

TABLE 1

Alkylating agents
Nitrogen mustards
mechlorethamine
cyclophosphamide
ifosfamide
melphalan
chlorambucil
Nitrosoureas
carmustine (BCNU)
lomustine (CCNU)
semustine (methyl-CCNU)
Ethylenimine/Methyl-melamine
thriethylenemelamine (TEM)
triethylene thiophosphoramide
(thiotepa)
hexamethylmelamine
(HMM, altretamine)
Alkyl sulfonates
busulfan
Triazines
dacarbazine (DTIC)
Antimetabolites
Folic Acid analogs
methotrexate
Trimetrexate
Pemetrexed
(Multi-targeted antifolate)
Pyrimidine analogs
5-fluorouracil
fluorodeoxyuridine
gemcitabine
cytosine arabinoside
(AraC, cytarabine)
5-azacytidine
2,2′-difluorodeoxy-cytidine
Purine analogs
6-mercaptopurine
6-thioguanine
azathioprine
2′-deoxycoformycin
(pentostatin)
erythrohydroxynonyl-adenine (EHNA)
fludarabine phosphate
2-chlorodeoxyadenosine
(cladribine, 2-CdA)
Type I Topoisomerase Inhibitors
camptothecin
topotecan
irinotecan
Biological response modifiers
G-CSF
GM-CSF
Differentiation Agents
retinoic acid derivatives
Hormones and antagonists
Adrenocorticosteroids/antagonists
prednisone and equiv-alents
dexamethasone
ainoglutethimide
Progestins
hydroxyprogesterone caproate
medroxyprogesterone acetate
megestrol acetate
Estrogens
diethylstilbestrol
ethynyl estradiol/equivalents
Antiestrogen
tamoxifen
Androgens
testosterone propionate
fluoxymesterone/equivalents
Antiandrogens
flutamide
gonadotropin-releasing
hormone analogs
leuprolide
Nonsteroidal antiandrogens
flutamide
Natural products
Antimitotic drugs
Taxanes
paclitaxel
Vinca alkaloids
vinblastine (VLB)
vincristine
vinorelbine
Taxotere ® (docetaxel)
estramustine
estramustine phosphate
Epipodophylotoxins
etoposide
teniposide
Antibiotics
actimomycin D
daunomycin (rubido-mycin)
doxorubicin (adria-mycin)
mitoxantroneidarubicin
bleomycin
splicamycin (mithramycin)
mitomycinC
dactinomycin
aphidicolin
Enzymes
L-asparaginase
L-arginase
Radiosensitizers
metronidazole
misonidazole
desmethylmisonidazole
pimonidazole
etanidazole
nimorazole
RSU 1069
EO9
RB 6145
SR4233
nicotinamide
5-bromodeozyuridine
5-iododeoxyuridine
bromodeoxycytidine
Miscellaneous agents
Platinium coordination complexes
cisplatin
Carboplatin
oxaliplatin
Anthracenedione
mitoxantrone
Substituted urea
hydroxyurea
Methylhydrazine derivatives
N-methylhydrazine (MIH)
procarbazine
Adrenocortical suppressant
mitotane (o,p′-DDD)
ainoglutethimide
Cytokines
interferon (α, β, γ)
interleukin-2
Photosensitizers
hematoporphyrin derivatives
Photofrin ®
benzoporphyrin derivatives
Npe6
tin etioporphyrin (SnET2)
pheoboride-a
bacteriochlorophyll-a
naphthalocyanines
phthalocyanines
zinc phthalocyanines
Radiation
X-ray
ultraviolet light
gamma radiation
visible light
infrared radiation
microwave radiation

“Chk1 inhibitor” means any agent, whether now known or after-discovered, whether naturally occurring or man-made, that is capable of at least partially abrogating cell cycle checkpoint activity of Chk1. Such agents include, but are not limited to, small molecule compounds, biologics, and antisense agents.

Abrogation of cell cycle checkpoint is achieved when the cellular checkpoint mechanism(s) is (are) overcome sufficiently to allow a cell to pass from the cell cycle phase in which it is halted by the Chk1 activator to the next phase in the cell cycle or to allow a cell to pass directly to cell death. Without wishing to be bound by theory, it is believed that abrogation of the cell cycle checkpoint permits cells to carry damage or imperfections, including damage induced by the Chk1 activator that might otherwise have been repaired, to subsequent cell cycle phases, thereby inducing or promoting cell death. Cell death may occur by any mechanism, including apoptosis and mitotic catastrophe. In one embodiment, the Chk1 activator and the Chk1 inhibitor each influence the same target phase, with the Chk1 activator arresting the cells in the target phase, and the Chk1 inhibitor abrogating that arrest. If more than one Chk1 inhibitor is used, the Chk1 inhibitors may be co-administered or administered at separate times as determined by the attending physician or laboratory technician. One way to assess Chk1 inhibitor activity is by assessing Chk1 activity, as described in Example 13 below.

Chk1 inhibitors useful in the present invention include, but are not limited to, those described or claimed in the following publications, the entire disclosures of which are incorporated herein by reference:

Aryl- and heteroaryl-substituted urea compounds described in any one of the following co-owned, co-pending patent applications: U.S. patent application Ser. No. 10/087,715 (patent family member of International Patent Publication No.: WO 2002/070494), U.S. Provisional Patent Application Nos.: 60/583,080, 60/585,292, and 60/602,968; Diaryl urea compounds (described in US20040014765); US Patent Publication No. US2003/199511; US Patent Publication No. 2004/0014765; WO03/101444; Methylxanthines and related compounds (described in Fan et al., Cancer Res. 55:1649-54. 1995); Ureidothiphenes (described in International Patent Publication No. WO03/029241 and WO 03/028731); N-pyrrolopyridinyl carboxamides (described in International Patent Publication No. WO03/028724); Antisense Chk1 oligonucleotides (described in International Patent Publication No. WO01/57206 and U.S. Pat. No. 6,211,164); Chk1 receptor antagonists (described in International Patent Publication No. WO00/16781); Heteroaromatic carboxamide derivatives (described in International Patent Publication No. WO03/037886); Aminothiophenes (described in International Patent Publication No. WO03/029242); (Indazolyl) benzimidazoles (described in International Patent Publication No. WO03/004488); Benzimidazole quinolinones (described in US Patent Publication No. 20040092535 and WO04/018419), Heterocyclic-hydroxyimino-fluorenes (described in International Patent Publication No. WO02/16326); Scytoneman skeleton containing derivatives (scytonemin) (described in U.S. Pat. No. 6,495,586); Heteroarylbenzamides (described in International Patent Publication No. WO01/53274); Indazole compounds (described in International Patent Publication No. WO01/53268); Indolacarbazoles (described in Tenzer et al., supra); Chromane deriviatives (described in International Patent Publication No. WO02/070515); Paullones (described in Schultz, et al., J. Med. Chem., Vol: 2909-2919. 1999); Indenopyrazoles (described in International Patent Publication No WO99/17769); Flavones (described in Sedlacek et al., Int J. Oncl. 9:1143-1168. 1996); Peptide derivatives of peptide-loop of serine threonine kinases (described in International Patent Publication No. WO98/53050); Oxindoles (described in International Patent Publication No. WO03/051838); Diazepinoindolones (described in International Patent Publication No. WO 2004/063198); Pyrimidines (described in International Patent Publication No. WO 2004/048343); Urea compounds (described in International Patent Publication No. WO 2004/014876); and Pyrrolocarbazoles, benzofuroisoindoles, and azacyclopentafluorenes (described in International Patent Publication No. WO 2003/091255).

I. Diarylurea Compounds as Described in WO02070494, Including:

i) A compound of formula: 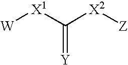
wherein X1 is null, —O—, —S—, —CH2-, or —N(R1)-;

X2 is —O—, —S—, or —N(R1)-; Y is O or S; or ═Y represents two hydrogen atoms attached to a common carbon atom; W is selected from the group consisting of heteroaryl, aryl, heterocycloalkyl, cycloalkyl, and C13 alkyl substituted with a heteroaryl or aryl group;

Z is selected front the group consisting of hydro, aryl, and heteroaryl; wherein said aryl groups of W and Z are optionally substituted with one to four substituents represented by R2, said heteroaryl groups of W and Z are optionally substituted with one to four substituents represented by R5, and said heterocycloalkyl and cycloalkyl groups of W are optionally substituted with one to two substituents represented by R6;

R1 is selected from the group consisting of hydro, C1-6alkyl, C2-6alkenyl, C2-6alkynyl, and aryl;

R2 is selected from the group consisting of halo, optionally substituted C1-6alkyl, C2-6alkenyl, OCF3, NO2, CN, NC, N(R3)2, OR3, CO2R3, C(O)N(R3)2, C(O)R3, N(R1)COR3, N(R1)C(O)OR3, N(R3)C(O)OR3, N(R3)C(O)C1-3alkyleneC(O)R3, N(R3)C(O)C1-3alkyleneC(O)OR3, N(R3)C(O)C1-3alkyleneOR3, N(R3)C(O)C1-3alkyleneNHC(O)—OR3, N(R3)C(O)C1-3alkyleneSO2NR3, C1-3alkyleneOR3, and SR3;

R3 is selected from the group consisting of hydro, C1-6alkyl, C2-6alkenyl, cycloalkyl, aryl, heteroaryl, SO2R4, C1-6alkyl substituted with one or more of halo, hydroxy, aryl, heteroaryl, heterocycloalkyl, N(R4)2, and SO2R4, C1-3alkylenearyl, C1-3alkyleneheteroaryl, C1-3alkyleneC3-8heterocycloalkyl, C1-3alkyleneSO2aryl, optionally substituted C1-3alkyleneN(R4)2, OCF3, C1-3alkyleneN(R4)3+, C3-8heterocycloalkyl, and CH(C1 3alkyleneN(R4)2)2, or two R3 groups are taken together to form an optionally substituted 3- to 6-membered aliphatic ring;

R4 is selected from the group consisting of hydro, C1-6alkyl, cycloalkyl, aryl, heteroaryl, C1-3-alkylenearyl, and SO2C1-6alkyl, or two R4 groups are taken together to form an optionally substituted 3- to 6-membered ring;

R5 is selected from the group consisting of C1-6alkyl, aryl, N(R3)2, OR3, halo, N3, CN, C1-3alkylenearyl, C1-3alkyleneN(R3)2, C(O)R3, and

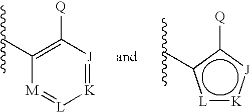

R6 is selected from the group consisting of halo and C1-6alkyl; and pharmaceutically acceptable salts, prodrugs, or solvates thereof.

ii) A compound of Formula: 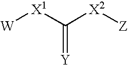
wherein X1 is null, —O—, —S—, —CH2-, or —N(R1)-;

X2 is —O—, —S—, or —N(R1)-; Y is O or S; or ═Y represents two hydrogen atoms attached to a common carbon atom; W is selected from the group consisting of heteroaryl, aryl, heterocycloalkyl, cycloalkyl, and C13 alkyl substituted with a heteroaryl or aryl group;

Z is selected from the group consisting of hydro, aryl, and heteroaryl; wherein said aryl groups of W and Z are optionally substituted with one to four substituents represented by R2, said heteroaryl groups of W and Z are optionally substituted with one to four substituents represented by R5, and said heterocycloalkyl and cycloalkyl groups of W are optionally substituted with one to two substituents represented by R6;

R1 is selected from the group consisting of hydro, C1-6alkyl, C2-6alkenyl, C2-6alkynyl, and aryl;

R2 is selected from the group consisting of halo, optionally substituted C1-6alkyl, C2-6alkenyl, OCF3, NO2, CN, NC, N(R3)2, OR3, CO2R3, C(O)N(R3)2, C(O)R3, N(R1)COR3, N(R1)C(O)OR3, N(R3)C(O)OR3, N(R3)C(0)C1-3alkyleneC(O)R3, N(R3)C(O) C1-3alkyleneC(O)OR3, N(R3)C(O)C1-3alkyleneOR3, N(R3)C(O)C1-3alkyleneNHC(O)—OR3, N(R3)C(O)C1-3alkyleneSO2NR3, C1-3alkyleneOR3, and SR3;

R3 is selected from the group consisting of hydro, C1-6alkyl, C2-6alkenyl, cycloalkyl, aryl, heteroaryl, SO2R4, C1-6alkyl substituted with one or more of halo, hydroxy, aryl, heteroaryl, heterocycloalkyl, N(R4)2, and SO2R4, C1-3alkylenearyl, C1-3alkyleneheteroaryl, C1-3alkyleneC3-8heterocycloalkyl, C1-3alkyleneSO2aryl, optionally substituted C1-3alkyleneN(R4)2, OCF3, C1-3alkyleneN(R4)3+, C3-8 heterocycloalkyl, and CH(C1-3alkyleneN(R4)2)2, or two R3 groups are taken together to form an optionally substituted 3- to 6-membered aliphatic ring;

R4 is selected from the group consisting of hydro, C1-6alkyl, cycloalkyl aryl, heteroaryl, C1-3-alkylenearyl, and SO2C1-6alkyl, or two R4 groups are taken together to form an optionally substituted 3-to 6-membered ring;

R5 is selected from the group consisting of C1-6alkyl, aryl, N(R3)2, OR3, halo, N3, CN, C1-3alkylenearyl, C1-3alkyleneN(R3)2, C(O)R3, and

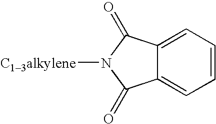

R6 is selected from the group consisting of halo and C1-6alkyl; and pharmaceutically acceptable salts, prodrugs, or solvates thereof.

iii) A compound of Formula: 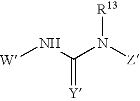
wherein Y′ is O or S;

W′ is selected from the group consisting of

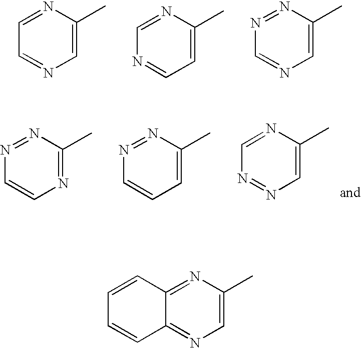

optionally substituted with from one to four substituents selected from the group consisting of C1-6alkyl, aryl, N(R7)2, OR7, N3, CN, C(O)R7, C1-3alkylenearyl, C1-3alkyleneN (R12)2, 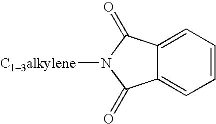
and halo;

Z′ is selected from the group consisting of: 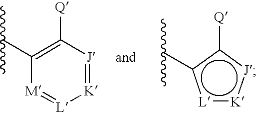
wherein:

Q′ is selected from the group consisting of hydro, OR7, SR7, and N (R7)2, with the proviso that Q′ is hydro only when at least one of J′, K′, L′, and M′ is N, O, or S;

J′ is selected from the group consisting of CR8, NR8, O, and S;

K′ is selected from the group consisting of CR9, NR9, O, and S; L′ is selected from the group consisting of CR10, NR10, O, and S;

M′ is selected from the group consisting of CR11, NR11, O, and S, with the proviso that Z is different from a pyridone;

wherein: R7, independently, is selected from the group consisting of hydro, C1-6alkyl, C2-6alkenyl, cycloalkyl, aryl, heteroaryl, SO2R12, C1-6alkyl substituted with one or more of halo, hydroxy, aryl, heteroaryl, heterocycloalkyl, N(R12)2, and SO2R12, C1-3alkylenearyl, C1-3alkyleneheteroaryl, C1-3alkyleneC3-8heterocycloalkyl, C1-3alkyleneSO2aryl, optionally substituted C1-3alkyleneN (R12)2, OCF3, C1-3alkyleneN (R12)3+, C3-8heterocycloalkyl, and CH(C1-3alkyleneN(R12)2)2, or two R7 groups are taken together to form an optionally substituted 3- to 6-membered aliphatic ring;

R8, R9, and R10 are each independently selected from the group consisting of null, hydro, halo, optionally substituted C1-6alkyl, C2-6alkenyl, OCF3, NO2, CN, NC, N(R7)₂, OR, CO2R7, C(O)N(R7)2, C(O)R7, N(R13)COR7, N(R13)C(O)OR7, N(R7)C(O)OR7, N(R7)C(O)C1-3alkyleneC(O)R7, N(R7)C(O)C1-3alkylene-C(O)OR7, N(R7)C(O)C1-3alkyleneOR7, NR7)C(O)C1-3alkyleneNHC(O)OR7, N(R7)C(O)C1-3alkyleneSO2NR7, CF3, C1-3alkyleneN(R12)SO2aryl, C1-3alkyleneN(R12)SO2heteroaryl, C1-3alkyleneOC1-3alkylenearyl, C1-3alkyleneN(R12)C1-3alkylenearyl, C1-3alkyleneN(R12)C1-3alkyleneheteroaryl, C1-3alkyleneN(R12)C(O)R7, C1-3alkyleneN(R12)C(O)C1-3-alkyleneOR2, C1-3alkyleneN(R12)C(O) aryl, C1-3alkylene-N(R12)C(O)C1-3alkyleneN (R12)2, C1-3alkyleneN(R12)C(O)-heteroaryl, C1-3alkyleneOR7, and SR7, wherein R7 is as defined above;

R11 is selected from the group consisting of null, hydro, optionally substituted C1-6alkyl, and halo;

R12 is selected from the group consisting of hydro, C1-6alkyl, cycloalkyl, aryl, heteroaryl, C1-3alkylenearyl, and SO2C1-6alkyl, or two R12 groups are taken together to form an optionally substituted 3- to 6-membered ring; and

R13 is selected from the group consisting of hydro, C1-6alkyltC2-6alkenyl, C2-6alkynyl, and aryl; provided that when Q′ is hydro or OCH3, at least one of R8, R9, and R10 is different from hydro, CH3, OCH3, and halo, and pharmaceutically acceptable salts, prodrugs, or solvates thereof.

iv) A compound of Formula:

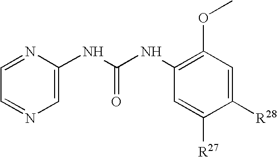

wherein R²⁷ and R²⁸ are

| R²⁷ | R²⁸ |
|---|---|
| H | 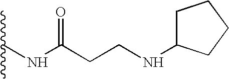 |
| H | 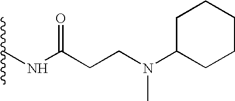 |
| H | 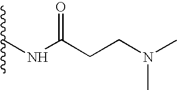 |
| CH₃ | H |
| H | 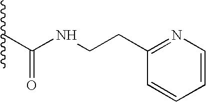 |
| H | 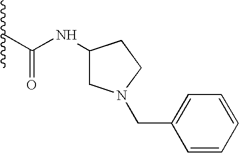 |
| 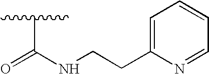 | H |
| 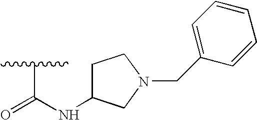 | H |

or

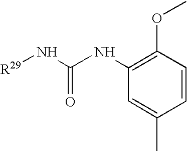

wherein R²⁹ is

R²⁹

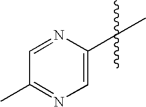

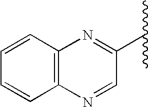

v) A compound of formula: 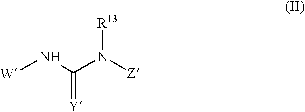
wherein Y′ is O or S;

W′ is selected from the group consisting of

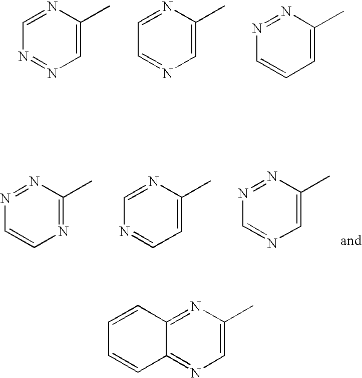

optionally substituted with from one to four substituents selected from the group consisting of

C1-6alkyl, aryl, N(R7)2, OR7, N3, CN, C(O)R7, C1-3alkylenearyl, C1-3alkyleneN(R12)2, 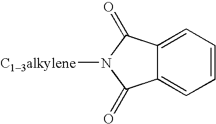
and halo;

Z′ is selected from the group consisting of: 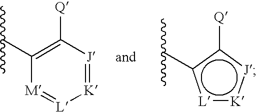
wherein:

Q′ is selected from the group consisting of hydro, OR⁷, SR⁷, and N(R⁷)₂,

with the proviso that Q′ is hydro only when at least one of J′, K′, L′, and M′ is N, O, or S;

J′ is selected from the group consisting of CR⁸, NR⁸, O, and S;

K′ is selected from the group consisting of CR⁹, NR⁹, O, and S; L′ is selected from the group consisting of CR¹⁰, NR¹⁰, O, and S;

M′ is selected from the group consisting of CR¹¹, NR¹¹, O, and S, with the proviso that Z is different from a pyridone;

wherein: R⁷, independently, is selected from the group consisting of hydro, C₁₋₆alkyl, C₂₋₆alkenyl, cycloalkyl, aryl, heteroaryl, SO₂R¹², C₁₋₆alkyl substituted with one or more of halo, hydroxy, aryl, heteroaryl, heterocycloalkyl, N(R¹²)₂, and SO₂R¹², C₁₋₃alklylenearyl, C₁₋₃alkyleneheteroaryl, C₁₋₃alkyleneC₃₋₈heterocycloalkyl, C₁₋₃alkyleneSO₂aryl, optionally substituted C₁₋₃alkyleneN(R¹²)₂, OCF3, C₁₋₃alkyleneN(R¹²)₃+, C₃₋₈heterocycloalkyl, and CH(C₁₋₃alkyleneN(R¹²)₂)₂, or two R⁷ groups are taken together to form an optionally substituted 3- to 6-membered aliphatic ring;

R⁸, R⁹, and R¹⁰ are each independently selected from the group consisting of null, hydro, halo, optionally substituted C₁₋₆alkyl, C₂₋₆alkenyl, OCF₃, NO₂, CN, NC, N(R⁷)₂, OR⁷, CO₂R⁷, C(O)N(R⁷)₂, C(O)R⁷, N(R¹³)COR⁷, N(R¹³) C(O)OR⁷, N(R⁷)C(O)OR⁷, N(R⁷)C(O)C₁₋₃alkyleneC(O)R⁷, N(R⁷)C(O)C₁₋₃alkyleneC(O)OR⁷, N(R⁷)C(O)C₁₋₃alkyleneOR⁷, N(R⁷)C(O)C₁₋₃alkyleneNHC(O)OR⁷, N(R⁷)C(O)C₁₋₃alkyleneSO₂NR⁷, C₁₋₃alkyleneOR⁷, and SR⁷, wherein R⁷ is as defined above;

R¹¹ is selected from the group consisting of null, hydro, optionally substituted C₁₋₆alkyl, and halo;

R¹² is selected from the group consisting of hydro, C₁₋₆alkyl, cycloalkyl, aryl, heteroaryl, C₁₋₃alkylenearyl, and SO₂C₁₋₆alkyl, or two R¹² groups are taken together to form an optionally substituted 3- to 6-membered ring; and R¹³ is selected from the group consisting of hydro, C₁₋₆alkyl, C₂₋₆alkenyl, C₂₋₆alkynyl, and aryl; provided that when Q′ is hydro or OCH₃, at least one of R⁸, R⁹, and R¹⁰ is different from hydro, CH₃, OCH₃; and halo, and pharmaceutically acceptable salts, prodrugs, or solvates thereof.

II. Urea Compounds Described in US20040014765, Including:

i) A compound of the formula:

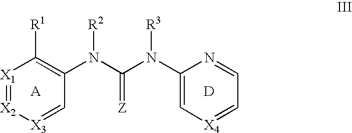

or a pharmaceutically acceptable salt thereof, wherein:

X₁-X₃ are independently CH or N, that provided that X₁-X₃ are not all N;

X₄ is CH or N; Z is O, S, or N—CN;

Ring A is optionally substituted at any substitutable carbon by R⁴;

R¹ is -T-NH₂, —V-T-NH₂, -T-NHR^{x}, —V-T-NHR^{x};

T is a C₁₋₆ straight or branched alkylidene chain that is optionally interrupted by —O—, —S—, —N(R⁵)—, —S(O)—, —SO₂—, —C(O)—, —OC(O)—, —N(R⁵)C(O)—, —C(O)N(R⁵)—, —SO₂N(R⁵)—, or —N(R⁵)SO₂—,

wherein the alkylidene chain or a portion thereof is optionally part of a 3-6 membered ring system;

V is —O—, —S—, —N(R⁵)—, —S(O)—, —SO₂—, —C(O)—, —OC(O)—, —N(R⁵)C(O)—, —C(O)N(R⁵)—, —SO₂N(R⁵)—, or —N(R⁵)SO₂—;

R² and R³ are each independently selected from hydrogen, C₁₋₆ alkyl optionally substituted with —N(R⁸)₂, —C(═O)R, —CO₂R, or SO₂R, or R² and R³ taken together with their intervening atoms form an optionally substituted 5-6 membered ring;

each R⁴ is independently selected from halo, —OR—, —SR, —CN, —NO₂, —N(R⁵)₂, —N(R⁵)C(O)R, —N(R⁵)CO₂R, —N(R⁵)C(O)N(R⁵)₂, —C(O)N(R⁵)₂, —C(O)R⁵, —OC(O)N(R⁵)₂, —CO₂R, —SO₂R, —S(O)R, —SO₂N(R⁵)₂, —N(R⁵)SO₂R, or an optionally substituted group selected from C₁₋ₛ aliphatic, aryl, aralkyl, heterocyclyl, heterocycloalkyl, heteroaryl, or heteroaralkyl, or two ortho R⁴s, taken together with the ortho carbon atoms to which they are bonded, form an optionally substituted five or six membered phenyl, pyridyl or heterocyclyl fused to Ring A;

each R⁵ is independently selected from hydrogen, C₁₋₆ aliphatic, —CO₂R, —SO₂R, or —C(O)R, or two R⁵ on the same nitrogen taken together with the nitrogen form a 5-8 membered heteroaryl or heterocycle ring having 1-4 heteroatoms selected from N, O, or S; each R⁸ is independently a C₁₋₃ alkyl or, taken together with the nitrogen atom to which they are bonded, a 5-7 membered nitrogen containing heterocycle;

Ring D is optionally substituted by C₁₋₄ aliphatic or haloaliphatic, —OR⁷, —SR⁷, —C(O)R⁷, —CO₂R⁷, —SO₂R⁷, —CN, —C(O)N(R⁷)₂, —N(R⁷)C(O)(C₁₋₂ alkyl), or —N(R⁷)₂ and is optionally fused to an optionally substituted phenyl or optionally substituted cyclohexyl ring; each R⁷ is independently selected from hydrogen or an optionally substituted C₁₋₃ aliphatic or —N(R⁷)₂ is a nitrogen-containing heterocyclyl; each R is independently selected from hydrogen or an optionally substituted group selected from C₁₋₆ aliphatic, aryl, aralkyl, heteroaryl, or heteroaralkyl-butyl; and R^{x} is C₁-C₈ alkyl.

ii) A compound of the formula:

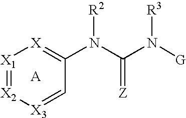

or a pharmaceutically acceptable, salt thereof, wherein: X is CR¹;

X₁-X₃ are CH;

Z is O

Ring A is optionally substituted at any substitutable carbon by R⁴;

R¹ is V-T-R⁶;

T is a C₂₋₄ alkylidene chain;

V is —O—;

R² and R³ are each hydrogen;

each R⁴ is independently selected from halo, —OR, —SR, —CN, —NO₂, —N(R⁵)₂, —N(R⁵)C(O)R, —N(R⁵)CO₂R, —N(R⁵)C(O)N(R⁵)₂, —C(O)N(R⁵)₂, —OC(O)N(R⁵)₂, —CO₂R, —SO₂R, —S(O)R, —SO₂N(R⁵)₂, —N(R⁵)SO₂R, or an optionally substituted group selected from C₁₋₈ aliphatic, aryl, aralkyl, heterocyclyl, heterocyclealkyl, heteroaryl, or heteroaralkyl, or two ortho R⁴s, taken together with the ortho carbon atoms to which they are bonded, form an optionally substituted five or six membered phenyl, pyridyl or heterocyclyl fused to Ring A;

each R⁸ is independently a C₁₋₃ alkyl or, taken together with the nitrogen atom to which they are bonded, a 5-7 membered nitrogen containing heterocycle;

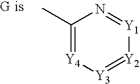

Y₁₋₄ are each independently selected from CH or nitrogen, provided that Ring B has no more than three nitrogen atoms and Y₁ and Y₂ are not both N, said Ring B being optionally substituted by C₁₋₄ aliphatic or haloaliphatic, OR⁷, —SR⁷, —C(O)R⁷, —CO₂R⁷, —SO₂R⁷, —CN, —C(O)N(R⁷)₂, —N(R⁷)C(O)(C₁₋₂ alkyl), or —N(R⁷)₂; each R⁷ is independently selected from hydrogen or an optionally substituted C₁₋₃ aliphatic or —N(R⁷)₂ is a nitrogen-containing heterocyclyl; and each R is hydrogen.

III. Chk1 Inhibiting Compounds described in US20040092535, Including”

i) A compound of formula:

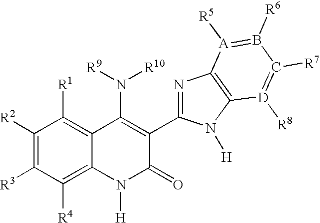

and a tautomer, a pharmaceutically acceptable salt, a pharmaceutically acceptable salt of the tautomer, or mixtures thereof,

wherein: A, B, C, and D are independently selected from the group consisting carbon and nitrogen;

R¹ is selected from the group consisting of —H, —F, —Cl, —Br, —I, —CN, —NO₂, substituted and unsubstituted alkyl groups having from 1 to 12 carbon atoms., substituted and unsubstituted alkenyl groups having from 1 to 12 carbon atoms, substituted and unsubstituted alkynyl groups having from 1 to 8 carbon atoms, substituted and unsubstituted heterocyclyl groups, substituted and unsubstituted heterocyclylalkyl groups, —SH, substituted and unsubstituted —S-alkyl groups, substituted and unsubstituted —S(—O)₂—O-alkyl groups, substituted and unsubstituted —S(—O)₂-alkyl groups, substituted and unsubstituted —S(—O)-alkyl groups, —S(—O)—NH₂, substituted and unsubstituted —S(—O)—N(H)(alkyl) groups, substituted and unsubstituted —S(—O)—N(alkyl)₂ groups, —OH, substituted and unsubstituted alkoxy groups, substituted and unsubstituted aryloxy groups, substituted and unsubstituted arylalkoxy groups, substituted and unsubstituted heterocyclyloxy groups, substituted and unsubstituted heterocyclylalkoxy groups, —NH₂, substituted and unsubstituted —N(H)(alkyl) groups, substituted and unsubstituted —N(alkyl) groups, substituted and unsubstituted —N(H)(heterocyclyl) groups, substituted and unsubstituted —N(alkyl)(heterocyclyl) groups, substituted and unsubstituted —N(heterocyclyl)₂ groups, substituted and unsubstituted —N(H)(heterocyclylalkyl) groups, substituted and unsubstituted —N(alkyl)(heterocyclylalkyl) groups, substituted and unsubstituted —N(heterocyclylalkyl)₂ groups, substituted and unsubstituted —N(H)—C(—O)-alkyl groups, substituted and unsubstituted —N(H)—C(—O)heterocyclyl groups, substituted and unsubstituted —N(H)—C(—O)-heterocyclylalkyl groups, substituted and unsubstituted —N(H)—S(—O)-alkyl groups, substituted and unsubstituted —C(—O)-alkyl groups, substituted and unsubstituted —C(—O)-heterocyclyl groups, substituted and unsubstituted —C(—O)-heterocyclylalkyl groups, —C(—O)—NH₂, substituted and unsubstituted —C(—O)—N(H)(alkyl) groups, substituted and unsubstituted —C(—O)—N(alkyl)₂ groups, substituted and unsubstituted —C(—O)—N(H)(aralkyl) groups, substituted and unsubstituted —C(—O)—N(H)(heterocyclyl) groups, —C(—O)—N(H)(heterocyclylalkyl) groups, —CO₂H, substituted and unsubstituted —C(—O)—O-alkyl groups, substituted and unsubstituted —C(—O)—O-heterocyclyl groups, and substituted and unsubstituted —C(—O)—O-heterocyclylalkyl groups;

R² and R³ are independently selected from the group consisting of —H, —F, —Cl, —Br, —I, —CN, —NO₂, substituted and unsubstituted alkyl groups having from 1 to 12 carbon atoms, substituted and unsubstituted alkenyl groups having from 1 to 12 carbon atoms, substituted and unsubstituted alkynyl groups having from 1 to 8 carbon atoms, substituted and unsubstituted aryl groups, substituted and unsubstituted aralkyl groups, substituted and unsubstituted heterocyclyl groups, substituted and unsubstituted heterocyclylalkyl groups, —SH, substituted and unsubstituted —S-alkyl groups, substituted and unsubstituted —S-aryl groups, substituted and unsubstituted —S-aralkyl groups, substituted and unsubstituted —S(—O)₂—O-alkyl groups, substituted and unsubstituted —S(—O)₂-alkyl groups, substituted and unsubstituted —S(—O)₂-heterocyclyl groups, substituted and unsubstituted —S(—O)-alkyl groups, substituted and unsubstituted —S(—O)-heterocyclyl groups, —S(—O)₂—NH₂, substituted and unsubstituted —S(—O)₂—N(H)(alkyl) groups, substituted and unsubstituted —S(—O)₂—N(alkyl)₂ groups, substituted and unsubstituted —S(—O)₂—N(H)(aryl) groups, substituted and unsubstituted —S(—O)₂—N(alkyl)(aryl) groups, substituted and unsubstituted —S(—O)₂—N(aryl)₂ groups, substituted and unsubstituted —S(—O)₂—N(H)(aralkyl) groups, substituted and unsubstituted —S(—O)₂—N(alkyl)(aralkyl) groups, substituted and unsubstituted —S(—O)2-N(aralkyl)₂ groups, —OH, substituted and unsubstituted alkoxy groups, substituted and unsubstituted aryloxy groups, substituted and unsubstituted arylalkoxy groups, substituted and unsubstituted heterocyclyloxy groups, substituted and unsubstituted heterocyclylalkoxy groups, —NH₂, substituted and unsubstituted —N(H)(alkyl) groups, substituted and unsubstituted —N(alkyl)₂ groups, substituted and unsubstituted —N(H)(aryl) groups, substituted and unsubstituted —N(alkyl)(aryl) groups, substituted and unsubstituted —N(aryl)₂ groups, substituted and unsubstituted —N(H)(aralkyl) groups, substituted and unsubstituted —N(alkyl)(aralkyl) groups, substituted and unsubstituted —N(aralkyl)₂ groups, substituted and unsubstituted —N(H)(heterocyclyl) groups, substituted and unsubstituted —N(alkyl)(heterocyclyl) groups, substituted and unsubstituted —N(heterocyclyl)₂ groups, substituted and unsubstituted, —, N(H)(heterocyclylalkyl) groups, substituted and unsubstituted —N(alkyl)(heterocyclylalkyl) groups, substituted and unsubstituted —N(heterocyclylalkyl)₂ groups, substituted and unsubstituted —N(H)—S(—O)₂-alkyl groups, substituted and unsubstituted —N(H)—S(—O)₂-aryl groups, substituted and unsubstituted —N(H)—S(—O)₂-aralkyl groups, substituted and unsubstituted —N(H)—S(—O)₂-heterocyclyl groups, substituted and unsubstituted —N(H)—S(—O)₂-heterocyclylalkyl groups, substituted and unsubstituted —N(H)—C(—O)-alkyl groups, substituted and unsubstituted —N(H)—C(—O)-aryl groups, substituted and unsubstituted —N(H)—C(—O)-aralkyl groups, substituted and unsubstituted —N(H)—C(—O)-heterocyclyl groups, substituted and unsubstituted —N(H)—C(—O)-heterocyclylalkyl groups, substituted and unsubstituted —N(alkyl)-C(—O)-alkyl groups, substituted and unsubstituted —N(alkyl)-C(—O)-aryl groups, substituted and unsubstituted —N(alkyl)-C(—O)-aralkyl groups, substituted and unsubstituted —N(alkyl)-C(—O)-heterocyclyl groups, substituted and unsubstituted —N(alkyl)-C(—O)-heterocyclylalkyl groups, substituted and unsubstituted —N(alkyl)-S(—O)₂-alkyl groups, substituted and unsubstituted —N(alkyl)-S(—O)₂-aryl groups, substituted and unsubstituted —N(alkyl)-S(—O)₂-aralkyl groups, substituted and unsubstituted —N(alkyl)-S(—O)₂-heterocyclyl groups, substituted and unsubstituted —N(alkyl)-S(—O)₂-heterocyclylalkyl groups, —N(H)—C(—O)—NH₂, substituted and unsubstituted —N(H)—C(—O)—N(H)(alkyl) groups, substituted and unsubstituted —N(H)—C(—O)—N(alkyl)₂ groups, substituted and unsubstituted —N(H)—C(—O)—N(H)(aryl) groups, substituted and unsubstituted —N(H)—C(—O)—N(alkyl)(aryl) groups, substituted and unsubstituted —N(H)—C(—O)—N(aryl)₂ groups, substituted and unsubstituted —N(H)—C(—O)—N(H)(aralkyl) groups, substituted and unsubstituted —N(H)—C(—O)—N(alkyl)(aralkyl) groups, substituted and unsubstituted —N(H)—C(—O)—N(aralkyl)₂ groups, substituted and unsubstituted —N(H)—C(—O)—N(H)(heterocyclyl) groups, substituted and unsubstituted —N(H)—C(—O)—N(alkyl)(heterocyclyl) groups, substituted and unsubstituted —N(H)—C(—O)—N(heterocyclyl)₂ groups, substituted and unsubstituted —N(H)—C(—O)—N(H)(heterocyclylalkyl) groups, substituted and unsubstituted —N(H)—C(—O)—N(alkyl)(heterocyclylalkyl) groups, substituted and unsubstituted —N(H)—C(—O)—N(heterocyclylalkyl)₂ groups, substituted and unsubstituted —N(alkyl)-C(—O)—NH₂ groups, substituted and unsubstituted —N(alkyl)-C(—O)—N(H)(alkyl) groups, substituted and unsubstituted —N(alkyl)-C(—O)—N(alkyl)₂ groups, substituted and unsubstituted —N(alkyl)-C(—O)—N(H)(aryl) groups, substituted and unsubstituted —N(alkyl)-C(—O)—N(alkyl)(aryl) groups, substituted and unsubstituted —N(alkyl)-C(—O)—N(aryl)₂ groups, substituted and unsubstituted —N(alkyl)-C(—O)—N(H)(aralkyl) groups, substituted and unsubstituted —N(alkyl)C(—O)—N(alkyl)(aralkyl) groups, substituted and unsubstituted —N(alkyl)-C(—O)—N(aralkyl)₂ groups, substituted and unsubstituted —N(alkyl)-C(—O)—N(H)heterocyclyl) groups, substituted and unsubstituted —N(alkyl)-C(—O)—N(alkyl)(heterocyclyl) groups, substituted and unsubstituted —N(alkyl)-C(—O)—N(heterocyclyl)₂ groups, substituted and unsubstituted —N(alkyl)-C(—O)—N(H)heterocyclylalkyl) groups, substituted and unsubstituted —N(alkyl)-C(—O)—N(alkyl)(heterocyclylalkyl) groups, substituted and unsubstituted —N(alkyl)-C(—O)—N(heterocyclylalkyl) groups, substituted and unsubstituted —C(—O)-alkyl groups, substituted and unsubstituted —C(—O)-aryl groups, substituted and unsubstituted —C(—O)-aralkyl groups, substituted and unsubstituted —C(—O)-heterocyclyl groups, substituted and unsubstituted —C(—O)-heterocyclylalkyl groups, —C(—O)—NH₂, substituted and unsubstituted —C(—O)—N(H)(alkyl) groups, substituted and unsubstituted —C(—O)—N(alkyl)₂ groups, substituted and unsubstituted —C(—O)—N(H)(aryl) groups, substituted and unsubstituted —C(—O)—N(alkyl)(aryl) groups, substituted and unsubstituted —C(—O)—N(aryl)₂ groups, substituted and unsubstituted —C(—O)N(H)(aralkyl) groups, substituted and unsubstituted —C(—O)—N(alkyl)(aralkyl) groups, substituted and unsubstituted —C(—O)—N(aralkyl)₂ groups, substituted and unsubstituted —C(—O)—N(H)(heterocyclyl) groups, substituted and unsubstituted —C(—O)—N(alkyl)(heterocyclyl) groups, substituted and unsubstituted —C(—O)—N(heterocyclyl)₂ groups, substituted and unsubstituted —C(—O)—N(H)(heterocyclylalkyl) groups, substituted and unsubstituted —C(—O)—N(alkyl)(heterocyclylalkyl) groups, substituted and unsubstituted —C(—O)—N(heterocyclylalkyl)₂ groups, —CO₂H, substituted and unsubstituted —C(—O)—O-alkyl groups, substituted and unsubstituted —C(—O)—O-aryl groups, substituted and unsubstituted —C(—O)—O-heterocyclyl groups, and substituted and unsubstituted —C(—O)—O-heterocyclylalkyl groups;

R⁴ is selected from the group consisting of —H, —F, —Cl, —Br, —I, —CN, —NO₂, substituted and unsubstituted alkyl groups having from 1 to 12 carbon atoms, substituted and unsubstituted alkenyl groups having from 1 to 8 carbon atoms, substituted and unsubstituted alkynyl groups having from 1 to 8 carbon atoms, —SH, substituted and unsubstituted —S-alkyl groups, substituted and unsubstituted —S(—O)₂—O-alkyl groups, substituted and unsubstituted —S(—O)₂-alkyl groups, substituted and unsubstituted —S(—O)-alkyl groups, —S(—O)₂—NH₂, substituted and unsubstituted —S(—O)₂—N(H)(alkyl) groups, substituted and unsubstituted —S(—O)₂—N(alkyl)₂ groups, —OH, substituted and unsubstituted alkoxy groups, —NH₂, substituted and unsubstituted —N(H)(alkyl) groups, substituted and unsubstituted —N(alkyl)₂ groups, substituted and unsubstituted —N(H)—C(—O)-alkyl groups, substituted and unsubstituted —N(H)—S(—O)-alkyl groups, —C(—O)—NH2, substituted, and unsubstituted —C(—O)—N(H)(alkyl) groups, substituted and unsubstituted —C(—O)—N(alkyl)₂ groups, and substituted and unsubstituted —C(—O)—O-alkyl groups;

R⁵ and R⁸ are independently selected from the group consisting of —H, —F, —Cl, —Br, —I, —CN, —NO₂, substituted and unsubstituted straight and branched chain alkyl groups having from 1 to 8 carbon atoms, substituted and unsubstituted alkenyl groups having from 1 to 8 carbon atoms, substituted and unsubstituted alkynyl groups having from 1 to 8 carbon atoms, substituted and unsubstituted heterocyclyl groups, —SH, substituted and unsubstituted —S-alkyl groups, substituted and unsubstituted —S(—O)₂—O-alkyl groups, substituted and unsubstituted —S(—O)₂-alkyl groups, substituted and unsubstituted —S(—O)-alkyl groups, —S(—O)₂—NH₂, substituted and unsubstituted —S(—O)₂—N(H)(alkyl) groups, substituted and unsubstituted —S(—O)₂—N(alkyl)₂ groups, —OH, substituted and unsubstituted alkoxy groups, —NH₂, substituted and unsubstituted —N(H)(alkyl) groups, substituted and unsubstituted —N(alkyl)₂ groups, substituted and unsubstituted —N(H)—C(—O)-alkyl groups, substituted and unsubstituted —N(H)—S(—O)alkyl groups, —C(—O)—NH₂, substituted and unsubstituted —C(—O)—N(H)(alkyl) groups, substituted and unsubstituted —C(—O)—N(alkyl)₂ groups, and substituted and unsubstituted —C(—O)—O-alkyl groups; or R¹ may be absent if A is nitrogen; or R⁸ may be absent if D is nitrogen;

R⁶ and R⁷ are independently selected from the group consisting of —H, —F, —Cl, —Br, —I, —NO2, —CN, substituted and unsubstituted alkyl groups having from 1 to 12 carbon atoms, substituted and unsubstituted alkenyl groups having from 1 to 12 carbon atoms, substituted and unsubstituted alkynyl groups having from 1 to 8 carbon atoms, substituted and unsubstituted heterocyclyl groups, substituted and unsubstituted heterocyclylalkyl groups, —SH, substituted and unsubstituted —S-alkyl groups, substituted and unsubstituted —S(—O)₂—O-alkyl groups, substituted and unsubstituted —S(—O)₂-alkyl groups, substituted and unsubstituted —S(—O)₂-heterocyclyl groups; substituted and unsubstituted —S(—O)-alkyl groups, substituted and unsubstituted —S(—O)-heterocyclyl groups, —S(—O)₂—NH₂, substituted and unsubstituted —S(—O)₂—N(H)(alkyl) groups, substituted and unsubstituted —S(—O)₂—N(alkyl)₂ groups, substituted and unsubstituted —S(—O)₂—N(H)(heterocyclyl) groups, substituted and unsubstituted —S(—O)₂—N(alkyl)(heterocyclyl) groups, substituted and unsubstituted —S(—O)₂—N(heterocyclyl)₂ groups, substituted and unsubstituted —S(—O)₂—N(H)(heterocyclylalkyl) groups, substituted and unsubstituted —S(—O)₂—N(alkyl)(heterocyclylalkyl) groups, substituted and unsubstituted —S(—O)₂—N(heterocyclylalkyl) groups, —OH, substituted and unsubstituted alkoxy groups, substituted and unsubstituted aryloxy groups, substituted and unsubstituted arylalkoxy groups, substituted and unsubstituted heterocyclyloxy groups, substituted and unsubstituted heterocyclylalkoxy groups, —NH₂, substituted and unsubstituted —N(H)(alkyl) groups, substituted and unsubstituted —N(alkyl)₂ groups, substituted and unsubstituted —N(H)(aryl) groups, substituted and unsubstituted —N(alkyl)(aryl) groups, substituted and unsubstituted —N(aryl)₂ groups, substituted and unsubstituted —N(H)(aralkyl) groups, substituted and unsubstituted —N(alkyl)(aralkyl) groups, substituted and unsubstituted —N(aralkyl)₂ groups, substituted and unsubstituted —N(H)(heterocyclyl) groups, substituted and unsubstituted —N(alkyl)(heterocyclyl) groups, substituted and unsubstituted —N(heterocyclyl)₂ groups, substituted and unsubstituted —N(H)(heterocyclylalkyl) groups, substituted and unsubstituted —N(alkyl)(heterocyclylalkyl) groups, substituted and unsubstituted —N(heterocyclylalkyl)₂ groups, substituted and unsubstituted —N(H)—S(—O)₂-alkyl groups, substituted and unsubstituted —N(H)—(—O)₂-heterocyclyl groups, substituted and unsubstituted —N(H)—S(—O)₂-heterocyclylalkyl groups, substituted and unsubstituted —N(H)—C(—O)alkyl groups, substituted and unsubstituted —N(H)—C(—O)-heterocyclyl groups, substituted and unsubstituted —N(H)—C(—O)-heterocyclylalkyl groups, substituted and unsubstituted —N(alkyl)-C(—O)-alkyl groups, substituted and unsubstituted —N(alkyl)-C(—O)-heterocyclyl groups, substituted and unsubstituted —N(alkyl)-C(—O)-heterocyclylalkyl groups, substituted and unsubstituted —N(alkyl)-S(—O)₂-alkyl groups, substituted and unsubstituted —N(alkyl)-S(—O)₂-heterocyclyl groups, substituted and unsubstituted —N(alkyl)-S(—O)₂-heterocyclylalkyl groups, substituted and unsubstituted —C(—O)-alkyl groups, substituted and unsubstituted —C(—O)-heterocyclyl groups, substituted and unsubstituted —C(—O)-heterocyclylalkyl groups, —C(—O)—NH₂, substituted and unsubstituted —C(—O)—N(H)(alkyl) groups, substituted and unsubstituted —C(—O)—N(alkyl)₂ groups, substituted and unsubstituted —C(—O)—N(H)(aryl) groups, substituted and unsubstituted —C(—O)—N(alkyl)(aryl) groups, substituted and unsubstituted —C(—O)—N(aryl)₂ groups, substituted and unsubstituted —C(—O)—N(H)(aralkyl) groups, substituted and unsubstituted —C(—O)—N(alkyl)(aralkyl) groups, substituted and unsubstituted —C(—O)—N(aralkyl)₂ groups, substituted and unsubstituted —C(—O)—N(H)(heterocyclyl) groups, substituted and unsubstituted —C(—O)—N(alkyl)(heterocyclyl) groups, substituted and unsubstituted —C(—O)—N(heterocyclyl)₂ groups, substituted and unsubstituted —C(—O)—N(H)(heterocycylalkyl) groups, substituted and unsubstituted —C(—O)—N(alkyl)(heterocyclylalkyl) groups, substituted and unsubstituted —C(—O)—N(heterocyclylalkyl)₂ groups, —CO₂H, substituted and unsubstituted —C(—O)—O-alkyl groups, substituted and unsubstituted —C(—O)—O-heterocyclyl groups, and substituted and unsubstituted —C(—O)—O-heterocyclylalkyl groups; or R⁶ may be absent if B is nitrogen; or R⁷ may be absent if C is nitrogen;

R⁹ is selected from the group consisting of —H, substituted and unsubstituted alkyl groups having from 1 to 12 carbon atoms, substituted and unsubstituted aryl groups, substituted and unsubstituted aralkyl groups, substituted and unsubstituted heterocyclyl groups, substituted and unsubstituted heterocyclylalkyl groups, substituted and unsubstituted heterocyclylaminoalkyl groups, substituted and unsubstituted alkoxy groups, and —NH₂, or R⁹ and R¹⁰ join together to form one or more rings, each having 5, 6, or 7 ring members; and

R¹⁰ is —H, or R⁹ and R¹⁰ join together to form one or more rings, each having 5, 6, or 7 ring members.

ii) A compound of formula:

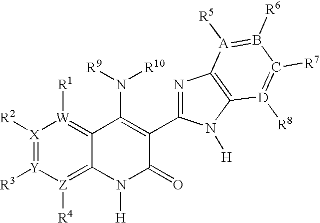

and a tautomer, a pharmaceutically acceptable salt of the compound, a pharmaceutically acceptable salt of the tautomer, or mixtures thereof,

wherein: A, B, C, and D are independently selected from the group consisting of carbon and nitrogen;

W, X, Y, and Z are independently selected from the group consisting of carbon and nitrogen and at least one of W, X, Y, and Z is a nitrogen;

R¹ is selected from the group consisting of —H, —F, —Cl, —Br, —I, substituted and unsubstituted straight and branched chain alkyl groups having from 1 to 8 carbon atoms, substituted and unsubstituted alkenyl groups having from 1 to 8 carbon atoms, substituted and unsubstituted alkynyl groups having from 1 to 8 carbon atoms, —CN, —NO₂, —OH, —SH, substituted and unsubstituted alkoxy groups, substituted and unsubstituted —S-alkyl groups, substituted and unsubstituted —S(—O)₂—O-alkyl groups, substituted and unsubstituted —S(—O)₂-alkyl groups, substituted and unsubstituted —S(—O)-alkyl groups, —S(—O)—NH₂, substituted and unsubstituted —S(—O)—N(H)(alkyl) groups, substituted and unsubstituted —S(—O)N(alkyl)₂ groups, —C(—O)—NH₂, substituted and unsubstituted —C(—O)—N(H)(allyl) groups, substituted and unsubstituted —C(—O)—N(alkyl)₂ groups, substituted and unsubstituted —C(—O)—O-alkyl groups, —NH₂, substituted and unsubstituted —N(H)(alkyl) groups, substituted and unsubstituted —N(allyl)₂ groups, substituted and unsubstituted —N(H)—C(—O)-alkyl groups, and substituted and unsubstituted —N(H)—S(—O)alkyl groups; or R¹ may be absent if W is nitrogen;

R² is selected from the group consisting of —H, —F, —Cl, —Br, —I, —NO₂, —CN, —NH₂, —CO₂H, —OH, substituted and unsubstituted straight and branched chain alkyl groups having from 1 to 8 carbon atoms, substituted and unsubstituted cycloalkenyl groups, substituted and unsubstituted cycloalkyl groups, substituted and unsubstituted alkoxy groups, substituted and unsubstituted —N(H)(alkyl) groups, substituted and unsubstituted —N(alkyl)₂ groups, substituted and unsubstituted heterocyclyl groups, substituted and unsubstituted aryl groups, substituted and unsubstituted alkenyl groups having from 1 to 8 carbon atoms, substituted and unsubstituted alkynyl groups having from 1 to 8 carbon atoms, —SH, substituted and unsubstituted —S-alkyl groups, substituted and unsubstituted —S(—O)₂—O-alkyl groups, substituted and unsubstituted —S(—O)₂-alkyl groups, substituted and unsubstituted —S(—O)₂-heterocyclyl groups, substituted and unsubstituted —S(—O)-alkyl groups, substituted and unsubstituted —S(—O)-heterocyclyl groups, —S(—O)—NH₂, substituted and unsubstituted —S(—O—N(H)(alkyl) groups, substituted and unsubstituted —S(—O)—N(alkyl)₂ groups, —C(—O)—NH₂, substituted and unsubstituted —C(—O)—N(H)(alkyl) groups, substituted and unsubstituted —C(—O)—N(alkyl)₂ groups, substituted and unsubstituted —C(—O)-alkyl groups, substituted and unsubstituted —C(—O)-heterocyclyl groups, substituted and unsubstituted —C(—O)—O-alkyl groups, substituted and unsubstituted —N(H)—C(—O)-alkyl groups, substituted and unsubstituted —N(H)—C(—O)-heterocyclyl groups, substituted and unsubstituted —N(H)—S(—O)-alkyl groups, substituted and unsubstituted —N(H)—S(—O)-heterocyclyl groups, —N(alkyl)-C(—O)-alkyl groups, substituted and unsubstituted —N(alkyl)-C(—O)heterocyclyl groups, substituted and unsubstituted —N(alkyl)-S(—O)-alkyl groups, substituted and unsubstituted —N(alkyl)-S(—O)-heterocyclyl groups, —N(H)—C(—O)—NH₂, substituted and unsubstituted —N(H)—C(—O)—N(H)(alkyl) groups, substituted and unsubstituted —N(H)—C(—O)—N(alkyl)₂ groups, —N(alkyl)-C(—O)—NH₂, substituted and unsubstituted —N(alkyl)-C(—O)—N(H)(alkyl) groups, and substituted and unsubstituted —N(alkyl)-C(—O)—N(alkyl)₂ groups; or R² and R³ may join together to form a cyclic group when X and Y are both carbon; or R² may be absent if X is nitrogen;

R³ is selected from the group consisting of —H, —F, —Cl, —Br, —I, —OH, substituted and unsubstituted straight and branched chain alkyl groups having from 1 to 8 carbon atoms, substituted and unsubstituted alkoxy groups, —CO₂H, —CN, substituted and unsubstituted —N(H)(alkyl) groups, substituted and unsubstituted —N(H)(cycloalkyl) groups, substituted and unsubstituted —N(alkyl)₂ groups, substituted and unsubstituted heterocyclyl groups, substituted and unsubstituted aryl groups, substituted and unsubstituted —C(—O)-heterocyclyl groups, substituted and unsubstituted —C(—O)-alkyl groups, substituted and unsubstituted —C(—O)—N(H)(alkyl) groups, substituted and unsubstituted —C(—O)—N(alkyl)₂ groups, —C(—O)—NH₂ groups, substituted and unsubstituted —C(—O)—N(M)(heterocyclyl) groups, substituted and unsubstituted —C(—O)—N(H)(aryl) groups, substituted and unsubstituted alkenyl groups having from 1 to 8 carbon atoms, substituted and unsubstituted alkynyl groups having from 1 to 8 carbon atoms, —NO₂, —SH, substituted; and unsubstituted —S-alkyl groups, substituted and unsubstituted —S(—O)₂—O-alkyl groups, substituted and unsubstituted —S(—O)₂alkyl groups, substituted and unsubstituted —S(—O)₂-heterocyclyl groups, substituted and unsubstituted —S(—O)-alkyl groups, substituted and unsubstituted —S(—O)-heterocyclyl groups, —S(—O)—NH₂, substituted and unsubstituted —S(—O)—N(H)(alkyl) groups, substituted and unsubstituted —S(—O)—N(alkyl)₂ groups, substituted and unsubstituted —C(—O)—O-alkyl groups, —NH₂, substituted and unsubstituted —N(H)—C(—O)-alkyl groups, substituted and unsubstituted —N(H)—C(—O)-heterocyclyl groups, substituted and unsubstituted —N(H)—S(—O)alkyl groups, substituted and unsubstituted —N(H)—S(—O)-heterocyclyl groups, substituted and unsubstituted —N(alkyl)-C(—O)-alkyl groups, substituted and unsubstituted —N(alkyl)-C(—O)-heterocyclyl groups, substituted and unsubstituted —N(alkyl)-S(—O)-alkyl groups, substituted and unsubstituted —N(alkyl)-S(—O)-heterocyclyl groups, —N(H)—C(—O)—NH₂, substituted and unsubstituted —N(H)—C(—O)—N(H)(alkyl) groups, substituted and unsubstituted —N(H)—C(—O)—N(alkyl)₂ groups, —N(alkyl)-C(—O)—NH₂, substituted and unsubstituted —N(alkyl)-C(—O)N(H)(alkyl) groups, and substituted and unsubstituted —N(alkyl)-C(—O)—N(alkyl)₂ groups; or R² and R³ may join together to form a cyclic group when X and Y are both carbon; or R³ may be absent if Y is nitrogen;

R⁴ is selected from the group consisting of —H, —F, —Cl, —Br, —I, substituted and unsubstituted straight and branched chain alkyl groups having from 1 to 8 carbon atoms, substituted and unsubstituted alkenyl groups having from 1 to 8 carbon atoms, substituted and unsubstituted alkynyl groups having from 1 to 8 carbon atoms, —CN, —NO₂, —OH, —SH, substituted and unsubstituted alkoxy groups, substituted and unsubstituted —S-alkyl groups, substituted and unsubstituted —S(—O)₂—O-alkyl groups; substituted and unsubstituted —S(—O)₂-alkyl groups, substituted and unsubstituted —S(—O)-alkyl groups, —S(—O)—NH₂, substituted and unsubstituted —S(—O)—N(H)(alkyl) groups, substituted and unsubstituted —S(—O)—N(alkyl) groups, —C(—O)—NH₂, substituted and unsubstituted —C(—O)—N(H)(alkyl) groups, substituted and unsubstituted —C(—O)N(alkyl)₂ groups, substituted and unsubstituted —C(—O)—O-alkyl groups, —NH₂, substituted and unsubstituted —N(H)(alkyl) groups, substituted and unsubstituted —N(alkyl)₂ groups, substituted and unsubstituted —N(H)—C(—O)-alkyl groups and substituted and unsubstituted —N(H)—S(—O)-alkyl groups; or R⁴ may be absent if Z is nitrogen

R⁵ is selected from the group consisting of —H, —F, —Cl, —Br, —I, substituted and unsubstituted straight and branched chain alkyl groups having from 1 to 8 carbon atoms, substituted and unsubstituted heterocyclyl groups, substituted and unsubstituted alkenyl groups having from 1 to 8 carbon atoms, substituted and unsubstituted alkynyl groups having from 1 to 8 carbon atoms, —CN, —NO₂, —OH, —SH, substituted and unsubstituted alkoxy groups, substituted and unsubstituted —S-alkyl groups, substituted and unsubstituted —S(—O)₂—O-alkyl group's, substituted and unsubstituted —S(—O)₂-alkyl groups, substituted and unsubstituted S(—O)-alkyl groups, —S(—O)—NH₂, substituted and unsubstituted —S(—O)—N(H)(alkyl) groups; substituted and unsubstituted —S(—O)—N(alkyl)₂ groups, —C(—O)—NH₂, substituted and unsubstituted —C(—O)—N(H)(alkyl) groups, substituted and unsubstituted —C(—O)—N(alkyl)₂ groups, substituted and unsubstituted —C(—O)—O-alkyl groups, —NH₂, substituted and unsubstituted —N(H)(alkyl) groups, substituted and unsubstituted —N(alkyl)₂ groups, substituted and unsubstituted —N(H)—C(—O)alkyl groups, and substituted and unsubstituted —N(H)—S(—O)-alkyl groups; or R⁵ may be absent if A is nitrogen;

R⁶ is selected from the group consisting of —H, —Cl, —F, —Br, —OH, substituted and unsubstituted heterocyclyl groups, substituted and unsubstituted —N(H)(alkyl) groups, substituted and unsubstituted —N(H)(heterocyclyl) groups, substituted and unsubstituted —N(alkyl)(heterocyclyl) groups, substituted and unsubstituted alkoxy groups, substituted and unsubstituted alkyl groups having from 1 to 8 carbon atoms, substituted and unsubstituted alkenyl groups having from 1 to 8 carbon atoms, substituted and unsubstituted alkynyl groups having from 1 to 8 carbon atoms, —CN, —NO₂, —OH, —SH, substituted and unsubstituted —S-alkyl groups, substituted and unsubstituted —S(—O)₂—O-alkyl groups, substituted and unsubstituted —S(—O)₂-alkyl groups, substituted and unsubstituted —S(—O)₂-heterocyclyl groups,

substituted and unsubstituted —S(—O)-alkyl groups, substituted and unsubstituted —S(—O)-heterocyclyl groups, —S(—O)—NH₂, substituted and unsubstituted —S(—O)—N(H)(alkyl) groups, substituted and unsubstituted —S(—O)—N(alkyl)₂ groups, —C(—O)NH₂, substituted and unsubstituted —C(—O)—N(H)(alkyl) groups, substituted and unsubstituted —C(—O)—N(alkyl)₂ groups, substituted and unsubstituted —C(—O)-alkyl groups, substituted and unsubstituted —C(—O)-heterocyclyl groups, substituted and unsubstituted —C(—O)—O-alkyl groups, —NH₂, substituted and unsubstituted —N(alkyl)₂ groups, substituted and unsubstituted —N(H)—C(—O)-alkyl groups, substituted and unsubstituted —N(H)—C(—O)-heterocyclyl groups, substituted and unsubstituted —N(alkyl)-C(—O)-alkyl groups, substituted and unsubstituted —N(alkyl)-C(—O)-heterocyclyl groups, substituted and unsubstituted —N(H)—S(—O)-alkyl groups, substituted and unsubstituted —N(H)—S(—O)-heteroeyclyl groups, substituted and unsubstituted —N(alkyl)-S(—O)-alkyl groups, and substituted and unsubstituted —N(alkyl)-S(—O)-heterocyclyl groups; or R⁶ may be absent if B is nitrogen;

R⁷ is selected from the group consisting of —H, —Cl, —F, —Br, —OH, substituted and unsubstituted heterocyclyl groups, substituted and unsubstituted —N(H)(alkyl) groups, substituted and unsubstituted —N(heterocyclyl) groups, substituted and unsubstituted —N(alkyl)(heterocyclyl) groups, substituted and unsubstituted alkoxy groups, substituted and unsubstituted alkyl groups having from 1 to 8 carbon atoms, substituted and unsubstituted alkenyl groups having from 1 to 8 carbon atoms, substituted and unsubstituted alkynyl groups having from 1 to 8 carbon atoms, —CN, —NO₂, —OH, —SH, substituted and unsubstituted —S-alkyl groups, substituted and unsubstituted —S(—O)₂—O-alkyl groups, substituted and unsubstituted —S(—O)₂-alkyl groups, substituted and unsubstituted —S(—O)₂-heterocyclyl groups, substituted and unsubstituted —S(—O)-alkyl groups, substituted and unsubstituted —S(—O)-heterocyclyl groups, —S(—O)—NH₂, substituted and unsubstituted —S(—O)—N(H)(alkyl) groups, substituted and unsubstituted —S(—O)—N(alkyl)₂ groups, —C(—O)—NH₂, substituted and unsubstituted —C(—O)—N(H)(alkyl) groups, substituted and unsubstituted —C(—O)—N(alkyl)₂ groups, substituted and unsubstituted —C(—O)-alkyl groups, substituted and unsubstituted —C(—O)-heterocyclyl groups, substituted and unsubstituted —C(—O)—O-alkyl groups, —NH₂, substituted and unsubstituted —N(alkyl)₂ groups, substituted and unsubstituted —N(H)—C(—O)-alkyl groups, substituted and unsubstituted —N(H)—C(—O)-heterocyclyl groups, substituted and unsubstituted —N(alkyl)-C(—O)-alkyl groups, substituted and unsubstituted —N(alkyl)-C(—O)-heterocyclyl groups, substituted and unsubstituted —N)—S(—O)-alkyl groups, substituted and unsubstituted —N(H)—S(—O)-heterocyclyl groups, substituted and unsubstituted —N(alkyl)-S(—O)-alkyl groups, and substituted and unsubstituted —N(alkyl)-S(—O)-heterocyclyl groups; or R⁷ may be absent if C is nitrogen;

R⁸ is selected from the group consisting of —H, —F, —Cl, —Br, —I, substituted and unsubstituted straight and branched chain alkyl groups having from 1 to 8 carbon atoms, substituted and unsubstituted heterocyclyl groups, substituted and unsubstituted alkenyl groups having from 1 to 8 carbon atoms, substituted and unsubstituted alkynyl groups having from 1 to 8 carbon atoms, —CN, —NO₂, —OH, —SH, substituted and unsubstituted alkoxy groups, substituted and unsubstituted —S-alkyl groups, substituted and unsubstituted —S(—O)₂—O-alkyl groups, substituted and unsubstituted —S(—O)₂-alkyl groups, substituted and unsubstituted —S(—O)-alkyl groups, —S(—O)—NH₂, substituted and unsubstituted —S(—O)—N(H)(allyl) groups, substituted and unsubstituted —S(—O)—N(alkyl)₂ groups, —C(—O)—NH₂, substituted and unsubstituted —C(O)—N(H)(alkyl) groups, substituted and unsubstituted —C(—O)—N(alkyl)₂ groups, substituted and unsubstituted —C(—O)—O-alkyl groups, —NH₂, substituted and unsubstituted —N(H)(alkyl) groups, substituted and unsubstituted —N(alkyl)₂ groups, substituted and unsubstituted —N(H)—C(—O)-alkyl groups, and substituted and unsubstituted —N(H)—S(—O)-alkyl groups; or R⁸ may be absent if D is nitrogen;

R⁹ is selected from the group consisting of substituted and unsubstituted heterocyclyl groups, substituted and unsubstituted aryl groups, substituted and unsubstituted alkoxy groups, —NH₂, substituted and unsubstituted cycloalkyl groups, and substituted and unsubstituted straight and branched chain alkyl groups having from 1 to 8 carbon atoms, or R⁹ and R¹⁰ join together to form a ring having 5, 6, or 7 ring members; and

R¹⁰ is —H, or R⁹ and R¹⁰ join together to form a ring having 5, 6, or 7 ring members.

ii) A compound of formula: 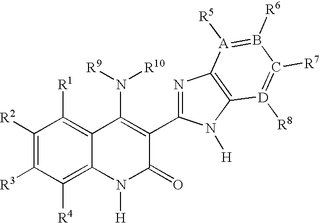
where, A, B, C, and D are independently selected from the group consisting of carbon and nitrogen;

A is selected from the consisting of —H, —F, —Cl, —Br, —I, —CN, —NO₂, substituted and unsubstituted alkyl groups having from 1 to 12 carbon atoms, substituted and unsubstituted alkenyl groups having from 1 to 12 carbon atoms, substituted and unsubstituted alkynyl groups having from 1 to 8 carbon atoms, substituted and unsubstituted heterocyclyl groups, —OH, substituted and unsubstituted alkoxy groups, substituted and unsubstituted aryloxy groups, substituted and unsubstituted arylalkoxy groups, substituted and unsubstituted heterocyclyloxy groups, substituted and unsubstituted heterocyclylalkoxy groups, —SH, substituted and unsubstituted —S-alkyl groups, —NH₂, substituted and unsubstituted —N(H)(alkyl); groups, substituted and unsubstituted —N(alkyl)₂ groups, substituted and unsubstituted —N(H)(heterocyclyl) groups, substituted and unsubstituted —N(alkyl)(heterocyclyl) groups, substituted and unsubstituted —N(heterocyclyl)₂ groups, substituted and unsubstituted —N(H)(heterocyclylalkyl) groups, substituted and unsubstituted —N(alkyl)(heterocyclylalkyl) groups, and substituted and unsubstituted —N(heterocyclylalkyl)₂ groups;

R² and R³ are independently selected from the group consisting of —H, —F, —Cl, —Br, —I, —NO₂, —CN, substituted and unsubstituted alkyl groups having from 1 to 12 carbon atoms, substituted and unsubstituted alkenyl groups having from 1 to 12 carbon atoms, substituted and unsubstituted alkynyl groups having from 1 to 8 carbon atoms, substituted and unsubstituted aryl groups, substituted and unsubstituted aralkyl groups, substituted and unsubstituted heterocyclyl groups, substituted and unsubstituted heterocyclylalkyl groups, —SH, substituted and unsubstituted —S-alkyl groups, substituted and unsubstituted —S(—O)₂—O-alkyl groups, substituted and unsubstituted —S(—O)₂-alkyl groups, substituted and unsubstituted —S(—O)₂-heterocyclyl groups, substituted and unsubstituted —S(—O)-alkyl groups, substituted and unsubstituted —S(—O)-heterocyclyl groups, —S(—O)₂—NH₂, substituted and unsubstituted —S(—O)₂—N(H)(alkyl) groups, substituted and unsubstituted —S(—O)₂—N(alkyl)₂ groups, substituted and unsubstituted —S(—O)₂—N(H)(aryl) groups, substituted and unsubstituted —S(—O)₂—N(alkyl)(aryl) groups, substituted and unsubstituted —S(—O)₂—N(aryl)₂ groups, substituted and unsubstituted —S(—O)₂—N(H)(aralkyl) groups, substituted and unsubstituted —S(—O)₂—N(alkyl)(aralkyl) groups, substituted and unsubstituted —S(—O)₂—N(aralkyl)₂ groups, —OH, substituted and unsubstituted alkoxy groups, substituted and unsubstituted aryloxy groups, substituted and unsubstituted arylalkoxy groups, substituted and unsubstituted heterocyclyloxy groups, substituted and unsubstituted heterocyclylalkoxy groups, —NH₂, substituted and unsubstituted —N(H)(alkyl) groups, substituted and unsubstituted —N(alkyl)₂ groups, substituted, and unsubstituted —N(H)(aryl) groups, substituted and unsubstituted —N(alkyl)(aryl) groups, substituted and unsubstituted —N(aryl)₂ groups, substituted and unsubstituted —N)(H)(aralkyl) groups, substituted and unsubstituted —N(alkyl)(aralkyl) groups, substituted and unsubstituted —N(aralkyl)₂ groups, substituted and unsubstituted —N(H)(heterocyclyl) groups, substituted and unsubstituted —N(alkyl)(heterocyclyl) groups, substituted and unsubstituted —N(heterocyclyl)₂ groups, substituted and unsubstituted —N(H)(heterocyclylalkyl) groups, substituted and unsubstituted —N(alkyl)(heterocyclylalkyl) groups, substituted and unsubstituted —N(heterocyclylalkyl)₂ groups, substituted and unsubstituted —N(H)—S(—O)₂-alkyl groups, substituted and unsubstituted —N(H)—S(—O)₂-aryl groups, substituted and unsubstituted —N(H)—S(—O)₂-aralkyl groups, substituted and unsubstituted —N(H)—S(—O)₂-heterocyclyl groups, substituted and unsubstituted —N(H)—S(—O)₂-heterocyclylalkyl groups, substituted and unsubstituted —N(H)—C(—O)-alkyl groups, substituted and unsubstituted —N(H)—C(—O)-aryl groups, substituted and unsubstituted —N(H)—C(—O)-aralkyl groups, substituted and unsubstituted —N(H)—C(—O)-heterocyclyl groups, substituted and unsubstituted —N(H)—C(—O)-heterocyclylalkyl groups, substituted and unsubstituted —N(alkyl)-C(—O)-alkyl groups, substituted and unsubstituted —N(alkyl)-C(—O)-aryl groups, substituted and unsubstituted —N(alkyl)-C(—O)-aralkyl groups, substituted and unsubstituted —N(alkyl)-C(—O)-heterocyclyl groups, substituted and unsubstituted —N(alkyl)-C(—O)-heterocyclylalkyl groups, substituted and unsubstituted —N(alkyl)-S(—O)-alkyl groups, substituted and unsubstituted —N(alkyl)-S(—O)-aryl groups, substituted and unsubstituted —N(alkyl)-S(—O)-aralkyl groups, substituted and unsubstituted —N(alkyl)-S(—O)-heterocyclyl groups, substituted and unsubstituted —N(alkyl)-S(—O)-heterocyclylalkyl groups, —N(H)—C(—O)—NH₂, substituted and unsubstituted —N(H)—C(—O)—N(H)(alkyl) groups, substituted and unsubstituted —N(H)—C(—O)—N(alkyl)₂ groups, substituted and unsubstituted —N(H)—C(—O)—N(H)(aryl) groups; substituted and unsubstituted —N(H)—C(—O)—N(alkyl)(aryl) groups, substituted and unsubstituted —N(H)—C(—O)—N(aryl)₂ groups, substituted and unsubstituted —N(H)—C(—O)—N(H)(aralkyl) groups, substituted and unsubstituted —N(H)—C(—O)—N(alkyl)(aralkyl) groups, substituted and unsubstituted —N(H)—C(—O)—N(aralkyl)₂ groups, substituted and unsubstituted —N(H)—C(—O)—N(H)(heterocyclyl) groups; substituted and unsubstituted —N(H)—C(—O)—N(alkyl)(heterocyclyl) groups, substituted and unsubstituted —N(H)—C(—O)—N(heterocyclyl)₂ groups, substituted and unsubstituted —N(H)—C(—O)—N(H)(heterocyclylalkyl) groups, substituted and unsubstituted —N(H)—C(—O)—N(alkyl)(heterocyclylalkyl) groups, substituted and unsubstituted —N(H)—C(—O)—N(heterocyclylalkyl)₂ groups, substituted and unsubstituted —N(alkyl)-C(—O)—NH₂ groups, substituted and unsubstituted —N(alkyl)-C(—O)—N(H)(alkyl) groups substituted and unsubstituted —N(alkyl)-C(—O)—N(alkyl)₂ groups, substituted and unsubstituted —N(alkyl)-C(—O)—N(H)(aryl) groups, substituted and unsubstituted —N(alkyl)-C(—O)—N(alkyl)(aryl) groups, substituted and unsubstituted —N(alkyl)-C(—O)—N(aryl)₂ groups, substituted and unsubstituted —N(alkyl)-C(—O)—N(H)(aralkyl) groups, substituted and unsubstituted —N(alkyl)-C(—O)—N(alkyl)(aralkyl) groups, substituted and unsubstituted —N(alkyl)-C(—O)—N(aralkyl)₂ groups, substituted and unsubstituted —N(alkyl)-C(—O)—N(H)(heterocyclyl) groups, substituted and unsubstituted —N(alkyl)-C(—O)—N(alkyl)(heterocyclyl) groups, substituted and unsubstituted —N(alkyl)-C(—O)—N(heterocyclyl)₂ groups, substituted and unsubstituted —N(alkyl)-C(—O)—N(H)(heterocyclylalkyl) groups, substituted and unsubstituted —N(alkyl)-C(—O)—N(alkyl)(heterocyclylalkyl) groups, substituted and unsubstituted —N(alkyl)-C(—O)—N(heterocyclylalkyl)₂ groups, substituted and unsubstituted —C(—O)-alkyl groups, substituted and unsubstituted —C(—O)-aryl groups, substituted and unsubstituted —C(—O)-aralkyl groups, substituted and unsubstituted —C(—O)-heterocyclyl groups, substituted and unsubstituted —C(—O)-heterocyclylalkyl groups, —C(—O)—NH₂, substituted and unsubstituted —C(—O)—N(H)(alkyl) groups, substituted and unsubstituted —C(—O)—N(alkyl)₂ groups, substituted and unsubstituted —C(—O)—N(H)(aryl) groups, substituted and unsubstituted —C(—O)—N(alkyl)(aryl) groups, substituted and unsubstituted —C(—O)—N(aryl)₂ groups, substituted and unsubstituted —C(—O)—N(H)(aralkyl) groups, substituted and unsubstituted —C(—O)—N(alkyl)(aralkyl) groups, substituted and unsubstituted —C(—O)—N(aralkyl)₂ groups, substituted and unsubstituted —C(—O)N(H)(heterocyclyl) groups, substituted and unsubstituted —C(—O)—N(alkyl)heterocyclyl) groups, substituted and unsubstituted —C(—O)—N(heterocyclyl)₂ groups, substituted and unsubstituted —C(—O)—N(H)(heterocyclylalkyl) groups, substituted and unsubstituted —C(—O)—N(alkyl)(heterocyclylalkyl) groups, substituted and unsubstituted —C(—O)—N(heterocyclylalkyl)₂ groups, —CO₂H, substituted and unsubstituted —C(—O)—O-alkyl groups, substituted and unsubstituted —C(—O)—O-aryl groups, substituted and unsubstituted —C(—O)—O-heterocyclyl groups, add substituted and unsubstituted —C(—O)—O-heterocyclylalkyl groups;

R⁴ is selected from the group consisting of —H and substituted and unsubstituted alkyl groups having from 1 to 12 carbon atoms;

R⁵ and R⁸ are independently selected from the group consisting of —H, substituted and unsubstituted alkyl groups having from 1 to 12 carbon atoms, substituted and unsubstituted alkenyl groups having from 1 to 12 carbon atoms, substituted and unsubstituted heterocyclyl groups; or R⁵ may be absent if A is nitrogen; or R⁸ may be absent if D is nitrogen;

R⁶ and R⁷ are independently selected from the group consisting of —H, —F, —Cl, —Br, —I, —NO₂, —CN, substituted and unsubstituted alkyl groups having from 1 to 12 carbon atoms, substituted and unsubstituted alkenyl groups having from 1 to 12 carbon atoms, substituted and unsubstituted alkynyl groups having from 1 to 8 carbon atoms, substituted and unsubstituted heterocyclyl groups, substituted and unsubstituted heterocyclylalkyl groups, —SH, substituted and unsubstituted —S-alkyl groups, substituted and unsubstituted —S(—O)₂—O-alkyl groups, substituted and unsubstituted —S(—O)₂-alkyl groups, substituted and unsubstituted —S(—O)₂-heterocyclyl groups, substituted and unsubstituted —S(—O)-alkyl groups, substituted and unsubstituted —S(—O)heterocyclyl groups, —S(—O)₂—NH₂, substituted and unsubstituted —S(—O)₂—N(H)(alkyl) groups, substituted and unsubstituted —S(—O)₂—N(alkyl)₂ groups, substituted and unsubstituted —S(—O)₂—N(H)(heterocyclyl) groups, substituted and unsubstituted —S(—O)₂—N(alkyl)(heterocyclyl) groups, substituted and unsubstituted —S(—O)₂—N(heterocyclyl)₂ groups, substituted and unsubstituted —S(—O)₂—N)(heterocyclylalkyl) groups, substituted and unsubstituted —S(—O)₂—N(alkyl)(heterocyclylalkyl) groups, substituted and unsubstituted —S(—O)₂—N(heterocyclylalkyl)₂ groups, —OH, substituted and unsubstituted alkoxy groups, substituted and unsubstituted aryloxy groups, substituted and unsubstituted arylalkoxy groups, substituted and unsubstituted heterocyclyloxy groups, substituted and unsubstituted heterocyclylalkoxy groups, —NH₂, substituted and unsubstituted —N(H)(alkyl) groups, substituted and unsubstituted —N(alkyl)₂ groups, substituted and unsubstituted —N(H)(aryl) groups, substituted and unsubstituted —N(alkyl)(aryl) groups, substituted and unsubstituted —N(aryl)₂ groups, substituted and unsubstituted —N(H)(aralkyl) groups, substituted and unsubstituted —N(alkyl)(aralkyl) groups, substituted and unsubstituted —N(aralkyl)₂ groups, substituted and unsubstituted —N(H)heterocyclyl) groups, substituted and unsubstituted —N(alkyl)(heterocyclyl) groups, substituted and unsubstituted —N(heterocyclyl)₂ groups, substituted and unsubstituted —N(H)(heterocyclylalkyl) groups, substituted and unsubstituted —N(alkyl)(heterocyclylalkyl) groups, substituted and unsubstituted —N(heterocyclylalkyl)₂ groups, substituted and unsubstituted —N(H)—S(—O)₂-alkyl groups, substituted and unsubstituted —N(H)—S(—O)₂-heterocyclyl groups, substituted and unsubstituted —N(H)—S(—O)₂-heterocyclylalkyl groups, substituted and unsubstituted —N(H)—C(—O)-alkyl groups, substituted and unsubstituted —N(H)—C(—O)heterocyclyl groups, substituted and unsubstituted —N(H)—C(—O)-heterocyclylalkyl groups, substituted and unsubstituted —N(alkyl)-C(—O)-alkyl groups, substituted and unsubstituted —N(alkyl)-C(—O)-heterocyclyl groups, substituted and unsubstituted —N(alkyl)-C(—O)-heterocyclylalkyl groups, substituted and unsubstituted —N(alkyl)-S(—O)₂-alkyl groups, substituted and unsubstituted —N(alkyl)-S(—O)₂-heterocyclyl groups, substituted and unsubstituted —N(alkyl)-S(—O)₂-heterocyclylalkyl groups, substituted and unsubstituted —C(—O)-alkyl groups, substituted and unsubstituted —C(—O)-heterocyclyl groups, substituted and unsubstituted —C(—O)-heterocyclylalkyl groups, —C(—O)—NH₂, substituted and unsubstituted —C(—O)—N(H)(alkyl) groups, substituted and unsubstituted —C(—O)—N(alkyl)₂ groups, substituted and unsubstituted —C(—O)—N(H)(aryl) groups, substituted and unsubstituted —C(—O)—N(alkyl)(aryl) groups, substituted and unsubstituted —C(—O)—N(aryl)₂ groups, substituted and unsubstituted —C(—O)—N(H)(aralkyl) groups, substituted and unsubstituted —C(—O)—N(alkyl)(aralkyl) groups, substituted and unsubstituted —C(—O)—N(aralkyl)₂ groups, substituted and unsubstituted —C(—O)—N(H)(heterocyclyl) groups, substituted and unsubstituted —C(—O)—N(alkyl)(heterocyclyl) groups, substituted and unsubstituted —C(—O)—N(heterocyclyl)₂ groups, substituted and unsubstituted —C(—O)—N(H)(heterocyclylalkyl) groups, substituted and unsubstituted —C(—O)—N(alkyl)(heterocyclylalkyl) groups, substituted and unsubstituted —C(—O)—N(heterocyclylalkyl)₂ groups, —CO₂H, substituted and unsubstituted —C(—O)—O-alkyl groups, substituted and unsubstituted —C(—O)—O-heterocyclyl groups, and substituted and unsubstituted —C(—O)—O-heterocyclylalkyl groups; or R⁶ may be absent if B is nitrogen; or R⁷ may be absent if C is nitrogen;

R⁹ is selected from the group consisting of —H, substituted and unsubstituted alkyl groups having from 1 to 12 carbon atoms, substituted and substituted aryl groups, substituted and unsubstituted aralkyl groups, substituted and unsubstituted heterocyclyl groups, substituted and unsubstituted heterocyclylalkyl groups, substituted and unsubstituted heterocyclylaminoalkyl groups, substituted and unsubstituted alkoxy groups, and —NH₂; or R⁹ and R¹⁰ join together to form one or more rings, each having 5, 6, or 7 ring members; and

R¹⁰ is —H, or R⁹ and R¹⁰ join together to form one or more rings, each having 5, 6, or 7 ring members.

IV. Diazepinoindolone Compounds Described in International Patent Publication WO2004063198, Including:

i) A compound of the formula:

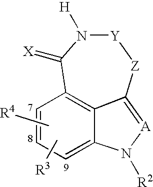

wherein: X is ═O or ═S; A is ═CR¹— or ═N—;

the group —Y-Z- has the formula —O—CH₂— or —N═CH—;

R¹ is:

(a)(C₁-C₈)alkyl;

(b) —C(═O)—R⁵;

(c) —C(═O)—NR⁶R⁷; or

(d) R³⁵, or R³⁶, (C₂-C₈) alkenyl, or (C₂-C₈) alkynyl {wherein each of said (C₂-C₈)alkenyl or (C₂-C₈)alkynyl is unsubstituted or substituted with one to four substituents independently selected from the group consisting of F, Cl, OH, —NH₂, R⁴⁰, and R⁴²};

R² is

(a) H, OH, or (C₁-C₈)alkyl;

(b) —C(═O)—R⁸;

(c) —(C═S)—R⁹ or —(C═S)—NR¹⁰R¹¹; or

(d) R³⁸ or R³⁹;

R³ is

(a) (C₁-C₈)alkyl;

(b) —C(═O)—R¹²;

(c) —C(═O)—NR¹³R¹⁴;

(d) —NR¹⁵—C(═O)—R¹⁶;

(e) —NR¹⁷—SO₂R¹⁸;

(f) —NR¹⁹—SOₙ—NR²⁰R²¹ {wherein n is 1 or 2};

(g) —NR²²—(C═S)—R²³ or —NR²²—(C═S)—NR²³R²⁴;

(h) R³⁶, (C₂-C₈)alkenyl, or (C₂-C₈)alkynyl {wherein each of said R³ (C₂-C₈)alkenyl or (C₂-C₈)alkynyl is unsubstituted or substituted with one to four substituents independently selected from the group consisting of —(C═O)—O—(C₁-C₈)alkyl, —O—(C═O)—(C₁-C₈)alkyl, —(C═O)—(C₁-C₈)alkyl, R⁴⁰, R⁴¹, and R⁴²};

(i) R³⁷, —NH₂, —NH((C₂-C₈)alkenyl), —NH((C₂-C₈)alkynyl), —N((C₁-C₈)alkyl)((C₂-C₈)alkenyl), or —N((C₁-C₈)alkyl)((C₂-C₈)alkynyl) {wherein each of said R²⁶ (C₂-C₈)alkenyl or (C₂-C₈)alkynyl is unsubstituted or substituted with one to four substituents independently selected from the group consisting of R⁴⁰, R⁴¹, and R⁴²}; or

(j) R³⁸;

R⁴ is selected from the group consisting of H, F, Br, Cl, and (C₁-C₈)alkyl;

R⁵ is selected from the group consisting of H, (C₁-C₈)alkyl, (C₁-C₈)alkyl-O—, and R³⁶;

Each R⁶ and R⁷ are independently selected from the group consisting of H, (C₁-C₈)alkyl, and R³⁶;

R⁸ is selected from the group consisting of (C₁-C₈)alkyl, (C₂-C₈)alkenyl, (C₂-C₈)alkynyl, —NH₂, R³⁶, and R³⁷;

Each of R⁹, R¹⁰ and R¹¹ are independently selected from the group consisting of H, (C₁-C₈)alkyl, and R³⁶;

R¹² is selected from the group consisting of H, OH, (C₁-C₈)alkyl, (C₁-C₈)alkyl-O—, and R³⁶;

R¹³ is H or (C₁-C₈)alkyl;

R¹⁴ is selected from the group consisting of H, (C₁-C₈)alkyl, —CH₂—(C═O)—O—(C₁-C₈)alkyl, and R³⁶,

R¹⁵ is H or (C₁-C₈)alkyl;
R¹⁶ is selected from the group consisting of H, (C₁-C₈)alkyl, (C₂-C₈)alkenyl, (C₂-C₈)alkynyl, —NH₂, R³⁶, and R³⁷; wherein each of said R¹⁵ and R¹⁶ (C₂-C₈)alkenyl or (C₂-C₈)alkynyl is unsubstituted or substituted with one to four substituents independently selected from the group consisting of R⁴⁰, R⁴¹, and R⁴²;

R¹⁷ is selected from the group consisting of H, (C₁-C₈)alkyl, and R³⁶;

R¹⁸ is (C₁-C₈)alkyl or R³⁶;

R¹⁹, R²⁰, and R²¹ are independently selected from the group consisting of H, (C₁-C₈)alkyl, and R³⁶;

R²², R²³ and R²⁴ are independently selected from the group consisting of H, (C₁-C₈)alkyl, and R³⁶;

R²⁵ is H or (C₁-C₈)alkyl;

R²⁶ is selected from the group consisting of —C(═O)—O—C(CH₃)₃, (C₁-C₈)alkyl, (C₃-C₁₀)cycloalkyl, (C₂-C₁₀)heterocyclyl, (C₆-C₁₀)aryl, and (C₁-C₁₀)heteroaryl; or R²⁵ and R²⁶ may optionally be taken together with the nitrogen to which they are attached to form a 5 to 8-membered heteroaryl or heterocyclyl ring;

R²⁷ is selected from the group consisting of (C₁-C₈)alkyl, (C₃-C₁₀)cycloalkyl, (C₂-C₁₀) heterocyclyl, (C₆-C₁₀)aryl, and (C₁-C₁₀) heteroaryl;

R²⁸ is selected from the group consisting of (C₁-C₈)alkyl, (C₃-C₁₀)cycloalkyl, (C₂-C₁₀) heterocyclyl, (C₆-C₁₀)aryl, and (C₁-C₁₀) heteroaryl;

R²⁹ is H or (C₁-C₈)alkyl;

R³⁰ is (C₁-C₈)alkyl, (C₃-C₁₀)cycloalkyl, (C₂-C₁₀)heterocyclyl, (C₆-C₁₀)aryl, or (C₁-C₁₀)heteroaryl; or R²⁹ and R³⁰ may optionally be taken together with the nitrogen to which they are attached to form a 5 to 8-membered heteroaryl or heterocyclyl ring;

R³¹ is H or (C₁-C₈)alkyl;

R³² is independently selected from the group consisting of (C₁-C₈)alkyl, (C₃-C₁₀)cycloalkyl, (C₂-C₁₀)heterocyclyl, (C₆-C₁₀)aryl, and (C₁-C₁₀)heteroaryl; or R³¹ and R³² may optionally be taken together with the nitrogen to which they are attached to form a 5 to 8-membered heteroaryl or heterocyclyl ring;

R³³ is (C₁-C₈)alkyl, (C₃-C₁₀)cycloalkyl, (C₂-C₁₀)heterocyclyl, (C₆-C₁₀)aryl, or (C₁-C₁₀)heteroaryl;

R³⁴ is (C₁-C₈)alkyl, (C₃-C₁₀)cycloalkyl, (C₂-C₁₀)heterocyclyl, (C₆-C₁₀), or (C₁-C₁₀)heteroaryl;

Each R³⁵ is independently selected from the group consisting of H, F, Cl, Br, I, CN, OH, NO₂, —NH₂, —NH—C(═O)—O—C(CH₃)₃, and CF₃;

Each R³⁶ is independently selected from the group consisting of (C₃-C₁₀)cycloalkyl, (C₂-C₁₀)heterocyclyl, (C₆-C₁₀)aryl, and (C₁-C₁₀)heteroaryl;

Each R³⁷ is independently selected from the group consisting of:

(c) —NR²⁵R²⁶; and

(d) R²⁷—O—;

R³⁸ is R²⁸—SOₙ—; wherein n is 0, 1, or 2 when —SOₙ— is bonded to R²¹ via an R²⁸ carbon atom, or wherein n is 1 or 2 when —SOₙ— is bonded to R²⁸ via an R⁸ ring nitrogen atom;

R³⁹ is R²⁹R³⁰N—SOₙ—; wherein n is 1 or 2; wherein each of said (C₁-C₈)alkyl, wherever it occurs in any of said R¹(a)-(d), R²(a)-(d), R³(a)-(j), R⁴, R³⁷, R³⁸, or R³⁹, is unsubstituted or substituted with one to four substituents independently selected from the group consisting of (C₂-C₈)alkenyl, R⁴⁰, R⁴¹, and R⁴²; wherein each of said (C₃-C₁₀)cycloalkyl, (C₂-C₁₀)heterocyclyl, (C₆-C₁₀)aryl, or (C₁-C₁₀)heteroaryl, wherever it occurs in said R³⁶, R³⁷, R³⁸, or R³⁹, is independently unsubstituted or substituted with one to four substituents independently selected from R⁴⁰;

R⁴⁰ is selected from the group consisting of (C₁-C₈)alkyl, R⁴¹, R⁴², and R⁴³;

Each R⁴¹ is independently selected from the group consisting of F, Cl, Br, I, CN, OH, NO₂, —NH₂, —NH—C(—O)—O—C(CH₃)₃, COOH, —C(═O)(C₁-C₈)alkyl, —C(═O)—O—(C₁-C₈)alkyl, —NH—SO₂—(C₁-C₈)alkyl, —NH—SO₂—(C₆-C₁₀)aryl, and CF₃;

Each R⁴² is independently selected from the group consisting of (C₃-C₁₀)cycloalkyl, (C₂-C₁₀)heterocyclyl, (C₆-C₁₀)aryl, and (C₁-C₁₀)heteroaryl;

Each R⁴³ is independently selected from the group consisting of:

(c) —NR³¹R³²;

(d) R³³—O—; and

(c) R³⁴—SOₙ—; wherein n is 0, 1, or 2 when —SOₙ— is bonded to R³⁴ via an R³⁴ carbon atom; or wherein n is 1 or 2 when —SOₙ— is bonded to R³⁴ via an R³⁴ ring nitrogen atom;

wherein each of said (C₁-C₈)alkyl, wherever it occurs in any of R⁴⁰ is independently unsubstituted or substituted with one to four substituents independently selected from the group consisting of R⁴ and R⁴⁵;

wherein each of said (C₃-C₁₀)cycloalkyl, (C₂-C₁₀)heterocyclyl, (C₆-C₁₀)aryl, or (C₁-C₁₀)heteroaryl, wherever it occurs in any of said R⁴² or R⁴³, is independently unsubstituted or substituted with one to four substituents independently selected from the group consisting of R⁴⁷ selected from the group consisting of (C₁-C₈)alkyl, R⁴¹, and R⁴⁵;

Each R⁴⁴ is independently selected from the group consisting of F, Cl, Br, I, CN, OH, NO₂, —NH₂, —CF₃, —C(═NH)—NH₂, —C(—NH)—NH—OH, —C(═NH)—NH—O—(C₁-C₈)alkyl, —(C═(O)—O—(C₁-C₈)alkyl, —O—(C═O)—(C₁-C₈)alkyl, —(C═O)—(C₁-C₈)alkyl, —(C═O)—NH₂, —C(═O)—NH(C₁-C₈)alkyl, —(C—O)—N<[(C₁-C₈)alkyl]₂, —NH—(C═O)—(C₁-C₈)alkyl, R³⁷, and R³⁸;

Each R⁴⁵ is independently selected from the group consisting of (C₃-C₁₀)cycloalkyl, (C₂-C₁₀)heterocyclyl, (C₆-C₁₀)aryl, and (C₁-C₁₀)heteroaryl;

wherein each of said (C₁-C₈)alkyl wherever it occurs in any of said R⁴ or R⁴⁵ is independently unsubstituted or substituted with one to four substituents independently selected from the group consisting of R⁴⁶ and R⁴⁷;

wherein each of said (C₃-C₁₀)cycloalkyl, (C₂-C₁₀)heterocyclyl, (C₆-C₁₀)aryl, or (C₁-C₁₀)heteroaryl, wherever it occurs in any of said R⁴³ or R⁴⁴ is independently unsubstituted or substituted with one to four substituents independently selected from the group consisting of (C₁-C₈)alkyl, R⁴⁶ and R⁴⁷;

Each R⁴⁶ is independently selected from the group consisting of F, Cl, Br, I, CN, OH, NO₂, —C(═NH)—NH₂, —C(═NH)—NH—OH, —C(═NH)—NH—O—(C₁-C₈)alkyl, —(C═O)—(C₁-C₈)alkyl, —O—(C═O)—(C₁-C₈)alkyl, —(C═O)—(C₁-C₈)alkyl, —(C—O)—NH₂, —(C═O)—NH(C₁-C₈)alkyl, —(C═O)—N<[(C₁-C₈)alkyl]₂, —NH—(C═O)—(C₁-C₈)alkyl, —C(═NH)—NH₂, —C(═NH)—NH—OH, —C(═NH)—NH—O—(C₁-C₈)alkyl, —(C═O)—O—(C₁-C₈)alkyl, —O—(C═O)—(C₁-C₈)alkyl, —(C═O)—(C₁-C₈)alkyl, —(C═O)—NH₂, —(C═O)—NH(C₁-C₈)alkyl, —(C═O)—N>[(C₁-C₈)alkyl]₂, —NH—(C═O)—(C₁-C⁸)alkyl, R³⁷, and R³⁸; and

Each R⁴⁷ is independently selected from the group consisting of (C₃-C₁₀)cycloalkyl; (C₂-C₁₀)heterocyclyl, (C₆-C₁₀)aryl, and (C₁-C₁₀)heteroaryl; or a pharmaceutical acceptable salt thereof;

V) Pyrimidine Compounds Described in International Patent Publication WO2004048343, Including:

i) A compound of formula:

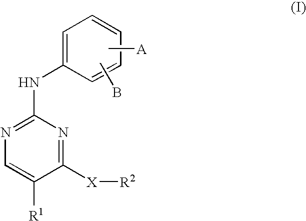

in which A or B in each case independently of one another represent cyano, halogen, hydrogen, hydroxy, aryl or the group —NO₂, —NH₂, —NR³R⁴, —C₁₋₆alkyl-NR³R⁴, —N(C₁₋₆-hydroxyalkyl)₂, —NH—C(NH)—CH₃, —NH(CO)—R⁵, —NHCOOR⁶, —NR⁷—(CO)—NR⁸R⁹, —NR⁷—(CS)—NR⁸R⁹, —COOR⁵, —CO—NR⁸R⁹, —CONH—C₁₋₆-alkyl-COOH, —SO₂—CH₃, 4 bromo-1-methyl-1H-pyrazolo-3yl or represent C₁₋₆-alkyl

optionally substituted in one or more places, the same way or differently with halogen, hydroxy, cyano or with the group —COOR⁵, —CONR⁸R⁹, —NH₂, —NH—SO₂—CH₃, —NR⁸R⁹, —NH—(CO)—R⁵, —NR⁷—(CO)—NR⁸R⁹, —SO₂—NHR³, —O—(CO)—R⁵ or —O—(CO)—C1-6-alkyl-R⁵;

X represents an oxygen atom or the group —NH— or —NR³R⁴;

R¹ represents hydrogen, halogen, hydroxymethyl, C₁₋₆-alkyl, cyano or the group —COOH, —COO-iso-propyl, —NO₂, —NH—(CO)—(CH₂)₂—COOH or —NH—(CO)—(CH₂)₂—COO—C₁₋₆-alkyl, whereby the C₁₋₆-alkyl can optionally be substituted in one or more places, in the same way or differently with halogen;

R² represents hydrogen or the group —NH—(CO)-aryl or C₁₋₆-alkyl optionally substituted in one or more places, the same way or differently with cyano, hydroxy, aryl, heteroaryl, C₃₋₆-heterocycloalkyl ring, which can optionally be interrupted with one or more nitrogen atoms, or substituted with the group —NR⁸R⁹, —NH—(CO)—NR⁸R⁹, —NH—(CO)—S—C₁₋₆-alkyl, —NH—(CS)—NR⁸R⁹, —NH—(CO)O—CH₂-phenyl, —NH—(CO)H, —NH(CO)—R⁵, —NH(CO)—OR⁵, —(CO)NH—NH₂, —(CO)—NH—CH₂—(CO)—NH₂, —(CO)—NH—C₁₋₆alkyl-COOH,

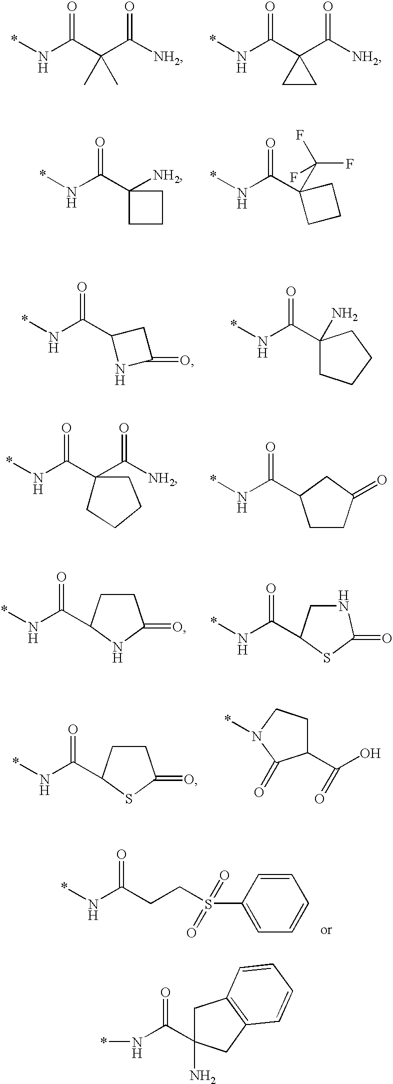

whereby the aryl or the heteroaryl can optionally be substituted in one or more places, the same or differently with halogen, hydroxy, C₁₋₆-alkyl, —NH₂, —NH—(CO)—CH₂—NH₂, —NO₂, —(CO)—C(CH₂)—C₂H₅, —COOR⁶, —COOC(CH₃)₃, or represents C₃-alkinyl;

R³ or R⁴ in each case independently of one another represent hydrogen or C₁₋₆-alkyl optionally substituted in one or more places, the same way or differently with hydroxy, phenyl or hydroxyphenyl, or

R³ and R⁴ together form a C₃₋₄-heterocycloalkyl ring containing at least one nitrogen atom and optionally can be interrupted by one or more oxygen and/or sulfur atoms and/or can be interrupted by one or more —(CO)— groups in the ring and/or optionally can contain one or more possible double bonds in the ring, whereby the C₃₋₆-heterocycloalkyl ring can optionally be substituted with C₁-alkyl, C₁₋₆-alkyl-COOH or C₁₋₆-alkyl-NH₂;

R⁵ represents hydrogen, C₁₋₆-alkyl, C₁₋₆-alkoxy, C₂₋₆-alkenyl, C₃₋₆-cycloalkyl ring, aryl, heteroaryl, the group —(CO)—NH₂ or C₃₋₆ heterocycloalkyl ring that can optionally be interrupted with one or more nitrogen and/or oxygen and/or sulfur atoms and/or can be interrupted by one or more —(CO)— groups in the ring and/or optionally can contain one or more possible double bonds in the ring and C₁₋₆-alkyl, C₂₋₆-alkenyl, C₃₋₆-cycloalkyl ring, C₃₋₆ heterocycloalkyl ring defined above, aryl or heteroaryl can optionally be substituted in one or more places, the same way or differently with halogen, hydroxy, C₁₋₆-alkyl, C₁₋₆-alkoxy, C₃₋₆-cycloalkyl ring, C₃₋₆ heterocycloalkyl ring defined above, aryl, heteroaryl or with the group NR⁸R⁹, —NO₂, —NR—(CO)—R⁵, —NH(CO)—C₁₋₆-alkyl-NH—(CO)—C₁₋₆-alkyl, —NR⁷—(CO)—NR⁸R⁹, —CO—CH₃, —COOH—, CO—NR⁸R⁹, —SO₂-aryl, —SH, —S—C₁₋₆-alkyl, —SO₂—NR⁸R⁹, whereby aryl itself can optionally be substituted in one or more places, the same way or differently with halogen, hydroxy, C₁₋₆-alkyl of C₁₋₆-alkoxy,

R⁶ represents C₁₋₆-alkyl, C₂₋₆-alkenyl or phenyl, whereby C₁₋₆-alkyl may optionally be substituted with C₃₋₆-heterocycloalkyl ring that can optionally be interrupted with one or more nitrogen and/or oxygen and/or sulfur atoms and/or can be interrupted by one or more —(CO)— groups in the ring and/or optionally can contain one or more possible double bonds in the ring;

R⁷ represents hydrogen or C₁₋₆-alkyl;

R⁸ or R⁹ in each case independently of one another represent hydrogen, C₁₋₆-alkyl, C₂₋₆-alkenyl, C₃₋₆-cycloalkyl, aryl or heteroaryl or the group R¹⁰,

whereby C₁₋₆-alkyl, C₂₋₆-alkenyl, C₃₋₆-cycloalkyl, aryl or heteroaryl can optionally be substituted in one or more places, the same way or differently with halogen, heteroaryl, hydroxy, —C₁₋₆alkoxy, hydroxy-C₁₋₆-alkoxy or the group —COOH, —NO₂, —NR⁸R⁹, —N(C₁₋₆-alkyl)₂ or with a C₃₋₆-heterocycloalkyl ring can optionally be interrupted with one or more nitrogen and/or oxygen and/or sulfur atoms and/or can be interrupted by one or more —(CO)— groups in the ring and/or optionally can contain one or more possible double bonds in the ring, or

R⁸ and R⁹ together form a C₃₋₄-heterocycloalkyl ring containing at least one nitrogen atom and optionally can be interrupted by one or more oxygen and/or sulfur atoms and/or can be interrupted by one or more —(CO)— groups in the ring and/or optionally can contain one or more possible double bonds in the ring, whereby the C₃₋₆-heterocycloalkyl ring can optionally be substituted in one or more places, the same way or differently with hydroxy or the group —NR⁸R⁹, —NH(CO)—R⁵, hydroxy-C₁₋₆-alkyl or —COOH; and

R¹⁰ represents —SO₂-aryl, —SO2-heteroaryl or —SO2-NH2 or —SO2-C1-6-alkyl,

whereby the aryl can be substituted with —C₁₋₆-alkyl, with the following provisos: whereby when X represents-NR³R⁴ then R² does not represent a substituent,

whereby when A and B represent hydrogen, X represents —NH— and R² represents C₁₋₆-alkyl, then R¹ represents —NH—(CO)—CH(NH₂)—(CH₂)₂—COOH or —NH—(CO)—CH(NH₂)—(CH₂)₂—COOC₂H₅;

whereby when A represents —(CO)—OC₂H₅ or hydroxy, B represents hydrogen, X represents oxygen, R¹ represents halogen, then R² represents C3-alkinyl;

whereby when A represents —(CO)—OC₂H₅ or hydroxy, B represents hydrogen, X represents —NH—, R¹ represents —NO₂, then R² represents C₃-alkinyl;

whereby when A represents —(CO)—OCH₃, then X represents oxygen, R¹ represents halogen, R² represents C₃-alkinyl and B represents —NH₂, —NHC₂H₄OH, —N(C₂H₄OH)₂, —NH—(CO)—CH₂—O(CO)CH₃,

whereby when A represents (CO)—OCH₃, then X represents —NH—, R¹ represents halogen, R² represents —C₂H₄-imidazolyl and B represents —NH₂;

whereby when A represents —NHSO₂CH₃, then X represents —NH—, R¹ represents halogen, R² represents —C₂H₄-imidazolyl;

whereby when R1 represents COO-iso-propyl, then X represents —NH, R² represents C₃-alkinyl and A or B independently of one another represent the group —NO₂ or —NH—(CO)—CF₃;

whereby when R¹ represents halogen, X represents —NH, B represents hydrogen, and R² represents C₁-alkyl substituted with —NH₂, then A represents —NH—(CO)—C₆-cycloakyl-NH₂;

whereby when R¹ represents halogen, X represents —NH, B represents —S—CH₃ and R² represents imidazolyl, then A represents the group

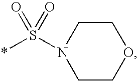

as well as all related isotopes, diastereomers, enantiomers, solvates, polymorphs or pharmaceutical acceptable salts thereof

VI. Diaryl Urea Compounds as Described in International Patent Publication WO2004014876, Including.

i) A compound of formula:

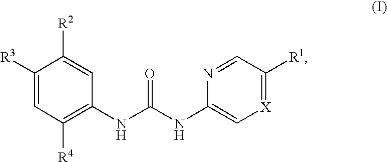

or a therapeutically acceptable salt thereof, wherein X is —N— or —CH—;

R¹ is selected from the group consisting of hydrogen, alkoxy, alkyl, amino, carboxy, cyano, halo, hydroxy, and hydroxyalkyl;

R² is selected from the group consisting of alkoxy, alkyl, alkylcarbonyl, amino, cyano, halo, and nitro;

R³ is selected from the group consisting of hydrogen, alkoxy, alkyl, amino, aminoalkyl, aminocarbonyl, arylalkyl, cyano, nitro, —CO₂R⁵, —COR⁵, and —SR⁵;

R⁴ is selected from the group consisting of —(CHR⁶)ₘOR⁷, and —(CH₂)ₙNR⁸R⁹;

R⁵ is selected from the group consisting of hydrogen, alkenyl, alkyl, aryl, arylalkyl, cycloalkyl, and (cycloalkyl)alkyl;

R⁶ is selected from the group consisting of hydrogen, alkyl, aryl, and heteroaryl;

R⁷ is selected from the group consisting of hydrogen, alkenyl, alkoxyalkoxyalkyl, alkoxyalkyl, alkoxycarbonylalkyl, alkylsulfanylalkyl, alkynyl, aminoalkyl, arylalkyl, arylcarbonylalkyl, aryloxyalkyl, arylsulfanylalkyl, cycloalkenyl, (cycloalkenyl)alkyl, cycloalkyl, (cycloalkyl)alkyl, heteroarylalkoxyalkyl, heteroarylalkyl, (heterocyclyl)alkoxyalkyl, (heterocyclyl)alkyl, and hydroxyalkyl;

R⁸ and R⁹ are independently selected from the group consisting of hydrogen, alkenyl, alkoxyalkyl, alkyl, alkylsulfanylalkyl, alkynyl, aminoalkyl, arylalkyl, cycloalkenyl, (cycloalkenyl)alkyl, cycloalkyl, (cycloalkyl)alkyl, heteroarylalkyl, heterocyclyl)alkyl, and hydroxyalkyl;

m is 0-6; provided that when R⁷ is hydrogen m is other than 0; and

n is 0-6; provided that when R⁸ and R⁹ are both hydrogen, n is other than 0.

VII. Diaryl Urea Compounds as Described in International Patent Publication WO2003101444, Including:

i) A compound of formula:

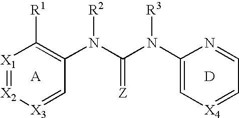

or a pharmaceutically acceptable salt thereof, wherein: X₁-X₃ are independently CH or N, that provided that X₁-X₃ are not all N; X₄ is CH or N; Z is O, S, or N—CN; Ring A is optionally substituted at any substitutable carbon by R⁴;

R¹ is -T-NH₂, —V-T-NH₂, -T-NHR^{x}, —V-T-NHR^{x}; T is a C₁₋₆ straight or branched alkylidene chain that is optionally interrupted by —O—, —S—, —N(R⁵)—, —S(O)—, —SO₂—, —C(O)—, —OC(O)—, —N(R⁵)C(O)—, —C(O)N(R⁵)—, —SO₂N(R⁵)—, or —N(R⁵)SO₂—, wherein the alkylidene chain or a portion thereof is optionally part of a 3-6 membered ring system; V is —O—, —S—, —N(R⁵)—, —S(O)—, —SO₂—, —C(O)—, —OC(O)—, —N(R⁵)C(O)—, —C(O)N(R⁵)—, —SO₂N(R⁵)—, or —N(R⁵)SO₂—;

R² and R³ are each independently selected from hydrogen, C₁₋₆ alkyl optionally substituted with —N(R⁸)₂, —C(═O)R, —CO₂R, or SO₂R, or R₂ and R₃ taken together with their intervening atoms form an optionally substituted an optionally substituted 5-6 membered ring;

each R⁴ is independently selected from halo, —OR, —SR, —CN, —NO₂, —N(R⁵)₂, —N(R⁵)C(O)R, —N(R⁵)CO₂R, —N(R⁵)C(O)N(R⁵)₂, —C(O)N(R⁵)₂, —C(O)R⁵, —OC(O)N(R⁵)₂, —CO₂R, —SO₂R, —S(O)R, —SO₂N(R⁵)₂, —N(R⁵)SO₂R, or an optionally substituted group selected from C₁₋₈ aliphatic, aryl, aralkyl, heterocyclyl, heterocyclealkyl, heteroaryl, or heteroaralkyl, or two ortho R⁴s, taken together with the ortho carbon atoms to which they are bonded, form an optionally substituted five or six membered phenyl, pyridyl or heterocyclyl fused to Ring A;

each R⁵ is independently selected from hydrogen, C₁₋₆ aliphatic, —CO₂R, —SO₂R, or —C(O)R, or two R⁵ on the same nitrogen taken together with the nitrogen form a 5-8 membered heteroaryl or heterocycle ring having 1-4 heteroatoms selected from N, O, or S;

each R⁸ is independently a C₁₋₃ alkyl or, taken together with the nitrogen atom to which they are bonded, a 5-7 membered nitrogen containing heterocycle;

Ring D is optionally substituted by C₁₋₄ aliphatic or haloaliphatic, —OR⁷, —SR⁷, —C(O)R⁷, —CO₂R⁷, —SO₂R⁷, —CN, —C(O)N(R⁷)₂, —N(R⁷)C(O)(C₁₋₂ alkyl), or —N(R⁷)₂ and is optionally fused to an optionally substituted phenyl or optionally substituted cyclohexyl ring;

each R⁷ is independently selected from hydrogen or an optionally substituted C₁₋₃ aliphatic or —N(R⁷)₂ is a nitrogen-containing heterocyclyl;

each R is independently selected from hydrogen or an optionally substituted group selected from C₁₋₆ aliphatic, aryl, aralkyl, heteroaryl, or heteroaralkyl-butyl; and

R^{x} is C1-C8 alkyl.

ii) A compound of formula:

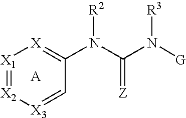

or a pharmaceutically acceptable salt thereof, wherein: X is CR¹; X₁-X₃ are CH; Z is O; Ring A is optionally substituted at any substitutable carbon by R⁴;

R¹ is V-T-R⁶, T is a C₂₋₄ alkylidene chain; V is —O—;

R² and R³ are each hydrogen;

each R⁴ is independently selected from halo, —OR, —SR, —CN, —NO₂, —N(R⁵)₂, —N(R⁵)C(O)R, —N(R⁵)CO₂R, —N(R⁵)C(O)N(R⁵)₂, —C(O)N(R⁵)₂, —OC(O)N(R⁵)₂, —CO₂R, —SO₂R, —S(O)R, —SO₂N(R⁵)₂, —N(R⁵)SO₂R,

or an optionally substituted group selected from C₁₋₈ aliphatic, aryl, aralkyl, heterocyclyl, heterocyclealkyl, heteroaryl, or heteroaralkyl, or two ortho R⁴s, taken together with the ortho carbon atoms to which they are bonded, form an optionally substituted five or six membered phenyl, pyridyl or heterocyclyl fused to Ring A;

each R⁵ is independently selected from hydrogen, C₁₋₆ aliphatic, CO₂R, —SO₂R, or —C(O)R, or two R⁵s on the same nitrogen taken together with the nitrogen form a 5-8 membered heteroaryl or heterocycle ring having 1-4 heteroatoms selected from N, O, or S;

R⁶—NH₂;

Each R⁸ is indepently a C₁₋₃ alkyl or, taken together with the nitrogen atom to which they are bounded, a 5-7 membered nitrogen containing heterocycle;

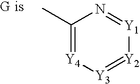

Y₁₋₄ are each independently selected from CH or nitrogen, provided that Ring B has no more than three nitrogen atoms and Y₁ and Y₂ are not both N, said Ring B being optionally substituted by C₁₋₄ aliphatic or haloaliphatic, —OR⁷, —SR⁷, —C(O)R⁷, —CO₂R⁷, —SO₂R⁷, —CN, —C(O)N(R⁷)₂, —N(R⁷)C(O)(C₁₋₂ alkyl), or —N(R⁷)₂;

each R⁷ is independently selected from hydrogen or an optionally substituted C₁₋₃ aliphatic or —N(R⁷)₂ is a nitrogen-containing heterocyclyl; and each R is hydrogen;

VIII. Pyrrolocarbazole Compounds as Described in International Patent Publication WO2003091255, Including:

i) A compound of formula 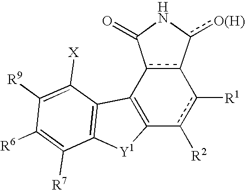
wherein each dashed line represents an optional bond;

R¹ is hydrogen, halogen, alkyl, NR⁵R⁶ or an aryl or heteroaryl ring optionally substituted with up to five substituents selected from

halogen, alkyl, haloalkyl, hydroxyl, nitro, cyano, C(O)R³, OR³, S(O)mR³, NR³R⁴, OC(O)R³, NR³(CO)OR⁴, CH₂NR³R⁴, CH²OR³, COOR³, CONR³R⁴, NR³COR⁴, SO₂NR³R⁴, CONHSO₂R³, NR³S(O)ₘR⁴, NHCONR³R⁴, NR³CONHR⁴; or a cycloalkyl or cycloalkenyl ring optionally substituted with up to five substituents

selected from, halogen, alkyl, haloalkyl, hydroxyl, nitro, cyano, C(O)R³, OR³, S(O)ₘR³, NR³R⁴, OC(O)R³, NR³(CO)OR⁴, CH₂NR³R⁴, CH₂OR³COOR³, CONR³R⁴, NR³COR⁴, SO₂NR³R⁴, CONHSO₂R³, NR³S(O)ₘR⁴, NHCONR³R⁴, NR³CONHR⁴; or a heterocyclic ring optionally substituted with up to five substituents selected from,

halogen, alkyl, haloalkyl, hydroxyl, nitro, cyano, C(O)R³, OR³, S(O)ₘR³, NR³R⁴, OC(O)R³, NR³(CO)OR⁴, CH₂NR³R⁴, CH₂OR³, COOR³, CONR³R⁴, NR³COR⁴, SO₂NR³R⁴, CONHSO₂R³, NR³S(O)ₘR⁴NHCONR³R⁴, NR³CONHR⁴;

m is 0-2; X is hydrogen or halogen;

Y¹ is O, S(O)m, or NR¹⁰;

R⁹ is hydrogen, hydroxyl, halogen, NR³C(O)R⁴, NHCONR³R⁴, (C═NR³)NHR⁴, NH(C═NR³)NHR⁴, NH(C═NH)NR³R⁴, NH(C═O)OR³, NR⁵R⁶, (CR⁵R⁶), Z;

r is 0-6;

R², R⁷, R⁸ and R¹⁰ are in each instance independently selected from ((CR⁵R⁶)ₙT)a(CR¹¹R¹²)_{b})-Z wherein the sum n, a and b is in each instance less than 10;

T may be absent, or, when present, is in each instance independently selected from O, CONR³, CONHSO₂, S(O)ₘ, NR³, NR³—O, O—S(O)ₘ, S(O)ₘO, NR³—S(O)₂, or S(O)₂—NR³;

n is in each instance independently 0-6; a is in each instance independently 0-6; b is in each instance independently 0-6;

Z is selected from hydrogen, halogen, alkyl, haloalkyl, cycloalkyl, cycloalkenyl, heterocyclyl, aryl, heteroaryl, cyano, nitro, hydroxy, C(O)R³, CONHSO₂R³, SR³, S(O)ₘR³, OSO₂R³, NR³R⁴, CO₂R³, CONR³R⁴, NR³COR⁴, SO₂NR³R⁴, OPO(OR¹)(OR⁴), CH═CR³R⁴, CCR³, (C═NR³)NHR⁴, NH(C═NR³)NHR⁴, NH(C═NH)NR³R⁴,

wherein the alkyl, cycloalkyl, cycloalkenyl, heterocyclyl, aryl or heteroaryl group may be substituted with up to four groups independently selected from halogen, alkyl, hydroxyl, nitro, cyano, OR³, S(O)ₘR³, NR³R⁴, OC(O)R³, NR³(CO)OR⁴, C(O)R³, COOR³, CONR³R⁴, NR³COR⁴, SO₂NR³R⁴, CONHSO₂R³, NR³S(O)ₘR⁴, CH₂NR³R⁴, CH₂OR³, NHCONR³R⁴, NR³CONHR⁴;

R⁵, R⁶, R¹¹ and R¹² are in each instance independently selected from hydrogen, hydroxyl, alkyl, haloalkyl, cycloalkyl, cycloalkenyl, aryl, heteroaryl, heterocyclyl, halogen, cyano, nitro, CH₂NR³R⁴, CH₂OR³, C(O)R³, OR³, S(O)ₘR³, NR³R⁴, COOR³, CONR³R⁴, SO₂NR³R⁴, NHCONR³R⁴, NR³CONHR⁴;

wherein the alkyl, haloalkyl, cycloalkyl, cycloalkenyl, heterocyclyl, aryl or heteroaryl group may be substituted with up to four groups independently selected from halogen, alkyl, hydroxyl, nitro, cyano, OR³, S(O)ₘR³, NR³R⁴, OC(O)R³, NR³(CO)OR⁴, C(O)R³, COOR³, CONR³R⁴, NR³COR⁴, SO₂NR³R⁴, CONHSO₂R³, NR³S(O)ₘR⁴, NHCONR³R⁴, NR³CONHR⁴;

R⁵, R⁶, R¹¹ and R¹² together with the carbon atom to which they are attached may form a carbonyl group; or together with the carbon or heteroatom to which they are attached may form a cycloalkyl or heterocyclyl group,

said carbonyl, cycloalkyl or heterocyclyl group may be substituted with up to four groups independently selected from halogen, hydroxyl, nitro, cyano, alkyl, haloalkyl, alkyl, nitro, cyano. OR³, S(O)ₘR³, NR³R⁴, OC(O)R³, NR³(CO)OR⁴, C(O)R³, COOR³, CONR³R⁴, NR³COR⁴, NR³COR⁴, SO₂NR³R⁴, CONHSO₂R³, NR³S(O)ₘR⁴, NHCONR³R⁴, NR³CONH⁴;

R³, R⁴ are independently selected from hydrogen, alkyl, haloalkyl or a substituted or unsubstituted carbocyclic group

selected from cycloalkyl, cycloalkenyl, heterocyclyl, aryl, and heteroaryl, wherein the said alkyl, or a substituted group may be substituted with up to 4 groups selected from halogen, hydroxyl, nitro, cyano, alkyl, haloalkyl, alkyloxy, carboxy, COOH, CONH₂, NHCOCH₃, N(CH₃)₂, NHCH₃, thiomethyl, thioethyl, SOCH₃, SO₂CH₃;

R³ and R⁴ together with the carbon atom or heteroatom to which they are attached may form a cycloalkyl or heterocyclyl group substituted with up to four groups independently selected from halogen, hydroxyl, nitro, cyano, alkyl, haloalkyl, alkyloxy, formyl, carboxy, acetyl, CH₂NH₂, CH₂OH, COOH, CONH₂, NHCOCH₃, N(CH₃)₂, thiomethyl, thioethyl, SOCH₃, SO₂CH₃, alkoxycarbonyl, alkylcarbonyl, alkynylamino, aminoalkyl, aminoalkylcarbonyl, amino, mono- or dialkylamino, or

R³ and R⁴ together with the nitrogen to which they are attached may form a heterocyclic ring containing 3-8 members, up to four of which members are optionally carbonyl groups or heteroatoms independently selected from,

oxygen, sulfur, S(O), S(O)₂, and nitrogen, wherein the carbocyclic group is unsubstituted or substituted with up to four groups independently selected from halogen, hydroxy, hydroxyalkyl, alkyl, haloalkyl, alkoxy, alkoxycarbonyl, alkylcarbonyl, alkynylamino, aminoalkyl, aminoalkylcarbonyl, amino, mono- or dialkylamino.

IX Ureidothiophenes as Described in International Patent Publication WO2003/029241, Including:

i) A compound of formula:

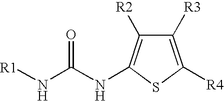

wherein:

R1 is selected from the group consisting of H, C₁₋₂ alkyl, XH, XCH₃, C₁₋₂ alkyl-XH, C₁₋₂ alkyl-XCH₃, C(O)NH₂, C(O)NHCH₃, and C(O)—C₁₋₂ alkyl; X is selected from the group consisting of O, S, and NH;

R2 is selected from the group consisting of C(O)R⁵, CO₂R⁵, C(O)NHR⁵, C(O)NHC(═NH)R⁵, C(O)NHC(—NH)NR⁵R⁶, C(O)NHC(O)R⁵, C(O)NHC(O)NR⁵R⁶, SO₂R⁵, S(O)R⁵, SO₃R⁵, and PO₃R⁵R⁶;

R⁵ and R⁶ are, independently, selected from the group consisting of hydrogen, C₁₋₁₀ alkyl, C₁₋₁₀ alkanoyl, C₂₋₁₀ alkenyl, C₂₋₁₀ alkynyl, C₃₋₁₀ cycloalkyl, C₀₋₆alkylaryl, C₀₋₆ alkylheterocyclyl, and C₀₋₆ alkylheteroaryl, or R⁵ and R⁶ taken together with the nitrogen to which they are attached, may optionally form a ring having 3 to 7 carbon atoms, optionally containing 1, 2, or 3 heteroatoms selected from nitrogen, sulfur, oxygen, or nitrogen, substituted with hydrogen, C₁₋₆alkyl or (CH₂)₀₋₃aryl, such that any of the foregoing may be optionally substituted by one or more of group A and on any position;

R3 is H or halogen;

R4 is aryl or heteroaryl optionally substituted by one or more of group A and on any position;

A is selected from the group consisting of C₁₋₁₀ alkyl, C₁₋₁₀ alkanoyl, C₂₋₁₀ alkenyl, C₂₋₁₀ alkynyl, C₃₋₁₀ cycloalkyl, C₀₋₆ alkylaryl, C₀₋₆ alkylheterocyclyl, C₀₋₆ alkylheteroaryl, C(═NH)R⁷, COR⁷, CONR⁷R⁸, CON(O)R⁷R⁸, CONR⁷R⁸R⁹Y, CO₂R⁷, C(O)SR⁷, C(S)R⁷, cyano, trifluoromethyl, NR⁷R⁸, N(O)R⁷R⁸, NR⁷R⁸R⁹Y, NR⁷COR⁷, NR⁷CONR⁷R⁸, NR⁷CON(O)R⁷R⁸, NR⁷CONR⁷R⁸R⁹Y, NR⁷CO₂R⁷, NR⁷C(O)SR⁷, NR⁷SO₂R⁷, NR⁷SO2NR⁷R⁸, nitro, OR⁷, OCF₃, aryloxy, heteroaryloxy, SR⁷, S(O)R⁷, S(O)₂R⁷, SCF₃, S(O)CF₃, S(O)₂CF₃, SO₂NR⁷R⁸, SO₃R⁷, PO₃R⁷R⁸, and halo,

wherein C₁₋₁₀ alkyl, C₁₋₁₀ alkanoyl, C₂₋₁₀ alkenyl, C₂₋₁₀ alkynyl, C₃₋₁₀ cycloalkyl, C₀₋₆ alkylaryl, C₀₋₆ alkylheterocyclyl, C₀₋₆ alkylheteroaryl, (CH₂)₀₋₆ heteroaryl, aryloxy, and heteroaryloxy may be optionally substituted by one or more of group D and on any position;

Y is an organic or inorganic anion;

D is selected from the group consisting of C₁₋₁₀ alkyl, C₁₋₁₀ alkanoyl, C₂₋₁₀ alkenyl, C₂₋₁₀ alkynyl, C₃₋₁₀ cycloalkyl, C₀₋₆₁alkylaryl, C₀₋₆ alkylheterocyclyl, C₀₋₆ alkylheteroaryl; C(═NH)R⁷, COR⁷, CONR⁷R⁸, CON(O)R⁷R⁸, CONR⁷R⁸R⁹Y, CO₂R⁷, C(O)SR⁷, C(S)R⁷, cyano, trifluoromethyl, NR⁷R⁸, N(O)R⁷R⁸, NR⁷R⁸R⁹Y, NR⁷COR⁷, NR⁷CONR⁷R⁸, NR⁷CON(O)R⁷R⁸, NR⁷CONR⁷R⁸R⁹Y, NR⁷CO₂R⁷, NR⁷C(O)SR⁷, NR⁷SO₂R⁷, NR⁷SO2NR⁷R⁸, nitro, OR⁷, OCF₃, aryloxy, heteroaryloxy, SR⁷, S(O)R⁷, S(O)₂R⁷, SCF₃, S(O)CF₃, S(O)₂CF₃, SO₂NR⁷R⁸, SO₃R⁷, PO₃R⁷R⁸, and halo,

wherein C₁₋₁₀ alkyl, C₁₋₁₀ alkanoyl, C₂₋₁₀ alkenyl, C₂₋₁₀ alkynyl, C₃₋₁₀ cycloalkyl, C₀₋₆ alkylaryl, C₀₋₆alkylheterocyclyl, C₀₋₆ alkylheteroaryl, (CH₂)₀₋₆ heteroaryl, aryloxy, and heteroaryloxy may be optionally substituted by one or more of group E and on any position;

R⁷, R⁸, and R⁹ are independently selected from the group consisting of hydrogen, C₁₋₁₀ alkyl, C₁₋₁₀ alkanoyl, C₂₋₁₀ alkenyl, C₂₋₁₀ alkynyl, C₃₋₁₀ cycloalkyl, C₀₋₆ alkylaryl, C₀₋₆ alkylheterocyclyl, and C₀₋₆ alkylheteroaryl,

or R⁷ and R⁵ taken together with the nitrogen to which they are attached may optionally form a ring having 3 to 7 carbon atoms optionally containing 1, 2, or 3 heteroatoms selected from nitrogen, sulfur, oxygen, or nitrogen, substituted with hydrogen, C₁₋₆ alkyl or (CH₂)₀₋₃ aryl, wherein any of the foregoing may be substituted by one or more of group E and on any position;

E is selected from the group consisting of C₁₋₁₀ alkyl, C₁₋₁₀ alkanoyl, C₂₋₁₀ alkenyl, C₂₋₁₀ alkynyl, C₃₋₁₀ cycloalkyl, C₀₋₆ alkylaryl, C₀₋₆ alkylheterocyclyl, C₀₋₆ alkylheteroaryl, C(═NH)R¹⁰, COR¹⁰, CONR¹⁰R¹¹, CON(O)R¹⁰R¹¹, CONR¹⁰R¹¹R¹²Y, CO₂R¹⁰, C(O)SR¹⁰, C(S)R¹⁰, cyano, trifluoromethyl, NR¹⁰R¹¹, N(O)R¹⁰R¹¹, NR¹⁰R¹¹R¹²Y, NR¹⁰COR¹⁰, NR¹⁰CONR¹⁰R¹¹, NR¹⁰CON(O)R¹⁰R¹¹, NR¹⁰CONR¹⁰R¹¹R¹²Y, NR¹⁰CO₂R¹⁰, NR¹⁰C(O)SR¹⁰, NR¹⁰SO₂R¹⁰, NR¹⁰SO₂NR¹⁰R¹¹, nitro, OR¹⁰, OCF₃, aryloxy, heteroaryloxy, SR¹⁰, S(O)R¹⁰, S(O)₂R¹⁰, SCF₃, S(O)CF₃, S(O)₂CF₃, SO₂NR¹⁰R¹¹, SO₃R¹⁰, PO₃R¹⁰R¹¹, and halo,

wherein C₁₋₁₀ alkyl, C₁₋₁₀ alkanoyl, C₂₋₁₀ alkenyl, C₂₋₁₀ alkynyl, C₃₋₁₀ cycloalkyl, C₀₋₆ alkylaryl, C₀₋₆ alkylheterocyclyl, C₀₋₆ alkylheteroaryl may be substituted by one or more of C(═NH)R¹⁰, COR¹⁰, CONR¹⁰R¹¹, CON(O)R¹⁰R¹¹, CONR¹⁰R¹¹R¹²Y, CO₂R¹⁰, C(O)SR¹⁰, C(S)R¹⁰, cyano, trifluoromethyl, NR¹⁰R¹¹, N(O)R¹⁰R¹¹, NR¹⁰R¹¹R¹²Y, NR¹⁰COR¹⁰, NR¹⁰CONR¹⁰R¹¹, NR¹⁰CON(O)R¹⁰R¹¹, NR¹⁰CONR¹⁰R¹¹R¹²Y, NR¹⁰CO₂R¹⁰, NR¹⁰C(O)SR¹⁰, NR¹⁰SO₂R¹⁰, NR¹⁰SO₂NR¹⁰R¹¹, nitro, OR¹⁰, OCF₃, aryloxy, heteroaryloxy, SR¹⁰, S(O)R¹⁰, S(O)₂R¹⁰, SCF₃, S(O)CF₃, S(O)₂CF₃, SO₂NR¹⁰R¹¹, SO₃R¹⁰, PO₃R¹⁰R¹¹, or halo, and on any position;

R¹⁰, R¹¹, and R¹² are independently, selected from the group consisting of hydrogen, C₁₋₁₀ alkyl, C₁₋₁₀ alkanoyl, C₂₋₁₀ alkenyl, C₂₋₁₀ alkynyl, C₃₋₁₀ cycloalkyl, C₀₋₆ alkylaryl, C₀₋₆ alkylheterocyclyl, and C₀₋₆ alkylheteroaryl, or R¹⁰ and R¹¹ taken together with the nitrogen to which they are attached complete a ring having 3 to 7 carbon atoms optionally containing 1, 2, or 3 heteroatoms selected from nitrogen, sulfur, oxygen, or nitrogen, substituted with hydrogen, C₁₋₆ alkyl or (CH₂)₀₋₃ aryl;

or a pharmaceutically acceptable inorganic or organic salt, esters, or other prodrug.

ii) A compound of formula:

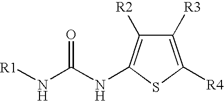

wherein:

R1 is selected from the group consisting of H, C₁₋₂ alkyl, XH, XCH₃, C₁₂ alkyl-XH, C₁₋₂ alkyl-XCH₃, C(O)NH₂, C(O)NHCH₃, and C(O)—C₁₋₂ alkyl,

provided that when R1 is H, R2 is not CONH₂, or provided that when R1 is C₁₋₂ alkyl, R2 is not CONH₂; with the preferred substitution being H or CH₃; X is selected from the group consisting of O, S, and NH;

R² is selected from the group consisting of C(O)R⁵, CO₂R⁵, C(O)NHR⁵, C(O)NHC(═NH)R⁵, C(O)NHC(═NH)NR⁵R⁶, C(O)NHC(O)R⁵, C(O)NHC(O)NR⁵R⁶, SO₂R⁵, S(O)R⁵, SO₃R⁵, and PO₃R⁵R⁶;

R⁵ and R⁶ are, independently, selected from the group consisting of hydrogen, C₁₋₁₀ alkyl, C₁₋₁₀ alkanoyl, C₂₋₁₀ alkenyl, C₂₋₁₀ alkynyl, C₃₋₁₀ cycloalkyl, C₀₋₆ alkylaryl, C₀₋₆ alkylheterocyclyl, and C₀₋₆ alkylheteroaryl, or R⁵ and R⁶ taken together with the nitrogen to which they are attached may optionally form a ring having 3 to 7 carbon atoms, optionally containing 1, 2, or 3 heteroatoms selected from nitrogen, sulfur, oxygen, or nitrogen, substituted with hydrogen, C₁₋₆ alkyl or (CH₂)₀₋₃aryl, such that any of the foregoing may be optionally substituted by one or more of group A and on any position;

R3 is H or halogen; with the preferred substitution being H;

R4 is aryl or heteroaryl optionally substituted by one or more of group A and on any position, provided that when R2 is CO₂R⁵ or CONH₂, R4 is not phenyl, or provided that when R1 is H, R4 is not 4-pyridyl;

A is selected from the group consisting of C₁₋₁₀ alkyl, C₁₋₁₀ alkanoyl, C₂₋₁₀ alkenyl, C₂₋₁₀ alkynyl, C₃₋₁₀ cycloalkyl, C₀₋₆ alkylaryl, C₀₋₆ alkylheterocyclyl, C₀₋₆ alkylheteroaryl, C(═NH)R⁷, COR⁷, CONR⁷R⁸, CON(O)R⁷R⁸, CONR⁷R⁸R⁹Y, CO₂R⁷, C(O)SR⁷, C(S)R⁷, cyano, trifluoromethyl, NR⁷R⁸, N(O)R⁷R⁸, NR⁷R⁸R⁹Y, NR⁷COR⁷, NR⁷CONR⁷R⁸, NR⁷CON(O)R⁷R⁸, NR⁷CONR⁷R⁸R⁹Y, NR⁷CO₂R⁷, NR⁷C(O)SR⁷, NR⁷SO₂R⁷, NR⁷SO2NR⁷R⁸, nitro, OR⁷, OCF₃, aryloxy, heteroaryloxy, SR⁷, S(O)R⁷, S(O)₂R⁷, SCF₃, S(O)CF₃, S(O)₂CF₃, SO₂NR⁷R⁸, SO₃R⁷, PO₃R⁷R⁸, and halo, wherein C₁₋₁₀ alkyl, C₁₋₁₀ alkanoyl, C₂₋₁₀ alkenyl, C₂₋₁₀ alkynyl, C₃₋₁₀ cycloalkyl, C₀₋₆ alkylaryl, C₀₋₆ alkylheterocyclyl, C₀₋₆ alkylheteroaryl, (CH₂)₀₋₆heteroaryl, aryloxy, and heteroaryloxy may be optionally substituted by one or more of group D and on any position;

Y is an organic or inorganic anion;

D is selected from the group consisting of C₁₋₁₀ alkyl, C₁₋₁₀ alkanoyl, C₂₋₁₀ alkenyl, C₂₋₁₀ alkynyl, C₃₋₁₀ cycloalkyl, C₀₋₆ alkylaryl, C₀₋₆ alkylheterocyclyl, C₀₋₆ alkylheteroaryl, C(═NH)R⁷, COR⁷, CONR⁷R⁸, CON(O)R⁷R⁸, CONR⁷R⁸R⁹Y, CO₂R⁷, C(O)SR⁷, C(S)R⁷, cyano, trifluoromethyl, NR⁷R⁸, N(O)R⁷R⁸, NR⁷R⁸R⁹Y, NR⁷COR⁷, NR⁷CONR⁷R⁸, NR⁷CON(O)R⁷R⁸, NR⁷CONR⁷R⁸R⁹Y, NR⁷CO₂R⁷, NR⁷C(O)SR⁷, NR⁷SO₂R⁷, NR⁷SO2NR⁷R⁸, nitro, OR⁷, OCF₃, aryloxy, heteroaryloxy, SR⁷, S(O)R⁷, S(O)₂R⁷, SCF₃, S(O)CF₃, S(O)₂CF₃, SO₂NR⁷R⁸, SO₃R⁷, PO₃R⁷R⁸, and halo,

wherein C₁₋₁₀ alkyl, C₁₋₁₀ alkanoyl, C₂₋₁₀ alkenyl, C₂₋₁₀ alkynyl, C₃₋₁₀ cycloalkyl, C₀₋₆ alkylaryl, C₀₋₆ alkylheterocyclyl, C₀₋₆ alkylheteroaryl, (CH₂)₀₋₆ heteroaryl, aryloxy, and heteroaryloxy may be optionally substituted by one or more of group E and on any position;

R⁷, R⁸, and R⁹ are independently selected from the group consisting of hydrogen, C₁₋₁₀ alkyl, C₁₋₁₀ alkanoyl, C₂₋₁₀ alkenyl, C₂₋₁₀ alkynyl, C₃₋₁₀ cycloalkyl, C₀₋₆ alkylaryl, C₀₋₆ alkylheterocyclyl, and C₀₋₆ alkylheteroaryl,

or R⁷ and R⁸ taken together with the nitrogen to which they are attached may optionally form a ring having 3 to 7 carbon atoms optionally containing 1, 2, or 3 heteroatoms selected from nitrogen, sulfur, oxygen, or nitrogen, substituted with hydrogen, C₁₋₆ alkyl or (CH₂)₀₋₃ aryl, wherein any of the foregoing may be substituted by one or more of group E and on any position;

E is selected from the group consisting of C₁₋₁₀ alkyl, C₁₋₁₀ alkanoyl, C₂₋₁₀ alkenyl, C₂₋₁₀ alkynyl, C₃₋₁₀ cycloalkyl, C₀₋₆ alkylaryl, C₀₋₆ alkylheterocyclyl, C₀₋₆ alkylheteroaryl, C(═NH)R¹⁰, COR¹⁰, CONR¹⁰R¹¹, CON(O)R¹⁰R¹¹, CONR¹⁰R¹¹R¹²Y, CO₂R¹⁰, C(O)SR¹⁰, C(S)R¹⁰, cyano, trifluoromethyl, NR¹⁰R¹¹, N(O)R¹⁰R¹¹, NR¹⁰R¹¹R¹²Y, NR¹⁰COR¹⁰, NR¹⁰CONR¹⁰R¹¹, NR¹⁰CON(O)R¹⁰R¹¹, NR¹⁰CONR¹⁰R¹¹R¹²Y, NR¹⁰CO₂R¹⁰, NR¹⁰C(O)SR¹⁰, NR¹⁰SO₂R¹⁰, NR¹⁰SO₂NR¹⁰R¹¹, nitro, OR¹⁰, OCF₃, aryloxy, heteroaryloxy, SR¹⁰, S(O)R¹⁰, S(O)₂R¹⁰, SCF₃, S(O)CF₃, S(O)₂CF₃, S(O)₂NR¹⁰R¹¹, SO₃R¹⁰, PO₃R¹⁰R¹¹, and halo,

wherein C₁₋₁₀ alkyl, C₁₋₁₀ alkanoyl, C₂₋₁₀ alkenyl, C₂₋₁₀ alkynyl, C₃₋₁₀ cycloalkyl, C₀₋₆ alkylaryl, C₀₋₆ alkylheterocyclyl, C₀₋₆alkylheteroaryl may be substituted by one or more of C(═NH)R¹⁰, COR¹⁰, CONR¹⁰R¹¹, CON(O)R¹⁰R¹¹, CONR¹⁰R¹¹R¹²Y, CO₂R¹⁰, C(O)SR¹⁰, C(S)R¹⁰, cyano, trifluoromethyl, NR¹⁰R¹¹, N(O)R10R¹¹, NR¹⁰R¹¹R¹²Y, NR¹⁰COR¹⁰, NR¹⁰CONR¹⁰R¹¹, NR¹⁰CON(O)R¹⁰R¹¹, NR¹⁰CONR¹⁰R¹¹R¹²Y, NR¹⁰CO₂R¹⁰, NR¹⁰C(O)SR¹⁰, NR¹⁰SO₂R¹⁰, NR¹⁰SO₂NR¹⁰R¹¹, nitro, OR¹⁰, OCF₃, aryloxy, heteroaryloxy, SR¹⁰, S(O)R¹⁰, S(O)₂R¹⁰, SCF₃, S(O)CF₃, S(O)₂CF₃, SO₂NR¹⁰R¹¹, SO₃R¹⁰, PO₃R¹⁰R¹¹, or halo, and on any position;

R¹⁰, R¹¹, and R¹² are independently, selected from the group consisting of hydrogen, C₁₋₁₀ alkyl, C₁₋₁₀ alkanoyl, C₂₋₁₀ alkenyl, C₂₋₁₀ alkynyl, C₃₋₁₀ cycloalkyl, C₀₋₆ alkylaryl, C₀₋₆ alkylheterocyclyl, and C₀₋₆ alkylheteroaryl, or R¹⁰ and R¹¹ taken together with the nitrogen to which they are attached complete a ring having 3 to 7 carbon atoms optionally containing 1, 2, or 3 heteroatoms selected from nitrogen, sulfur, oxygen, or nitrogen, substituted with hydrogen, C₁₋₆ alkyl or (CH₂)₀₋₃aryl;

or a pharmaceutically acceptable inorganic or organic salt, esters, or other prodrug of said compound.

X. Ureidothiophene Compounds as Described in International Patent Publication WO2003028731, Including:

i) A compound of formula:

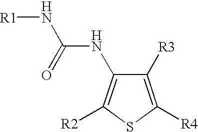

wherein: R1 is selected from the group consisting of H, C₁₋₂ alkyl, XH, XCH₃; C₁₋₂ alkyl-XH, C₁₋₂ alkyl-XCH₃, C(O)NH₂, C(O)NHCH₃, and C(O)—C₁₋₂ alkyl; X is selected from the group consisting of O, S, and NH;

R² is selected from the group consisting of C(O)R⁵, CO₂R⁵, C(O)NHR⁵, C(O)NHC(═NH)R⁵, C(O)NHC(═NH)NR⁵R⁶, C(O)NHC(O)R⁵, C(O)NHC(O)NR⁵R⁶, SO₂R⁵, S(O)R⁵, SO₃R⁵, and PO₃R⁵R⁶; R¹ and R⁶ are, independently, selected from the group consisting of hydrogen, C₁₋₁₀ alkyl, C₁₋₁₀ alkanoyl, C₂₋₁₀ alkenyl, C₂₋₁₀ alkynyl, C₃₋₁₀ cycloalkyl, C₀₋₆ alkylaryl, C₀₋₆ alkylheterocyclyl, and C₀₋₆ alkylheteroaryl,

or R⁵ and R⁶, taken together with the nitrogen to which they are attached, may optionally form a ring having 3 to 7 carbon atoms optionally containing 1, 2, or 3 heteroatoms selected from nitrogen, sulfur, oxygen, or nitrogen, substituted with hydrogen, C₁₋₆ alkyl or (CH₂)₀₋₃aryl,

wherein any of the foregoing may be optionally substituted by one or more of group A and on any position;

R3 is H or halogen;

R4 is aryl or heteroaryl optionally substituted by one or more of group A and on any position;

A is selected from the group consisting of C₁₋₁₀ alkyl, C₁₋₁₀ alkanoyl, C₂₋₁₀ alkenyl, C₂₋₁₀ alkynyl, C₃₋₁₀ cycloalkyl, C₀₋₆ alkylaryl, C₀₋₆ alkylheterocyclyl, C₀₋₆ alkylheteroaryl, C(═NH)R⁷, COR⁷, CONR⁷R⁸, CON(O)R⁷R⁸, CONR⁷R⁸R⁹Y, CO₂R⁷, C(O)SR⁷, C(S)R⁷, cyano, trifluoromethyl, NR⁷R⁸, N(O)R⁷R⁸, NR⁷R⁸R⁹Y, NR⁷COR⁷, NR⁷CONR⁷R⁸, NR⁷CON(O)R⁷R⁸, NR⁷CONR⁷R⁸R⁹Y, NR⁷CO₂R⁷, NR⁷C(O)SR⁷, NR⁷SO₂R⁷, NR⁷SO2NR⁷R⁸, nitro, OR⁷, OCF₃, aryloxy, heteroaryloxy, SR⁷, S(O)R⁷, S(O)₂R⁷, SCF₃, S(O)CF₃, S(O)₂CF₃, SO₂NR⁷R⁸, SO₃R⁷, PO₃R⁷R⁸, and halo,

wherein C₁₋₁₀ alkyl, C₁₋₁₀ alkanoyl, C₂₋₁₀ alkenyl, C₂₋₁₀ alkynyl, C₃₋₁₀ cycloalkyl, C₀₋₆ alkylaryl, C₀₋₆ alkylheterocyclyl, C₀₋₆ alkylheteroaryl, (CH₂)₀₋₆ heteroaryl, aryloxy, and heteroaryloxy may be optionally substituted by one or more of group D and on any position;

Y is an organic or inorganic anion;

D is selected from the group consisting of C₁₋₁₀ alkyl, C₁₋₁₀ alkanoyl, C₂₋₁₀ alkenyl, C₂₋₁₀ alkynyl, C₃₋₁₀ cycloalkyl, C₀₋₆ alkylaryl C₀₋₆ alkylheterocyclyl, C₀₋₆ alkylheteroaryl, C(═NH)R⁷, COR⁷, CONR⁷R⁸, CON(O)R⁷R⁸, CONR⁷R⁸R⁹Y, CO₂R⁷, C(O)SR⁷, C(S)R⁷, cyano, trifluoromethyl, NR⁷R⁸, N(O)R⁷R⁸, NR⁷R⁸R⁹Y, NR⁷COR⁷, NR⁷CONR⁷R⁸, NR⁷CON(O)R⁷R⁸, NR⁷CONR⁷R⁸R⁹Y, NR⁷CO₂R⁷, NR⁷C(O)SR⁷, NR⁷SO₂R⁷, NR⁷SO₂NR⁷R⁸, nitro, OR⁷, OCF₃, aryloxy, heteroaryloxy, SR⁷, S(O)R⁷, S(O)₂R⁷, SCF₃, S(O)CF₃, S(O)₂CF₃, S(O)₂NR⁷R⁸, SO₃R⁷, PO₃R⁷R⁸, and halo,

wherein C₁₋₁₀ alkyl, C₁₋₁₀ alkanoyl, C₂₋₁₀ alkenyl, C₂₋₁₀ alkynyl, C₃₋₁₀ cycloalkyl, C₀₋₆ alkylaryl, C₀₋₆ alkylheterocyclyl, C₀₋₆ alkylheteroaryl, (CH₂)₀₋₆ heteroaryl, aryloxy, and heteroaryloxy may be optionally substituted by one or more of group E and on any position;

R⁷, R⁸, and R⁹ are, independently, selected from the group consisting of hydrogen, C₁₋₁₀ alkyl, C₁₋₁₀ alkanoyl, C₂₋₁₀ alkenyl, C₂₋₁₀ alkynyl, C₃₋₁₀ cycloalkyl, C₀₋₆ alkylaryl, C₀₋₆ alkylheterocyclyl, and C₀₋₆ alkylheteroaryl, or R⁷ and R⁸, taken together with the nitrogen to which they are attached, may optionally form a ring having 3 to 7 carbon atoms, optionally containing 1, 2, or 3 heteroatoms selected from nitrogen, sulfur, oxygen, or nitrogen, substituted with hydrogen, C₁₋₆ alkyl or (CH₂)₀₋₃ aryl, wherein any of the foregoing may be optionally substituted by one or more of group E and on any position;

E is selected from the group consisting of C₁₋₁₀ alkyl, C₁₋₁₀ alkanoyl, C₂₋₁₀ alkenyl, C₂₋₁₀ alkynyl, C₃₋₁₀ cycloalkyl, C₀₋₆ alkylaryl, C₀₋₆ alkylheterocyclyl, C₀₋₆ alkylheteroaryl, C(═NH)R¹⁰, COR¹⁰CONR¹⁰R¹¹, CON(O)R¹⁰R¹¹, CONR¹⁰R¹¹R¹²Y, CO₂R¹⁰, C(O)SR¹⁰, C(S)R¹⁰, cyano, trifluoromethyl, NR¹⁰R¹¹, N(O)R¹⁰R¹¹, NR¹⁰R¹¹R¹²Y, NR¹⁰COR¹⁰, NR¹⁰CONR¹⁰R¹¹, NR¹⁰CON(O)R¹⁰R¹¹, NR¹⁰CONR¹⁰R¹¹R¹²Y, NR¹⁰CO₂R¹⁰, NR¹⁰C(O)SR¹⁰, NR¹⁰SO₂R¹⁰, NR¹⁰SO₂NR¹⁰R¹¹, nitro, OR¹⁰, OCF₃, aryloxy, heteroaryloxy, SR¹⁰, S(O)R¹⁰, S(O)₂R¹⁰, SCF₃, S(O)CF₃, S(O)₂CF₃, SO₂NR¹⁰R¹¹, SO₃R¹⁰, PO₃R¹⁰R¹¹, and halo,

wherein C₁₋₁₀ alkyl, C₁₋₁₀ alkanoyl, C₂₋₁₀, alkenyl, C₂₋₁₀ alkynyl, C₃₋₁₀ cycloalkyl, C₀₋₆ alkylaryl, C₀₋₆ alkylheterocyclyl, C₀₋₆ alkylheteroaryl may be optionally substituted by one or more of C(═NH)R¹⁰, COR¹⁰, CONR¹⁰R¹¹, CON(O)R¹⁰R¹¹, CONR¹⁰R¹¹R¹²Y, CO₂R¹⁰, C(O)SR¹⁰, C(S)R¹⁰, cyano, trifluoromethyl, NR10R¹¹, N(O)R¹⁰R¹¹, NR¹⁰R¹¹R¹²Y, NR¹⁰COR¹⁰, NR¹⁰CONR¹⁰R¹¹, NR¹⁰CON(O)R¹⁰R¹¹, NR¹⁰CONR¹⁰R¹¹R¹²Y, NR¹⁰CO₂R¹⁰, NR¹⁰C(O)SR¹⁰, NR¹⁰SO₂R¹⁰, NR¹⁰SO₂NR¹⁰R¹¹, nitro, OR¹⁰, OCF₃, aryloxy, heteroaryloxy, SR¹⁰, S(O)R¹⁰, S(O)₂R¹⁰, SCF₃, S(O)CF₃, S(O)₂CF₃, SO₂NR¹⁰R¹¹, SO₃R¹⁰, PO₃R¹⁰R¹¹, or halo, and on any position;

R¹⁰, R¹¹, and R¹² are, independently, selected from the group consisting of hydrogen, C₁₋₁₀ alkyl, C₁₋₁₀ alkanoyl, C₂₋₁₀ alkenyl, C₂₋₁₀ alkynyl, C₃₋₁₀ cycloalkyl, C₀₋₆ alkylaryl, C₀₋₆ alkylheterocyclyl, and C₀₋₆ alkylheteroaryl, or R¹⁰ and R¹¹, taken together with the nitrogen to which they are attached, forms a ring having 3 to 7 carbon atoms optionally containing 1, 2, or 3 heteroatoms selected from nitrogen, sulfur, oxygen, or nitrogen, substituted with hydrogen, C₁₋₄ alkyl or (CH₂)₀₋₃aryl; or a pharmaceutically acceptable inorganic or organic salt, esters, or other prodrug of said compound.

ii) A compound of formula: 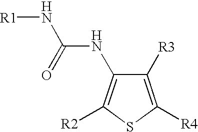
wherein:

R1 is selected from the group consisting of H₂ C₁₋₂ alkyl, XH, XCH₃, C₁₋₂ alkyl-XH, C₁₋₂ alkyl-XCH₃, C(O)NH₂, C(O)NHCH₃, and C(O)—C₁₋₂ alkyl,

provided that when R1 is H, R2 is not CONH₂, or provided that when R1 is C₁₋₂ alkyl, R2 is not CONH₂; with the preferred substitution being H or CH₃; X is selected from the group consisting of O, S, and NH;

R2 is selected from the group consisting of C(O)R⁵, CO₂R⁵, C(O)NHR⁵, OC(O)NHC(═NH)R⁵, C(O)NHC(═NH)NR⁵R⁶, C(O)NHC(O)R⁵, C(O)NHC(O)NR⁵R⁶, SO₂R⁵, S(O)R⁵, SO₃R⁵, and PO₃R⁵R⁶; R⁵ and R⁶ are, independently, selected from the group consisting of

hydrogen, C₁₋₁₀ alkyl, C₁₋₁₀ alkanoyl, C₂₋₁₀ alkenyl, C₂₋₁₀ alkynyl, C₃₋₁₀ cycloalkyl, C₀₋₆ alkylaryl, C₀₋₆ alkylheterocyclyl, and C₀₋₆ alkylheteroaryl, or R⁵ and R⁶, taken together with the nitrogen to which they are attached, may optionally form a ring having 3 to 7-carbon atoms optionally containing 1, 2, or 3 heteroatoms selected from nitrogen, sulfur, oxygen, or nitrogen, substituted with hydrogen, C₁₋₆ alkyl or (CH₂)₀₋₃aryl, wherein any of the foregoing may be optionally substituted by one or more of group A and on any position;

R³ is H or halogen; with the preferred substitution being H;

R⁴ is aryl or heteroaryl optionally substituted by one or more of group A and on any position, provided that when R1 is CH₃ and R2 is CO₂R⁵, R4 is not phenyl, or provided that when R1 is H, R4 is not 4-pyridyl;

A is selected from the group consisting of C₁₋₁₀ alkyl, C₁₋₁₀ alkanoyl, C₂₋₁₀ alkenyl, C₂₋₁₀ alkynyl, C₃₋₁₀ cycloalkyl, C₀₋₆ alkylaryl, C₀₋₆ alkylheterocyclyl, C₀₋₆ alkylheteroaryl, C(═NH)R⁷, COR⁷, CONR⁷R⁸, CON(O)R⁷R⁸, CONR⁷R⁸R⁹Y, CO₂R⁷, C(O)SR⁷, C(S)R⁷, cyano, trifluoromethyl, NR⁷R⁸, N(O)R⁷R⁸, NR⁷R⁸R⁹Y, NR⁷COR⁷, NR⁷CONR⁷R⁸, NR⁷CON(O)R⁷R⁸, NR⁷CONR⁷R⁸R⁹Y, NR⁷CO₂R⁷, NR⁷C(O)SR⁷, NR⁷SO₂R⁷, NR⁷SO2NR⁷R⁸, nitro, OR⁷, OCF₃, aryloxy, heteroaryloxy, SR⁷, S(O)R⁷, S(O)₂R⁷, SCF₃, S(O)CF₃, S(O)₂CF₃, SO₂NR⁷R⁸, SO₃R⁷, PO₃R⁷R⁸, and halo,

wherein C₁₋₁₀ alkyl, C₁₋₁₀ alkanoyl C₂₋₁₀ alkenyl, C₂₋₁₀ alkynyl, C₃₋₁₀ cycloalkyl, C₀₋₆ alkylaryl, C₀₋₆ alkylheterocyclyl, C₀₋₆ alkylheteroaryl, (CH₂)₀₋₆heteroaryl, aryloxy, and heteroaryloxy may be optionally substituted by one or more of group D and on any position;

Y is an organic or inorganic anion;

D is selected from the group consisting of C₁₋₁₀ alkyl, C₁₋₁₀ alkanoyl, C₂₋₁₀ alkenyl, C₂₋₁₀ alkynyl, C₃₋₁₀ cycloalkyl, C₀₋₆ alkylaryl, C₀₋₆ alkylheterocyclyl, C₀₋₆ alkylheteroaryl, C(═NH)R⁷, COR⁷, CONR⁷R⁸, CON(O)R⁷R⁸, CONR⁷R⁸R⁹Y, CO₂R⁷, C(O)SR⁷, C(S)R⁷, cyano, trifluoromethyl, NR⁷R⁸, N(O)R⁷R⁸, NR⁷R⁸R⁹Y, NR⁷COR⁷, NR⁷CONR⁷R⁸, NR⁷CON(O)R⁷R⁸, NR⁷CONR⁷R⁸R⁹Y, NR⁷CO₂R⁷, NR⁷C(O)SR⁷, NR⁷SO₂R⁷, NR⁷SO₂NR⁷R⁸, nitro, OR⁷, OCF₃, aryloxy, heteroaryloxy, SR⁷, S(O)R⁷, S(O)₂R⁷, SCF₃, S(O)CF₃, S(O)₂CF₃, SO₂NR⁷R⁸, SO₃R⁷, PO₃R⁷R⁸, and halo,

wherein C₁₋₁₀ alkyl, C₁₋₁₀ alkanoyl, C₂₋₁₀ alkenyl, C₂₋₁₀ alkynyl, C₃₋₁₀ cycloalkyl, C₀₋₆ alkylaryl, C₀₋₆ alkylheterocyclyl, C₀₋₆ alkylheteroaryl, (CH₂)₀₋₆heteroaryl, aryloxy, and heteroaryloxy may be optionally substituted by one or more of group E and on any position;

R⁷, R⁸, and R⁹ are, independently, selected from the group consisting of hydrogen, C₁₋₁₀ alkyl, C₁₋₁₀ alkanoyl, C₂₋₁₀ alkenyl, C₂₋₁₀ alkynyl, C₃₋₁₀ cycloalkyl, C₀₋₆ alkylaryl, C₀₋₆ alkylheterocyclyl, and C₀₋₆ alkylheteroaryl,

or R⁷ and R⁸, taken together with the nitrogen to which they are attached, may optionally form a ring having 3 to 7 carbon atoms, optionally containing 1, 2, or 3 heteroatoms selected from nitrogen, sulfur, oxygen, or nitrogen, substituted with hydrogen, C₁₋₆ alkyl or (CH₂)₀₋₃ aryl, wherein any of the foregoing may be optionally substituted by one or more of group E and on any position;

E is selected from the group consisting of C₁₋₁₀ alkyl, C₁₋₁₀ alkanoyl, C₂₋₁₀ alkenyl, C₂₋₁₀ alkynyl, C₃₋₁₀ cycloalkyl, C₀₋₆ alkylaryl, C₀₋₆ alkylheterocyclyl, C₀₋₆ alkylheteroaryl, C(═NH)R¹⁰, COR¹⁰, CONR¹⁰R¹¹, CON(O)R¹⁰R¹¹, CONR¹⁰R¹¹R¹²Y, CO₂R¹⁰, C(O)SR¹⁰, C(S)R¹⁰, cyano, trifluoromethyl, NR¹⁰R¹¹, N(O)R¹⁰R¹¹, NR¹⁰R¹¹R¹²Y, NR¹⁰COR¹⁰, NR¹⁰CONR¹⁰R¹¹, NR¹⁰CON(O)R¹⁰R¹¹, NR¹⁰CONR¹⁰R¹¹R¹²Y, NR¹⁰CO₂R¹⁰, NR¹⁰C(O)SR¹⁰, NR¹⁰SO₂R¹⁰, NR¹⁰SO₂NR¹⁰R¹¹, nitro, OR¹⁰, OCF₃, aryloxy, heteroaryloxy, SR¹⁰, S(O)R¹⁰, S(O)₂R¹⁰, SCF₃, S(O)CF₃, S(O)₂CF₃, SO₂NR¹⁰R¹¹, SO₃R¹⁰, PO₃R¹⁰R¹¹, and halo, wherein C₁₋₁₀ alkyl, C₁₋₁₀ alkanoyl, C₂₋₁₀ alkenyl, C₂₋₁₀ alkynyl, C₃₋₁₀ cycloalkyl, C₀₋₆ alkylaryl, C₀₋₆ alkylheterocyclyl, C₀₋₆ alkylheteroaryl may be optionally substituted by one or more of

C(═NH)R¹⁰, COR¹⁰, CONR¹⁰R¹¹, CON(O)R¹⁰R¹¹, CONR¹⁰R¹¹R¹²Y, CO₂R¹⁰, C(O)SR¹⁰, C(S)R¹⁰, cyano, trifluoromethyl, NR¹⁰R¹¹, N(O)R¹⁰R¹¹, NR¹⁰R¹¹R¹²Y, NR¹⁰COR¹⁰, NR¹⁰CONR¹⁰R¹¹, NR¹⁰CON(O)R¹⁰, R¹¹, NR¹⁰CONR¹⁰R¹¹R¹²Y, NR¹⁰CO₂R¹⁰, NR¹⁰C(O)SR¹⁰, NR¹⁰SO₂R¹⁰, NR¹⁰SO₂NR¹⁰R11, nitro, OR¹⁰, OCF₃, aryloxy, heteroaryloxy, SR¹⁰, S(O)R¹⁰, S(O)₂R¹⁰, SCF₃, S(O)CF₃, S(O)₂CF₃, SO₂NR¹⁰R¹¹, SO₃R¹⁰, PO₃R¹⁰R¹¹, or halo, and on any position;

R¹⁰, R¹¹, and R¹² are, independently, selected from the group consisting of hydrogen, C₁₋₁₀ alkyl, C₁₋₁₀ alkanoyl, C₂₋₁₀ alkenyl, C₂₋₁₀ alkynyl, C₃₋₁₀ cycloalkyl, C₀₋₆ alkylaryl, C₀₋₆ alkylheterocyclyl, and C₀₋₆ alkylheteroaryl;

or R¹⁰ and R¹¹, taken together with the nitrogen to which they are attached, forms a ring having 3 to 7 carbon atoms optionally containing 1, 2, or 3 heteroatoms selected from nitrogen, sulfur, oxygen, or nitrogen, substituted with hydrogen, C₁₋₆ alkyl or (CH₂)₀₋₃aryl; or, a pharmaceutically acceptable inorganic or organic salt, esters, or other prodrug of said compound.

XI. Heterocyclic Compounds as Described in US Patent Publication 2003199511, Including:

i) A compound of formula:

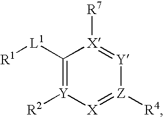

or a therapeutically acceptable salt thereof, wherein

X is selected from the group consisting of C(R⁸) and N; wherein R⁸ is selected from the group consisting of hydrogen, alkyl, amino, carboxy, cyano, halo, hydroxy, and amido;

X′ is selected from the group consisting of C and N;

Y is selected from the group consisting of C and N;

Y′ is selected from the group consisting of C(R⁹) and N; wherein R⁹ is selected from the group consisting of hydrogen and -L²-L³ (R³)(R⁶);

Z is selected from the group consisting of C and N; provided that 0, 1, or 2 of X, X′, Y, Y′, and Z are N;

L¹ is selected from the group consisting of a bond, —O—, —NR⁵, alkenyl, alkynyl, —C(O)—, —S—, —S(O)—, —S(O)₂—, —S(O)₂N(R)⁵—, —N(R⁵)S(O)₂—, —C(R¹²)₂—, —C(R¹²)₂N(R⁵)—, —N(R⁵)C(O)—, and —C(O)N(R⁵)—; wherein each group is drawn with its left end attached to R¹ and its right end attached to the aromatic ring;

L² is selected from the group consisting of a bond, —O—, —C(R¹²)₂—, —S—, —N(R⁵)—, —N(R⁵)C(O)—, and —C(O)N(R⁵)—;

L³ is selected from the group consisting of a bond, alkylidene and alkylene, wherein the alkylidene and the alkylene are optionally substituted with one or two substituents independently selected from the group consisting of alkoxy, amino, cyano, and hydroxy;

R¹ is selected from the group consisting of aryl, heteroaryl, and heterocycle;

R² and R⁴ are independently absent or selected from the group consisting of hydrogen, alkenyl, alkyl, alkynyl, amino, aryl, arylalkynyl, cyano, cyanoalkenyl, halo, heteroaryl, heterocycle, hydroxyalkyl, and nitro; or

R² and L¹, together with the carbon atoms to which they are attached, form a ring selected from the group consisting of aryl, heteroaryl, and heterocycle; or

R⁴ and L², together with the carbon atoms to which they are attached, form a ring selected from the group consisting of aryl, heteroaryl, and heterocycle;

provided that when L³ is alkylidene, R⁴ and L², together with the carbon atoms to which they are attached, form a ring selected from the group consisting of aryl, heteroaryl, and heterocycle;

R³ is absent or selected from the group consisting of hydrogen, aryl, arylalkoxy, arylalkylamino, arylalkylthio, aryloxy, arylthio, cycloalkyl, heteroaryl, heteroarylalkoxy, heteroaryloxy, and heterocycle;

R⁶ is selected from the group consisting of hydrogen, aryl, arylalkoxy, arylalkylamino, arylalkylthio, aryloxy, arylthio, cycloalkyl, heteroaryl, heteroarylalkoxy heteroaryloxy, and heterocycle; provided that when L¹ and L² are bonds, at least one of R³ and R⁶ is other than hydrogen;

R⁵ is selected from the group consisting of hydrogen, alkyl, alkylcarbonyl, alkylsulfonyl, arylcarbonyl, arylsulfonyl, and heteroarylsulfonyl;

R⁷ is absent or selected from the group consisting of hydrogen, alkyl, cyanoalkenyl, and -L²-L3(R³)(R⁶); or

R⁷ and L¹, together with the carbon atoms to which they are attached, form a ring selected from the group consisting of aryl, heteroaryl, and heterocycle; and

Each R¹² is selected from the group consisting of hydrogen, alkenyl, alkyl, alkynyl, amino, aryl, cyano, halo, heteroaryl, heterocycle, and nitro.

ii) A compound of formula:

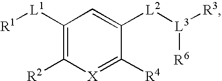

or a therapeutically acceptable salt thereof, wherein

L¹ is selected from the group consisting of a bond, —O—, —N(R⁵)—, alkenyl, alkynyl, —N(R⁵)C(O)—, and —C(O)N(R⁵)—;

L² is selected from the group consisting of a bond, —O—, —N(R⁵)—, —N(R⁵)C(O)—, and —C(O)N(R⁵)—;

L³ is selected from the group consisting of a bond, alkylidene, and alkylene, wherein the alkylidene and the alkylene are optionally substituted with one or two substituents independently selected from the group consisting of amino, cyano, and hydroxy;

R¹ is selected from the group consisting of aryl, heteroaryl, and heterocycle;

R² and R⁴ are independently selected from the group consisting of hydrogen, alkenyl, alkynyl, arylalkynyl, amino, cyano, cyanoalkenyl, halo, hydroxyalkyl, and heteroaryl; wherein the heteroaryl is selected from the group; consisting of furyl, pyrazinyl, thiazolyl, and thienyl; or

R² and L¹, together with the carbon atoms to which they are attached, form a ring selected from the group consisting of dihydropyrrolyl, pyrazolyl, and phenyl; or

R⁴ and L², together with the carbon atoms to which they are attached, form a ring selected from the group consisting of dihydropyrrolyl, phenyl, pyridinyl, and pyrrolyl; wherein the ring can be optionally substituted with oxo,

provided that when L³ is alkylidene, R⁴ and L², together with the carbon atoms to which they are attached, form a ring selected from the group consisting of dihydropyrrolyl, phenyl, pyridinyl, and pyrrolyl; wherein the ring can be optionally substituted with oxo;

R³ is absent or selected from the group consisting of hydrogen, aryl, arylalkoxy, arylalkylthio, aryloxy, arylthio, cycloalkyl, heteroaryl, heteroarylalkoxy, heteroaryloxy, and heterocycle;

R⁶ are independently selected from the group consisting of hydrogen, aryl, arylalkoxy, arylalkylthio, aryloxy, arylthio, cycloalkyl, heteroaryl, and heteroarylalkoxy, heteroaryloxy, and heterocycle; provided that when L¹ and L² are bonds, at least one of R³ and R⁶ is other than hydrogen;

R⁵ is selected from the group consisting of hydrogen, alkyl, alkylcarbonyl, alkylsulfonyl, arylcarbonyl, arylsulfonyl, and heteroarylsulfonyl; and

X is selected from the group consisting of C(R⁸) and N;

wherein R⁸ is selected from the group consisting of hydrogen, amino, carboxy, cyano, and halo.

iii) A compound of formula:

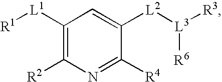

or a therapeutically acceptable salt thereof, wherein

L¹ is selected from the group consisting of a bond, —O—, —N(R⁵)—, alkenyl, alkynyl, and —N(R⁵)C(O)—;

L² is selected from the group consisting of a bond, —O—, —N(R⁵)—, —N(R⁵)C(O)—, and —C(O)N(R⁵)—;

L³ is alkylene, wherein the alkylene is substituted with one or two substituents independently selected from the group consisting of amino and hydroxy;

R¹ is selected from the group consisting of aryl, heteroaryl, and heterocycle;

R² and R⁴ are independently selected from the group consisting of hydrogen and halo;

R³ and R⁶ are independently selected from the group consisting of hydrogen, aryl, arylalkoxy, and heteroaryl; provided that when L¹ and L² are bonds, at least one of R³ and R⁶ is other than hydrogen; and

R⁵ is selected from the group consisting of hydrogen and alkyl.

XII. Heterocyclic Compounds as described in U.S. Patent Publication US2003162785, Including:

i) A compound of formula:

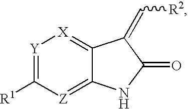

or a therapeutically acceptable salt thereof,

wherein X is selected from the group consisting of —N— and —CR^{x}—;

Y is selected from the group consisting of —N— and —CR^{y}—;

Z is selected from the group consisting of —N— and —CR^{z}—; with the proviso that at least one of Y and Z is other than —N—; one of R^{x}, R^{y}, R^{z}, and R¹ is selected from the group consisting of aryl and heterocycle and the others are hydrogen; and

R² is selected from the group consisting of heterocycle and aryl; with the proviso that when R² is heterocycle the heterocycle is other than imidazolyl.

XIII. N-pyrrolopyridinyl Compounds as Described in International Patent Publication WO03028724, Including:

i) A compound of formula: 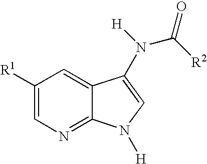
wherein:

R¹ is aryl or heteroaryl,

wherein aryl or heteroaryl may optionally be substituted by one or more of group A and on any position with the exception that R¹ is not 3,4-dichlorophenyl,

A is selected from the group consisting of C₁₋₁₀ alkyl, C₁₋₁₀ alkanoyl, C₂₋₁₀ alkenyl, C₂₋₁₀ alkynyl, C₃₋₁₀ cycloalkyl, C₀₋₆ alkylaryl, C₀₋₆ alkylheterocyclyl, C₀₋₆ alkylheteroaryl, C(═NH)R³, COR³, CONR³R⁴, CON(O)R³R⁴, CO₂R³, C(O)SR³, C(S)R³, cyano, trifluoromethyl, NR³R⁴, N(O)R³R⁴, NR³COR⁴, NR³CONR⁴R⁵, NR³CON(O)R⁴R⁵, NR³CO₂R³, NR³C(O)SR³, NR³SO₂R³, nitro, OR³, OCF₃, aryloxy, heteroaryloxy, SR³, S(O)R³, S(O)₂R³, SCF₃, S(O)CF₃, S(O)₂CF₃, SO₂NR³R⁴, SO₃R³, PO₃R³R⁴, and halo,

wherein C₁₋₁₀ alkyl, C₁₋₁₀ alkanoyl, C₂₋₁₀ alkenyl, C₂₋₁₀ alkynyl, C₃₋₁₀ cycloalkyl, C₀₋₅ alkylaryl, C₀₋₅ alkylheterocyclyl, C₀₋₅ alkylheteroaryl, (CH₂)₀₅ heteroaryl, aryloxy, and heteroaryloxy may be optionally substituted by one or more of group B and on any position;

B is selected from the group consisting of C₁₋₁₀ alkyl, C₁₋₁₀ alkanoyl, C₂₋₁₀ alkenyl, C₂₋₁₀ alkynyl, C₃₋₁₀ cycloalkyl, C₀₋₅ alkylaryl, C₀₋₅ alkylheterocyclyl, C₀₋₆alkylheteroaryl, C(═NH)R³, COR³, CONR³R⁴, CON(O)R³R⁴, CO₂R³, C(O)SR³, C(S)R³, cyano, trifluoromethyl, NR³R⁴, N(O)R³R⁴, NR³COR⁴, NR³CONR⁴R⁵, NR³CON(O)R⁴R⁵, NR³CO₂R³, NR³C(O)SR³, NR³SO₂R³, nitro, OR³, OCF₃, aryloxy, heteroaryloxy, SR³, S(O)R³, S(O)₂R³, SCF₃, S(O)CF₃, S(O)₂CF₃, SO₂NR³R⁴, SO₃R³, PO₃R³R⁴, and halo,

wherein C₁₋₁₀ alkyl, C₁₋₁₀ alkanoyl, C₂₋₁₀ alkenyl, C₂₋₁₀ alkynyl, C₃₋₁₀ cycloalkyl, C₀₋₆ alkylaryl, C₀₋₆ alkylheterocyclyl, C₀₋₆ alkylheteroaryl, (CH₂)₀₋₆heteroaryl, aryloxy, and heteroaryloxy may be optionally substituted by one or more of group C and on any position;

R³, R⁴, and R⁵ are independently selected from the group consisting of hydrogen, C₁₋₁₀ alkyl, C₁₋₁₀ alkanoyl, C₂₋₁₀ alkenyl, C₂₋₁₀ alkynyl, C₃₋₁₀ cycloalkyl, C₀₋₆ alkylaryl, C₀₋₆ alkylheterocyclyl, and C₀₋₆ alkylheteroaryl;

or R³ and R⁴ taken together with the nitrogen to which they are attached form a ring having 3 to 7 carbon atoms optionally containing 1, 2, or 3 heteroatoms selected from nitrogen, sulfur, oxygen, or nitrogen, substituted with hydrogen, C₁₋₆ alkyl or (CH₂)₀₋₃aryl, wherein any of the foregoing may be optionally substituted by one or more of group C and on any position;

C is selected from the group consisting of C₁₋₁₀ alkyl, C₁₋₁₀ alkanoyl, C₂₋₁₀ alkenyl, C₂₋₁₀ alkynyl, C₃₋₁₀ cycloalkyl, C₀₋₆ alkylaryl, C₀₋₆ alkylheterocyclyl, C₀₋₆ alkylheteroaryl, C(═NH)R⁶, COR⁶, CONR⁶R⁷, CON(O)R⁶R⁷, CO₂R⁶, C(O)SR⁶, C(S)R⁶, cyano, trifluoromethyl, NR⁶R⁷, N(O)R⁶R⁷, NR⁶COR⁶, NR⁶CONR⁷R⁸, NR⁶CON(O)R⁷R⁸, NR⁶CO₂R⁶, NR⁶C(O)SR⁶, NR⁶SO₂R⁶, nitro, OR⁶, OCF₃, aryloxy, heteroaryloxy, SR⁶, S(O)R⁶, S(O)₂R⁶, SCF₃, SOCF₃, S(O)₂CF₃, SO₂NR⁶R⁷, SO₃R⁶, PO₃R⁶R⁷, and halo, wherein C₁₋₈alkyl, C₁₋₈ alkanoyl, C₂₋₈ alkenyl, C₂₋₈ alkynyl, C₃₋₈ cycloalkyl, C₀₋₆ alkylaryl, C₀₋₆ alkylheterocyclyl, C₀₋₆ alkylheteroaryl may be optionally substituted by one or more of

C(═NH)R⁶, COR⁶, CONR⁶R⁷, CON(O)R⁶R⁷, CO₂R⁶, C(O)SR⁶, C(S)R⁶, cyano, trifluoromethyl, NR⁶R⁷, N(O)R⁶R⁷, NR⁶COR⁶, NR⁶CONR⁷R⁸, NR⁶CON(O)R⁷R⁸, NR⁶CONR⁶R⁷R⁸Y, NR⁶CO₂R⁶, NR⁶C(O)SR⁶, NR⁶SO₂R⁶, nitro, OR⁶, aryloxy, heteroaryloxy, SR⁶, S(O)R⁶, S(O)₂R⁶, SO₂NR⁶R⁷, SO₃R⁶, PO₃R⁶R⁷, or halo, and on any position;

R⁶, R⁷, and R⁸ are independently selected from the group consisting of hydrogen, C₁₋₁₀ alkyl, C₁₋₁₀ alkanoyl, C₂₋₁₀ alkenyl, C₂₋₁₀ alkynyl, C₃₋₁₀ cycloalkyl, C₀₋₆ alkylaryl, C₀₋₆ alkylheterocyclyl, and C₀₋₆ alkylheteroaryl;

or R⁷ and R⁵ taken together with the nitrogen to which they are attached form a ring having 3 to 7 carbon atoms optionally containing 1, 2, or 3 heteroatoms selected from nitrogen, sulfur, oxygen, or nitrogen, substituted with, hydrogen, C₁₋₆ alkyl or (CH₂)₀₋₃aryl;

R² is selected from the group consisting of C₁₋₈ alkyl, C₂₋₈ alkenyl, C₃₋₆ cycloalkyl, OR⁹, NR¹⁰R¹¹, phenyl, pyridyl, pyridazinyl, pyrimidinyl, pyrazolinyl, thiazinyl, pyrrolyl, furyl, thienyl, pyrazolyl, imidazolyl, triazolyl, oxazolyl, isoxazolyl, thiazolyl, isothiazolyl and thiadiazolyl,

wherein alkyl and alkenyl and cycloalkyl may optionally be substituted with one of more of group D and at any position and wherein phenyl may be optionally substituted at positions 3-, 4, and 5-with one to three of group E and

wherein pyridyl, pyridazinyl, pyrimidinyl, pyrazolinyl, thiazinyl, pyrrolyl, furyl, thienyl, pyrazolyl, imidazolyl, triazolyl, oxazolyl, isoxazolyl, thiazolyl, isothiazolyl and thiadiazolyl may optionally be substituted by one or more of group F and at any position,

with the preferred substitution being n-propyl or pyridyl or pyrazolinyl, with the more preferred substitution being 3-pyridyl

R⁹ is hydrogen or C₁₋₆ alkyl, wherein any of the foregoing groups are optionally substituted with one or more of group D and at any position, with the exception that R⁹ is not tert-butyl;

R¹⁰ is selected from the group consisting of hydrogen, methyl and ethyl; R¹¹ is selected from the group consisting of hydrogen, C₁₋₆ alkyl, C₂₋₈ alkenyl and C₃₋₆ cycloalkyl,

wherein any of the foregoing groups are optionally substituted with one or more of group D and at any position;

R¹⁰, and R¹¹ taken together with the nitrogen to which they are attached may form a ring having 3 to 7 carbon atoms optionally containing 1, 2, or 3 heteroatoms selected from nitrogen, sulfur, oxygen, or nitrogen, substituted with hydrogen or C₁₋₆ alkyl;

D is selected from the group consisting of C₁₋₆ alkyl, C₂₋₈ alkenyl, C₃₋₆ cycloalkyl, OR¹², OC(O)NR¹²R¹³, NR¹⁴SO₂R¹²R¹³, NR¹⁴C(O)OR¹², NR¹⁴C(O)NR¹²R¹³, halo, cyano, trifluoromethyl, SR¹², S(O)R¹², SO₂R¹², SO₃R¹², SO₂NR¹²R¹³, C(O)SR¹², CONR¹²R¹³ and PO₃R¹²;

R¹², R¹³, R¹⁴ are independently selected from the group consisting of hydrogen, C₁₋₃ alkyl, C₂₋₃ alkanoyl, C₂₋₃ alkenyl, C₂₋₃ alkynyl, and C₃₋₅ cycloalkyl; or R¹² and R¹³ taken together with the nitrogen to which they are attached form a ring having 3 to 7 carbon atoms optionally containing 1, 2, or 3 heteroatoms selected from nitrogen, sulfur, oxygen, or nitrogen, substituted with hydrogen or C₁₋₃ alkyl;

E is selected from the group consisting of C₁₋₄ alkyl, o R¹⁵ and NR¹⁵R¹⁶, with the exception that R² is not 3, 4-dimethoxyphenyl or 3-methoxyphenyl,

F is selected from the group consisting of C₁₋₆ alkyl, C₂₋₈ alkenyl, C₃₋₆ cycloalkyl, OR¹², OC(O)NR¹²R¹³, NR²²R¹³, NR¹⁴SO₂R¹²R¹³, NR¹⁴C(O)OR¹², NR¹⁴C(O)NR¹²R¹³, halo, cyano, trifluoromethyl, SR¹², S(O)R¹², SO₂R¹², SO₃R¹², SO₂NR¹²R¹³, C(O)SR¹², CONR¹²R¹³ and PO₃R¹²;

R¹⁵ and R¹⁶ are independently selected from the group consisting of hydrogen, C₁₋₃ alkyl C₂₋₃ alkanoyl, C₂₋₃ alkenyl, C₂₋₃ alkynyl, and C₃₋₅ cycloalkyl; or R¹⁵ and R¹⁶ taken together with the nitrogen to which they are attached form a ring having 3 to 7 carbon atoms optionally containing 1, 2, or 3 heteroatoms selected from nitrogen, sulfur, oxygen, or nitrogen, substituted with hydrogen or C₁₋₃ alkyl.

IX. Indazolyl Compounds as Described in International Patent Publication WO03004488, Including:

a compound having the structure below, a tautomer of the compound, a pharmaceutically acceptable salt of the compound, or a pharmaceutically acceptable salt of the tautomer: 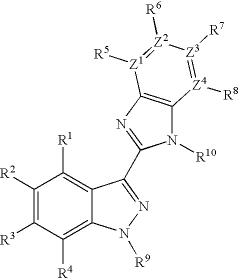
wherein

Z¹, Z², Z³, and Z⁴ are independently selected from C or N;

R¹ is selected from the group consisting of —H, —F, —Cl, and —Br; R² is selected from the group consisting of —H, —F, —Cl, —Br, —C≡N, —NO₂, —CO₂H, substituted and unsubstituted amino groups, substituted and unsubstituted alkyl groups, substituted and unsubstituted —C(═O)O-alkyl groups, substituted and unsubstituted —C(═O)O-aryl groups, substituted and unsubstituted —C(═O)O-heteroaryl groups, substituted and unsubstituted-C(═O)N(E)-alkyl groups, substituted and unsubstituted —C(═O)N(H)-aryl groups, substituted and unsubstituted —C(═O)N(H)-heterocyclyl groups, substituted and unsubstituted —N(H)C(═O)-alkyl groups, substituted and unsubstituted —N(H)C(═O)-aryl groups, substituted and unsubstituted —N(H)C(═O)-heterocyclyl groups, substituted and unsubstituted —N(H)C(═O)N(R)-alkyl groups, substituted and unsubstituted —N(H)C(═O)N(H)-aryl groups, substituted and unsubstituted —N(H)-heterocyclyl groups, substituted and unsubstituted alkoxy groups, substituted and unsubstituted arylalkoxy groups, and substituted and unsubstituted heterocyclylalkoxy groups;

R³ is selected from the group consisting of —H, —F, —Cl, —Br, and substituted and unsubstituted alkoxy groups; R⁴ is —H; R⁵ is selected from the group consisting of —H, —F, —Cl, substituted and unsubstituted alkyl groups, substituted and unsubstituted alkoxy groups, substituted and unsubstituted amino groups, substituted and unsubstituted alkylamino groups, substituted and unsubstituted dialkylamino groups, and substituted and unsubstituted heterocyclyl groups, or R⁵ is absent if Z¹ is N;

R⁶ is selected from the group consisting of —H, —F, —Cl, —Br, —CF₃, —CO₂H, substituted and unsubstituted alkyl groups, substituted and unsubstituted alkoxy groups including substituted and unsubstituted heterocyclylalkoxy groups, substituted and unsubstituted-arylalkoxy groups, and substituted and unsubstituted alkoxyalkoxy groups; substituted and unsubstituted heterocyclyl groups including substituted and unsubstituted heterocyclylheterocyclyl groups, substituted and unsubstituted arylheterocyclyl groups, and substituted and unsubstituted cycloalkylheterocyclyl groups; substituted and unsubstituted heterocyclyloxy groups, substituted and unsubstituted aryloxy groups, substituted and unsubstituted amino groups including substituted and unsubstituted dialkylamino groups, substituted and unsubstituted (alkyl)(heterocyclyl)amino groups, substituted and unsubstituted heterocyclylalkylamino groups, substituted and unsubstituted arylalkylamino groups, and substituted and unsubstituted heterocyclylamino groups; substituted and unsubstituted —C(═O)N(H)-alkyl groups, substituted and unsubstituted —C(═O)N(H)aryl groups, and substituted and unsubstituted —C(═O)N(H)-heterocyclyl groups; or R⁶ is absent if Z² is N;

R⁷ is selected from the group consisting of —H, —F, —Cl, —Br, —CF₃, —CO₂H, substituted and unsubstituted alkyl groups, substituted and unsubstituted alkoxy groups including substituted and unsubstituted heterocyclylalkoxy groups, substituted and unsubstituted arylalkoxy groups, and substituted and unsubstituted alkoxyalkoxy groups; substituted and unsubstituted heterocyclyl groups including substituted and unsubstituted heterocyclylheterocyclyl groups, substituted and unsubstituted arylheterocyclyl groups, and substituted and unsubstituted cycloalkylheterocyclyl groups; substituted and unsubstituted heterocyclyloxy groups, substituted and unsubstituted aryloxy groups, substituted and unsubstituted amino groups including substituted and unsubstituted dialkylamino groups, substituted and unsubstituted (alkyl)(heterocyclyl)amino groups, and substituted and unsubstituted heterocyclylalkylamino groups, substituted and unsubstituted arylalkylamino groups, substituted and unsubstituted heterocyclylamino groups; substituted and unsubstituted —C(═O)₇N(H)-alkyl groups, substituted and unsubstituted —C(—O)N(H)-aryl groups, and substituted and unsubstituted —C(═O)N(H)-heterocyclyl groups; or R⁷ is absent if Z³ is N;

R⁸ is selected from the group consisting of —H, —F, —Cl, substituted and unsubstituted alkyl groups, substituted and unsubstituted alkoxy groups, substituted and unsubstituted amino groups, substituted and unsubstituted alkylamino groups, substituted and unsubstituted dialkylamino groups, and substituted and unsubstituted heterocyclyl groups, or R⁸ is absent if Z⁴ is N;

R⁹ is —H; and

R¹⁰ is —H, and further wherein at least one of R¹, R², R³, R⁵, R⁶, R⁷ or R⁸ is not —H.

ii) A compound having the structure below, a tautomer of the compound, a pharmaceutically acceptable salt of the compound, or a pharmaceutically acceptable salt of the tautomer: 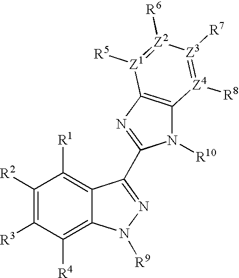
wherein

Z¹, Z², Z³, and Z⁴ are independently selected from C or N;

R¹ is selected from —H, —F, —Cl, —Br, —NO₂, —C≡N, —C(═O)—O-alkyl groups, —OH, substituted and unsubstituted arylalkoxy groups, substituted and unsubstituted heterocyclyloxy groups, substituted and unsubstituted alkoxyalkyl groups, substituted and unsubstituted arylalkoxyalkyl groups, substituted and unsubstituted aryloxy groups, substituted and unsubstituted —N(H)C(═O)-aryl groups, substituted and unsubstituted —N(H)—C(═O)-alkyl groups, substituted and ursubstituted —N(H)—SO₂-alkyl groups, substituted and unsubstituted —N(H)—SO₂-aryl groups; —N(H)—SO₂—CF₃ groups, substituted and unsubstituted —N(H)—SO₂-heterocyclyl groups, substituted and unsubstituted heterocyclyl groups, substituted and unsubstituted alkyl groups, substituted and unsubstituted alkoxy groups, substituted and unsubstituted amino groups, substituted and unsubstituted —C(═O)—N(H)-alkyl groups, substituted and unsubstituted —C(═O)—N—(H)-alkyl-heterocyclyl groups, substituted and unsubstituted (alkyl)(alkyl)aminoalkyl groups, substituted and unsubstituted (alkyl)(aryl)aminoalkyl groups, substituted and unsubstituted (alkyl) (heterocyclyl)aminoalkyl groups, substituted and unsubstituted (alkyl)(arylalkyl)aminoalkyl groups, substituted and unsubstituted (alkyl)(heterocyclylalkyl)aminoalkyl groups, substituted and unsubstituted -alkyl-N(alkyl)-C(═O)-alkyl groups, substituted and unsubstituted -alkyl-N(alkyl)-C(═O)aryl groups, substituted and unsubstituted -alkyl-N(alkyl)-C(═O)-heterocyclyl groups, substituted and unsubstituted -alkyl-N(alkyl)-C(═O)-alkyl-aryl groups, substituted and unsubstituted -alkyl-N(alkyl)-C(═P)-alkyl-heterocyclyl groups, substituted and unsubstituted alkylaminoalkyl groups, substituted and unsubstituted arylaminoalkyl groups, substituted and unsubstituted heterocyclylaminoalkyl groups, substituted and unsubstituted arylalkylaminoalkyl groups, substituted and unsubstituted heterocyclylalkylaminoalkyl groups, substituted and unsubstituted -alkyl-N(H)—C(═O)-alkyl groups, substituted and unsubstituted -alkyl-N(H)—C(═O)-aryl groups, substituted and unsubstituted -alkyl-N(H)—C(═O)-heterocyclyl groups, substituted and unsubstituted -alkyl-N(H)—C(═O)-alkyl-aryl groups, and substituted and unsubstituted -alkyl-N(H)—C(═O)-alkyl-heterocyclyl groups;

R² is selected from —H, —F, —Cl, —Br, —C≡N, —NO₂, —CO₂H, —OH, substituted and unsubstituted guanidinyl groups, substituted and unsubstituted amino groups, substituted and unsubstituted alkyl groups, substituted and unsubstituted —C(═O)O-alkyl groups, substituted and unsubstituted —C(═O)O-aryl groups, substituted and unsubstituted —C(═O)O-heteroaryl groups, substituted and unsubstituted —C(═O)N(H)-alkyl groups, substituted and unsubstituted —C(═O)—N(H)-alkyl-heterocyclyl groups, substituted and unsubstituted —C(O)N(H)-aryl groups, substituted and unsubstituted —C(═O)N(H)-heterocyclyl groups, substituted and, unsubstituted —N(H)C(═O)-alkyl groups, substituted and unsubstituted —N(H)C(═O)-aryl groups, substituted and unsubstituted —N(H)C(═O)-heterocyclyl groups, substituted and unsubstituted —N(H)C(—O)N(H)-alkyl groups, substituted and unsubstituted —N(H)C(═O)N(H)-aryl groups, substituted and unsubstituted —N(H)C(═O)N(H)-heterocyclyl groups, substituted and unsubstituted —N(H)—(SO₂)-alkyl groups, substituted and unsubstituted —N(H)—(SO₂)-aryl groups, —N(H)—(SO₂)—CF₃ groups, substituted and unsubstituted —N(H)—(SO₂)-heterocyclyl groups, substituted and unsubstituted —N(H)-heterocyclyl groups, substituted and unsubstituted heterocyclyl groups, substituted and unsubstituted alkoxy groups, substituted and unsubstituted arylalkoxy groups, substituted and unsubstituted aryloxy groups, substituted and unsubstituted alkoxyalkyl groups, substituted and unsubstituted arylalkoxyalkyl groups, substituted and unsubstituted heterocyclyloxy, substituted and unsubstituted heterocyclylalkoxy groups, substituted and unsubstituted (alkyl)(alkyl)aminoalkyl groups, substituted and unsubstituted (alkyl)(aryl) aminoalkyl groups, substituted and unsubstituted (alkyl)(heterocyclyl)aminoalkyl groups substituted and unsubstituted (alkyl)(arylalkyl)aminoalkyl groups, substituted and unsubstituted (alkyl)(heterocyclylalkyl)aminoalkyl groups, substituted and unsubstituted -alkyl-N(alkyl)-C(═O)-alkyl groups, substituted and unsubstituted -alkyl-N(alkyl)-C(═O)-aryl groups, substituted and unsubstituted -alkyl-N(alkyl)-C(═O)-heterocyclyl groups, substituted and unsubstituted -alkyl-N(alkyl)-C(═O)-alkyl-aryl groups, substituted and unsubstituted -alkyl-N(alkyl)-C(═O)-alkyl-heterocyclyl groups, substituted and unsubstituted alkylaminoalkyl groups, substituted and unsubstituted arylaminoalkyl groups, substituted and unsubstituted heterocyclylaminoalkyl groups substituted and unsubstituted arylalkylaminoalkyl groups, substituted and unsubstituted heterocyclylalkylaminoalkyl groups, substituted and unsubstituted -alkyl-N(H)—C(═O)-alkyl groups, substituted and unsubstituted -alkyl-N(H)—C(═O)-aryl groups, substituted and unsubstituted -alkyl-N(H)—C(═O)-heterocyclyl groups, substituted and unsubstituted -alkyl-N(H)—C(═O)-alkyl-aryl groups, and substituted and unsubstituted -alkyl-N(H)—C(═O)-alkyl-heterocyclyl groups; or R² and R³ are a group of formula —OCH₂O— such that R² and R³ define a fused 5-membered ring that includes 2 oxygen atoms;

R³ is selected from —H, —F, —Cl, —Br, —CF₃, —CN, substituted and unsubstituted alkyl groups, substituted and unsubstituted amino groups, substituted and unsubstituted alkoxy groups, substituted and unsubstituted —C(═O)—O-alkyl groups, substituted and unsubstituted arylalkoxy groups, substituted and unsubstituted saturated heterocyclyloxy groups, substituted and unsubstituted alkoxyalkyl groups, substituted and unsubstituted: arylalkoxyalkyl groups, substituted and unsubstituted saturated heterocycyl groups, substituted and unsubstituted —N(H)—C(═O)-alkyl groups, substituted and unsubstituted —N(H)—C(═O)-aryl groups, substituted and unsubstituted —N(H)—(SO₂)-alkyl groups substituted and unsubstituted —N(H)—(SO₂)-aryl groups, —N(H)—(SO₂)—CF₃ groups, substituted and unsubstituted —N(H)—(SO₂)-heterocyclyl groups, substituted and unsubstituted —N(H)C(═O)N(H)-alkyl groups, substituted and unsubstituted —N(H)C(═O)N(H)-aryl groups, substituted and unsubstituted (alkyl)(alkyl)aminoalkyl groups, substituted and unsubstituted (alkyl)(aryl)aminoalkyl groups, substituted and unsubstituted (alkyl) (heterocyclyl) aminoalkyl groups substituted and unsubstituted (alkyl) (arylalkyl)aminoalkyl groups, substituted and unsubstituted (alkyl) (heterocyclylalkyl)aminoalkyl groups, substituted and unsubstituted -alkyl-N(alkyl)-C(═O)-alkyl groups, substituted and unsubstituted-alkyl-N(alkyl)-C(═O)-aryl groups, substituted and unsubstituted alkyl-N(alkyl)-C(═O)-heterocyclyl groups, substituted and unsubstituted -alkyl-N(alkyl)-C(═O)-alkyl-aryl groups, and substituted and unsubstituted -alkyl-N(alkyl)-C(═O)-alkyl-heterocyclyl groups;

R⁴ is —H, —F, —Br, —Cl, —NO₂, —C≡N, —C(═O)—O-alkyl groups, —OH, substituted and unsubstituted arylalkoxy groups, substituted and unsubstituted heterocyclyloxy groups, substituted and unsubstituted alkoxyalkyl groups, substituted and unsubstituted arylalkoxyalkyl groups, substituted and unsubstituted aryloxy groups, substituted and unsubstituted —N(H)—C(═O)-aryl groups, substituted and unsubstituted —N(H)—C(═O)-alkyl groups, substituted and unsubstituted —N(H)—SO₂-alkyl groups, substituted and unsubstituted —N(H)—SO₂-aryl groups, —N(H)—SO₂—CF₃ groups, substituted and unsubstituted-N(H)—SO₂-heterocyclyl groups, substituted and unsubstituted heterocyclyl groups, substituted and unsubstituted amino groups, substituted and unsubstituted alkyl groups, substituted and unsubstituted alkoxy groups, substituted and unsubstituted-C(═O)—N(H)-alkyl groups, substituted and unsubstituted-C(═O)—N(H)-alkyl-heterocyclyl groups, substituted and unsubstituted (alkyl)(alkyl)aminoalkyl groups, substituted and unsubstituted (alkyl) (aryl)aminoalkyl groups, substituted and unsubstituted (alkyl)(heterocyclyl)aminoalkyl groups, substituted and unsubstituted (alkyl)(arylalkyl)aminoalkyl groups, substituted and unsubstituted (alkyl)(heterocyclylalkyl)aminoalkyl groups, substituted and unsubstituted -alkyl-N (alkyl)-C(═O)-alkyl groups, substituted and unsubstituted -alkyl-N(alkyl)C(═O)-aryl groups, substituted and unsubstituted-alkyl-N(alkyl)-C(═O)-heterocyclyl groups, substituted and unsubstituted -alkyl-N(alkyl)-C(═O)-alkyl-aryl groups, substituted and unsubstituted -alkyl-N(alkyl)-C(═O)-alkyl-heterocyclyl groups, substituted and unsubstituted alkylaminoalkyl groups, substituted and unsubstituted arylaminoalkyl groups, substituted and unsubstituted heterocyclylaminoalkyl groups substituted and unsubstituted arylalkylaminoalkyl groups, substituted and unsubstituted heterocyclylakylaminoalkyl groups, substituted and unsubstituted -alkyl-N(H)—C(═O)-alkyl groups, substituted and unsubstituted -alkyl-N(H)—C(═O)-aryl groups, substituted and unsubstituted -alkyl-N(H)—C(═O)-heterocyclyl groups, substituted and unsubstituted -alkyl-N(H)—C(═O)-alkyl-aryl groups, and substituted and unsubstituted -alkyl-N(H)—C(═O)-alkyl-heterocyclyl groups;

R⁵ is selected from —H, —F, —Cl, substituted and unsubstituted alkyl groups, substituted and unsubstituted alkoxy groups, substituted and unsubstituted amino groups, substituted and unsubstituted alkylamino groups, substituted and unsubstituted dialkylamino groups, and substituted and unsubstituted heterocyclyl groups, or R⁵ is absent if Z¹ is N;

R⁶ is selected from —H, —F, —Cl, —Br, —CF₃, —CO₂H, substituted and unsubstituted alkyl groups, substituted and unsubstituted alkoxy groups including substituted and unsubstituted heterocyclylalkoxy groups, substituted and unsubstituted arylalkoxy groups, and substituted and unsubstituted alkoxyalkoxy groups; substituted and unsubstituted heterocyclyl groups including substituted and unsubstituted heterocyclylheterocyclyl groups, substituted and unsubstituted arylheterocyclyl groups; substituted and unsubstituted alkylheterocyclyl groups, and substituted and unsubstituted cycloalkylheterocyclyl groups; substituted and unsubstituted heterocyclyloxy groups, substituted and unsubstituted aryloxy groups, substituted and unsubstituted amino groups including substituted and unsubstituted dialkylamino groups, substituted and unsubstituted (alkyl)(heterocyclyl)amino groups, substituted and unsubstituted heterocyclylalkylamino groups, substituted and unsubstituted arylalkylamino groups, and substituted and unsubstituted heterocyclylamino groups; substituted and unsubstituted —C(═O)N(H)-alkyl groups, substituted and unsubstituted —C(═O)N(H)-aryl groups, substituted and unsubstituted —C(═O)N(H)-heterocyclyl groups, substituted and unsubstituted —C(═O)N(alkyl)(heterocyclyl) groups, and substituted and unsubstituted —C(═O)-heterocyclyl groups; or R⁶ is absent if Z² is N;

R⁷ is selected from —H, —F, —Cl, —Br, —CF₃, —CO₂H, substituted and unsubstituted alkyl groups, substituted and unsubstituted alkoxy groups including substituted and unsubstituted heterocyclylalkoxy groups, substituted and unsubstituted arylalkoxy groups, and substituted and unsubstituted alkoxyalkoxy groups; substituted and unsubstituted heterocyclyl groups including substituted and unsubstituted heterocyclylheterocyclyl groups, substituted and unsubstituted arylheterocyclyl groups, substituted and unsubstituted alkylheterocyclyl groups, and substituted and unsubstituted cycloalkylheterocyclyl groups; substituted and unsubstituted heterocyclyl groups, substituted and unsubstituted aryloxy groups, substituted and unsubstituted amino groups including substituted and unsubstituted dialkylamino groups, substituted and unsubstituted (alkyl)(heterocyclyl) amino groups, substituted and unsubstituted heterocyclylalkylamino groups, substituted and unsubstituted arylalkylamino groups, and substituted and unsubstituted heterocyclylamino groups; substituted and unsubstituted —C(═O)N(H)-alkyl groups, substituted and unsubstituted —C(═O)N(H)-aryl groups, substituted and unsubstituted —C(═O)N(H)-heterocyclyl groups, substituted and unsubstituted —C(═O)N(alkyl)(heterocyclyl) groups, and substituted and unsubstituted —C(═O)-heterocyclyl groups; or R⁷ is absent if Z³ is N;

R⁸ is selected from —H, —F, —Cl, substituted and unsubstituted alkyl groups, substituted and unsubstituted alkoxy groups, substituted and unsubstituted amino groups, substituted and unsubstituted alkylamino groups, substituted and unsubstituted dialkylamino groups, and substituted and unsubstituted heterocyclyl groups, or R⁸ is absent if Z⁴ is N;

R⁹ is —H; and

R¹⁰ is selected from the group consisting of —H, and substituted and unsubstituted alkyl groups, and further wherein at least one of R¹, R², R³, R⁴, R⁵, R⁶, R⁷ or R⁸ is not —H.

iii) A compound having the structure below, a tautomer of the compound, a pharmaceutically acceptable salt of the compound, or a pharmaceutically acceptable salt of the tautomer: 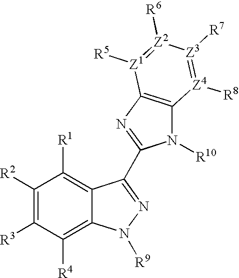
wherein

R¹ is selected from —H, —F, —Cl, —Br, —NO₂, —C≡N, —C(═O)—O-alkyl groups, —OH, substituted and unsubstituted arylalkoxy groups, substituted and unsubstituted heterocyclyloxy groups, substituted and unsubstituted alkoxyalkyl groups, substituted and unsubstituted arylalkoxyalkyl groups, substituted and unsubstituted aryloxy groups, substituted and unsubstituted —N(H)—C(═O)-aryl groups, substituted and unsubstituted —N(H)—C(═O)-alkyl groups, substituted and unsubstituted —N(H)—SO₂-alkyl groups, substituted and unsubstituted —N(H)—SO₂-aryl groups, —N(H)SO₂—CF₃ groups, substituted and unsubstituted —N(H)—SO₂-heterocyclyl groups, substituted and unsubstituted heterocyclyl groups, substituted and unsubstituted alkyl groups, substituted and unsubstituted alkoxy groups, substituted and unsubstituted amino groups, substituted and unsubstituted —C(═O)—N(H)-alkyl groups, substituted and unsubstituted —C(═O)—N(H)-alkyl-heterocyclyl groups, substituted and unsubstituted (alkyl)(alkyl)aminoalkyl groups, substituted and unsubstituted (alkyl)(aryl)aminoalkyl groups, substituted and unsubstituted (alkyl)(heterocyclyl)aminoalkyl groups substituted and unsubstituted (alkyl)(arylalkyl)-aminoalkyl groups, substituted and unsubstituted (alkyl)(heterocyclylalkyl)aminoalkyl groups, substituted and unsubstituted -alkyl-N(alkyl)-C(═O)-alkyl groups, substituted and unsubstituted -alkyl-N (alkyl)-C(═O)-aryl groups, substituted and unsubstituted -alkyl-N (alkyl)-C(═O)-heterocyclyl groups, substituted and unsubstituted -alkyl-N(H)—C(═O)-alkyl aryl groups, substituted and unsubstituted -alkyl-N(alkyl)-C(═O)-alkyl-heterocyclyl groups, substituted and unsubstituted alkylaminoalkyl groups, substituted and unsubstituted arylaminoalkyl groups, substituted and unsubstituted heterocyclylaminoalkyl groups substituted and unsubstituted arylalkylaminoalkyl groups, substituted and unsubstituted heterocyclylalkylaminoalkyl groups, substituted and unsubstituted -alkyl-N(H)—C(═O)-alkyl groups, substituted and unsubstituted -alkyl-N(H)—C(═O)-aryl groups, substituted and unsubstituted -alkyl-N(H)—C(═O)heterocyclyl groups, substituted and unsubstituted -alkyl-N(H)C(═O)-alkyl-aryl groups, and substituted and unsubstituted -alkyl-N(H)—C(═O)-alkyl-heterocyclyl groups;

R² is selected from —H, —F, —Cl, —Br, —C≡N, —NO₂, —CO₂H, —OH, substituted and unsubstituted guanidinyl groups, substituted and unsubstituted amino groups, substituted and unsubstituted alkyl groups, substituted and unsubstituted —C(═O)O-alkyl groups, substituted and unsubstituted —C(═O)O-aryl groups, substituted and unsubstituted —C(═O)O-heteroaryl groups, substituted and unsubstituted —C(═O)N(H)-alkyl groups, substituted and unsubstituted —C(—O)—N(H)-alkyl-heterocyclyl groups, substituted and unsubstituted —C(═O)N(H)-aryl groups, substituted and unsubstituted —C(═O)N(H)-heterocyclyl groups, substituted and unsubstituted —N(H)C(═O)-alkyl groups, substituted and unsubstituted —N(H)C(═O)-aryl groups, substituted and unsubstituted —N(H)C(═O)-heterocyclyl groups, substituted and unsubstituted —N(H)C(═O)N(H)-alkyl groups, substituted and unsubstituted —N(H)C(═O)N(H)-aryl groups, substituted and unsubstituted —N(H)C(—O)N(H)-heterocyclyl groups, substituted and unsubstituted —N(H)—(SO₂)-alkyl groups, substituted and unsubstituted —NR)—(SO₂)-aryl groups, —N(H)—(SO₂)—CF₃ groups, substituted and unsubstituted —N(H)—(SO₂)-heterocyclyl groups, substituted and unsubstituted —N(H)-heterocyclyl groups, substituted and unsubstituted heterocyclyl groups, substituted and unsubstituted alkoxy groups, substituted and unsubstituted arylalkoxy groups, substituted and unsubstituted aryloxy groups, substituted and unsubstituted alkoxyalkyl groups, substituted and unsubstituted arylalkoxyalkyl groups, substituted and unsubstituted heterocyclyloxy, substituted and unsubstituted heterocyclylalkoxy groups, substituted and unsubstituted (alkyl)(alkyl)aminoalkyl groups, substituted and unsubstituted (alkyl)(aryl)aminoalkyl groups, substituted and unsubstituted (alkyl)(heterocyclyl)aminoalkyl groups substituted and unsubstituted (alkyl)(arylalkyl)aminoalkyl groups, substituted and unsubstituted (alkyl)(heterocyclylalkyl)aminoalkyl groups, substituted and unsubstituted -alkyl-N(alkyl)-C(═O)-alkyl groups, substituted and unsubstituted -alkyl-N(alkyl)-C(═O)-aryl groups, substituted and unsubstituted -alkyl-N(alkyl)-C(═O)-heterocyclyl groups, substituted and unsubstituted -alkyl-N(alkyl)-C(═O)-alkyl-aryl groups, substituted and unsubstituted -alkyl-N(alkyl)-C(═O)-alkyl-heterocyclyl groups, substituted and unsubstituted alkylaminoalkyl groups, substituted and unsubstituted arylaminoalkyl groups, substituted and unsubstituted heterocyclylaminoalkyl groups substituted and unsubstituted arylalkylaminoalkyl groups, substituted and unsubstituted heterocyclylalkylaminoalkyl groups, substituted and unsubstituted alkyl-N(H)—C(═O)-alkyl groups, substituted and unsubstituted -alkyl-N(H)—C(═O)-aryl groups, substituted and unsubstituted -alkyl-N(H)—C(═O)-heterocyclyl groups, substituted and unsubstituted -alkyl-N(H)—C(═O)-alkyl-aryl groups, and substituted and unsubstituted -alkyl-N(H)—C(═O)-alkyl-heterocyclyl groups; or R² and R³ are a group of formula —OCH₂O— such that R² and R³ define a fused 5-membered ring that includes 2 oxygen atoms;

R³ is selected from —H; —F, —Cl, —Br, —CF₃, —C≡N, —NO₂, —CO₂H, substituted and unsubstituted amino groups, substituted and unsubstituted alkyl groups; substituted and unsubstituted alkoxy groups, substituted and unsubstituted ; C(═O)—O-alkyl groups, substituted and unsubstituted arylalkoxy groups, substituted and unsubstituted heterocyclyloxy groups, substituted and unsubstituted alkoxyalkyl groups, substituted and unsubstituted arylalkoxyalkyl groups, substituted and unsubstituted aryloxy group, substituted and unsubstituted heterocycyl groups, substituted and unsubstituted —N(H)—C(═O)-alkyl groups, substituted and unsubstituted —N—C(═O)-aryl groups, substituted and unsubstituted —N(H)—(SO₂)-alkyl groups substituted and unsubstituted —N(H)—(SO₂)-aryl groups, —N(H)—(SO₂)—CF₃ groups, substituted and unsubstituted —N(H)—(SO₂)-heterocyclyl groups, substituted and unsubstituted —N(H)C(═O)N(H)-alkyl groups, substituted and unsubstituted —N(H)C(═O)N(H)-aryl groups, substituted and unsubstituted —C(═O)—N(H)-alkyl groups, substituted and unsubstituted —C(═O)—N(H)-alkyl-heterocyclyl groups, substituted and unsubstituted (alkyl)(alkyl)aminoalkyl groups, substituted and unsubstituted (alkyl)(aryl)aminoalkyl groups, substituted and unsubstituted (alkyl)(heterocyclyl)aminoalkyl groups substituted and unsubstituted (alkyl)(arylalkyl)aminoalkyl groups, substituted and unsubstituted (alkyl)(heterocyclylalkyl)aminoalkyl groups, substituted and unsubstituted -alkyl-N(alkyl)-C(═O)-alkyl groups, substituted and unsubstituted -alkyl-N(alkyl)-C(═O)-aryl groups, substituted and unsubstituted -alkyl-N(alkyl)-C(═O)-heterocyclyl groups, substituted and unsubstituted -alkyl-N(alkyl)-C(═O)-alkyl-aryl groups, substituted and unsubstituted -alkyl-N(alkyl)-C(═O)-alkyl-heterocyclyl groups, substituted; and unsubstituted alkylaminoalkyl groups, substituted and unsubstituted arylaminoalkyl groups, substituted and unsubstituted heterocyclylaminoalkyl groups substituted and unsubstituted arylalkylaminoalkyl groups, substituted and unsubstituted heterocyclylalkylaminoalkyl groups, substituted and unsubstituted -alkyl-N(H)—C(═O)-alkyl groups, substituted and unsubstituted -alkyl-N(H)—C(═O)-aryl groups, substituted and unsubstituted -alkyl-N(H)—C(═O)-heterocyclyl groups, substituted and unsubstituted -alkyl-N(H)—C(═O)-alkyl-aryl groups, and substituted and unsubstituted -alkyl-N(H)—C(═O)-alkyl-heterocyclyl groups;

R⁴ is selected from —H, —F, —Br, —Cl, —NO₂, —C≡N, —C(═O)—O-alkyl groups, —OH, substituted and unsubstituted arylalkoxy groups, substituted and unsubstituted heterocyclyloxy groups, substituted and unsubstituted alkoxyalkyl groups, substituted and unsubstituted arylalkoxyalkyl groups, substituted and unsubstituted aryloxy groups, substituted and unsubstituted —N(H)—C(═O)-aryl groups, substituted and unsubstituted —N(H)—C(═O)-alkyl groups, substituted and unsubstituted —N(H)—SO₂-alkyl groups, substituted and unsubstituted —N(H)—SO₂-aryl groups, —N(H)—SO₂—CF₃ groups, substituted and unsubstituted —N(H)—SO₂-heterocyclyl groups, substituted and unsubstituted heterocyclyl groups, substituted and unsubstituted amino groups, substituted and unsubstituted alkyl groups, substituted and unsubstituted alkoxy groups, substituted and unsubstituted —C(═O)—N(H)-alkyl groups, substituted and unsubstituted —C(═O)N(H)-alkyl-heterocyclyl groups, substituted and unsubstituted (alkyl)(alkyl)aminoalkyl groups, substituted and unsubstituted (alkyl) (aryl)aminoalkyl groups, substituted and unsubstituted (alkyl)(heterocyclyl)aminoalkyl groups substituted and unsubstituted (alkyl)(arylalkyl)aminoalkyl groups, substituted and unsubstituted (alkyl)(heterocyclylalkyl)aminoalkyl groups, substituted and unsubstituted -alkyl-N(alkyl)-C(═O)-alkyl groups, substituted and unsubstituted -alkyl-N(alkyl)-C(═O)-aryl groups, substituted and unsubstituted -alkyl-N(alkyl)-C(═O)-heterocyclyl groups, substituted and unsubstituted -alkyl-N(alkyl)-C(═O)-alkyl-aryl groups, substituted and unsubstituted -alkyl-N(alkyl)-C(═O)-alkyl-heterocyclyl groups, substituted and unsubstituted alkylaminoalkyl groups, substituted and unsubstituted arylaminoalkyl groups, substituted and unsubstituted heterocyclylaminoalkyl groups substituted and unsubstituted arylalkylaminoalkyl groups, substituted and unsubstituted heterocyclylalkylaminoalkyl groups, substituted and unsubstituted -alkyl-N(H)—C(═O)-alkyl groups, substituted and unsubstituted -alkyl-N(H)—C(═O)-aryl groups, substituted and unsubstituted -alkyl-N(H)—C(═O)-heterocyclyl groups, substituted and unsubstituted -alkyl-N(H)—C(—O)-alkyl-aryl: groups, and substituted and unsubstituted -alkyl-N(H)—C(═O)-alkyl-heterocyclyl groups;

R⁵ is selected from —H, —P, —Cl, substituted and unsubstituted alkyl groups, substituted and unsubstituted alkoxy groups, substituted and unsubstituted amino groups, substituted and unsubstituted alkylamino groups, substituted and unsubstituted dialkylamino groups, and substituted and unsubstituted heterocyclyl groups, or R⁵ is absent if Z¹ is N;

R⁶ is selected from —H, —F, —Cl, —Br, —CF₃, —CO₂H, substituted and unsubstituted alkyl groups, substituted and unsubstituted alkoxy groups including substituted and unsubstituted heterocyclylalkoxy groups, substituted and unsubstituted arylalkoxy groups, and substituted and unsubstituted alkoxyalkoxy groups; substituted and unsubstituted heterocyclyl groups including substituted and unsubstituted heterocyclylheterocyclyl groups, substituted and unsubstituted arylheterocyclyl groups, substituted and unsubstituted alkylheterocyclyl groups, and substituted and unsubstituted cycloalkylheterocyclyl groups; substituted and unsubstituted heterocyclyloxy groups, substituted and unsubstituted aryloxy groups, substituted and unsubstituted amino groups including substituted and unsubstituted dialkylamino groups, substituted and unsubstituted (alkyl)(heterocyclyl)amino groups, substituted and unsubstituted heterocyclylalkylamino groups, substituted and unsubstituted arylalkylamino groups, and substituted and unsubstituted heterocyclylamino groups; substituted and unsubstituted —C(═O)N(H)-alkyl groups, substituted and unsubstituted —C(═O)N(H)-aryl groups, substituted and unsubstituted —C(═O)N(H)-heterocyclyl groups, substituted and unsubstituted —C(═O)N(alkyl) (heterocyclyl) groups, and substituted and unsubstituted —C(═O)-heterocyclyl groups; or R⁶ is absent if Z² is N;

R⁷ is selected from —H, —F, —Cl, —Br, —CF₃, —CO₂H, substituted and ursubstituted alkyl groups, substituted and unsubstituted alkoxy groups including substituted and unsubstituted heterocyclylalkoxy groups, substituted and unsubstituted arylalkoxy groups, and substituted and unsubstituted alkoxyalkoxy groups; substituted and unsubstituted heterocyclyl groups including substituted and unsubstituted heterocyclylheterocyclyl groups, substituted and unsubstituted arylheterocyclyl groups, substituted and unsubstituted alkylheterocyclyl groups, and substituted and unsubstituted cycloalkylheterocyclyl groups; substituted and unsubstituted heterocyclyloxy groups, substituted and unsubstituted aryloxy groups, substituted and unsubstituted amino groups including substituted and unsubstituted dialkylamino groups, substituted and unsubstituted (alkyl)(heterocyclyl)amino groups, substituted and unsubstituted heterocyclylalkylamino groups, substituted and unsubstituted arylalkylamino groups, and substituted and unsubstituted heterocyclylamino groups; substituted and unsubstituted —C(═O)N(H)-alkyl groups, substituted and unsubstituted —C(—O)N(H)-aryl groups, substituted and unsubstituted —C(═O)NR-heterocyclyl groups, substituted and unsubstituted —C(═O)N(alkyl)(heterocyclyl) groups, and substituted and unsubstituted —C(═O)-heterocyclyl groups; or R7 is absent if Z³ is N;

R⁸ is selected from —H, —F, —Cl, substituted and unsubstituted alkyl groups, substituted and unsubstituted alkoxy groups, substituted and unsubstituted amino groups, substituted and unsubstituted alkylamino groups, substituted and unsubstituted dialkylamino groups, and substituted and unsubstituted heterocyclyl groups, or R⁸ is absent if Z⁴ is N;

R⁹ is —H; and

R¹⁰ is selected from the group consisting of —H, and substituted and unsubstituted alkyl groups, and further wherein at least one of Z² or Z³ is C and at least one of R⁶ or R⁷ is selected from the group consisting of —Br, —CO₂H, substituted and unsubstituted heterocyclylalkoxy groups, substituted and unsubstituted arylalkoxy groups, substituted and unsubstituted-alkoxyalkoxy groups, substituted and unsubstituted heterocyclylheterocyclyl groups, substituted and unsubstituted arylheterocyclyl groups, substituted and unsubstituted cycloalkylheterocyclyl groups, substituted and unsubstituted heterocyclyloxy groups, substituted and unsubstituted aryloxy groups, substituted and unsubstituted (alkyl)(heterocyclyl)amino groups, substituted and unsubstituted heterocyclylalkylamino groups, substituted and unsubstituted arylalkylamino groups, substituted and unsubstituted heterocyclylamino groups, substituted and unsubstituted —C(═O)N(H)-aryl groups, substituted and unsubstituted —C(═O)N(H)-heterocyclyl groups, substituted and unsubstituted —C(═O)N(alkyl)(heterocyclyl) groups, and substituted and unsubstituted —C(═O)-heterocyclyl groups.

iv) A compound having the structure below, a tautomer of the compound, a pharmaceutically acceptable salt of the compound, or a pharmaceutically acceptable salt of the tautomer: 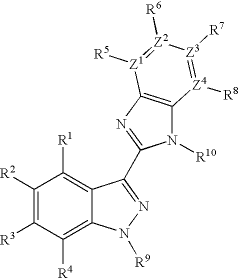
wherein

Z¹, Z², Z³, and Z⁴ are independently selected from C or N;

R¹ is selected from —H, —F, —Cl, —Br, —NO₂, —C≡N, —C(═O)—O-alkyl groups, —OH, substituted and unsubstituted arylalkoxy groups, substituted and unsubstituted heterocyclyloxy groups, substituted and unsubstituted alkoxyalkyl groups, substituted and unsubstituted arylalkoxyalkyl groups, substituted and unsubstituted aryloxy groups, substituted and unsubstituted —N(H)—C(═O)-aryl groups, substituted and unsubstituted —N(H)—C(═O)-alkyl groups, substituted and unsubstituted —N(H)—SO₂-alkyl groups, substituted and unsubstituted —N(H)—SO₂-aryl groups, —N(H)—SO₂—CF₃ groups, substituted and unsubstituted —N(H)—SO₂heterocyclyl groups, substituted and unsubstituted heterocyclyl groups, substituted and unsubstituted-alkyl groups, substituted and unsubstituted alkoxy groups, substituted and unsubstituted amino groups, substituted and unsubstituted —C(═O)—N (H)-alkyl groups, substituted and unsubstituted —C(═O)—N(H)-alkyl-heterocyclyl groups, substituted and unsubstituted (alkyl)(alkyl)aminoalkyl groups, substituted and unsubstituted (alkyl)(aryl)aminoalkyl groups, substituted and unsubstituted (alkyl)(heterocyclyl)aminoalkyl groups, substituted and unsubstituted (alkyl)(arylalkyl)aminoalkyl groups, substituted and unsubstituted (alkyl)(heterocyclylalkyl)aminoalkyl groups, substituted and unsubstituted -alkyl-N(alkyl)-C(═O)alkyl groups, substituted and unsubstituted -alkyl-N(alkyl)-C(═O)-aryl groups, substituted and unsubstituted -alkyl-N(alkyl)-C(═O)-heterocyclyl groups, substituted and unsubstituted -alkyl-N(alkyl)-C(═O)-alkyl-aryl groups, substituted and unsubstituted -alkyl-N (alkyl)-C(═O)alkyl-heterocyclyl groups, substituted and unsubstituted alkylaminoalkyl groups, substituted and unsubstituted arylaminoalkyl groups, substituted and unsubstituted heterocyclylaminoalkyl groups, substituted and unsubstituted arylalkylaminoalkyl groups, substituted and unsubstituted heterocyclylalkylaminoalkyl groups, substituted and unsubstituted -alkyl-N(H)—C(═O)-alkyl groups, substituted and unsubstituted -alkyl-N(H)—C(═O)-aryl groups, substituted and unsubstituted -alkyl-N(H)—C(═O)-heterocyclyl groups, substituted and unsubstituted -alkyl-N(H)—C(═O)-alkyl-aryl groups, and substituted and unsubstituted -alkyl-N(H)—C(═O)-alkyl-heterocyclyl groups;

R² is selected from —H, —F, —Cl, —Br, —C≡N, —NO₂, —CO₂H, —OH, substituted and unsubstituted guanidinyl groups, substituted and unsubstituted amino groups, substituted and unsubstituted alkyl groups, substituted and unsubstituted —C(═O)O-alkyl groups, substituted and unsubstituted —C(═O)O-aryl groups, substituted and unsubstituted —C(═O)O-heteroaryl groups, substituted and unsubstituted —C(═O)N(H)-alkyl groups, substituted and unsubstituted —C(═O)—N(H)-alkyl-heterocyclyl groups, substituted and unsubstituted —C(═O)N(H)-aryl groups, substituted and unsubstituted —C(═O)N(H)-heterocyclyl groups, substituted and unsubstituted-N(H)C(═O)-alkyl groups, substituted and unsubstituted —N(H)C(═O)aryl groups, substituted and unsubstituted —N(H)C(═O)-heterocyclyl groups, substituted and unsubstituted —N(H)C(═O)N(H)-alkyl groups, substituted and unsubstituted —N(H)C(═O)N(H)-aryl groups, substituted and unsubstituted —N(H)C(═O)N(H)-heterocyclyl groups, substituted and unsubstituted —N(H)—(SO₂)-alkyl groups, substituted and unsubstituted —N(H)(SO₂)-aryl groups, —N(H)—(SO₂)—CF₃ groups, substituted and unsubstituted —N(H)—(SO₂)-heterocyclyl groups, substituted and unsubstituted —N(H)-heterocyclyl groups, substituted and unsubstituted heterocyclyl groups, substituted and unsubstituted alkoxy groups, substituted and unsubstituted arylalkoxy groups, substituted and unsubstituted aryloxy groups, substituted and unsubstituted alkoxyalkyl groups, substituted and unsubstituted arylalkoxyalkyl groups, substituted and unsubstituted heterocyclyloxy, substituted and unsubstituted heterocyclylalkoxy groups, substituted and unsubstituted (alkyl)(alkyl)aminoalkyl groups, substituted and unsubstituted(alkyl)(aryl) aminoalkyl groups, substituted and unsubstituted (alkyl)(heterocyclyl)aminoalkyl groups substituted and unsubstituted (alkyl)(arylalkyl)aminoalkyl groups, substituted and unsubstituted (alkyl)(heterocyclylalkyl)aminoalkyl groups, substituted and unsubstituted -alkyl-N(alkyl)-C(═O)-alkyl groups, substituted and unsubstituted -alkyl-N(alkyl)-C(═O)-aryl groups, substituted and unsubstituted -alkyl-N(alkyl)C(═O)-heterocyclyl groups, substituted and unsubstituted -alkyl-N(alkyl)-C(═O)-alkyl-aryl groups, substituted and unsubstituted -alkyl-N(alkyl)-C(═O)-alkyl-heterocyclyl groups, substituted and unsubstituted alkylaminoalkyl groups, substituted and unsubstituted arylaminoalkyl groups, substituted and unsubstituted heterocyclylaminoalkyl groups, substituted and unsubstituted arylalkylaminoalkyl groups, substituted and unsubstituted heterocyclylalkylaminoalkyl groups, substituted and unsubstituted -alkyl-N(H)—C(═O)-alkyl groups, substituted and unsubstituted -alkyl-N(H)—C(═O)-aryl groups, substituted and unsubstituted -alkyl-N(H)—C(═O)-heterocyclyl groups, substituted and unsubstituted -alkyl-N(H)—C(═O)-alkyl-aryl groups, and substituted and unsubstituted -alkyl-N(H)—C(═O)-alkyl-heterocyclyl groups; or R² and R³ are a group of formula —OCH₂O— such that R² and R³ define a fused 5-membered ring that includes 2 oxygen atoms;

R³ is selected from —F, —Cl, —Br, —CF₃, —C≡N, substituted and unsubstituted alkyl groups, substituted and unsubstituted alkoxy groups, substituted and unsubstituted arylalkoxy groups, substituted and unsubstituted saturated heterocyclyloxy groups, substituted and unsubstituted alkoxyalkyl groups, substituted and unsubstituted arylalkoxyalkyl groups, substituted and unsubstituted saturated heterocycyl groups, substituted and unsubstituted —N(H)—C(═O)-alkyl groups, substituted and unsubstituted —N(H)—C(═O)-aryl groups, substituted and unsubstituted —N(H)—(SO₂)-alkyl groups substituted and unsubstituted —N(H)—(SO₂)-aryl groups, —N(H)—(SO₂)—CF₃ groups, substituted and unsubstituted —N(H)—(SO₂)-heterocyclyl groups, substituted and unsubstituted —N(H)C(═O)N(H)-alkyl groups, and substituted and unsubstituted —N(H)C(═O)N(H)-aryl;

R⁴ is —H, —F, —Br, —Cl, —NO₂, —C≡N, —C(═O)—O-alkyl groups, —OH, substituted and unsubstituted arylalkoxy groups, substituted and unsubstituted heterocyclyloxy groups, substituted and unsubstituted alkoxyalkyl groups, substituted and unsubstituted-arylalkoxyalkyl groups, substituted and unsubstituted aryloxy groups, substituted and unsubstituted —N(H)—C(═O)-aryl groups, substituted and unsubstituted —N(H)—C(═O)-alkyl groups, substituted and unsubstituted —N(H)—SO₂-alkyl groups, substituted and unsubstituted —N(H)—SO₂-aryl groups, —N(H)—SO₂—CF₃ groups, substituted and unsubstituted —N(H)—SO₂-heterocyclyl groups, substituted and unsubstituted heterocyclyl groups, substituted and unsubstituted amino groups, substituted and unsubstituted alkyl groups, substituted and unsubstituted alkoxy groups, substituted and unsubstituted —C(═O)—N(H)-alkyl groups, substituted and unsubstituted —C(═O)—N(H)-alkyl-heterocyclyl groups, substituted and unsubstituted (alkyl)(alkyl)aminoalkyl groups, substituted and unsubstituted (alkyl)(aryl)aminoalkyl groups, substituted and unsubstituted (alkyl)(heterocyclyl)aminoalkyl groups, substituted and unsubstituted (alkyl)(arylalkyl)aminoalkyl groups, substituted and unsubstituted (alkyl)(heterocyclylalkyl)aminoalkyl groups, substituted and unsubstituted -alkyl-N(alkyl)-C(═O)-alkyl groups, substituted and unsubstituted -alkyl-N(alkyl)-C(═O)-aryl groups, substituted and unsubstituted -alkyl-N(alkyl)-C(═O)-heterocyclyl groups, substituted and unsubstituted -alkyl-N(alkyl)-C(═O)-alkyl-aryl groups; substituted and unsubstituted -alkyl-N(alkyl)-C(═O)-alkyl-heterocyclyl groups, substituted and unsubstituted alkylaminoalkyl groups, substituted and unsubstituted arylaminoalkyl groups, substituted and unsubstituted heterocyclylaminoalkyl groups, substituted and unsubstituted arylalkylaminoalkyl groups, substituted and unsubstituted heterocyclylalkylaminoalkyl groups, substituted and unsubstituted -alkyl-N(H)—C(═O)-alkyl groups, substituted and unsubstituted -alkyl-N(H)—C(═O)-aryl groups, substituted and unsubstituted -alkyl-N(H)—C(═O)-heterocyclyl groups, substituted and unsubstituted -alkyl-N(H)—C(═O)-alkyl-aryl groups, and substituted and unsubstituted -alkyl-N(H)C(═O)-alkyl-heterocyclyl groups,

R⁵ is selected from —H, —F, —Cl, substituted and unsubstituted alkyl groups, substituted and unsubstituted alkoxy groups, substituted and unsubstituted amino groups, substituted and unsubstituted alkylamino groups, substituted and unsubstituted dialkylamino groups, and substituted and unsubstituted heterocyclyl groups, or R⁵ is absent if Z¹ is N; ;

R⁶ is selected from —H, —F, —Cl, —Br, —CF₃, —CO₂H, substituted and unsubstituted alkyl groups, substituted and unsubstituted alkoxy groups including substituted and unsubstituted heterocyclylalkoxy groups, substituted and unsubstituted arylalkoxy groups, and substituted and unsubstituted alkoxyalkoxy groups; substituted and unsubstituted heterocyclyl groups including substituted and unsubstituted heterocyclylheterocyclyl groups, substituted and unsubstituted arylheterocyclyl groups, substituted and unsubstituted alkylheterocyclyl groups, and substituted and unsubstituted cycloalkylheterocyclyl groups; substituted and unsubstituted heterocyclyloxy groups, substituted and unsubstituted aryloxy groups, substituted and unsubstituted amino groups including substituted and unsubstituted dialkylamino groups, substituted and unsubstituted (alkyl)(heterocyclyl)amino groups, substituted and unsubstituted heterocyclylalkylamino groups, substituted and unsubstituted arylalkylamino groups, and substituted and unsubstituted heterocyclylamino groups; substituted and unsubstituted —C(═O)NR-alkyl groups, substituted and unsubstituted —C(═O)N(H)-aryl groups, substituted and unsubstituted —C(═O)N(H)-heterocyclyl groups, substituted and unsubstituted —C(═O)N(alkyl)(heterocyclyl) groups, and substituted and unsubstituted —C(—O)-heterocyclyl groups; or R⁶ is absent if Z² is N;

R⁷ is selected from —H, —F, —Cl, —Br, —CF₃, —CO₂H, substituted and unsubstituted alkyl groups, substituted and unsubstituted alkoxy groups including substituted and unsubstituted heterocyclylalkoxy groups, substituted and unsubstituted arylalkoxy groups, and substituted and unsubstituted alkoxyalkoxy groups; substituted and unsubstituted heterocyclyl groups including substituted and unsubstituted heterocyclylheterocyclyl groups, substituted and unsubstituted arylheterocyclyl groups, substituted and unsubstituted alkylheterocyclyl groups, and substituted and unsubstituted cycloalkylheterocyclyl groups; substituted and unsubstituted heterocyclyloxy groups, substituted and unsubstituted aryloxy groups, substituted and unsubstituted amino groups including substituted and unsubstituted dialkylamino groups, substituted and unsubstituted (alkyl)(heterocyclyl)amino groups, substituted and unsubstituted heterocyclylalkylamino groups, substituted and unsubstituted arylalkylamino groups, and substituted and unsubstituted heterocyclylamino groups; substituted and unsubstituted —C(═O)N(H)-alkyl groups, substituted and unsubstituted —C(═O)N(H)-aryl groups, substituted and unsubstituted —C(═O)N(H)-heterocyclyl groups, substituted and unsubstituted —C(═O)N(alkyl)(heterocyclyl) groups, and substituted and unsubstituted —C(═O)-heterocyclyl groups; or R⁷ is absent if Z³ is N;

R⁸ is selected from —H, —F, —Cl, substituted and unsubstituted alkyl groups, substituted and unsubstituted alkoxy groups, substituted and unsubstituted amino groups, substituted and unsubstituted alkylamino groups, substituted and unsubstituted dialkylamino groups, and substituted and unsubstituted heterocyclyl groups, or R⁸ is absent if Z⁴ is N;

R⁹ is —H; and

R¹⁰ is selected from the group consisting of —H, and substituted and unsubstituted alkyl groups.

v) A compound having the structure below, a tautomer of the compound a pharmaceutically acceptable salt of the compound, or a pharmaceutically acceptable salt of the tautomer:

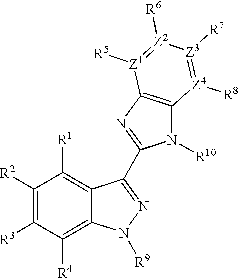

wherein

Z¹, Z², Z³, and Z⁴ are independently selected from C or N;

R¹ is selected from —H, —F, —Cl, —Br, —NO₂, —C≡N, —C(—O)—O-alkyl groups, —OH, substituted and unsubstituted arylalkoxy groups, substituted and unsubstituted heterocyclyloxy groups, substituted and unsubstituted alkoxyalkyl groups, substituted and unsubstituted arylalkoxyalkyl groups, substituted and unsubstituted aryloxy groups, substituted and unsubstituted —N(H)—C(═O)-aryl groups, substituted and unsubstituted, —N(H)—C(═O)-alkyl groups, substituted and unsubstituted —N(H)—SO₂-alkyl, groups, substituted and unsubstituted —N(H)—SO₂-aryl groups, —N(H)—SO₂—CF₃ groups, substituted and unsubstituted —N(H)—SO₂-heterocyclyl groups, substituted and unsubstituted heterocyclyl groups, substituted and unsubstituted alkyl groups, substituted and unsubstituted alkoxy groups, substituted and unsubstituted amino groups, substituted and unsubstituted —C(═O)—N(H)-alkyl groups, substituted and unsubstituted —C(═O)—N(H)-alkyl-heterocyclyl groups, substituted and unsubstituted (alkyl)(alkyl)aminoalkyl groups, substituted and unsubstituted (alkyl)(aryl)aminoalkyl groups, substituted and unsubstituted (alkyl)(heterocyclyl)aminoalkyl groups substituted and unsubstituted (alkyl)(arylalkyl)aminoalkyl groups, substituted and unsubstituted (alkyl)(heterocyclylalkyl)aminoalkyl groups, substituted and unsubstituted -alkyl-N(alkyl)-C(═O)-alkyl groups, substituted and unsubstituted -alkyl-N(alkyl)-C(═O)-aryl groups, substituted and unsubstituted -alkyl-N(alkyl)C(═O)-heterocyclyl groups, substituted and unsubstituted -alkyl-N(alkyl)-C(═O)-alkyl-aryl groups, substituted and unsubstituted -alkyl-N(alkyl)-C(═O)-alkyl-heterocyclyl groups, substituted and unsubstituted alkylaminoalkyl groups, substituted and unsubstituted arylaminoalkyl groups, substituted and unsubstituted heterocyclylaminoalkyl groups substituted and unsubstituted arylalkylaminoalkyl groups, substituted and unsubstituted heterocyclylalkylaminoalkyl groups, substituted and unsubstituted -alkyl-N(H)—C(═O)-alkyl groups, substituted and unsubstituted -alkyl-N(H)—C(═O)-aryl groups, substituted and unsubstituted -alkyl-N(H)—C(═O)-heterocyclyl groups, substituted and unsubstituted -alkyl-N(H)—C(═O)-alkyl-aryl groups, and substituted and unsubstituted -alkyl-N(H)—C(═O)-alkyl-heterocyclyl groups;

R² is selected from —F, —Cl, —Br, —C≡N, —NO₂, —CO₂H, —OH, substituted and unsubstituted guanidinyl groups, substituted and unsubstituted —C(═O)O-alkyl groups, substituted and unsubstituted —C(═O)O-aryl groups, substituted and unsubstituted —C(═O)O-heteroaryl groups, substituted and unsubstituted —C(═O)N(H)-alkyl groups, substituted and unsubstituted —C(═O)N(H)-aryl groups, substituted and unsubstituted —C(═O)N(H)-heterocyclyl groups, substituted and unsubstituted —N(H)C(═O)-alkyl groups, substituted and unsubstituted —N(H)C(═O)-aryl groups, substituted and unsubstituted —N(H)C(═O)-heterocyclyl groups, substituted and unsubstituted —N(H)C(═O)N(H)-alkyl groups, substituted and unsubstituted —N(H)C(═O)N(H)-aryl groups, substituted and unsubstituted —N(H)C(═O)N(H)-heterocyclyl groups, substituted and unsubstituted —N(H)—(SO₂)-alkyl groups, substituted and unsubstituted —N(H)—(SO₂)-aryl groups, —N(H)—(SO₂)—CF₃ groups, substituted and unsubstituted —N(H)—(SO₂)-heterocyclyl groups, substituted and unsubstituted alkoxy groups, substituted and unsubstituted arylalkoxy groups, substituted and unsubstituted aryloxy groups, substituted and unsubstituted heterocyclyloxy, and substituted and unsubstituted heterocyclylalkoxy groups; or R² and R³ are a group of formula —OCH₂O— such that R² and R⁴ define a fused 5-membered ring that includes 2 oxygen atoms;

R³ is selected from —H, —F, —Cl, —Br, —CF₃, —C≡N, —NO₂, —CO₂H, substituted and unsubstituted amino groups, substituted and unsubstituted alkyl groups, substituted and unsubstituted alkoxy groups, substituted and unsubstituted —C(═O)—O-alkyl groups, substituted and unsubstituted arylalkoxy groups, substituted and unsubstituted heterocyclyloxy groups, substituted and unsubstituted alkoxyalkyl groups, substituted and unsubstituted arylalkoxyalkyl groups, substituted and unsubstituted aryloxy group, substituted and unsubstituted heterocycyl groups, substituted and unsubstituted —N(H)—C(═O)-alkyl groups, substituted and unsubstituted —N(H)—C(═O)-aryl groups, substituted and unsubstituted —N(H)—(SO₂)-alkyl groups substituted and unsubstituted —N(H)—(SO₂)-aryl groups, —N(H)—(SO₂)—CF₃ groups, substituted and unsubstituted —N(H)—(SO₂)-heterocyclyl groups, substituted and unsubstituted —N(H)C(═O)N(H)-alkyl groups, substituted and unsubstituted —N(H)C(═O)N(H)-aryl groups, substituted and unsubstituted —C(═O)—N(H)-alkyl groups, substituted and unsubstituted —C(═O)—N(H)-alkyl-heterocyclyl groups, substituted and unsubstituted (alkyl)(alkyl)aminoalkyl groups, substituted and unsubstituted (alkyl)(aryl)aminoalkyl groups, substituted and unsubstituted (alkyl)(heterocyclyl)aminoalkyl groups substituted and unsubstituted (alkyl)(arylalkyl)aminoalkyl groups, substituted and unsubstituted (alkyl)(heterocyclylalkyl)aminoalkyl groups, substituted and unsubstituted -alkyl-N(alkyl)-C(═O)alkyl groups, substituted and unsubstituted -alkyl-N(alkyl)-C(═O)-aryl groups substituted and unsubstituted -alkyl-N(alkyl)-C(—O)-heterocyclyl groups, substituted and unsubstituted -alkyl-N(alkyl)-C(═O)-alkyl-aryl groups, substituted and unsubstituted -alkyl-N(alkyl)-C(═O)-alkyl-heterocyclyl groups, substituted and ursubstituted-alkylaminoalkyl groups, substituted and unsubstituted arylaminoalkyl groups, substituted and unsubstituted heterocyclylaminoalkyl groups, substituted and unsubstituted arylalkylaminoalkyl groups, substituted and unsubstituted heterocyclylalkylaminoalkyl groups, substituted and unsubstituted -alkyl-N(H)—C(═O)-alkyl groups, substituted and unsubstituted -alkyl-N(H)—C(═O)-aryl groups, substituted and unsubstituted -alkyl-N(H)—C(═O)-heterocyclyl groups, substituted and unsubstituted -alkyl-N(H)—C(═O)-alkyl-aryl groups, and substituted and unsubstituted -alkyl-N(H)—C(═O)-alkyl-heterocyclyl groups;

R⁴ —H, —F, —Br, —Cl, —NO₂, —C≡N, —C(═O)—O-alkyl groups, —OH, substituted and unsubstituted arylalkoxy groups, substituted and unsubstituted heterocyclyloxy groups, substituted and unsubstituted alkoxyalkyl groups, substituted and unsubstituted arylalkoxyalkyl groups, substituted and unsubstituted aryloxy groups, substituted and unsubstituted —N(H)—C(═O)aryl groups, substituted and unsubstituted —N(H)—C(═O)-alkyl groups, substituted and unsubstituted —N(H)—(SO₂)-alkyl groups, substituted and unsubstituted —N(H)—(SO₂)-aryl groups, —N(H)—(SO₂)—CF₃ groups, substituted and unsubstituted —N(H)—(SO₂)-heterocyclyl groups, substituted and unsubstituted-heterocyclyl groups, substituted and unsubstituted amino groups, substituted and unsubstituted alkyl groups, substituted and unsubstituted alkoxy groups, substituted and unsubstituted —C(═O)—N(H)-alkyl groups, substituted and unsubstituted —C(═O)—N(H)-alkyl-heterocyclyl groups, substituted and unsubstituted (alkyl)(alkyl)aminoalkyl groups, substituted and unsubstituted (alkyl)(aryl)aminoalkyl groups, substituted and unsubstituted (alkyl) (heterocyclyl)aminoalkyl groups substituted and unsubstituted (alkyl)(arylalkyl)aminoalkyl groups, substituted and unsubstituted (alkyl)(heterocyclylalkyl)aminoalkyl groups, substituted and unsubstituted -alkyl-N(alkyl)-C(—O)-alkyl groups, substituted and unsubstituted -alkyl-N(alkyl)C(═O)-aryl groups, substituted and unsubstituted -alkyl-N(alkyl)-C(═O)-heterocyclyl groups, substituted and unsubstituted -alkyl-N(alkyl)-C(═O)-alkyl-aryl groups, substituted and unsubstituted -alkyl-N(alkyl)-C(═O)-alkyl-heterocyclyl groups, substituted and unsubstituted alkylaminoalkyl groups, substituted and unsubstituted arylaminoalkyl groups, substituted and unsubstituted heterocyclylaminoalkyl groups, substituted and unsubstituted arylalkylaminoalkyl groups, substituted and unsubstituted heterocyclylalkylaminoalkyl groups, substituted and unsubstituted -alkyl-N(H)—C(═O)-alkyl groups, substituted and unsubstituted alkyl-N(H)—C(═O)-aryl groups, substituted and unsubstituted -alkyl-N(H)—C(═O)-heterocyclyl groups, substituted and unsubstituted -alkyl-N(H)—C(═O)-alkyl-aryl groups and substituted and unsubstituted -alkyl-N(H)—C(═O)-alkyl-heterocyclyl groups;

R⁵ is selected from —H, —F, —Cl, substituted and unsubstituted alkyl groups, substituted and unsubstituted alkoxy groups, substituted and unsubstituted ammo groups, substituted and unsubstituted alkylamino groups, substituted and unsubstituted dialkylamino groups, and substituted and unsubstituted heterocyclyl groups, or R⁵ is absent if Z¹ is N;

R¹ is selected from —H, —F, —Cl, —Br, —CF₃, —CO₂H, substituted and unsubstituted alkyl groups, substituted and unsubstituted alkoxy groups including substituted and unsubstituted heterocyclylalkoxy groups, substituted and unsubstituted arylalkoxy groups, and substituted and unsubstituted alkoxyalkoxy groups; substituted and unsubstituted heterocyclyl groups including substituted and unsubstituted heterocyclylheterocyclyl groups, substituted and unsubstituted arylheterocyclyl groups, substituted and unsubstituted alkylheterocyclyl groups, and substituted and unsubstituted cycloalkylheterocyclyl groups; substituted and unsubstituted heterocyclyloxy groups, substituted and unsubstituted aryloxy groups, substituted and unsubstituted amino groups including substituted and unsubstituted dialkylamino groups, substituted and unsubstituted (alkyl)(heterocyclyl)amino groups, substituted and unsubstituted heterocyclylalkylamino groups, substituted and unsubstituted arylalkylamino groups, and substituted and unsubstituted heterocyclylamino groups; substituted and unsubstituted —C(═O)N(H)-alkyl groups, substituted and unsubstituted —C(═O)N(H)-aryl groups, substituted and unsubstituted —C(═O)N(H)-heterocyclyl groups, substituted and unsubstituted —C(═O)N(alkyl)(heterocyclyl) groups, and substituted and unsubstituted —C(═O)-heterocyclyl groups; or R⁶ is absent if Z² is N;

R⁷ is selected from —H, —F, —Cl, —Br, —CF₃, —CO₂H, substituted and unsubstituted alkyl groups, substituted and unsubstituted alkoxy groups including substituted and unsubstituted heterocyclylalkoxy groups, substituted and unsubstituted arylalkoxy groups, and substituted and unsubstituted alkoxyalkoxy groups; substituted and unsubstituted heterocyclyl groups including substituted and unsubstituted heterocyclylheterocyclyl groups, substituted and unsubstituted arylheterocyclyl groups, substituted and unsubstituted alkylheterocyclyl groups, and substituted and unsubstituted cycloalkylheterocyclyl groups; substituted and unsubstituted heterocyclyloxy groups, substituted and unsubstituted aryloxy groups, substituted and unsubstituted amino groups including substituted and unsubstituted dialkylamino groups, substituted and unsubstituted (alkyl)(heterocyclyl)amino groups, substituted and unsubstituted heterocyclylalkylamino groups, substituted and unsubstituted arylalkylamino groups, and substituted and unsubstituted heterocyclylamino groups; substituted and unsubstituted —C(═O)N(H)-alkyl groups, substituted and unsubstituted —C(═O)N(H)-aryl groups, substituted and unsubstituted —C(═O)N(H)-heterocyclyl groups, substituted and unsubstituted —C(═O)N(alkyl)(heterocyclyl) groups, and substituted and unsubstituted —C(═O)-heterocyclyl groups; or R⁷ is absent if Z³ is N;

R⁸ is selected from —H, —F, —Cl, substituted and unsubstituted alkyl groups, substituted and unsubstituted alkoxy groups, substituted and unsubstituted amino groups, substituted and unsubstituted alkylamino groups, substituted and unsubstituted dialkylamino groups, and substituted and unsubstituted heterocyclyl groups, or R⁸ is absent if Z⁴ is N;

R⁹ is —H; and

R¹⁰ is selected from the group consisting of —H, and substituted and unsubstituted alkyl groups.

vi) A compound having the structure below, a tautomer of the compound a pharmaceutically acceptable salt of the compound, or a pharmaceutically acceptable salt of the tautomer: 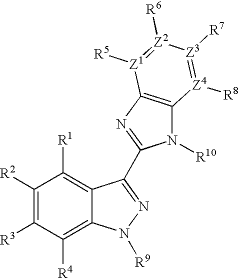
wherein

Z¹, Z², Z³, and Z⁴ are independently selected from C or N;

R¹ is selected from —H, —F, —Cl, —Br, —NO₂, —C≡N, —C(═O)—O-alkyl groups; —OH, substituted and unsubstituted arylalkoxy groups, substituted and unsubstituted heterocyclyloxy groups, substituted and unsubstituted alkoxyalkyl groups, substituted and unsubstituted arylalkoxyalkyl groups, substituted and unsubstituted aryloxy groups, substituted and unsubstituted —N(H)—C(═O)-aryl, groups, substituted and unsubstituted —N(H)—C(═O)-alkyl groups, substituted and unsubstituted —N(H)—(SO₂)-alkyl groups, substituted and unsubstituted —N(H)—(SO₂)-aryl groups, —N(H)—(SO₂)—CF₃ groups, substituted and unsubstituted —N(H)—(SO₂)-heterocyclyl groups, substituted and unsubstituted heterocyclyl groups, substituted and unsubstituted alkyl groups, substituted and unsubstituted alkoxy groups, substituted and unsubstituted amino groups, substituted and unsubstituted —C(═O)—N(H)-alkyl groups, substituted and unsubstituted —C(═O)—N(H)-alkyl-heterocyclyl, groups, substituted and unsubstituted (alkyl)(alkyl)aminoalkyl groups, substituted and unsubstituted (alkyl)(aryl)aminoalkyl groups, substituted and unsubstituted (alkyl)(heterocyclyl)aminoalkyl groups substituted and unsubstituted (alkyl)(arylalkyl)aminoalkyl groups, substituted and unsubstituted (alkyl)(heterocyclylalkyl)aminoalkyl groups, substituted and unsubstituted -alkyl-N(alkyl)-C(═O)-alkyl groups, substituted and unsubstituted -alkyl-N(alkyl)-C(═O)-aryl groups, substituted and unsubstituted -alkyl-N(alkyl)-C(═O)-heterocyclyl groups, substituted and unsubstituted -alkyl-N(alkyl)-C(═O)-alkyl-aryl groups, substituted and unsubstituted -alkyl-N(alkyl)-C(═O)-alkyl-heterocyclyl groups, substituted and unsubstituted alkylaminoalkyl groups, substituted and unsubstituted arylaminoalkyl groups, substituted and unsubstituted heterocyclylaminoalkyl groups substituted and unsubstituted arylalkylaminoalkyl groups, substituted and unsubstituted heterocyclylalkylaminoalkyl groups, substituted and unsubstituted -alkyl-N(H)—C(═O)alkyl groups, substituted and unsubstituted -alkyl-N(H)—C(═O)-aryl groups, substituted and unsubstituted -alkyl-N(H)—C(═O)-heterocyclyl groups, substituted and unsubstituted -alkyl-N(H)—C(═O)-alkyl-aryl groups, and substituted and unsubstituted -alkyl-N(H)—C(═O)-alkyl-heterocyclyl groups;

R² is selected from —H, —F, —Cl, —Br, —C≡N, —NO₂, —CO₂H, —OH, substituted and unsubstituted guanidinyl groups, substituted and unsubstituted amino groups, substituted and unsubstituted alkyl groups, substituted and unsubstituted —C(═O)O-alkyl groups, substituted and unsubstituted —C(═O)O-aryl groups, substituted and unsubstituted —C(═O)O-heteroaryl groups, substituted and unsubstituted —C(═O)N(H)-alkyl groups, substituted and unsubstituted —C(═O)—N(H)-alkyl-heterocyclyl groups, substituted and unsubstituted —C(—O)N(H)-aryl groups, substituted and unsubstituted —C(═O)N(H)-heterocyclyl groups, substituted and unsubstituted —N(H)C(═O)-alkyl groups, substituted and unsubstituted —N(H)C(═O)-aryl groups, substituted and unsubstituted —N(H)C(═O)-heterocyclyl groups, substituted and unsubstituted —N(H)C(═O)N(H)-alkyl groups, substituted and unsubstituted —N(H)C(═O)N(H)-aryl groups, substituted and unsubstituted —N(H)C(═O)N(H)-heterocyclyl groups, substituted and unsubstituted —N(H)—(SO₂)-alkyl groups, substituted and unsubstituted —N(H)—(SO₂)-aryl groups, —N(H)—(SO₂)—CF₃ groups, substituted and unsubstituted —N(H)—(SO₂)-heterocyclyl groups, substituted and unsubstituted —N(H)-heterocyclyl groups, substituted and unsubstituted heterocyclyl groups, substituted and unsubstituted alkoxy groups, substituted and unsubstituted arylalkoxy groups, substituted and unsubstituted aryloxy groups, substituted and unsubstituted alkoxyalkyl groups, substituted and unsubstituted arylalkoxyalkyl groups, substituted and unsubstituted heterocyclyloxy, substituted and unsubstituted heterocyclylalkoxy groups, substituted and unsubstituted (alkyl)(alkyl)aminoalkyl groups, substituted and unsubstituted (alkyl)(aryl)aminoalkyl groups, substituted and unsubstituted (alkyl)(heterocyclyl)aminoalkyl groups substituted and unsubstituted (alkyl)(arylalkyl)aminoalkyl groups, substituted and unsubstituted (alkyl)(heterocyclylalkyl)aminoalkyl groups, substituted and unsubstituted -alkyl-N(alkyl)-C(═O)-alkyl groups, substituted and unsubstituted -alkyl-N(alkyl)-C(═O)-aryl groups, substituted and unsubstituted -alkyl-N(alkyl)-C(═O)-heterocyclyl groups, substituted and unsubstituted -alkyl-N(alkyl)-C(—O)-alkyl-aryl groups, substituted and unsubstituted -alkyl-N(alkyl)-C(═O)-alkyl-heterocyclyl groups, substituted and unsubstituted alkylaminoalkyl groups, substituted and unsubstituted arylaminoalkyl groups, substituted and unsubstituted heterocyclylaminoalkyl groups substituted and unsubstituted arylalkylaminoalkyl groups, substituted and unsubstituted heterocyclylalkylaminoalkyl groups, substituted and unsubstituted -alkyl-N(H)—C(═O)-alkyl groups, substituted and unsubstituted -alkyl-N(H)—C(═O)-aryl groups, substituted and unsubstituted -alkyl-N(H)—C(═O)-heterocyclyl groups, substituted and unsubstituted -alkyl-N(H)—C(═O)-alkyl-aryl groups, and substituted and unsubstituted -alkyl-N(H)—C(═O)-alkyl-heterocyclyl groups; or R² and R³ are a group of formula —OCH₂O— such that R² and R³ define a fused 5-membered ring that includes 2 oxygen atoms;

R³ is selected from —H, —F, —Cl, —Br, —CF₃, —C≡N, —NO₂, —CO₂H, substituted and unsubstituted amino groups, substituted and unsubstituted alkyl groups, substituted and unsubstituted alkoxy groups, substituted and unsubstituted —C(═O)—O-alkyl groups, substituted and unsubstituted arylalkoxy groups, substituted and unsubstituted heterocyclyloxy groups, substituted and unsubstituted alkoxyalkyl groups, substituted and unsubstituted arylalkoxyalkyl groups, substituted and unsubstituted aryloxy group, substituted and unsubstituted heterocycyl groups, substituted and unsubstituted —N(H)—C(═O)-alkyl groups, substituted and unsubstituted —N(H)—C(═O)-aryl groups, substituted and unsubstituted —N(H)—(SO₂)-alkyl groups, substituted and unsubstituted —N(H)—(SO₂)-aryl groups, —N(H)—(SO₂)—CF₃ groups, substituted and unsubstituted —N(H)—(SO₂)-heterocyclyl groups, substituted and unsubstituted —N(H)C(═O)N(H)-alkyl groups, substituted and unsubstituted —N(H)C(═O)N(H)-aryl groups, substituted and unsubstituted —C(═O)—N(H)-alkyl groups, substituted and unsubstituted —C(═O)—N(H)-alkylheterocyclyl groups, substituted and unsubstituted (alkyl)(alkyl)aminoalkyl groups, substituted and unsubstituted (alkyl)(aryl)aminoalkyl groups, substituted and unsubstituted (alkyl)(heterocyclyl)aminoalkyl groups substituted and unsubstituted (alkyl)(arylalkyl)aminoalkyl groups, substituted and unsubstituted (alkyl)(heterocyclylalkyl)aminoalkyl groups, substituted and unsubstituted -alkyl-N(alkyl)-C(═O)-alkyl groups: substituted and unsubstituted -alkyl-N(alkyl)-C(═O)-aryl groups, substituted and unsubstituted -alkyl-N(alkyl)-C(═O)-heterocyclyl groups, substituted and unsubstituted -alkyl-N(alkyl)-C(═O)-alkyl-aryl groups, substituted and unsubstituted-alkyl-N(alkyl)-C(═O)-alkyl-heterocyclyl groups, substituted and unsubstituted alkylaminoalkyl groups, substituted and unsubstituted arylaminoalkyl groups, substituted and unsubstituted heterocyclylaminoalkyl groups, substituted and unsubstituted arylalkylaminoalkyl groups, substituted and unsubstituted heterocyclylalkylaminoalkyl groups, substituted and unsubstituted -alkyl-N(H)—C(═O)-alkyl groups, substituted and unsubstituted -alkyl-N(H)—C(═O)-aryl groups, substituted and unsubstituted -alkyl-N(H)—C(═O)-heterocyclyl groups, substituted and unsubstituted -alkyl-N(H)—C(═O)-alkyl-aryl groups, and substituted and unsubstituted -alkyl-N(H)—C(═O)alkyl-heterocyclyl groups;

R⁴ is —H, —F, —Br, —Cl, —NO₂, —C≡N, —C(═O)—O-alkyl groups, —OH, substituted and unsubstituted arylalkoxy groups, substituted and unsubstituted heterocyclyloxy groups, substituted and unsubstituted alkoxyalkyl groups, substituted and unsubstituted arylalkoxyalkyl groups, substituted and unsubstituted aryloxy groups, substituted and unsubstituted —N(H)—C(═O)-aryl groups, substituted and unsubstituted —N(H)—C(═O)-alkyl groups, substituted and unsubstituted —N(H)—(SO₂)alkyl groups, substituted and unsubstituted —N(H)—(SO₂)-aryl groups, —N(H)—(SO₂)—CF₃ groups, substituted and unsubstituted —N(H)—(SO₂)-heterocyclyl groups, substituted and unsubstituted heterocyclyl groups, substituted and unsubstituted amino groups, substituted and unsubstituted alkyl groups, substituted and unsubstituted alkoxy groups, substituted and unsubstituted —C(═O)—N(H)-alkyl groups, substituted and unsubstituted —C(═O)—N(H)-alkyl-heterocyclyl groups, substituted and unsubstituted (alkyl)(alkyl)aminoalkyl groups, substituted and unsubstituted (alkyl)(aryl)aminoalkyl groups, substituted and unsubstituted (alkyl)(heterocyclyl)aminoalkyl groups substituted and unsubstituted (alkyl)(arylalkyl)aminoalkyl groups, substituted and unsubstituted (alkyl)(heterocyclylalkyl)aminoalkyl groups, substituted and unsubstituted -alkyl-N(alkyl)-C(═O)-alkyl groups, substituted and unsubstituted-alkyl-N(alkyl)-C(═O)-aryl groups, substituted and unsubstituted -alkyl-N(alkyl)-C(═O)-heterocyclyl groups, substituted and unsubstituted -alkyl-N(alkyl)-C(═O)-alkyl-aryl groups, substituted and unsubstituted -alkyl-N(alkyl)-C(═O)-alkylheterocyclyl groups, substituted and unsubstituted alkylaminoalkyl groups, substituted and unsubstituted arylaminoalkyl groups, substituted and unsubstituted heterocyclylaminoalkyl groups substituted and unsubstituted arylalkylaminoalkyl groups, substituted and unsubstituted heterocyclylalkylaminoalkyl groups, substituted and unsubstituted -alkyl-N(H)—C(═O)alkyl groups, substituted and unsubstituted -alkyl-N(H)—C(═O)-aryl groups, substituted and unsubstituted -alkyl-N(H)—C(═O)-heterocyclyl groups, substituted and unsubstituted -alkyl-N(H)—C(═O)-alkyl-aryl groups, and substituted and unsubstituted -alkyl-N(H)—C(═(O)-alkyl-heterocyclyl groups;

R⁵ is selected from —H, —F, —Cl, substituted and unsubstituted alkyl groups, substituted and unsubstituted alkoxy groups, substituted and unsubstituted amino groups, substituted and unsubstituted alkylamino groups, substituted and unsubstituted-dialkylamino groups, and substituted and unsubstituted heterocyclyl groups, or R⁵ is absent if Z¹ is N;

R⁶ is selected from —H, —F, —Cl, —Br, —CF₃, —CO₂H, substituted and unsubstituted alkyl groups; substituted and unsubstituted alkoxy groups including substituted and unsubstituted hetereocycylalkoxy groups, substituted and unsubstituted arylalkoxy groups, and substituted and unsubstituted alkoxyalkoxy groups; substituted and unsubstituted heterocyclyl groups including substituted and unsubstituted hetetocyclylheterocyclyl groups, substituted and unsubstituted arylheterocyclyl groups, substituted and unsubstituted alkylheterocyclyl groups, and substituted and unsubstituted cycloalkylheterocyclyl; substituted and unsubstituted heterocyclyloxy groups, substituted and unsubstituted aryloxy groups, substituted and unsubstituted amino groups including substituted and unsubstituted dialkylamino groups, substituted and unsubstituted (alkyl)(heterocyclyl)amino groups, substituted and unsubstituted heterocyclylalkylamino groups, substituted and unsubstituted arylalkylamino groups, and substituted and unsubstituted heterocyclylamino groups; substituted and unsubstituted —C(═O)N(H)-alkyl groups, substituted and unsubstituted —C(═O)N(H)-aryl groups, substituted and unsubstituted —C(═O)N(H)-heterocyclyl groups, substituted and unsubstituted —C(═O)N(alkyl)(heterocyclyl) groups, and substituted and unsubstituted —C(═O)-heterocyclyl groups; or R⁶ is absent if Z² is N;

R⁷ is selected from —H, —F, —Cl, —Br, —CF₃, —CO₂H, substituted and unsubstituted alkyl groups, substituted and unsubstituted alkoxy groups including substituted and unsubstituted heterocyclylalkoxy groups, substituted and unsubstituted arylalkoxy groups, and substituted and unsubstituted alkoxyalkoxy groups; substituted and unsubstituted heterocyclyl groups including substituted and unsubstituted heterocyclylheterocyclyl groups, substituted and unsubstituted arylheterocyclyl groups, substituted and unsubstituted alkylheterocyclyl groups, and substituted and unsubstituted cycloalkylheterocyclyl groups; substituted and unsubstituted heterocyclyloxy groups, substituted and unsubstituted aryloxy groups, substituted and unsubstituted amino groups including substituted and unsubstituted dialkylamino groups, substituted and unsubstituted (alkyl)(heterocyclyl)amino groups, substituted and unsubstituted heterocyclylalkylamino groups, substituted and unsubstituted arylalkylamino groups, and substituted and unsubstituted heterocyclylamino groups; substituted and unsubstituted —C(O)N(H)-alkyl groups, substituted and unsubstituted —C(═O)N(H)-aryl groups, substituted and unsubstituted —C(═O)N(H)-heterocyclyl groups, substituted and unsubstituted —C(═O)N(alkyl)(heterocyclyl) groups, and substituted and unsubstituted —C(═O)-heterocyclyl groups; or R⁷ is absent if Z³ is N;

R⁸ is selected from —H, —F, —Cl, substituted and unsubstituted alkyl groups, substituted and unsubstituted alkoxy groups, substituted and unsubstituted amino groups, substituted and unsubstituted alkylamino groups, substituted and unsubstituted dialkylamino groups, and substituted and unsubstituted heterocyclyl groups, or R⁸ is absent if Z⁴ is N;

R⁹ is —H; and

R¹⁰ is selected from the group consisting of —H, and substituted and unsubstituted alkyl groups, and further wherein at least one of Z² or Z³ is C and at least one of R⁶ or R⁷ is selected from the group consisting of substituted and unsubstituted piperidinyl substituted heterocyclyl groups, substituted and unsubstituted heterocyclyl substituted piperidinyl groups, substituted and unsubstituted piperidinyl groups dimethylaminoalkyl substituted pyrrolidinyl groups, substituted and unsubstituted 3-alkyl substituted piperazinyl groups, substituted and unsubstituted 3,5-dialkyl substituted piperazinyl groups, substituted and unsubstituted N-hydroxyalkyl substituted piperazinyl groups, substituted and unsubstituted, 1,4-diazacycloheptyl groups, substituted and unsubstituted 1-aza-4-oxacycloheptane groups, substituted and unsubstituted N-ethylpiperazinyl groups, substituted and unsubstituted N-isopropylpiperazinyl groups, substituted and unsubstituted N-sec-butylpiperazinyl groups; substituted and unsubstituted N-2-pyridyl substituted piperazinyl groups, substituted and unsubstituted N-3-pyridyl substituted piperazinyl groups, substituted and unsubstituted N-4-pyridyl substituted piperazinyl groups, substituted and unsubstituted N(H)—CH₂-pyridyl groups, substituted and unsubstituted imidazolyl groups, substituted and unsubstituted 3-alkyl substituted morpholinyl groups, substituted and unsubstituted 3,5-dialkyl substituted morpholinyl groups, dialkylamino substituted pyrrolidinyl groups, pyrrolidinyl groups substituted with both dialkylamino and alkyl groups, substituted and unsubstituted 4-hydroxy substituted piperidinyl groups, substituted and unsubstituted 4-aryl substituted piperidinyl groups, substituted and unsubstituted 4-hydroxy-4-phenyl substituted piperidinyl groups, substituted and unsubstituted cyclohexylpiperazinyl groups, substituted and unsubstituted cyclopentylpiperazinyl groups, substituted and unsubstituted N-alkyl substituted diazabicycloalkane groups, substituted and unsubstituted —N(CH₃)(N-alkyl(4-piperidinyl)) groups, substituted and unsubstituted piperazinyl groups further substituted with a —C(—O)-alkyl group bonded to one of the N atoms of the piperazinyl group, substituted and unsubstituted —N(H)CH₂CH₂CH₂-imidazolyl groups, substituted and unsubstituted —N(H)CH₂CH₂CH₂-pyrrolidinyl groups, substituted and unsubstituted —N(H)CH₂CH₂CH₂-morpholinyl groups, substituted and unsubstituted —N(H)CH₂CH₂CH₂CH₂-piperazinyl groups, substituted and unsubstituted —N(H)CH₂CH₂CH₂-piperidinyl groups, substituted and unsubstituted —N(H)CH₂CH₂CH₂-pyridyl groups, substituted and unsubstituted —N(H)CH₂CH₂-imidazolyl groups, substituted and unsubstituted —N(H)CH₂CH₂-pyrrolidinyl groups, substituted and unsubstituted —N(H)CH₂CH₂-morpholinyl groups, substituted and unsubstituted —N(H)CH₂CH₂CH₂-piperazinyl groups; substituted and unsubstituted —N(H)CH₂C₂-piperidinyl groups, substituted and unsubstituted —N(H)CH₂CH₂-pyridyl groups, substituted and unsubstituted cyclobutylpiperazinyl groups, substituted and unsubstituted —OCH₂-pyrrolidinyl groups, substituted and unsubstituted —OCH₂CH₂-pyrrolidinyl groups, substituted and unsubstituted —OCH₂CH₂CH₂-pyrrolidinyl groups, substituted and unsubstituted piperazinyl groups further substituted with a —CH₂C(═O)—O-alkyl group bonded to one of the N atoms of the piperazinyl group, substituted and unsubstituted piperazinyl groups further substituted with a —C(═O)—O-alkyl group bonded to one of the N atoms of the piperazinyl group, substituted and unsubstituted hydroxypyrrolidinyl groups, substituted and unsubstituted hydroxypiperidinyl groups, substituted and unsubstituted —OCH₂-pyridyl groups, substituted and unsubstituted piperidinylamino groups, substituted and unsubstituted pyridyloxy groups with a —C(═O)—N(H)(alkyl) group bonded to a carbon atom of the pyridine ring of the pyridyloxy group, and substituted and unsubstituted pyridyloxy groups with a —C(═O)—N(alkyl)₂ group bonded to a carbon atom of the pyridine ring of the pyridyloxy group.

vii) A compound having the structure below, a tautomer of the compound, a pharmaceutically acceptable salt of the compound, or a pharmaceutically acceptable salt of the tautomer: 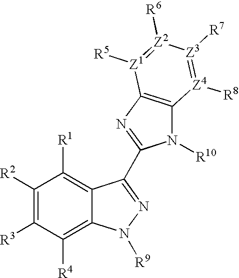
wherein
Z¹, Z², Z³, and Z⁴ are independently selected from C or N;

R¹ is selected from —H, —F, —Cl, —Br, —NO₂, —C≡N, —C(═O)—O-alkyl groups, —OH, substituted and unsubstituted arylalkoxy groups, substituted and unsubstituted heterocyclyloxy groups, substituted and unsubstituted alkoxyalkyl groups, substituted and unsubstituted arylalkoxyalkyl groups, substituted and unsubstituted aryloxy groups, substituted and unsubstituted —N(H)—C(═O)-aryl groups, substituted and unsubstituted —N(H)—C(═O)-allyl groups, substituted and unsubstituted —N(H)—(SO₂)-alkyl groups, substituted and unsubstituted —N(H)—(SO₂)-aryl groups, —N(H)—(SO₂)—CF₃ groups, substituted and unsubstituted —N(H)—(SO₂)-heterocyclyl groups, substituted and unsubstituted heterocyclyl groups, substituted and unsubstituted alkyl groups, substituted and unsubstituted alkoxy groups, substituted and unsubstituted amino groups, substituted and unsubstituted —C(═O)—N(H)-alkyl groups, substituted and unsubstituted —C(═O)—N(H)-alkyl-heterocyclyl groups, substituted and unsubstituted (alkyl)(alkyl)aminoalkyl groups, substituted and unsubstituted(alkyl)(aryl)aminoalkyl groups, substituted and unsubstituted (alkyl)(heterocyclyl)aminoalkyl groups substituted and unsubstituted (alkyl)(arylalkyl)aminoalkyl groups, substituted and unsubstituted (alkyl)(heterocyclylalkyl)aminoalkyl groups, substituted and unsubstituted -alkyl-N(alkyl)-C(═O)alkyl groups, substituted and unsubstituted -alkyl-N(alkyl)-C(═O)aryl groups, substituted and unsubstituted -alkyl-N(alkyl)-C(═O)-heterocyclyl groups, substituted and unsubstituted -alkyl-N(alkyl)-C(═O)-alkyl-aryl groups, substituted and unsubstituted -alkyl-N(alkyl)-C(═O)alkyl-heterocyclyl groups, substituted and unsubstituted alkylaminoalkyl groups, substituted and unsubstituted arylaminoalkyl groups, substituted and unsubstituted heterocyclylaminoalkyl groups, substituted and unsubstituted arylalkylaminoalkyl groups, substituted and unsubstituted heterocyclylalkylaminoalkyl groups, substituted and unsubstituted -alkyl-N(H)—C(═O)-alkyl groups, substituted and unsubstituted -alkyl-N(H)—C(═O)-aryl groups, substituted and unsubstituted -alkyl-N(H)—C(═O)-heterocyclyl groups, substituted and unsubstituted -alkyl-N(H)—C(═O)-alkyl-aryl groups, and substituted and unsubstituted -alkyl-N(H)—C(═O)-alkyl-heterocyclyl groups;

R² is selected from —H, —F, —Cl, —Br, —C≡N, —NO₂, —CO₂H, —OH, substituted and unsubstituted guanidinyl groups, substituted and unsubstituted amino groups, substituted and unsubstituted alkyl groups, substituted and unsubstituted —C(═O)O-alkyl groups, substituted and unsubstituted —C(═O)O-aryl groups, substituted and unsubstituted —C(═O)O-heteroaryl groups, substituted and unsubstituted —C(═O)N(H)-alkyl groups, substituted and unsubstituted —C(═O)—N)-alkyl-heterocyclyl groups, substituted and unsubstituted —C(═O)N(H)-aryl groups, substituted and unsubstituted —C(═O)N(H)-heterocyclyl groups, substituted and unsubstituted —N(H)C(═O)-alkyl groups, substituted and unsubstituted —N(H)C(═O)aryl groups, substituted and unsubstituted —N(H)C(═O)-heterocyclyl groups, substituted and unsubstituted —N(H)C(—O)N(M-alkyl groups, substituted and unsubstituted —N(H)C(═O)N(H)-aryl groups, substituted and unsubstituted —N(H)C(═O)N(H)-heterocyclyl groups, substituted and unsubstituted —N(H)—(SO₂)-alkyl groups, substituted and unsubstituted —N(H)—(SO₂)-aryl groups, —N(H)—(SO₂)—CF₃ groups, substituted and unsubstituted —N(H)—(SO₂)-heterocyclyl groups, substituted and unsubstituted —N(H)-heterocyclyl groups, substituted and unsubstituted heterocyclyl groups, substituted and unsubstituted alkoxy groups, substituted and unsubstituted arylalkoxy groups, substituted and unsubstituted aryloxy groups, substituted and unsubstituted alkoxyalkyl groups, substituted and unsubstituted arylalkoxyalkyl groups, substituted and unsubstituted heterocyclyloxy, substituted and unsubstituted heterocyclylalkoxy groups, substituted and unsubstituted (alkyl)(alkyl)aminoalkyl groups, substituted and unsubstituted (alkyl)(aryl)aminoalkyl groups, substituted and unsubstituted (alkyl)(heterocyclyl)aminoalkyl groups substituted and unsubstituted (alkyl)(arylalkyl)aminoalkyl groups, substituted and unsubstituted (alkyl)(heterocyclylalkyl)aminoalkyl groups, substituted and unsubstituted -alkyl-N(alkyl)-C(═O)-alkyl groups, substituted and unsubstituted -alkyl-N(alkyl)-C(═O)-aryl groups, substituted and unsubstituted -alkyl-N(alkyl)-C(═O)-heterocyclyl groups, substituted and unsubstituted alkyl-N(alkyl)-C(═O)-alkyl-aryl groups substituted and unsubstituted -alkyl-N(alkyl)-C(═O)-alkyl-heterocyclyl groups, substituted and unsubstituted alkylaminoalkyl groups, substituted and unsubstituted arylaminoalkyl groups, substituted and unsubstituted heterocyclylaminoalkyl groups, substituted and unsubstituted arylalkylaminoalkyl groups, substituted and unsubstituted heterocyclylalkylaminoalkyl groups, substituted and unsubstituted -alkyl-N(H)—C(═O)alkyl groups, substituted and unsubstituted -alkyl-N(H)—C(═O)-aryl groups, substituted and unsubstituted -alkyl-N(H)—C(═O)-heterocyclyl groups, substituted and unsubstituted -alkyl-N(H)—C(═O)-alkyl-aryl groups, and substituted and unsubstituted -alkyl-N(H)—C(═O)-alkyl-heterocyclyl groups; or R² and R³ are a group of formula —OCH₂O— such that R² and R³ define a fused 5-membered ring that includes 2 oxygen atoms;

R³ is selected from —H, —F, —Cl, —Br; —CF₃, —C≡N, —NO₂, —CO₂H, substituted and unsubstituted amino groups, substituted and unsubstituted alkyl groups; substituted and unsubstituted alkoxy groups, substituted and unsubstituted —C(═O)—O-alkyl groups, substituted and unsubstituted arylalkoxy groups, substituted and unsubstituted-heterocyclyloxy groups, substituted and unsubstituted alkoxyalkyl groups, substituted and unsubstituted arylalkoxyalkyl groups, substituted and unsubstituted aryloxy group, substituted and unsubstituted heterocycyl groups, substituted and unsubstituted, —N(H)—C(═O)-alkyl groups, substituted and unsubstituted —N(H)—C(═O)-aryl groups, substituted and unsubstituted —N(H)—(SO₂)-alkyl groups, substituted and unsubstituted —N(H)—(SO₂)-aryl groups, —N(H)—(SO₂)—CF₃ groups, substituted and unsubstituted —N(H)—(SO₂)-heterocyclyl groups, substituted and unsubstituted —N(H)C(═O)N(H)-alkyl groups, substituted and unsubstituted —N(H)C(═O)N(H)-aryl groups, substituted and unsubstituted —C(═O)—N(H)-alkyl groups, substituted and unsubstituted —C(═O)—N(H)-alkyl-heterocyclyl groups, substituted and unsubstituted (alkyl)(alkyl)aminoalkyl groups, substituted and unsubstituted (alkyl)(aryl)aminoalkyl groups, substituted and unsubstituted (alkyl)(heterocyclyl)aminoalkyl groups, substituted and unsubstituted (alkyl)(arylalkyl)aminoalkyl groups, substituted and unsubstituted (alkyl)(heterocyclylalkyl)aminoalkyl groups, substituted and unsubstituted -alkyl-N(alkyl)-C(═O)-alkyl groups; substituted and unsubstituted -alkyl-N(alkyl)-C(═O)-aryl groups, substituted and unsubstituted -alkyl-N(alkyl)-C(═O)-heterocyclyl groups, substituted and unsubstituted -alkyl-N(alkyl)-C(═O)-alkyl-aryl groups, substituted and unsubstituted -alkyl-N(alkyl)-C(═O)-alkyl-heterocyclyl groups, substituted and unsubstituted alkylaminoalkyl groups, substituted and unsubstituted arylaminoalkyl groups, substituted and unsubstituted heterocyclylaminoalkyl groups, substituted and unsubstituted arylalkylaminoalkyl groups, substituted; and unsubstituted heterocyclylalkylaminoalkyl groups; substituted and unsubstituted -alkyl-N(H)—C(═O)-alkyl groups, substituted and unsubstituted -alkyl-N(H)—C(═O)-aryl groups, substituted and unsubstituted -alkyl-N(H)—C(═O)-heterocyclyl groups, substituted and unsubstituted -alkyl-N(H)—C(═O)-alkyl-aryl groups, and substituted and unsubstituted -alkyl-N(H)—C(═O)-alkyl-heterocyclyl groups;

R⁴ is —H, —F, —Br, —Cl, —NO₂, —C≡N, —C(═O)—O-alkyl groups, —OH, substituted and unsubstituted arylalkoxy groups, substituted and unsubstituted heterocyclyloxy groups, substituted and unsubstituted alkoxyalkyl groups, substituted and unsubstituted-arylalkoxyalkyl groups, substituted and unsubstituted aryloxy groups, substituted and unsubstituted —N(H)—C(═O)-aryl groups, substituted and unsubstituted —N(H)—C(═O)-alkyl groups, substituted and unsubstituted —N(H)—(SO₂)-alkyl groups, substituted and unsubstituted —N(H)—(SO₂)-aryl groups, —N(H)—(SO₂)—CF₃ groups, substituted and unsubstituted —N(H)—(SO₂)-heterocyclyl groups, substituted and unsubstituted heterocyclyl groups, substituted and unsubstituted amino groups, substituted and unsubstituted alkyl groups, substituted and unsubstituted alkoxy groups, substituted and unsubstituted —C(═O)—N(H)-alkyl groups, substituted and unsubstituted —C(═O)—N(H)-alkyl-heterocyclyl groups, substituted and unsubstituted (alkyl)(alkyl)aminoalkyl groups, substituted and unsubstituted (alkyl)(aryl)aminoalkyl groups, substituted and unsubstituted (alkyl)(heterocyclyl) aminoalkyl groups substituted and unsubstituted (alkyl)(arylalkyl)aminoalkyl groups; substituted and unsubstituted (alkyl)(heterocyclylalkyl)aminoalkyl groups, substituted and unsubstituted -alkyl-N(alkyl)-C(═O)-alkyl groups, substituted and unsubstituted -alkyl-N(alkyl)-C(═O)-aryl groups, substituted and unsubstituted -alkyl-N(alkyl)-C(═O)-heterocyclyl groups, substituted and unsubstituted -alkyl-N(alkyl)-C(═O)-alkyl-aryl groups, substituted and unsubstituted -alkyl-N(alkyl)-C(═O)-alkyl-heterocyclyl groups, substituted and unsubstituted alkylaminoalkyl groups, substituted and unsubstituted arylaminoalkyl groups, substituted and unsubstituted heterocyclylaminoalkyl groups substituted and unsubstituted arylalkylaminoalkyl groups, substituted and unsubstituted heterocyclylalkylaminoalkyl groups, substituted and unsubstituted -alkyl-N(—H)—C(═O)-alkyl groups, substituted and unsubstituted -alkyl-N(H)—C(═O)-aryl groups, substituted and unsubstituted -alkyl-N(H)—C(═O)-heterocyclyl groups, substituted and unsubstituted -alkyl-N(H)—C(═O)-alkyl-aryl groups, and substituted and unsubstituted -alkyl-N(H)—C(═O)-alkyl-heterocyclyl groups;

R⁵ is selected from —H, —F, —Cl, substituted and unsubstituted alkyl groups, substituted and unsubstituted alkoxy groups, substituted and unsubstituted amino groups, substituted and unsubstituted alkylamino groups, substituted and unsubstituted dialkylamino groups, and substituted and unsubstituted heterocyclyl groups, or R⁵ is absent if Z¹ is N;

R⁶ is selected from —H, —F, —Cl, —Br, —CF₃, —CO₂H, substituted and unsubstituted alkyl groups, substituted and unsubstituted alkoxy groups including substituted and unsubstituted heterocyclylalkoxy groups, substituted and unsubstituted arylalkoxy groups, and substituted and unsubstituted alkoxyalkoxy groups; substituted and unsubstituted heterocyclyl groups including substituted and unsubstituted heterocyclylheterocyclyl groups, substituted and unsubstituted arylheterocyclyl groups, substituted and unsubstituted alkylheterocyclyl groups, and substituted and unsubstituted cycloalkylheterocyclyl groups; substituted and unsubstituted heterocyclyloxy groups, substituted and unsubstituted aryloxy groups, substituted and unsubstituted amino groups including substituted and unsubstituted dialkylamino groups, substituted and unsubstituted (alkyl)(heterocyclyl)amino groups, substituted and unsubstituted heterocyclylalkylamino groups, substituted and unsubstituted arylalkylamino groups, and substituted and unsubstituted heterocyclylamino groups; substituted and unsubstituted —C(═O)N(H)-alkyl groups, substituted and unsubstituted —C(═O)N(H)-aryl groups, substituted and unsubstituted —C(═O)N(H)-heterocyclyl groups; substituted and unsubstituted —C(═O)N(alkyl) (heterocyclyl) groups, and substituted and unsubstituted —C(═O)-heterocyclyl groups; or R⁶ is absent if Z² is N;

R⁷ is selected from —H, —F, —Cl, —Br, —CF₃, —CO₂H, substituted and unsubstituted alkyl groups, substituted and unsubstituted alkoxy groups including substituted and unsubstituted heterocyclylalkoxy groups, substituted and unsubstituted arylalkoxy groups, and substituted and unsubstituted alkoxyalkoxy groups; substituted and unsubstituted heterocyclyl groups including substituted and unsubstituted heterocyclylheterocyclyl groups, substituted and unsubstituted arylheterocyclyl groups, substituted and unsubstituted alkylheterocyclyl groups, and substituted and unsubstituted cycloalkylheterocyclyl groups; substituted and unsubstituted heterocyclyloxy groups, substituted and unsubstituted aryloxy groups, substituted and unsubstituted amino groups including substituted and unsubstituted dialkylamino groups, substituted and unsubstituted (alkyl)(heterocyclyl)amino groups, substituted and unsubstituted heterocyclylalkylamino groups, substituted and unsubstituted arylalkylamino groups, and substituted and unsubstituted heterocyclylamino groups; substituted and unsubstituted —C(═O)N(H)-alkyl groups, substituted and unsubstituted —C(═O)N(H)-aryl groups, substituted and unsubstituted —C(═O)N(H)-(heterocyclyl groups, substituted and unsubstituted —C(═O)N(alkyl)(heterocyclyl) groups, and substituted and unsubstituted —C(═O)-heterocyclyl groups; or R⁷ is absent if Z³ is N;

R⁸ is selected from —H, —F, —Cl, substituted and unsubstituted alkyl groups, substituted and unsubstituted alkoxy groups, substituted and unsubstituted amino groups, substituted and unsubstituted alkylamino groups, substituted and unsubstituted dialkylamino groups, and substituted and unsubstituted heterocyclyl groups, or R⁸ is absent if Z³ is N;

R⁹ is —H; and

R¹⁰ is selected from the group consisting of —H, and substituted and unsubstituted alkyl groups, and further wherein at least one of the following is true: (i) R¹ is selected from the group consisting of unsubstituted —NH₂ groups, and substituted and unsubstituted pyrrolidinylalkylamino groups; (ii) R² is selected from the group consisting of substituted and unsubstituted thiazolylalkylamino groups, substituted and unsubstituted pyrrolidinylalkylamino groups, and substituted and unsubstituted aminoalkylamino groups; or (iii) R³ is selected from the group consisting of substituted and unsubstituted thiazolylalkylamino groups, substituted and unsubstituted benzimidazolylalkylamino groups, substituted and unsubstituted imidazolylalkylamino groups, substituted and unsubstituted furanylalkylamino groups, and substituted and unsubstituted arylalkylamino groups.

XV. Indazole Compounds as Described in International Patent Publication WO01053268, Including:

i) A compound of formula:

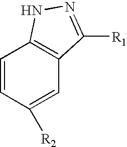

wherein R₁, is hydrogen or a substituted or unsubstituted alkyl, aryl, heteroaryl, carbocycle, or heterocycle group, or

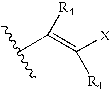

wherein R₄ is H or lower alkyl, and X is a substituted or -unsubstituted alkyl, aryl, heteroaryl, carbocycle, or heterocycle group ; and

R₂ is a substituted or unsubstituted alkyl aryl, heteroaryl, carbocycle, or heterocycle group, or 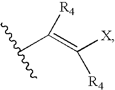
wherein

R₄ is H or lower alkyl, and X is a substituted or unsubstituted aryl, heteroaryl, carbocycle, or heterocycle group ; or a pharmaceutically acceptable salt of the compound; or a prodrug or pharmaceutical active metabolite of the compound, or a pharmaceutically acceptable salt of a prodrug or metabolite thereof

ii) A compound of formula: 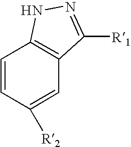
wherein R′₁, is a substituted or unsubstituted alkyl, aryl, heteroaryl, carbocycle, or heterocycle group or

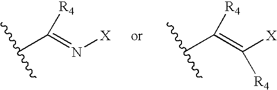

wherein each R₄ is individually H or lower alkyl, and X is a substituted or unsubstituted alkyl, aryl, heteroaryl, carbocycle, or heterocycle group; and R′₂ is a substituted or unsubstituted amino, nitro, alkenyl, alkyl, aryl, heteroaryl, carbocycle,

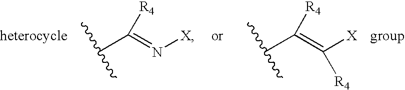

wherein the R₄ groups are independently H or lower alkyl, and X is selected from a substituted or unsubstituted alkyl, aryl, heteroaryl, carbocycle, or heterocycle group;

or a pharmaceutically acceptable salt of the compound; or a prodrug or pharmaceutically active metabolite of the compound, or a pharmaceutically acceptable salt of the prodrug or metabolite thereof

XVI. Chk1 Receptor Antagonists as Described in International Patent Publication WO00/016781, Including:

i) A compound of formula:

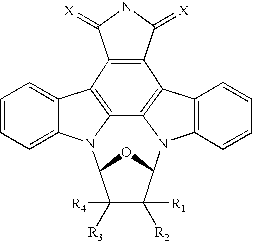

wherein X represents N, S or OH and R¹, R², R³, and R⁴ independently represent C₁₋₆ alkyl, OH, or SH or H.

XVII. Heteroaromatic Carboxamide Compounds as Described in International Patent Publication WO03/037886, Including:

i) A compound of formula:

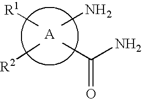

wherein A is a 5-membered heteroaromatic ring containing one or two heteroatoms independently selected from oxygen, nitrogen, or sulfur;

R¹ is selected from the group consisting of: hydrogen, halogen, cyano, nitro, —N(R³)₂, —CON(R³)₂, —COOR³, —NR³COR³, S(O)mR³, —SO₂N(R³)₂, —NR³SO₂R³, alkyl, trifluoromethyl, trifluoromethoxy, alkenyl, alkynyl, alkoxy, alkanoyl, substituted or unsubstituted aryl, and a substituted or unsubstituted 5- to 7-membered heteroaromatic ring containing one to three heteroatoms independently selected from

oxygen, nitrogen, or sulfur, wherein said substituent (s) are independently selected from the group consisting of: halogen, cyano, nitro, —N(R⁴)₂, —CON(R⁴)₂, —COOR⁴, —NR⁴COR⁴, S(O)mR⁴—SO₂N(R⁴)₂, —NR⁴SO₂R⁴, alkyl, trifluoromethyl, trifluoromethoxy, alkenyl, alkynyl, alkoxy, alkanoyl, aminoalkyl, and aryl;

R² is selected from the group consisting of: substituted or unsubstituted aryl, and a 5- to 7-membered substituted or unsubstituted heteroaromatic ring containing one to three heteroatoms independently selected from oxygen, nitrogen, or sulfur,

wherein said substituent(s) are independently selected from the group consisting of: halogen, cyano, nitro, —N(R⁴)₂, —CON(R4)₂, —COOR4, —NR4COR4, S(O)ₘR4, —SO2N(R4)₂, —NR⁴SO₂R⁴, alkyl, trifluoromethyl; trifluoromethoxy, alkenyl, alkynyl, alkoxy, alkanoyl, aminoalkyl, and aryl;

R¹ and R² can optionally be taken together form a 5 or 6 membered saturated or unsaturated ring optionally substituted with one or more substituent selected from the group consisting of: halogen, cyano, nitro, —N(R³)₂, —CON(R³)₂, —COOR³, —NR³COR³, S(O)ₘR³, —SO₂N(R³)₂, —NR³SO₂R³, alkyl, trifluoromethyl, trifluoromethoxy, alkenyl, alkynyl, alkoxy, alkanoyl, substituted or unsubstituted aryl, and a substituted or unsubstituted 5- to 7-membered heteroaromatic ring containing one to three heteroatoms independently selected from

oxygen, nitrogen, or sulfur, wherein said substituent(s) are independently selected from the group consisting of: halogen, cyano, nitro, —N(R⁴)₂, —CON(R⁴)₂, —COOR⁴, —NR⁴COR⁴, S(O)ₘR⁴, —SO₂N(R⁴)₂, —NR⁴SO₂R⁴, alkyl, trifluoromethyl, trifluoromethoxy, alkenyl, alkynyl, alkoxy, alkanoyl, aminoalkyl, and aryl;

R³ is selected from the group consisting of: hydrogen or alkyl;

R⁴ is selected from the group consisting of: hydrogen or alkyl; m is an integer 0, 1, or 2; and isomers, tautomers, carriers, prodrugs, pharmaceutically acceptable salts thereof.

ii) A compound of formula:

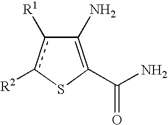

wherein,

R¹ is selected from the group consisting of: hydrogen, halogen, cyano, nitro, —N(R³)₂, —CON(R³)₂, —COOR³, —NR³COR³, S(O)ₘR³, —SO₂N(R³)₂, —NR³SO₂R³, alkyl, trifluoromethyl, trifluoromethoxy, alkenyl, alkynyl, alkoxy, alkanoyl, substituted or unsubstituted aryl, and a substituted or unsubstituted 5- to 7-membered heteroaromatic ring containing one to three heteroatoms independently selected from

oxygen, nitrogen, or sulfur, wherein said substituent(s) are independently selected from the group consisting of: halogen, cyano, nitro, —N(R⁴)₂, —CON(R⁴)₂, —COOR⁴, —NR⁴COR⁴, S(O)ₘ, —SO₂N(R⁴)₂, —NR⁴SO₂R⁴, alkyl, trifluoromethyl, trifluoromethoxy, alkenyl, alkynyl, alkoxy, alkanoyl, aminoalkyl, and aryl;

R² is selected from the group consisting of: substituted or unsubstituted aryl, and a 5- to 7-membered substituted or unsubstituted heteroaromatic ring containing one to three heteroatoms independently selected from

oxygen, nitrogen, or sulfur, wherein said substituent(s) are independently selected from the group consisting of: halogen, cyano, nitro, —N(R⁴)₂, —CON(R⁴)₂, —COOR⁴, —NR⁴COR⁴, S(O)ₘR⁴, —SO₂N(R⁴)₂, —NR⁴SO₂R⁴, alkyl, trifluoromethyl, trifluoromethoxy, alkenyl, alkynyl, alkoxy, alkanoyl, aminoalkyl, and aryl;

R¹ and R² can optionally be taken together form a 5 or 6 membered saturated or unsaturated ring optionally substituted with one or more substituent selected from the group consisting of: halogen, cyano, nitro, —N(R³)₂, —CON(R³)₂, —COOR³, from the group consisting of: halogen, cyano, nitro, —N(R⁴)₂, —CON(R⁴)₂, —COOR⁴, —NR⁴COR⁴, S(O)ₘR⁴, —SO₂N(R⁴)₂, —NR⁴SO₂R⁴, alkyl, trifluoromethyl, trifluoromethoxy, alkenyl, alkynyl alkoxy, alkanoyl, aminoalkyl, and aryl.

R³ is selected from the group consisting of: hydrogen or alkyl;

R⁴ is selected from the group consisting of hydrogen or alkyl; m is an integer 0, 1, or 2; and isomers, tautomers, carriers, prodrugs, pharmaceutically acceptable salts thereof.

XVIII. Aminothiophene Compounds as Described in International Patent Publication WO03/029242, Including:

i) A compound of formula:

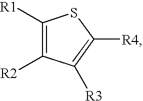

wherein,

R₁ is CONH₂, or SO₂NH₂; R₂ is NR₅R₆;

R₃ is H, or halogen; R₄ is aryl, or heteroaryl; R5 is H, or alkyl; provided that when R₁ is CONH₂, R₆ is selected from the group consisting of H, CO-alkyl, SO₂-alkyl, CONH₂, CONH-alkyl CONH-aryl, CONH-heteroaryl, CSNH₂, CSNH-alkyl, CSNH-aryl, CSNH-heteroaryl, SO₂NH₂, SO₂NH-alkyl, SO₂NH-aryl, and SO₂NH-heteroaryl;

When R₁, is SO₂NH₂, R₆ is CONH; and when R₁ is CONH, R₂ is not NHCONH₂;

and pharmaceutically acceptable salts, hydrates and solvated thereof.

XIX. Heterocyclic-hydroxyimino-fluorene Compounds as Described in International Patent Publication WO0216326, Including:

i) A compound of formula:

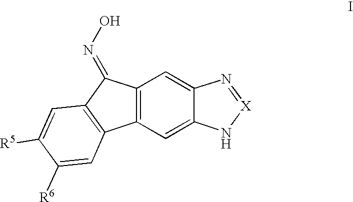

wherein: R⁵ and R⁶ are each independently hydrogen, halo, or a substituted or unsubstituted C₁-C₈ alkyl, C₁-C₈ alkoxy, aryl, heteroaryl, acyl, thioalkyl, sulfonyl, or sulfoxyl; and X is C—Y or N,

where Y is hydrogen, halo, NH₂, NO₂, or a substituted or unsubstituted alkyl, cycloalkyl, heterocycloalkyl, alkoxy, alkenyl, aryl, heteroaryl, aryloxy, alkylamino, dialkylamino, thioalkyl, acyl, sulfonyl, sulfoxide, or thioaryl; or a pharmaceutically acceptable prodrug of said compound, pharmaceutically active metabolite of said compound, or pharmaceutically acceptable salt of said compound or metabolite.

ii) A compound of formula:

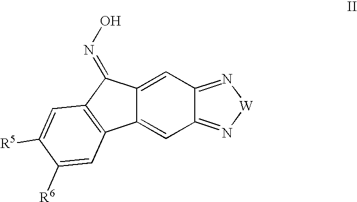

wherein: R⁵ and R⁶ are each independently hydrogen, halo, or a substituted or unsubstituted C₁-C₈ alkyl, C₁-C₈ alkoxy, aryl, heteroaryl, acyl, thioalkyl, sulfonyl, or sulfoxyl ; and

W is O or S; or a pharmaceutically acceptable prodrug of said compound, pharmaceutically active metabolite of said compound, or pharmaceutically acceptable salt of said compound or metabolite.

XX. Scytoneman Skeleton Containing Compounds as Described in U.S. Pat. No. 6,495,586, Including:

i) A compound of formula:

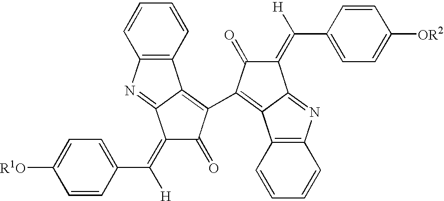

wherein R¹ and R² are independently H, an alkyl group having up to 5 carbon atoms, or —CO—(CH₂)n-CH3 where n=O to 16.

XXI. Heteroarylbenzamide Compounds as Described in International Patent Publication WO0153274, Including:

i) A compound of formula:

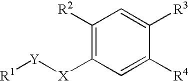

wherein: R¹ is a moiety represented by the formula

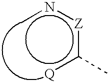

where

Z is selected from the group consisting of CH and NH, and Q is a moiety such that R¹ is a substituted or unsubstituted monocyclic or bicyclic heteroaryl which has at least two carbon atoms in the heteroaryl ring system;

X is selected from the group consisting of CH₂, O, S, and NH;

Y is selected from the group consisting of CH₂, O, and S, provided that at least one of X and Y is CH₂, or X and Y together with the bond there-between form a cyclopropyl;

R² and R³ are independently selected from the group consisting of hydrogen, methyl, halogen, trifluoromethyl, and cyano ; and

R⁴ is selected from the group consisting of

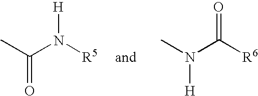

where R⁵ is selected from the group consisting of substituted and unsubstituted aryl, heteroaryl, cycloalkyl, heterocycloalkyl, O—R⁷, NR⁸R⁹, C₁-C₈ alkyl, and monocyclic heterocycloalkyl,

R⁶ is selected from the group consisting of substituted and unsubstituted aryl, heteroaryl, cycloalkyl, heterocycloalkyl, alkenyl, O—R⁷, C(O)R⁷, NR⁸R⁹, C₂-C₈ alkyl, and monocyclic heterocycloalkyl,

where R⁷ is selected from the group consisting of substituted and unsubstituted alkyl, cycloalkyl, heterocycloalkyl, aryl, and heteroaryl,

R⁸ is selected from the group consisting of hydrogen, and substituted and unsubstituted alkyl, and

R⁹ is selected from the group consisting of substituted and unsubstituted alkyl, aryl, heteroaryl cycloalkyl, and heterocycloalkyl;

or a pharmaceutically acceptable prodrug, pharmaceutically active metabolite, or pharmaceutically acceptable salt thereof.

ii) A compound of formula:

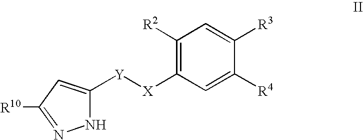

wherein:

X is selected from the group consisting of CH₂, O, and S;

Y is selected from the group consisting of CH₂ and S, provided that at least one of X and Y is CH₂;

R² and R³ are independently selected from the group consisting of hydrogen, methyl, fluorine, and chlorine;

R⁴ is selected from the group consisting of

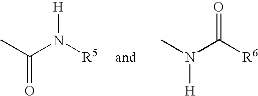

where R⁵ and R⁶ are each independently selected from the group consisting of substituted and unsubstituted aryl and heteroaryl; and

R¹⁰ is selected from the group consisting of substituted and unsubstituted alkenyl, aryl: heteroaryl, and HNR⁹,

where R⁹ is selected from the group consisting of substituted and unsubstituted alkyl, aryl, heteroaryl, cycloalkyl, and heterocycloalkyl;

or a pharmaceutically acceptable prodrug, pharmaceutically active metabolite, or pharmaceutically acceptable salt thereof.

iii) A compound of formula:

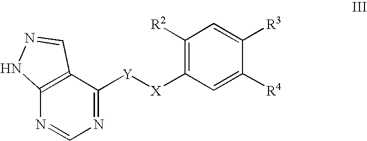

wherein:

X is selected from the group consisting of CH₂, O, S, and NH;

Y is selected from the group consisting of CH₂, O, and S, provided that at least one of X and Y is CH₂, or X and Y together with the bond there-between form a cyclopropyl;

R² and R³ are independently selected from the group consisting of hydrogen, methyl, halogen, trifluoromethyl, and cyano; and

R⁴ is selected from the group consisting of

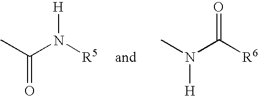

where R⁵ is selected from the group consisting of substituted and unsubstituted aryl, heteroaryl, cycloalkyl, heterocycloalkyl, O—R⁷, NR⁸R⁹, C₁-C₈ alkyl, and monocyclic heterocycloalkyl,

R⁶ is selected from the group consisting of substituted and unsubstituted aryl, heteroaryl, cycloalkyl, heterocycloalkyl, alkenyl, O—R⁷, C(O)R⁷, NR⁸R⁹, C₂-C₈ alkyl, and monocyclic heterocycloalkyl,

where R⁷ is selected from the group consisting of substituted and unsubstituted alkyl, cycloalkyl, heterocycloalkyl, aryl, and heteroaryl,

R⁸ is selected from the group consisting of hydrogen and substituted and unsubstituted alkyl, and

R⁹ is selected from the group consisting of substituted and unsubstituted alkyl, aryl, heteroaryl, cycloalkyl, and heterocycloalkyl;

or a pharmaceutically acceptable prodrug, pharmaceutically active metabolite, or pharmaceutically acceptable salt thereof.

XXII. Chromane Compounds as Described in International Patent Publication WO02070515, Including:

i) A compound of formula:

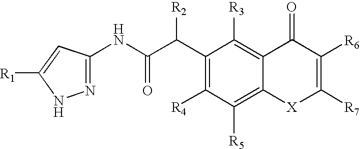

wherein

R₁ is a C₃-C₆ cycloalkyl group optionally substituted by a straight or branched C₁-C₆ alkyl or by aryl C₁-C₆ alkyl group;

R₂ is a hydrogen atom or a straight or branched C₁-C₆ alkyl or C₂-C₄ alkenyl group, each of which being optionally substituted by hydroxy, C₁-C₆ alkoxy, amino or C₁-C₆ alkylamino;

R₃, R₄ and R₅ are, each independently, hydrogen, halogen, hydroxy, amino or straight or branched C₁-C₆ alkyl, C₁-C₆ alkoxy or C₁-C₆ alkylamino;

R₆ and R₇ are, each independently, hydrogen, hydroxy, amino, aminocarbonyl, ureido, guanidyl, pyrrolidinyl optionally substituted by oxo groups, straight or branched C₁-C₆ alkyl optionally substituted by hydroxy or amino groups, straight or branched C₁-C₆ alkoxy, aryl or arylcarbonyl

optionally substituted by halogen, hydroxy, amino, straight or branched C1-C6 alkyl or C₁-C₆ alkoxy groups, or a group selected from alkylcarbonyl, alkylamino, alkylaminocarbonyl or arylalkyloxy wherein alkyl stands for straight or branched C₁-C₆alkyl;

X is an oxygen or sulfur atom or represents a group —N(R₈)—

wherein R₈ is hydrogen or a straight or branched C₁-C₆ alkyl or C₂-C₄ alkenyl group, each of which being optionally substituted by hydroxy, amino, C₁-C₆ alkoxy or C₁-C₆ alkylamino;

or a pharmaceutically acceptable salt thereof; provided that the compound is other than N-(5-cyclopropyl-1H-pyrazol-3-yl)-2-[2-(4-methoxyphenyl)-4-oxo-4H-chromen-6-yl]acetamide;

XII. Oxindole Compounds as Described in International Patent Publication WO03051838, Including:

i) A compound of formula:

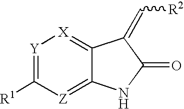

or a therapeutically acceptable salt thereof, wherein

X is selected from the group consisting of —N— and —CR^{X—;}

Y is selected from the group consisting of —N— and —CR^{Y}—;

Z is selected from the group consisting of —N— and —CR^{Z}—;

with the proviso that at least one of Y and Z is other than —N—;

one of R^{X}, R^{Y}, R^{Z}, and R¹ is selected from the group consisting of aryl and heterocycle and the others are hydrogen; and

R² is selected from the group consisting of heterocycle and aryl ; with the proviso that when R² is heterocycle the heterocycle is other than imidazolyl.

XXIV. Diarylurea Compounds are Described in U.S. Provisional Patent Application 60/583,080, Including:

i) A compound of formula:

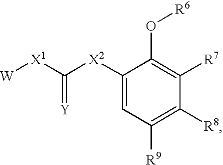

wherein X¹ is null, —O—, —S—, —CH₂—, or —N(R¹)—;

X² is —O—, —S—, or —N(R¹)—;

Y is O or S; or ═Y represents two hydrogen atoms attached to a common carbon atom;

W is selected from the group consisting of heteroaryl, aryl, heterocycloalkyl, cycloalkyl, and C₁₋₆alkyl substituted with a heteroaryl or aryl group, wherein said aryl group W is optionally substituted with one to four substituents represented by R², said heteroaryl group W is optionally substituted with one to four substituents represented by R⁵, and said heterocycloalkyl and cycloalkyl groups W are optionally substituted with one or two C₁₋₆alkyl substituents;

R¹ is selected from the group consisting of hydro, C₁₋₆alkyl, C₂₋₆alkenyl, C₂₋₆alkynyl, and aryl;

R² is selected from the group consisting of heteroaryl, halo, optionally substituted C₁₋₆alkyl, C₂₋₆alkenyl, OCF3, NO₂, CN, NC, N(R³)₂, OR³, CO₂R³, C(O)—N(R¹³)₂, C(O)R³, N(R¹)COR³, N(R¹)C(O)OR³, N(R¹)C(O)C₁₋₆alkyleneC(O)R³, N(R¹)C(O)C₁₋₆alkyleneC(O)OR³, N(R¹)C(O)C₁₋₆alkyleneOR³, N(R¹)C(O)—C₁₋₆alkyleneNHC(O)OR³, N(R¹)C(O)C₁₋₆-alkyleneSO₂NR³, C₁₋₆alkyleneOR³, and SR³;

R³ is selected from the group consisting of hydro, C₁₋₆alkyl, C₂₋₆alkenyl, cycloalkyl, aryl, heteroaryl, SO₂R⁴, halo, C₁₋₆alkyl substituted with one or more of halo, hydroxy, aryl, heteroaryl, heterocycloalkyl, N(R⁴)₂, and SO₂R⁴, C₁₋₆alkylenearyl, C₁₋₆alkyleneheteroaryl, C₁₋₆alkyleneC₃₋₈heterocycloalkyl, C₁₋₆alkyleneSO₂aryl, optionally substituted C₁₋₆alkyleneN(R⁴)₂, OCF₃, C₁₋₆alkyleneN(R⁴)₃⁺, C₃₋₈heterocycloalkyl, and CH(C₁₋₆alkyleneN(R⁴)₂)₂, or two R³ groups are taken together to form an optionally substituted 3- to 8-membered aliphatic ring;

R⁴ is selected from the group consisting of null, hydro, C₁₋₆alkyl, cycloalkyl, aryl, heteroaryl, alkylenearyl, and SO₂C₁₋₆alkyl, or two R⁴ groups are taken together to form an optionally substituted 3- to 8-membered ring;

R⁵ is selected from the group consisting of C₁₋₆alkyl, C₂₋₆alkynyl, aryl, heteroaryl, heterocycloalkyl, N(R³)₂, N(R¹)C(O)R³, N(R¹)CO₂R³, OR³, halo, N₃, CN, C₁₋₆alkylenearyl, C₁₋₆alkyleneN(R³)₂, C(O)R³, C(O)OR³, C(O)N(R³)₂, CF₃, and

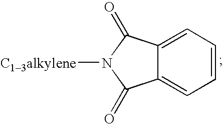

R⁶ is selected from the group consisting of hydro, C₁₋₆alkyl, C₂₋₆alkenyl, cycloalkyl, heterocycloalkyl, aryl, heteroaryl, SO₂R⁴, C₁₋₆alkyl substituted with one or more of halo, hydroxy, aryl, heteroaryl, heterocycloalkyl, N(R⁴)₂, and SO₂R⁴, C₁₋₆alkylenearyl, C₁₋₆alkyleneheteroaryl, C₁₋₆alkyleneC₃₋₈heterocycloalkyl, C₁₋₆alkyleneSO₂aryl, optionally substituted C₁₋₆alkyleneN(R⁴)₂, OCF3, C₁₋₆alkyleneN(R⁴)₃⁺, C₃₋₈heterocycloalkyl, and CH(C₁₋₆alkyleneN(R⁴)₂)₂;

R⁷ and R⁸, independently, are selected from the group consisting of hydro, OR³, C₁₋₆alkyl, halo, N(R³)₂, C(O)N(R³)₂, C₁₋₃alkylenearyl, CN, NO₂, C(O)OR¹¹, C(O)R¹¹, and SR¹¹;

R⁹ is —C≡C—R¹⁰ or —CF₃, or an R⁸ and an R⁹ group are taken together with the carbons to which they are attached to form a 5- or 6-membered carbocyclic aliphatic or aromatic ring system optionally containing one to three heteroatoms selected from the group consisting of O, NR⁴, and S;

R¹⁰ is selected from the group consisting of hydro, C₁₋₆alkyl, aryl, C₁₋₆alkylenearyl, heteroaryl, and C₁₋₆alkyleneheteroaryl;

R¹¹ is selected from the group consisting of hydro, C₁₋₆alkyl, C₂₋₆alkenyl, aryl, C₁₋₃alkylenearyl, —C₃₋₈cycloalkyl, and C₁₋₃alkyleneC₃₋₈cycloalkyl;

n is 1 or 2;

or a pharmaceutically acceptable salt, or prodrug, or solvate thereof.

A compound selected from
1-[5-ethynyl-2-(1-methyl-piperidin-3-ylmethoxy)-phenyl]-3-(5-methylpyrazin-2-yl)-urea;
1-[2-(2-dimethylamino-ethoxy)-5-ethynyl-phenyl]-3-(5-methyl-pyrazin-2-yl)-urea;
1-[5-ethynyl-2-(pyridin-3-ylmethoxy)-phenyl]-3-(5-methyl-pyrazin-2-yl)-urea;
1-[3-(1-methyl-piperidin-3-ylmethoxy)-5,6,7,8-tetrahydro-naphthalen-2-yl]-3-(5-methyl-pyrazin-2-yl)-urea;
1-[3-(1-methyl-piperidin-2-ylmethoxy)-5,6,7,8-tetrahydro-naphthalen-2-yl]-3-(5-methyl-pyrazin-2-yl)-urea;
(S)-1-(5-methyl-pyrazin-2-yl)-3-[2-(piperidin-3-ylmethoxy)-5-trifluoromethyl-phenyl]-urea;
(R)-1-(5-methyl-pyrazin-2-yl)-3-[2-(piperidin-3-ylmethoxy)-5-trifluoromethyl-phenyl]-urea;
1-[2-(1-methyl-piperidin-4-yloxy)-5-trifluoromethyl-phenyl]-3-(5-methyl-pyrazin-2-yl)-urea;
1-(5-methyl-pyrazin-2-yl)-3-[2-(piperidin-3-ylmethoxy)-5-trifluoromethyl-phenyl]-urea;
1-[2-(1-methyl-piperidin-3-ylmethoxy)-5-trifluoromethyl-phenyl]-3-(5-methyl-pyrazin-2-yl)-urea;
1-(5-methyl-pyrazin-2-yl)-3-[7-(pyridin-3-ylmethoxy)-2,3-dihydrobenzo[1,4]dioxin-6-yl]-urea;
1-[7-(2-dimethylamino-ethoxy)-2,3-dihydrobenzo[1,4]dioxin-6-yl]-3-(5-methyl-pyrazin-2-yl)-urea; and
1-[3-(2-dimethylamino-ethoxy)-5,6,7,8-tetrahydro-naphthalen-2-yl]-3-(5-methyl-pyrazin-2-yl)-urea.

XXV. Diarylurea Compounds as Described in U.S. Provisional Patent Application 60/585,292, Including:

i) A compound of formula

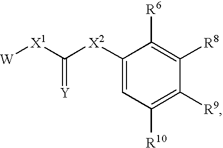

wherein X¹ is null, —O—, —S—, —CH₂—, or —N(R¹)—;

X² is —O—, —S—, or —N(R¹)—;

Y is O or S; or ═Y represents two hydrogen atoms attached to a common carbon atom;

W is selected from the group consisting of heteroaryl, aryl, heterocycloalkyl, cycloalkyl, and C₁₋₆alkyl substituted with a heteroaryl or aryl group, wherein said aryl group W is optionally substituted with one to four substituents represented by R², said heteroaryl group W is optionally substituted with one to four substituents represented by R⁵, and said heterocycloalkyl and cycloalkyl groups W are optionally substituted with one or two C₁₋₆alkyl substituents;

R¹ is selected from the group consisting of hydro, C₁₋₆alkyl, C₂₋₆alkenyl, C₂₋₆alkynyl, and aryl;

R² is selected from the group consisting of heteroaryl, halo, optionally substituted C₁₋₆alkyl, C₂₋₆alkenyl, OCF₃, NO₂, CN, NC, N(R³)₂, OR³, CO₂R³, C(O)N(R³)₂, C(O)R³, N(R¹)COR³, N(R¹)C(O)OR³, N(R¹)C(O)C₁₋₆alkyleneC(O)R³, N(R¹)C(O)C₁₋₆alkyleneC(O)OR³, N(R¹)C(O)C₁₋₆alkyleneOR³, N(R¹)C(O)C₁₋₆alkyleneNHC(O)OR³, N(R¹)C(O)C₁₋₆alkyleneSO₂NR³, C₁₋₆alkyleneOR³, and SR³;

R³ is selected from the group consisting of hydro, halo, C₁₋₆alkyl, C₂₋₆alkenyl, cycloalkyl, aryl, heteroaryl, CO₂R⁴, SO₂R⁴, C₁₋₆ alkyl substituted with one or more of halo, hydroxy, aryl, heteroaryl, heterocycloalkyl, N(R⁴)₂, and SO₂R⁴, C₁₋₆alkylenearyl, C₁₋₆alkyleneheteroaryl, C₁₋₆alkyleneC₃₋₈heterocycloalkyl, C₁₋₆-alkyleneSO₂aryl, optionally substituted C₁₋₆-alkyleneN(R⁴)₂, OCF₃, C₁₋₆alkyleneN(R⁴)₃⁺, C₃₋₈heterocycloalkyl, and CH(C₁₋₆alkyleneN(R⁴)₂)₂, or two R³ groups are taken together to form an optionally substituted 3- to 6-membered aliphatic ring;

R⁴ is selected from the group consisting of hydro, C₁₋₆alkyl, cycloalkyl, aryl, heteroaryl, C₁₋₆alkylenearyl, and SO₂C₁₋₆alkyl, or two R⁴ groups are taken together to form an optionally substituted 3- to 6-membered ring;

R⁵ is selected from the group consisting of C₁₋₆alkyl, aryl, heteroaryl, heterocycloalkyl, N(R³)₂, OR³, halo, N₃, CN, C₁₋₆alkylenearyl, C₁₋₆alkyleneN(R³)₂, C(O)R³, C(O)OR³, C(O)N(R³)₂, N(R¹)C(O)R³, N(R¹)C(O)OR³, CF₃, and

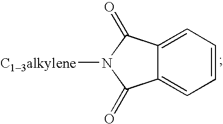

R⁶ is —C≡C—R⁷ or heteroaryl;

R⁷ is selected from the group consisting of hydro, C₁₋₆alkyl, aryl, C₁₋₆alkylenearyl, heteroaryl, C₁₋₆alkyleneheteroaryl, and alkoxy;

R⁸, R⁹, and R¹⁰, independently, are selected from the group consisting of halo, optionally substituted C₁₋₆-alkyl, C₂₋₆alkenyl, C₂₋₆alkynyl, OCF₃, CF₃, NO₂, CN, NC, N(R³)₂, OR³, CO₂R³, C(O)N(R³)₂, C(O)R³, N(R¹)COR³, N(R¹)C(O)OR³, N(R⁸)C(O)OR³, N(R¹)C(O)C₁₋₃alkyleneC(O)R³, N(R¹)C(O)C₁₋₃alkyleneC(O)OR³, N(R¹)C(O)C₁₋₃alkyleneOR³, N(R¹)C(O)C₁₋₃alkyleneNHC(O)OR³, N(R¹)C(O)—C₁₋₃alkyleneSO₂NR³, C₁₋₃alkyleneOR³, and SR³;

and a pharmaceutically acceptable salts, or prodrug, or solvate thereof.

A compound selected from: 1-(5-methyl-pyrazin-2-yl)-3-(5-methyl-2-pyridin-3-ylethynyl-phenyl)-urea, 1-(5-methyl-pyrazin-2-yl)-3-(5-methyl-2-pyridin-3-yl-phenyl)-urea, 1-(5-methyl-pyrazin-2-yl)-3-(5-methyl-2-pyridin-4-phenyl)-urea, 1-(5-methyl-pyrazine-2-yl)-3-(2-oxazol-5-yl-phenyl)-urea, 1-(5-methyl-pyrazin-2-yl)-3-(5-methyl-2-thiazol-2-yl-phenyl)urea, 1-[2-(4-dimethylaminomethyl-thiazol-2-yl)-5-methyl-phenyl]-3-(5-methyl-pyrazin-2-yl)-urea, and mixtures thereof.

XXVI. Diarylurea Compounds are Described in U.S. Provisional Patent Application 60/602,968, Including:

i) A compound of formula:

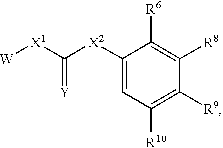

wherein X¹ is null, —O—, —S—, —CH₂—, or —N(R¹)—;

X² is —O—, —S—, or —N(R¹)—;

Y is O or S; or ═Y represents two hydrogen atoms attached to a common carbon atom;

W is selected from the group consisting of heteroaryl, aryl, heterocycloalkyl, cycloalkyl, and C₁₋₆alkyl substituted with a heteroaryl or aryl group, wherein (a) said aryl or heteroaryl group of group W is substituted with at least one of CF₃ and heteroaryl, (b) said aryl group of group W is optionally substituted with one to three substituents represented by R², and (c) said heteroaryl group of group W is optionally substituted with one to three substituents represented by R⁵;

R¹ is selected from the group consisting of hydro, C₁₋₆-alkyl, C₂₋₆alkenyl, C₂₋₆alkynyl, and aryl;

R² is selected from the group consisting of heteroaryl, halo, optionally substituted C₁₋₆alkyl, C₂₋₆alkenyl, OCF₃, NO₂, CN, NC, N(R³)₂, OR³, CO₂R³, C(O)N(R³)₂, C(O)R³, N(R¹)COR³, N(R¹)C(O)OR³, N(R¹)C(O)C₁₋₆alkyleneC(O)R³, N(R¹)C(O)C₁₋₆-alkyleneC(O)OR³, N(R¹)C(O)C₁₋₆alkyleneOR³, N(R¹)C(O)C₁₋₆alkyleneNHC(O)OR³, N(R¹)C(O)C₁₋₆alkyleneSO₂NR³, C₁₋₆alkyleneOR³, and SR³;

R³ is selected from the group consisting of hydro, halo, C₁₋₆alkyl, C₂₋₆alkenyl, cycloalkyl, aryl, heteroaryl, CO₂R⁴, SO₂R⁴, C₁₋₆alkyl substituted with one or more of halo, hydroxy, aryl, heteroaryl, heterocycloalkyl, N(R⁴)₂, and SO₂R⁴, C₁₋₆alkylenearyl, C₁₋₆alkyleneheteroaryl, C₁₋₆alkyleneC₃₋₈heterocycloalkyl, C₁₋₆alkyleneSO₂aryl, optionally substituted C₁₋₆alkyleneN(R⁴)₂, OCF₃, C₁₋₆alkyleneN(R⁴)₃⁺, C₃₋₈heterocycloalkyl, and CH(C₁₋₆alkyleneN(R⁴)₂)₂, or two R³ groups are taken together to form an optionally substituted 3- to 6-membered aliphatic ring;

R⁴ is selected from the group consisting of hydro, C₁₋₆alkyl, cycloalkyl, aryl, heteroaryl, C₁₋₆alkylenearyl, and SO₂C₁₋₆alkyl, or two R⁴ groups are taken together to form an optionally substituted 3- to 6-membered ring;

R⁵ is selected from the group consisting of C₁₋₆alkyl, aryl, heteroaryl, heterocycloalkyl, N(R³)₂, OR³, halo, N₃, CN, C₁₋₆alkylenearyl, C₁₋₆alkyleneN(R³)₂, C(O)R³, C(O)OR³, C(O)N(R³)₂, N(R¹)C(O)R³, N(R¹)C(O)OR³, CF₃, and

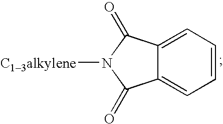

R⁶ is selected from the group consisting of OR¹¹, —C≡C—R⁷, and heteroaryl;

R⁷ is selected from the group consisting of hydro, C₁₋₆alkyl, aryl, C₁₋₆alkylenearyl, heteroaryl, C₁₋₆alkyleneheteroaryl, and alkoxy,

R⁸, R⁹, and R¹⁰, independently, are selected from the group consisting of hydro, halo, optionally substituted C₁₋₆-alkyl, C₂₋₆alkenyl, C₂₋₆alkynyl, OCF₃, CF₃, NO₂, CN, NC, N(R³)₂, OR³, CO₂R³, C(O)N(R³)₂, C(O)R³, N(R¹)COR³, N(R¹)C(O)OR³, N(R⁸)C(O)OR³, N(R¹)C(O)C₁₋₃alkyleneC(O)R³, N(R¹)C(O)C₁₋₆alkyleneC(O)OR³, N(R¹)C(O)C₁₋₃alkyleneOR³, N(R¹)C(O)C₁₋₆alkyleneNHC(O)OR³, N(R¹)C(O)C₁₋₆alkyleneSO₂NR³, C₁₋₆alkyleneOR³, and SR³;

R¹¹ is selected from the group consisting of hydro, C₁₋₆alkyl, C₂₋₆alkenyl, cycloalkyl, heterocycloalkyl, aryl, heteroaryl, SO₂R⁴, C₁₋₆alkyl substituted with one or more of halo, hydroxy, aryl, heteroaryl, N(R⁴)₂, and SO₂R⁴, C₁₋₆alkylenearyl, C₁₋₆alkyleneheteroaryl, C₁₋₆alkyleneC₃₋₈heterocycloalkyl, C₁₋₆alkyleneSO₂aryl, optionally substituted C₁₋₆alkyleneN(R⁴)₂, OCF₃, C₁₋₆alkyleneN(R⁴)₃⁺, C₃₋₈heterocycloalkyl, and CH(C₁₋₆alkyleneN(R⁴)₂)₂;

and a pharmaceutically acceptable salt, or prodrug, or solvate thereof.

A compound selected from:

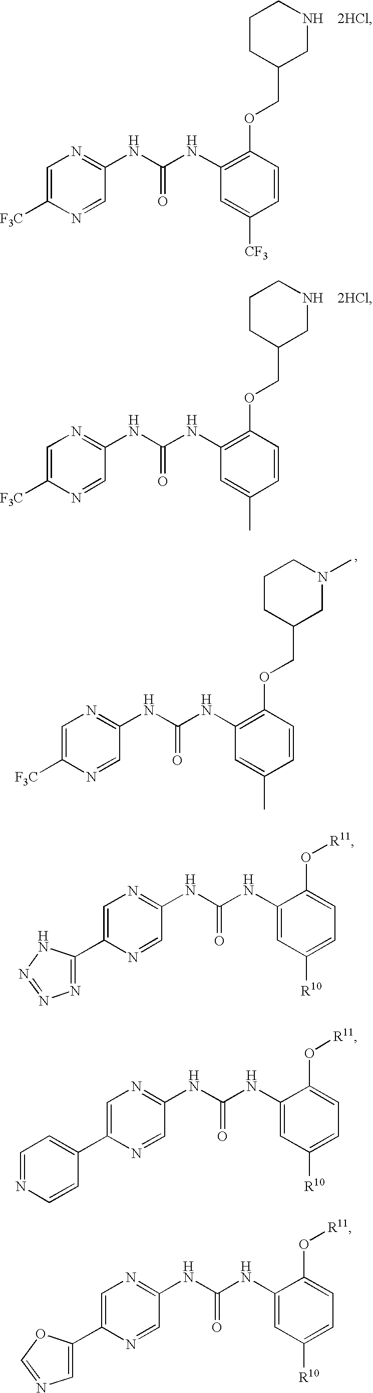

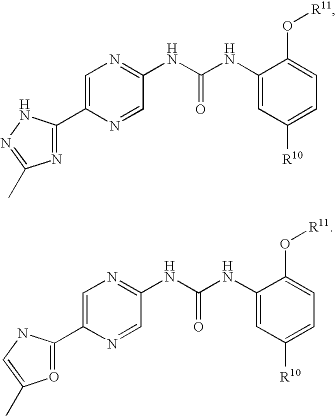

It is possible to compare the selectivity or specificity of a Chk1 inhibitor for Chk1 as against other kinases of interest by way of biochemical (acellular) tests to establish IC50 (defined below) for Chk1, as described elsewhere herein. Thus, selective Chk1 inhibitors may have a lower IC50 for Chk1 inhibition than for inhibition of other kinases of interest.

In certain embodiments, Chk1 inhibitors will not function as a chemotherapy agent when administered alone. A Chk1 inhibitor, in contrast, may act as a chemotherapy agent by virtue of its ability to inhibit additional protein kinases or enzymes that are required for cell growth. This may result in additional cellular effects that lead to side effects and/or a reduced therapeutic index.

In certain embodiments, Chk1 inhibitors useful according to the invention possess at least 20-fold selectivity in inhibiting Chk1 over the following protein kinases: protein kinase A, protein kinase C, cdc2 and pp60v-src. In other embodiments, Chk1 inhibitors as set out above exhibit at least 75-fold selectivity in inhibiting Chk1 over the following protein kinases: protein kinase A, protein kinase C, cdc2 and pp60v-src. In still other embodiments, Chk1 inhibitors set out above preferably demonstrate at least 75-fold selectivity against protein kinase A, protein kinase C, cdc2, pp60v-src and protein kinase B/Akt-1, p38MapK, ERK1, p70S6K, cdc2, cdk2, chk2 and the ab1 tyrosine kinase. “Fold selectivity” is a ratio of the IC50 of the Chk1 inhibitor for the comparison kinase divided by the IC50 of the Chk1 inhibitor for Chk1.

Active agents (e.g., Chk1 activator and/or Chk1 inhibitor) are employed in amounts effective to achieve their intended purpose. As used herein, a “therapeutically effective amount” or means an amount effective to inhibit development of, or to alleviate the existing symptoms of, the condition of the subject being treated. “Dose-effective to inhibit” means an amount effective to inhibit or prevent the proliferation of a population of aberrantly proliferating cells, in vivo or ex vivo. Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index, which is expressed as the ratio of LD50 to ED50. Compounds that exhibit high therapeutic indices (i.e., a toxic dose that is substantially higher than the effective dose) are preferred.

Inhibition of the checkpoint kinase typically is measured using a dose-response assay in which a sensitive assay system is contacted with a compound of interest over a range of concentrations, including concentrations at which no or minimal effect is observed, through higher concentrations at which partial effect is observed, to saturating concentrations at which a maximum effect is observed. Theoretically, such assays of the dose-response effect of inhibitor compounds can be described as a sigmoidal curve expressing a degree of inhibition as a function of concentration. The curve also theoretically passes through a point at which the concentration is sufficient to reduce activity of the checkpoint enzyme to a level that is 50% that of the difference between minimal and maximal enzyme activity in the assay. This concentration is defined as the Inhibitory Concentration (50%) or IC50 value. Determination of IC50 values preferably is made using conventional biochemical (acellular) assay techniques or cell-based assay techniques such as that illustrated herein.

Comparisons of the efficacy of inhibitors often are provided with reference to comparative IC50 values, wherein a higher IC50 indicates that the test compound is less potent, and a lower IC50 indicates that the compound is more potent, than a reference compound. Chk1 inhibitor compounds demonstrating-IC50 values of less than about 1000 nM, or less than about 250 nM, or less than about 100 nM, or less than about 50 nM, or less than about 20 nM, or less than about 1 nM, when measured using the dose-response assay, may be employed according to the invention.

The data obtained in such dose-response assays can be used as a factor in formulating a dosage range for use in humans. The dosage of such compounds preferably lies within a range of circulating concentrations that include the ED50 with little or no toxicity. The dosage can vary within this range depending upon the dosage form, and the route of administration utilized.

The exact formulation, route of administration, and dosage is chosen by the individual physician in view of the patient's condition. Dosage amount and interval can be adjusted individually to provide plasma levels of the active compound that are sufficient to maintain desired therapeutic effects. In general, however, doses employed for adult human treatment typically are in the range of 0.001 mg/kg to about 1000 mg/kg per day, in a range of about 0.1 mg/kg to about 500 mg/kg per dose.

The present invention may be applied to cell populations in vivo or ex vivo. “In vivo” means within a living subject, as within an animal or human. In this context, the invention may be used therapeutically in a subject to slow or stop the proliferation of aberrantly replicating cells. The invention may also be used as a prophylactic to prevent the occurrence or recurrence of aberrant cell proliferation or the manifestation of symptoms associated therewith. Other in vivo uses for which the invention may be therapeutic or preventative are described herein, or will be apparent to those skilled in the art.

“Ex vivo” means outside a living subject. Examples of ex vivo cell populations include in vitro cell cultures and biological samples such as fluid or tissue samples from humans or animals. Such samples may be obtained by methods well known in the art. Exemplary biological fluid samples include blood, cerebrospinal fluid, urine, saliva Exemplary tissue samples include tumors and biopsies thereof. In this context, the invention may be used; for a variety of purposes, including therapeutic and experimental. For example, the invention may be used ex vivo to determine the optimal schedule and/or dosing of administration of a Chk1 activator and Chk1 inhibitor for a given indication, cell type, patient, and other parameter. Information gleaned from such use may be used for experimental purposes or in the clinic to set protocol for in vivo treatment. Other ex vivo uses for which the invention may be suited are described below or will become apparent to those skilled in the art.

Chk1 activators useful in the invention increase the percentage of cells in their target phase of the somatic cell cycle (defined below). By way of background, cells in the somatic cell cycle typically cycle asynchronously. They are a dynamic population comprising cells in various phases of the cell cycle. The percentage of cells at any given phase in the cell cycle depends upon various factors, including, for example, cell type, environment, and cycle rate. Chk1 activators shift these proportions, increasing the percentage of cells in the target phase for the activator. This shift in percentage may be referred to herein as “synchronization,” “arrest,” or “piling up” in the target phase.

As indicated above, the “target phase” of a cell cycle means the phase at which a Chk1 activator will cause a percentage of cells to increase. Different Chk1 activators may have different target phases. For example, ionizing radiation has been shown to increase the percentage of certain cells at the G2 phase. Thus, the G2 phase may be referred to herein as the target phase for ionizing radiation for at least some cell types. The chemotherapeutic agents taxol and nocodazole have been shown to each increase the percentage of cells at the M phase. Thus, the M phase may be referred to as the target phase for taxol or nocodazole. Gemcitabine and low levels of camptothecin will each increase the percentage of cells at the S phase. Thus, the S phase may be referred to as the target phase for each of these chemotherapeutic agents. Any Chk1 activator having any target phase may be used in the present invention.

The proportion of cells in different phases of the cell cycle can be measured by those skilled in the art using any one of a variety of techniques. For example, a fluorescent DNA-binding dye, propidium iodide, can be used to distinguish cells in different cell cycle phases. Since cells in G2 have twice as much DNA as cells in G1, and S phase cells show an intermediate amount of DNA, the technique allows one to identify cells in different phases based on the DNA content of a cell. This method can be carried out on cell lines and tumor specimens (Cerra et al., Methods in Cell Biology, 33:1-12, 1990) Furthermore, cells in S phase can be labeled with the nucleotide analog, bromo-deoxyuridine (BrdU) and then fixed and stained with an fluorescent-tagged antibody to BrdU. Both of these methods employ fluorescence cytometry or fluorescence activity cell sorting (FACS) to quantify the proportion of cells staining with these fluorescent markers.

An additional method for identification of cells in different phases of the cell cycle includes staining the cells with antibodies to markers that are either specific or selective for cell cycle phases. An antibody to the phosphorylated serine residue of histone H3 is highly selective for mitotic cells. An antibody to phosphorylated serine 795 of the retinoblastoma protein, Rb, is selective for S phase cells (Connell-Crowley et al., Mol. Biol. Cell, 8:287-301, 1997). Staining of cells with these antibodies can be used to quantity the proportion of cells in these cell cycle phases by immuno-histochemistry or western blot analysis.

Another method for identification of cells in different phases of the cell cycle includes radioisotope labeling. For example, the ability of gemcitabine to arrest tumor cells in S phase may be assessed in multiple tumor types. Gandhi et al (J. Clin. Ocol., 20:665-73, 2002) discloses a method for assessing S phase arrest in acute myelogenous leukemia patients after treatment with gemcitabine. Patients received gemcitabine at a constant dose of 10/mg/m²/min for various durations of time and tumor cells isolated from blood of patients 24 hours after the start of therapy to determine the number of cells in S phase arrest. Cells may be plated in triplicate (2×10⁶) in RPMI-1640/10% Fetal bovine serum and 1 μCi of [³H]thymidine. Cells may then be allowed to incubate for 30 minutes, after which time thymidine incorporation may be measured. A decrease in radioisotope uptake after treatment with Chk1 activator indicates whether the cells are arrested in S phase, and the duration of the S phase arrest.

The first of the foregoing techniques was used to illustrate the influence of camptothecin, a well known chemotherapeutic agent that, in low doses, activates Chk1 at the S-phase, as shown in Table 2.

TABLE 2

| HT29 Cells: | G1 (%) | S (%) | G2/M* (%) |
|---|---|---|---|
| In the absence of Chk1 activator | 34.2 | 45.7 | 14.5 |
| (asynchronous) | | | |
| After low-level camptothecin treatment | 6.75 | 80.86 | 7 |

*Combined total in G2 + M phase.

Two cell samples, each containing the same human carcinoma cell line (HT29) were prepared. Using propidium iodide (PI) to monitor DNA content, the percentage of cells the G1, S, and G2/M phases of the cell cycle were measured before and after contact with low levels of camptothecin. (Because PI staining indicates total DNA content, this technique does not distinguish between cells in G2 vs. M phase. Accordingly, data reported in the G2/M column of Table 1 shows the total percentage of cells of the population in G2+M-phases.) The first sample was measured to establish the percentage of cells present in each phase asynchronous cell cycling, i.e., in the absence of Chk1 activator. Specifically, in the absence of Chk1 activator, 34.2% of the cells in the sample were in the G1 phase; 45.7% of the cells were in S phase; and 14.5% of the cells were in G2/M phase. The second sample was contacted with low levels of camptothecin (20 nM for 24 hours). At low levels, the target phase of camptothecin is S phase. As Table 1 shows, camptochecin increased the percentage of cells in S phase from 45.7% to more than 80%, and decreased the percentage of cells in the other phases.

In the present invention, Chk1 activator is contacted with the cell population in an amount and for a time sufficient to substantially synchronize cell cycle arrest at the target phase for the Chk1 activator used, prior to contacting the population with Chk1 inhibitor. Preferably, the cell population undergoes optimal synchronization prior to contact with Chk1 inhibitor. For optimal synchronization, a maximum percentage of cells in the population to are allowed to “pile up” or arrest in the target phase for the activator used, with a minimum percentage having progressed into mitosis. However, those skilled in the art will appreciate that lesser degrees of cell cycle synchronization prior to contact with the Chk1 inhibitor will provide some benefit. Thus, “substantial synchronization” includes any degree of synchronization of cell cycle arrest, including optimal, that results in a cytotoxic effect greater than that seen without use of Chk1 inhibitor, or greater than that seen with co-administration of Chk1 activator and inhibitor, or greater than that seen when the cells are contacted with Chk1 inhibitor prior to Chk1 activator. The degree of cell cycle arrest corresponding to or exceeding these references qualifies as “substantial synchronization” and is considered-within the scope of this invention.

Treatment with a Chk1 inhibitor according to the invention may follow at least about a 10% increase in the number of aberrantly proliferating cells in the target phase of the Chk1 activator used; optionally at least about 20%, at least about 50%, at least about 100%; at least about 150%; at least about 200%; at least about 250%; at least about 300%; at least about 350%; at least about 400% increase, at least about 450%, or at least about 500%, as compared to the number of aberrantly proliferating cells present in such phase in the absence of a Chk1 activator. These ranges are merely exemplary, however, and are dependent upon cell type, the particular Chk1 activator used, and other factors readily discernable to those skilled in the art. For example, the skilled artisan will appreciate that the maximum percent increase for any particular cell sample population of aberrantly proliferating cells will be limited by various factors, including percentage of cells present in the target phase of the population prior to Chk1 activator contact.

As indicated above, upon achieving substantial synchronization of cell cycle arrest in the cell population, the present invention calls for contacting the cell population with a Chk1 inhibitor in an amount and for a time sufficient to substantially abrogate the cell cycle arrest. The term “substantially abrogate” is used to indicate that complete abrogation of all arrested cells may not be necessary for efficacy. Those skilled in the art will appreciate that a sufficient degree of cell cycle checkpoint abrogation may be achieved to disrupt cell cycle checkpoint mechanisms and allow cells to pass to a subsequent phase in the cell cycle with unrepaired DNA damage sufficient to cause -cell death or otherwise slow or stop aberrant cell proliferation.

Those skilled in the art will appreciate how to convert information concerning cell-cycle synchronization and abrogation to practical use in the clinic or laboratory. For example, for any given -cell line, Chk1 activator, and Chk1 inhibitor, the dose and time to achieve-substantial cell cycle synchronization and substantial abrogation, respectively, may be measured ex vivo. Ex vivo measurements may then be applied to the clinic as a practical surrogate for direct measurement of the percentage of cells in various phases of the cell cycle.

In determining such measurements, those skilled in the art will appreciate that the duration of Chk1 activator contact with the cell population may, as, indicated above, be influenced by the cell type exhibiting unwanted cell proliferation. Like most cells, aberrantly proliferating cells do not cycle at a universal rate. Some types proliferate faster than others, i.e., have a faster doubling time. Thus, for example, treatment of a tumor cell type with a fast doubling time (e.g., pancreatic cancer or melanoma) may require shorter treatment with Chk1 activator to substantially synchronize cell cycle arrest, while treatment of a tumor with a slower doubling time (e.g., some colon, breast or prostate tumors) would require longer contact with Chk1 activator, all other things being equal, to induce substantially synchronous cell cycle arrest.

Times effective to allow substantial cell cycle synchronization by the Chk1 activator may vary from a few minutes up to 96 hours or more. In some embodiments, it may be preferable or desirable to administer Chk1 activator for up to several weeks or more, as determined by the attending physician or technician. Thus, Chk1 activator may contact the cell population for up to about 30 minutes, up to about 1 hour, up to about 2 hours, up to about 3 hours, up to about 4 hours, up to about 6 hours, up to about 12 hours, up to about 18 hours, up to about 24 hours, up to about 48 hours, up to about 72 hours or up to about 96 hours or more. Those skilled in the art will appreciate that the ranges of time expressed herein are merely exemplary; ranges and sub-ranges within those expressed are also within the scope of the invention.

Contact of the cell population with the Chk1 activator may occur in single doses or over a plurality of doses, according to methods well known in the art for the particular Chk1 activator or activators used. For example, the Chk1 activator may be given at a frequency of: 4 doses delivered as one dose per day at 4-day intervals (q4d×4); 4 doses delivered as one dose per day at 3-day intervals (q3d×4); 1 dose delivered per day at 5-day intervals (qd×5); one dose per week for 3 weeks (qwk3); 5 daily doses, with two days rest, and another 5 daily doses (5/2/5); or, any dose regimen determined to be appropriate for circumstance. Some time may optionally be allowed to lapse between the last dose of Chk1 activator to achieve substantial synchronization of cell cycle arrest prior to contact with the first dose of Chk1 inhibitor as necessary. Similar regimens may be used when Chk1 activator is chemotherapeutic or radiotherapeutic. Additional radiotherapeutic doses are well known to those of ordinary skill in the art.

Contact of the cell population with the Chk1 inhibitor may likewise occur at any dose and time sufficient to achieve substantial abrogation of the cell cycle checkpoint. Typically, though not necessarily, such times include up to about 72 to about 96 hours, depending upon various factors such as those discussed above. In some embodiments, it may be desirable or necessary to administer Chk1 inhibitor over a period of up to about several weeks or more, as determined by the attending physician or technician. Thus, Chk1 inhibitor may typically be administered for up to about 1 hour, up to about 2 hours, up to about 3 hours, up to about 4 hours, up to about 6 hours, up to about 12 hours, up to about 18 hours, up to about 24 hours, up to about 48 hours, or up to about 72 hours. Those skilled in the art will appreciate that the ranges of time expressed herein are merely exemplary; ranges and sub-ranges within those expressed are also within the scope of the invention.

The Chk1 inhibitor may be administered over a plurality of doses. For example, the Chk1 inhibitor may be given at a frequency of: 4 doses delivered as one dose per day at 4 day intervals (q4d×4); 4 doses delivered as one dose per day at 3-day intervals (q3d×4); 1 dose delivered per day at S-day intervals (qd×5); one dose per week for 3 weeks (qwk3); 5 daily doses, with two days rest, and another 5 daily doses (5/2/5); or, any dose regimen pre-determined to be appropriate for the circumstance.

Use of the invention is indicated in treatment of any condition involving aberrant cell proliferation, including cancerous and non-cancerous cell proliferation. In one aspect, treatment may be of any condition responsive to agents that activate cell cycle arrest or are responsive to inhibitors of cell cycle checkpoint proteins.

Cancers include tumors or neoplasms derived from growths of tissue cells wherein multiplication of cells is uncontrolled and progressive. Some such neoplasms are benign, but others are termed “malignant,” and can lead to death of the organism. Malignant neoplasms are distinguished from benign growths in that, in addition to exhibiting aggressive cellular proliferation, the malignant neoplasms, can invade surrounding tissues and metastasize. Moreover, malignant neoplasms are characterized by showing a greater loss of differentiation (greater “dedifferentiation”) and organization relative to one another and surrounding tissues. (This property is called “anaplasia”)

Cancers treatable by the present invention include solid tumors such as carcinomas and sarcomas. Carcinomas derive from epithelial cells which infiltrate (i.e., invade) surrounding tissues and give rise to metastases. Adenocarcinomas are carcinomas derived from glandular tissue, or from tissues that form recognizable glandular structures. Sarcomas are tumors whose cells are embedded in a fibrillar or homogeneous substance, like embryonic connective tissue. The invention also enables treatment of cancers of the myeloid or lymphoid systems, including leukemias, lymphomas, and other cancers that typically are not present as a tumor mass, but are distributed in the vascular or lymphoreticular systems.

Further contemplated are cancers including, but not limited to, myxoid and round cell carcinomas, human soft tissue sarcomas including Ewing's sarcoma, cancer metastases including lymphatic metastases, squamous cell carcinomas particularly of the head and neck, esophageal squamous cell carcinomas, oral carcinomas, blood cell malignancies, including multiple myelomas, leukemias, including acute lymphocytic leukemias, acute nonlymphocytic leukemias, chronic lymphocytic leukemias, chronic myelocytic leukemias, and hairy cell leukemias, effusion lymphomas (body cavity based lymphomas), thymic lymphoma lung cancers (including small cell carcinomas of the lungs, cutaneous T cell lymphomas, Hodgkin's lymphomas, non-Hodgkin's lymphomas, cancers of the adrenal cortex, ACTH-producing tumors, non-small cell lung cancers, breast cancers, including small cell carcinomas and ductal carcinomas), gastro-intestinal cancers (including stomach cancers, colon cancers, colorectal cancers, and polyps associated with colorectal neoplasias), pancreatic cancers, liver cancers, urological cancers (including bladder cancers, such as primary superficial bladder tumors, invasive transitional cell carcinomas of the bladder, and muscle-invasive bladder cancers), prostate cancers, malignancies of the female genital tract (including ovarian carcinomas, primary peritoneal epithelial neoplasms, cervical carcinomas, uterine endometrial cancers, vaginal cancers, cancers of the vulva, uterine cancers and solid tumors in the ovarian follicle), malignancies of the male genital tract (including testicular cancers and penile cancers), kidney cancers (including renal cell carcinomas), brain cancers (including intrinsic brain tumors, neuroblastomas, astrocytomas, gliomas, and metastatic tumor cell invasions in the central nervous system), bone cancers (including osteomas and osteosarcomas), skin cancers (including malignant melanomas, tumor progressions of human skin keratinocytes, basal cell carcinomas, and squamous cell cancers), thyroid cancers, retinoblastomas, peritoneal effusions, malignant pleural effusions, mesotheliomas, Wilms's tumors, gall bladder cancers, trophoblastic neo-plasms, hemangiopericytomas, and Kaposi's sarcomas.

As non-limiting examples, the method according to the invention may be adapted to the following uses of Chk1 activators (alone or in combination with other active agents):

Gemcitabine for the treatment of proliferative disorders including pancreatic cancer (e.g., locally advanced (nonresectable state II or stage r) or metastatic (stage 1V) adenocarcinoma of the pancreas); gemcitabine for the first-line treatment and for patients previously treated with a 5-FU-containing regimen; gemcitabine in combination with platinum coordination complexes (e.g., cisplatin) for the treatment non-small cell lung cancer (e.g., inoperable, locally advanced (stage IRA or IIIIB) or metastatic (stage 1V) non-small cell lung cancer);

Pemetrexed for the treatment of proliferative disorders including non-small lung cell carcinomas, solid tumors, malignant mesothelioma, urothelium, cervical cancer, recurrent endometrial cancer, peritoneal cancer, pleural mesothelioma, gall bladder cancer, breast cancer, and colorectal cancer;

Topotecan for the treatment of proliferative disorders including meningeal cancers, cervical cancer, ovarian cancer, epithelial cancer, esophageal cancer, fallopian tube cancer, primary peritoneal cancer, small cell lung cell cancer, prostate cancer, neuroblastomas, gliomas, solid tumors, acute myeloid leukemia, chromic myelogenous leukemia, advanced meylodysplastic syndromes, and rhabdomyosarcoma;

Irinotecan for the treatment of proliferative disorders including colorectal cancer, glioblastoma multiforme, solid tumors, breast cancer, penile cancer, liver cancer, metastatic gastric carcinoma, gastroesophageal junction adenocarcinoma, small bowel adenocarcinoma, rhabdomyosarcoma, urothelium cancer, stomach cancer, bladder cancer, kidney cancer, small cell lung cancer, pancreatic cancer, head and neck cancer, glioma, sarcoma, metastatic carcinoma of the colon or rectum;

Chlorambucil for the treatment of proliferative disorders including chronic lymphocytic leukemia, Hodgkin's lymphoma; non-Hodgkin's lymphoma, follicular lymphoma, chronic lymphocytic cancer;

Platinum coordination complexes, e.g., cisplatin, for the treatment of proliferative disorders including testicular cancer, ovarian cancer, bladder cancer, head and neck cancer, esophageal cancer, small cell and non-small cell lung cancer, non-Hodgkin's lymphoma, trophoblastic neoplasms; adrenal cortical cancer, anal cancer, bile duct cancer, bladder cancer, bone cancer, cervical cancer, endometrial cancer, gall bladder cancer, gastrointestinal carcinoid tumors, laryngeal cancer, hypopharyngeal cancer, liver cancer, lung cancer, small cell lung cancer, malignant mesothelioma, nasal cavity cancer, paranasal cancer, nasopharyngeal cancer, neuroblastoma, oral cavity cancer, oropharyngeal cancer, osteosarcoma, ovarian cancer, germ cell tumors of the ovary, pancreatic cancer, penile cancer, retinoblastoma, salivary gland cancer sarcoma, melanoma, stomach cancer, testicular cancer, thymus cancer, uterine sarcoma, vulvar cancer;

Carboplatin for the treatment of proliferative disorders including ovarian cancer, germ cell tumors, head and neck-cancer, small cell and non-small cell lung cancer, bladder cancer, relapsed and refractory acute leukemia, endometrial cancer;

Camptothecin for the treatment of proliferative disorders including stomach cancer, gastroesophageal junction cancer, soft tissue sarcoma, malignant glioma;

Etoposide for the treatment of proliferative disorders including small cell and other lung cancers, gastric cancer, germ cell tumors, adrenal cortical cancer, bone cancer, gastrointestinal carcinoid tumors, gestational trophoblastic disease, Hodgkin's disease, acute lymphocytic cancer, childhood leukemia, small cell lung cancer, lung carcinoid tumor, neuroblastoma, osteosarcoma, ovarian cancer, germ cell tumors of the ovary, prostate cancer, retinoblastoma, stomach cancer, testicular cancer, Wilm's Tumor;

Ara-C for the treatment of proliferative disorders including acute myeloid leukemia, high-risk meylodysplastic syndrome, CML, lymphoma, solid tumor, chronic lymphocytic leukemia, acute lymphocytic leukemia, acute non-lymphocytic leukemia, chronic myelocytic leukemia, precursor T-lymphoblastic lymphoma/leukemia, Burkitt-lumphoma;

Aphidocolin for ex vivo studies of proliferative disorders including breast cancer and acute myeloid leukemia;

Fludarabine for the treatment of proliferative disorders including chronic lymphocytic leukemia, follicular lymphoma, metastatic melanoma, renal cell carcinoma, acute myeloid leukemia, acute lymphoblastic leukemia, non-Hodkin's lymphoma, breast cancer, hairy cell leukemia, multiple myeloma, cervical cancer, vaginal cancer, leukemia, childhood leukemia, chronic granulomatous disease, mastocytosis, kidney cancer, urinary tract cancer, skin tumors, bladder cancer, basal cell carcinoma, adrenal carcinoma, esophageal and gastric cancer, hepatocellular cancer, ovarian cancer, B-cell leukemia, chronic lymphocytic leukemia, follicular lymphoma; and

Methotrexate for the treatment of proliferative disorders including gestational choriocarcinoma, chorioadenoma, destruens and hydatidiform moles, acute lymphocytic leukemia, meningeal leukemia, breast cancer, epidermoid cancers of the head and neck, advanced mycosis fungoides (cutaneous T-cell lymphoma), lung cancer (especially squamous cell and small cell types), non-Hodgkin's lymphomas; bladder cancer, bone cancer, breast cancer, esophageal cancer, gestational trophoblastic disease, laryngeal and hypopharyngeal cancer, acute lymphocytic leukemia, acute myeloid leukemia, small cell lung cancer, Burkitt's lymphoma, precursor T-lymphoblastic mesothelioma, nasal cavity and paranasal cancer, nasopharyngeal cancer, oral cavity and oropharyngeal cancer, osteosarcoma, penile cancer, salivary gland cancer, and stomach cancer.

The invention may also be used to treat conditions involving non-cancerous aberrantly proliferating cells. Such conditions include, but are not limited to, atherosclerosis, restenosis, vasculitis, nephritis, retinopathy, renal disease, proliferative skin disorders, psoriasis, keloid scarring, actinic keratosis, Stevens-Johnson Syndrome, rheumatoid arthritis (RA), systemic-onset juvenile chronic arthritis (JCA), osteoporosis, systemic lupus (SLE) erythmatosus, hyperproliferative diseases of the eye including epithelial down growth; proliferative vitreoretinopathy (PVR); diabetic retropathy, hemangio-proliferative diseases, ichthyosis, or papillomas.

Non-cancerous conditions treatable by the present invention may also include a variety of inflammation and inflammatory diseases, conditions, or disorders. Examples of such indications include, but are not limited to, rheumatoid arthritis, psoriasis, vitiligo, Wegener's granulomatosis, and SLE. Treatment of arthritis, Wegener's granulomatosis, and SLE often involves the use of immunosuppressive therapies, such as ionizing radiation, methotrexate, and cyclophosphamide. Psoriasis and vitiligo commonly are treated with ultraviolet radiation (UV) in combination with a psoralen. Such treatments typically induce, either directly or indirectly, DNA damage. Inhibition of Chk1 activity within the offending immune cells renders the cells more sensitive to control by these standard treatments. In general, Chk1 inhibitors useful in the invention may optionally be used to potentiate control of inflammatory disease cells when administered in combination with immunosuppressive drugs.

Animal models of some of the foregoing cancerous and non-cancerous conditions treatable by the present invention include for example: athymic nude mice injected with viable cancer cells from the HL60 cell line (human non-small cell lung cancer), athymic nude mice injected with Panc-01 human tumor cells (human pancreatic cancer), athymic nude mice injected with A375 human tumor cells (human melanoma), athymic nude mice injected with SKMES lung cancer cells (human lung cancer), athymic nude mice injected with SKOV-3.ip. ovarian carcinoma cells (human ovarian cancer), athymic nude mice injected with MDA-MB-361 breast cancer cells (human breast cancer), rats injected with 137-62 cells (breast cancer), and c56BL/Ka mice (cpdm/cpdm), (human psoriasis) (Gijbels et al., Exp. Dermatol., 9:351-358 (2000).

Chk1 inhibitor's of the invention are contemplated for use in a composition comprising Chk1 inhibitors in a pharmaceutically acceptable diluent or carrier. In one aspect, the pharmaceutical composition, comprises Chk1 inhibitors as set out above.

Formulations of the present invention can be administered in a standard manner for the treatment of the indicated diseases; such as orally, parenterally, transmucosally (e.g., sublingually or buccally), topically, transdermally, rectally, via inhalation (e.g., nasal or deep lung inhalation). Parenteral administration includes, but is not limited to intravenous, intra-arterial, intraperitoneal, subcutaneous, intramuscular, intrathecal and intra-articular. Parenteral administration also can be accomplished using a high pressure technique, like POWDERJECT™.

For oral administration, or for buccal administration, the composition can be in the form of tablets or lozenges formulated in conventional manner. For example, tablets and capsules for oral administration can contain conventional excipients such as binding agents (for example, syrup, acacia, gelatin, sorbitol, tragacanth, mucilage of starch, or polyvinylpyrrolidone), fillers (for example, lactose, sugar, microcrystalline cellulose, maize-starch, calcium phosphate, or sorbitol), lubricants (for example, magnesium stearate, stearic acid, talc, polyethylene glycol or silica), disintegrants (for example, potato starch or sodium starch glycolate), or wetting agents (for example, sodium lauryl sulfate). The tablets can be coated according to methods well known in the art.

Alternatively, the compounds of the present invention can be incorporated into oral liquid preparations such as aqueous or oily suspensions, solutions, emulsions, syrups, or elixirs, for example. Moreover, formulations containing these compounds can be presented as a dry product for constitution with water or other suitable vehicle before use. Such liquid preparations can contain conventional additives, for example suspending agents, such as sorbitol syrup, methyl cellulose, glucose/sugar syrup, gelatin, hydroxyethylcellulose, hydroxypropylmethylcellulose, carboxymethylcellulose, aluminum stearate gel, and hydrogenated edible fats; emulsifying agents, such as lecithin, sorbitan monooleate, or acacia; nonaqueous vehicles (which can include edible oils), such as almond oil, fractionated coconut oil, oily esters, propylene glycol, and ethyl alcohol; and preservatives, such as methyl or propyl p-hydroxybenzoate and sorbic acid.

Such preparations also can be formulated as suppositories, e.g., containing conventional suppository bases, such as cocoa butter or other glycerides. Compositions for inhalation typically can be provided in the form of absolution, suspension, or emulsion that can be administered as a dry powder or in the form of an aerosol using a conventional propellant, such as dichlorodifluoromethane or trichlorofluoromethane. Typical topical and transdermal formulations comprise conventional aqueous or nonaqueous vehicles, such as eye drops, creams, ointments, lotions, and pastes, or are in the form of a medicated plaster, patch, or membrane.

Additionally, compositions of the present invention can be formulated for parenteral administration by injection or continuous infusion. Formulations for injection can be in the form of suspensions, solutions, or emulsions in oily or aqueous vehicles, and can contain formulation agents, such as suspending, stabilizing, and/or dispersing agents. Alternatively, the active ingredient can be in powder form for constitution with a suitable vehicle (e.g., sterile, pyrogen-free water) before use.

A composition in accordance with the present invention also can be formulated as a depot preparation. Such long acting formulations can be administered by implantation (for example, subcutaneously or intramuscularly) or by intramuscular injection. Accordingly, the compounds of the invention can be formulated with suitable polymeric or hydrophobic materials (e.g., an emulsion in an acceptable oil), ion exchange resins, or as sparingly soluble derivatives (e.g., a sparingly soluble salt).

The compounds useful according to the invention may be conjugated or linked to auxiliary moieties that promote any property of the compounds that may be beneficial in methods of therapeutic use. Such conjugates can enhance delivery of the compounds to a particular anatomical site or region of interest (e.g., a tumor), enable sustained therapeutic concentrations of the compounds in target cells, alter pharmacokinetic and pharmacodynamic properties of the compounds, and/or improve the therapeutic index or safety profile of the compounds. Suitable auxiliary moieties include, for example, amino acids, oligopeptides, or polypeptides, e.g., antibodies such as monoclonal anti-bodies and other engineered antibodies; and natural or synthetic ligands to receptors in target cells or tissues. Other suitable auxiliaries include fatty acid or lipid-moieties, to promote biodistribution or uptake of the compound by target cells (see, e.g., Bradley et al., Clin. Cancer Res. (2001) 7:3229.

It is further contemplated that the method of the invention comprises administration of at least one agent to reduce side-effects resulting from treatment of the subject. In one aspect, the side-effect reducing-agent comprises at least one growth factor. In a related aspect, the side-effect reducing agent comprises at least one cytokine, at least one lymphokine, or at least one hematopoetic factor. Growth factors, cytokines, and hematopoetic factors useful in the methods of the invention include, but are not limited to, M-CSF, GM-CSF, TNF, IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IFN, TNF, G-CSF, Meg-CSF, GM-CSF, thrombopoietin, stem cell factor, erythropoietin, angiopoietins, including Ang-1, Ang-2, Ang-4, Ang-Y, and/or the human angiopoietin-like polypeptide, vascular endothelial growth factor (VEGF), angiogenin, bone morphogenic protein-1 (BMP-1), BMP-2, BMP-3, BMP-4, BMP-5, BMP-6, BMP-7, BMP-8, BMP-9, BMP-10, BMP-11, BMP-12, BMP-13, BMP-14, BMP-15, BMP receptor IA, BMP receptor IB, brain derived neurotrophic factor, ciliary neutrophic factor, ciliary neutrophic factor receptor cytokine-induced neutrophil chemotactic factor 1, cytokine-induced neutrophil chemotactic factor 2, cytokine-induced neutrophil chemotactic factor 2, endothelial cell growth factor, endothelin 1, epidermal growth factor, epithelial-derived neutrophil attractant, fibroblast growth factor (FGF) 4, FGF 5, FGF 6, FGF 7, FGF 8, FGF 8b, FGF 8c, FGF 9, FGF 10, FGF acidic, FGF basic, glial cell line-derived neutrophic factor receptor 1, glial cell line-derived neutrophic factor receptor 2, growth related protein, growth related protein, growth related protein, growth related protein, heparin binding epidermal growth factor, hepatocyte growth factor, hepatocyte growth factor receptor, insulin-like growth factor I, insulin-like growth factor receptor, insulin-like growth factor II, insulin-like growth factor binding protein, keratinocyte growth factor, leukemia inhibitory factor, leukemia inhibitory factor receptor, nerve growth factor nerve growth factor receptor, neurotrophin-3, neurotrophin-4, placenta growth factor, placenta growth factor 2, platelet-derived endothelial cell growth factor, platelet derived growth factor, platelet derived growth factor A chain, platelet derived growth factor AA, platelet derived growth factor AB, platelet derived growth factor B chain, platelet derived growth factor BB, platelet derived growth factor receptor, platelet derived growth factor receptor, pre-B cell growth -stimulating factor, stem cell factor, stem cell factor receptor, transforming growth factor (TGF), TGF, TGF 1, TGF 1.2, TGF 2, TGF 3, TGF 5, latent TGF 1, TGF, binding protein I, TGF binding protein A, TGF binding protein III, tumor necrosis factor receptor type I, tumor necrosis factor receptor type II, urokinase-type plasminogen activator receptor, vascular endothelial growth factor, and chimeric proteins and biologically or immunologically active fragments thereof .

### EXAMPLES

The following examples illustrate various non-limiting embodiments of the invention and/or provide support therefore. Example 1 compares the present invention to co-administration of Chk1 activator and Chk1 inhibitor in an art-recognized in vitro model. Example 2 provides a similar comparison using a mitotic index assay. Example 3 compares the present invention to co-administration of Chk1 activator and Chk1 inhibitor in an animal tumor model. Example 4 describes a sensitive assay that may be used to measure Chk1 inhibitor activity in animal models. Example 5 demonstrates that selective Chk1 inhibitors are capable of abrogating DNA damage-induced G2 and S phase, checkpoints. Example 6 demonstrates that Chk1 inhibitor is taken up by tumor cells in the presence of Chk1 activator in an art recognized xenograft tumor model. Example 7 describes the use of the previously exemplified assay to determine the effect of Chk1 inhibitors on cell cycle arrest. This assay is again used in Example 8 to provide an example of the determination of the optimal dose and time of Chk1 activator required to achieve selective cell cycle synchronization. Example 9 describes an assessment of the optimal contact time of a population of aberrantly proliferating cells with a Chk1-inhibitor to achieve substantial abrogation of cell cycle arrest. Example 10 describes an assessment of a dose response relationship between Chk1 inhibitor and abrogation of cell cycle arrest. Example 11 describes an assessment of optimal dose of Chk1 inhibitor for use in an embodiment of the invention. Example 12 describes an assay that may be used to determine whether an agent is a Chk1 activator. Example 13 describes an assay that may be used to monitor Chk1 activity in response to a Chk1 inhibitor.

### Example 1

### Contacting Aberrantly Proliferating Cells with Chk1 Inhibitor After Substantial Cell Cycle Synchronization by Chk1 Activator Showed Better Anti-Proliferative Activity than Co-Administration in a Non-Small Cell Lung Cancer Cancer Animal Model

A method of the invention provided an improved antiproliferative effect over co-administration in an art-recognized in vitro tumor model. In the experiment, gemcitabine was used as the Chk1 activator and a diaryl urea compound according to Keegan et al., PCT/US02/06452, was used as the selective Chk1 inhibitor. (The same Chk1 inhibitor was used in the examples 2-11.) The target phase of gemcitabine is the S phase of the cell cycle. A non-small cell lung tumor xenograft tumor model, H460, was the art-recognized in vitro tumor model.

Nude mice were engrafted with H460 tumor cells and allowed to grow to an average of 75 mm3. Tumor-bearing mice were then treated with vehicle, gemcitabine or gemcitabine plus 400 mg/kg selective Chk1 inhibitor. The gemcitabine was administered at a dose of 160 mg/kg q3d×3 either simultaneously with the Chk1 inhibitor (co-administration) or, according to the invention, 18 hours prior to the Chk1 inhibitor to allow for S phase synchronization.

Tumors were measured every 2-3 days. On day 10, the median tumor volume for the vehicle group was 10 times the starting volume, while the gemcitabine alone group was four times the starting volume. The tumor volume for the gemcitabine plus Chk1 inhibitor co-administration group was also four times the starting volume. The tumor volume for the gemcitabine followed by Chk1 inhibitor group was only 1.1 times the starting volume. This experiment demonstrates that pretreatment with gemcitabine in an amount and for a time sufficient to substantially synchronize the tumor cells prior to checkpoint release by the Chk1 inhibitor leads to greater anti-tumor activity than co-administration of the two agents together.

### Example 2

### Contacting Aberrantly Proliferating Cells with Chk1 Inhibitor after Substantial Cell Cycle Synchronization by Chk1 Activator Reduced Required Exposure Time To Chk1 Inhibitors in a Mitotic Index Assay

Chk1 inhibitors were tested in a cell-based proliferation assay for the ability to sensitize tumor cells to ionizing radiation or chemotherapy agents. Chk1 inhibitors were tested in combination with 5-FU, gemicitabine, ionizing radiation, camptothecin, etoposide, hydroxyurea, cisplatin, fludarabine, Ara-C and aphidicolin. For each experiment, a serial dilution of each compound in combination with a ten-point dilution of each chemotherapy agent was included, in order to determine the concentration of chemotherapeutic required to inhibit the growth of 90% (GI90) of the cells in the presence and absence of the Chk1 inhibitor. This ratio of GI90 in the absence of Chk1 inhibitor to that in the presence of Chk1 inhibitor is called the “fold sensitization.” Fold sensitization was plotted as a function of Chk1 inhibitor concentration and the amount of drug required to yield two-fold sensitization was calculated. The fold sensitization of Chk1 inhibitors to these chemotherapy agents is shown below (Table 3). This concentration is referred to as the “ECTFS” or, the Effective Concentration of Inhibitor required for yielding Two-Fold Sensitization. Another parameter analyzed was the fold sensitization achieved at the LD50 (the dose of compound alone that inhibits growth of 50% of cells) for the compound. These two values allow direct ranking of both the potency and toxicity of Chk1 inhibitors with respect to one another.

TABLE 3
CHK1 INHIBITORS SENSITIZE TUMOR
CELLS TO CHEMOTHERAPY AGENTS.

| Chemotherapy Agent | Fold Sensitization to Agent by Chk1 Inhibitor |
|---|---|
| Gemcitabine | 12 |
| 5-FU | 12 |
| Ara-C | 9 |
| Camptothecin | 5 |
| Cisplatin | 3 |
| Etoposide | 3 |

The sensitization assay described above was used to assess the ability of the Chk1 inhibitors to promote cell death after contact with a selective Chk1 inhibitor according to an embodiment of the invention. This in vitro assay is believed to correlate to anti-tumor activity of the Chk1 inhibitors in vivo. The sensitization studies indicated that, in the samples tested, if gemcitabine and the Chk1 inhibitor were dosed simultaneously, the exposure time required for a Chk1 inhibitor to yield maximal sensitization (14 fold sensitization) was approximately 24 hours. However, if cells were treated first with gemcitabine for approximately 2 hours and the cells allowed approximately 24 hours to arrest in S phase before treating with the Chk1 inhibitor, as little as 4-6 hours of inhibitor exposure led to maximum sensitization (over 12-fold sensitization). In contrast, simultaneous treatment of gemcitabine and the Chk1 inhibitor for 6 hours resulted in no sensitization in the samples tested. These data suggest that allowing aberrantly proliferating cells to substantially synchronize cell cycle arrest before administering Chk1 inhibitor reduces the required time of exposure to Chk1 inhibitors to result in tumor cell death in combination with a Chk1 activating agent.

### Example 3

### Contacting Aberrantly Proliferating Cells with Chk1 Inhibitor after Substantial Cell Cycle Synchronization by Chk1 Activator Showed Better Anti-Proliferative Activity than Co-Administration in a Colon Cancer Animal Model

Nude mice were engrafted with HT29 colon carcinoma cells and tumors were grown to 200 mm³ for 10 days. The HT-29 tumor-bearing mice were treated with vehicle, 600 mg/kg Chk1 inhibitor (p.o.), 160 mg/kg gemcitabine (i.p.) or the co-administration of gemcitabine and Chk1 inhibitor. Alternatively, mice were pretreated according to the invention with gemcitabine for 24 hours, dosed with Chk1 inhibitor on day 2, and allowed to rest on day 3. The treatment regimen was repeated four times. This dosing strategy combined the MTD dosing for gemcitabine (160 mg/kg q3d×4, i.e. 4 doses delivered as one dose per day at 3-day intervals) with a gemcitabine pretreatment strategy.

Tumors were measured every 2-3 days until they reached 1200 mg and then the animals were sacrificed. Median tumor growth delay, survival benefit and tumor regressions were measured. The median time for tumors to grow from 200 mm3 to 800 mm3 was 14.5 days longer in the animals treated with gemcitabine then Chk1 inhibitor compared to animals treated with gemcitabine alone. The survival benefit was 15 days greater in mice treated with the combination therapy over gemcitabine alone.

In summary, substantial synchronization of the tumor cells in S-phase by gemcitabine followed by checkpoint release via the Chk1 inhibitor resulted in a significant improvement in the anti-tumor activity. Whereas-co-administration resulted in a 4 day growth delay as described in Example 6, pretreatment with gemcitabine according to the invention resulted in a 14.5 day tumor growth delay.

### Example 4

### A Sensitive Assay to Measure Chk1 Inhibitor Activity in Animal Models

The following sensitive assay was developed to measure Chk1 inhibitor activity in rodent tumor models. In particular, the assay may be used, inter alia, to measure the ability of Chk1 inhibitors to block Chk1 function in the tumor model, and to allow for assessment of conditions that facilitate Chk1 inhibitors' access to the molecular target.

The ability of selective Chk1 inhibitors to abrogate a chemotherapy-induced checkpoint was measured using a quantitative immunofluourescent assay that measures mitotic index by monitoring histone H3 phosphorylation on serine 10 (H3-P), a mitosis-specific event (Ajiro et al., J Biol. Chem. 271:13197-201. 1996; Goto et al., J Biol Chem. 274:25543-9, 1999). The assay protocol was as follows. Tumors from rodents treated or untreated with Chk1 activator (in the present study, chemotherapy agent) and/or Chk1 inhibitor, were excised and paraffin embedded. The tumors are cut into 6 micron thick slices and mounted on glass slides. The paraffin was removed from the slides by 3 minute successive treatments with xylene, 100% ethanol, 95% ethanol, 70% ethanol and deionized water. The slides are then heated to 95° C. in 10 mM sodium citrate for 10 minutes followed by a 20 minute cooling step. The slides are blocked for 30 minutes with Block buffer (20% normal human serum and 2% bovine serum albumin in phosphate buffered saline containing 0.05% Triton X-100 (PBST)). The anti-phospho histone H3 antibody (Upstate Biotech, Cat. #06-570) is diluted 1:200 in the Block buffer and incubated with the slides for one hour. The slides are washed 3 times 5 minutes in PBST. The secondary antibody, donkey anti-rabbit rhodamine (Jackson, cat #711-295-152) was added for 30 minutes. The slides were then washed twice in PBST and 75 μM of 0.1 μM/ml DAPI (Sigma) in PBS is added and allowed to stain for 30 minutes. The slides were then washed two more times in PBST and mounted with Vectashield (Vector, cat # H-1400). Slides were viewed using fluorescence microscopy. The percentage of cells stained with H3-P antibody relative to total (DAPI stained) cells were quantified using Metamorph software (Universal Imaging Corporation, Version 4.6).

### Example 5

### Selective Chk1 Inhibitors Abrogate DNA Damage-Induced G2 and S Phase Checkpoints

Previous studies have demonstrated that selective Chk1 inhibitors substantially abrogate the DNA damage-induced G2/M and S phase checkpoints. In the former, DNA damage was induced by ionizing radiation (IR), whose target phase is the G2 phase. In the latter, DNA damage was induced by chemotherapeutic agents whose target phase is the S phase. See published U.S. patent application 2003/0069284 and references cited therein.

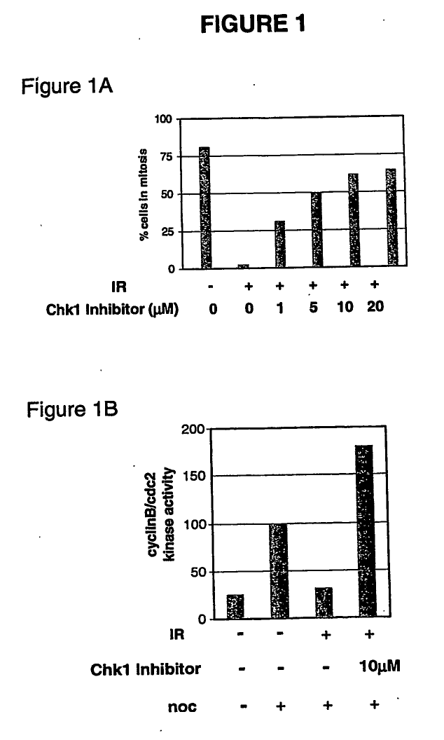

Briefly, the Chk1 inhibitor abrogation of IR-induced G2 DNA damage checkpoint was assayed by mitotic index experiments. Approximately 1×10⁶ HeLa cells were irradiated with 800 rads and incubated for 7 hours at 37° C. Because these cells are functionally p53 negative, they arrest exclusively in G2. Nocodazole was then added to a concentration of 0.5 μg/1 mL and incubated for 15 hours at 37° C. (The addition of nocodazole was designed to trap any cells that progressed through the G2 arrest in mitosis thus preventing them from further progressing into G1 and allowing for quantification of M phase cells.) A selective Chk1 inhibitor was added for 8 hours, and the cells harvested by centrifugation, washed once with PBS, then resuspended in 2.5 mL 75 mM KCl and centrifuged again. The cells then were fixed in 3 mL of freshly prepared cold, acetic acid:methanol (1:3) and incubated on ice for 20 minutes. Cells were pelleted, the fix solution was aspirated and the cells were resuspended in 0.5 mL of PBS. Mitotic spreads were prepared by pipeting 100 μL of the fixed cells onto a glass microscope slide and flooding the sample with 1 ml of fix solution. Slides were then air dried, stained with Wrights stain (Sigma, St. Louis, Mo.) for 1 minute, -followed by one wash in water and one wash in 50% methanol. The presence of condensed chromosomes and lack of nuclear envelope identified mitotic cells. The selective Chk1 inhibitors (diarylurea compounds according to US 2003/0069284) tested resulted in an increase in the number of mitotic cells in the presence of irradiation, thereby demonstrating abrogation of the IR-induced G2 arrest (FIG. 1A). This checkpoint abrogation results in an enhancement in the activity of CyclinB/cdc2, which is required for progression of cells into mitosis. Cells treated with IR followed by Chk1 inhibitor thus progress into mitosis with damaged DNA. These experiments confirm the hypothesis that Chk1 is involved in the IR-induced G2.

### Example 5A

### Chk1 Inhibitors Abrogate the DNA Damage-Induced G2 Checkpoint

As FIG. 1 illustrates, Chk1 inhibitors abrogate the DNA damage-induced G2 checkpoint in HeLa cells. FIG. 1A illustrates that IR and Chk1 inhibitor treated cells show increased CyclinB/cdc2 kinase activity. Activity is shown as a percent relative to nocodazole (noc)-treated cells. FIG. 1B illustrates mitotic index experiments demonstrating that Chk1 inhibitors allow HeLa cells to progress through the irradiation (IR)-induced G2 checkpoint. These data show a dose-dependent effect of the Chk1 inhibitor arrest and that selective inhibitors of Chk1 allow cells to continue cycling in the presence of DNA damage.

### Example 5B

### Chk1 Inhibitors Abrogate the DNA Damage-Induced S-Phase Checkpoint

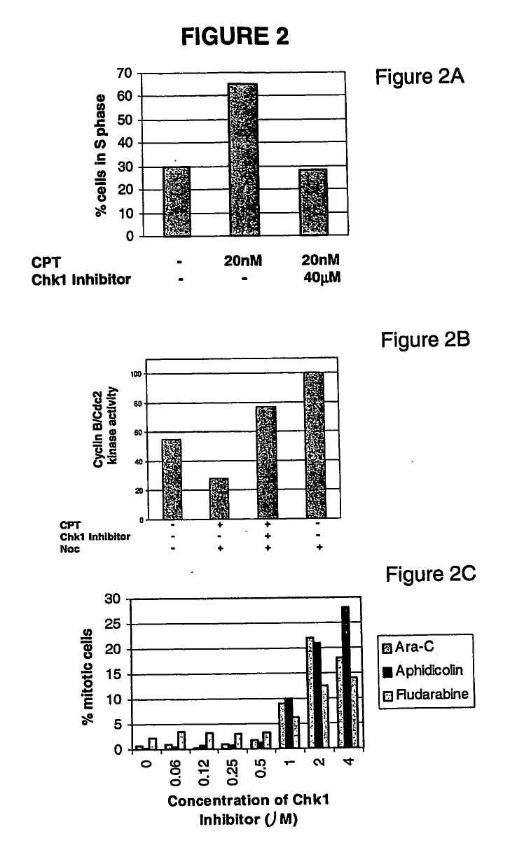

As illustrated in FIG. 2, selective Chk1 inhibitors abrogate the S phase checkpoint induced by Chk1 activators whose target phase is the S-phase: camptothecin (CPT) (FIGS. 2A and 2B), Ara-C, gemcitibine, fludarabine and aphidicolin in HT29 colon carcinoma cells (FIG. 2C ). The S phase abrogation was induced by these agents in a dose-dependent manner and resulted in entry into mitosis despite DNA damage, resulting in cell death. Microscopic analysis of mitotic cells treated with Chk1 inhibitor suggested that the chromosomes were improperly aligned on the mitotic spindles. Without wishing to be bound by theory, one hypothesis suggests that premature entry into mitosis results in defects in attachment of microtubules to kinetocores, inducing a spindle checkpoint and metaphase arrest, ultimately leading to death caused by mitotic catastrophe.)

Thus, HT29 colon carcinoma cells were treated with 20 nM CPT in the presence and absence of a Chk1 inhibitor. A. Cells were pulse-labeled with BrdU and % BrdU-staining cells quantified. B. HT29 cells were treated with CPT in the presence and absence of a Chk1 inhibitor. Cells were also treated with nocodazole (noc) to trap cells in mitosis. Cells that progressed out of S phase into mitosis were measured by CyclinB/cdc2 kinase activity. C. HT29 cells were treated with 20 mM Ara-C, 20 mM fludaribine or 10 mg/mL aphidicolin, each with a Chk1 inhibitor. Mitotic cells were defined as percent cells that stained positive with histone H3 antibodies. The data shows that selective Chk1 inhibitors abrogate the S phase checkpoint induced by Chk1 activators whose target phase is the S phase.

### Example 6

### Chk1 Inhibitor is Taken Up by Tumor Cells in the Presence of Chk1 Activator in a Xenograft Tumor Model

In a xenograft-tumor model, nude mice were engrafted with HT29 colon carcinoma tumors on the flank and allowed to grow to 200 mm³. Mice were then treated with either vehicle, 300 mg/kg Chk1 inhibitor, 20 mg/kg gemcitabine or co-administered with 300 mg/kg Chk1 inhibitor and 20 mg/kg gemcitabine two times, three days apart on Days 1 and 4. Treatment of tumor-bearing mice by co-administration of Chk1 inhibitor and gemcitabine resulted in a four-day growth delay in tumors compared to gemcitabine alone.

To assess the diffusion of Chk1 inhibitors into tumor tissue, plasma and tissue levels of Chk1 inhibitor were measured. Using an Alzet pump, 500 mg/kg Chk1 inhibitor was administered to HT29 tumor-bearing mice in a continuous delivery system over a 24 hour period. Plasma samples were taken and then tumors, kidney, liver, spleen and lung were harvested. Time points were collected at 1, 2, 4, 8 and 24 hours. Tissues were extracted and levels of Chk1 inhibitor were quantified. This experiment demonstrated that the Chk1 inhibitor showed penetration into normal and tumor tissue and reached a level of approximately 15 μM in tumor tissue and peaked in spleen tissue at 8 hours at approximately 20 μM. Thus, Chk1 inhibitors were readily taken up by the proliferating cells and deemed useful, in conjunction with Chk1 activating chemotherapeutic agents, as therapies for the treatment of proliferative diseases.

### Example 7

### Use of H3-P Assay to Determine the Effect of Chk1 Inhibitors on Cell Cycle Arrest

The effect of selective Chk1 inhibitors on Chk1 activator induced cell cycle arrest may be assessed using the assay described above. In this example, gemcitabine was used in mice bearing HT29 tumors.

Mice bearing HT29 tumors were treated with vehicle, 100 mg/kg gemcitabine for 48 hours, or 100 mg/kg gemcitabine for 48 hours followed by the addition of Chk1 inhibitor for 24 hours. Tumors were removed, embedded in paraffin and HT29 tumor slices were stained with antibody against H3-P. Mice pretreated with gemicitabine for 48 hours followed by a 24-hour Chk1 inhibitor treatment demonstrated abrogation of the S phase checkpoint, showing approximately 4% mitotic cells, compared to approximately 4% in gemcitabine-treated mice. This experiment demonstrated that the Chk1 inhibitor allows S phase arrested tumor cells to progress out of the gemcitabine-induced cell cycle arrest and into mitosis.

Using this assay, the scheduling and timing of gemcitabine and Chk1 inhibitors may be optimized. The assay also allows, inter alia, for the measurement of biologically efficacious doses of Chk1 inhibitors and optimization of the Chk1 activator dose and/or pretreatment time.

### Example 8

### Use of H3-P Assay to Determine Optimal Dose and Time to Achieve Cell Cycle Synchronization by Chk1 Activator

In a non-limiting embodiment, the H3-P assay discussed above may be used to determine an optimal degree of cell cycle arrest by Chk1 activator. In the present example, the Chk1 activator was gemcitabine, whose target phase is S phase. The animal model was HT29 tumor-bearing mice.

HT29 tumor-bearing mice were treated with 100 mg/kg gemcitabine intraperitoneally (i.p.) and mice were harvested at 1 hr, 2 hr, 4 hr, 6 hr, 12 hr, 24 hr, 48 hr and 72 hr. Tumors from these animals were resected, paraffin embedded and stained with an antibody to H3-P followed by a counter-stained with DAPI. The percentage of mitotic cells (positive to H3-P) was quantified at each time point. The data indicated that most cells arrested in S phase between 12 and 24 hr after gemcitabine administration, with a mitotic index of approximately 1.5, compared to an index of approximately 3 at the 1-6 hr. time points.

To confirm that low H3-P staining corresponds to S phase arrest, tumors were also stained with an S phase marker, phosphorylated Rb-Pser795. Tumor slices taken in the experiment above were stained with the Rb-Pser795 antibody (Cell Signaling Cat# 9301S) and the number of positive staining cells quantified. The results demonstrated that there were more Rb-P staining cells at, 24, 48 and 72 hours than at earlier timepoints. Taken together, these data indicate that the optimal S phase arrest induced by gemcitabine in HT29 tumors occurred in the particular sample tested at 24-48 hours post-gemcitabine treatment.

The kinetics of S phase arrest in response to gemcitabine varies in tumors depending on their doubling time. The human non-small cell lung carcinoma, H460, and the rat breast cancer 137-62 tumors, which have faster doubling times than HT29 tumors (4.5 and 2 days respectively, compared to 10 days or HT29) show reduced H3-P staining at earlier times than HT29 tumors. In an experiment similar to that described above for HT29 cells, H460 and 137-62 were treated with gemcitabine and tumors were harvested at various timepoints. In both tumor types, the lowest H3-P staining is at 12 hours (compared to 48 hr in HT29 cells) and the cells exited S phase arrest at 24 hours in 137-62 cells and 48 hours in H460 cells.

These results suggest that faster growing tumors cycle around into S phase and arrest more rapidly than slower growing tumors. Furthermore, the faster the doubling time of the tumor, the faster they enter back into the cell cycle after gemcitabine arrest. Thus, the optimal gemcitabine pretreatment time may vary depending on the doubling time of the tumor. The fairly broad range of observed pretreatment times that resulted in an S phase arrest suggests that it will be practical to translate this regime to the clinic or laboratory.

### Example 9

### An Assessment of Optimal Contact Time with Chk1 Inhibitor Following Substantial Cell Cycle Synchronization

This example illustrates an assessment of the effects of Chk1 inhibitors on kinetics of the abrogation of the cell cycle arrest following substantial synchronization by Chk1 activator. In the present non-limiting example, a cell population comprising human colon carcinoma cell line HT29 was treated with 20 μM gemcitabine for two hours, the gemcitabine washed out, and cells allowed to substantially synchronize at S phase. After 18 hours, the cells were then treated with Chk1 inhibitor and time points taken from 30 minutes to 24 hours. Results showed that progression through the S phase checkpoint started at 2 hours and peaked at 8 hours, with approximately 80% of cells in mitosis. Levels of cells entering into mitosis dropped off by 24 hours, presumably because the cells began to die. These data suggest that the optimal time of exposure of HT29 cells to Chk1 inhibitor after gemcitabine-induced S phase arrest in the samples tested was 6-8 hours. It was observed that some cell lines that are sensitized to Chk1 inhibitors and gemcitabine (such as the 137-62 breast cell carcinoma) enter into mitosis after S phase arrest with this chemotherapy treatment. However, based on the cell sensitization data gathered, it is believed likely that in these cells the Chk1 inhibitors allow abrogation of the cell cycle checkpoint, but rather than progress into mitosis, they progress out of S phase and then die via apoptosis.

### Example 10

### An Assessment of Dose Response of Chk1 Inhibitor Abrogation Following Substantial Cell Cycle Synchronization

To determine whether checkpoint abrogation by selective Chk1 inhibitor was dose-dependent, HT29 tumor-bearing mice were pretreated with gemcitabine and 32 hours later dosed with increasing doses of selective Chk1 inhibitor. After 18 hours, tumors were harvested and stained for H3-P as described above. Results indicated that entry into mitosis after checkpoint abrogation is dose dependent, with about 5% of cells in mitosis at 100 mg/kg of Chk1 inhibitor, increasing to approximately 11% at 400 mg/kg. The response is saturated at 400 mg/kg. These data confirm a dose-dependent response to Chk1 inhibitor up to a saturation point.

### Example 11

### Dose Response of Tumors Treated with Chk1 Inhibitors and Gemcitabine

To determine an efficacious dose of Chk1 inhibitor following gemcitabine treatment and whether the dose-dependent checkpoint abrogation correlated with anti-tumor activity, a dose response experiment was performed.

Nude mice were engrafted with HT29 tumor cells and tumors allowed to develop for 10 days. The tumors at the start were approximately 100 mm3. Animals were treated with gemcitabine at the MID (160 mg/kg) followed by Chk1 inhibitor at 50 mg/kg, 200 mg/kg or 400 mg/kg administered as in Example 1. Gemcitabine pretreatment time was 32 hours in this experiment, as the cell-based assay indicated this timepoint was optimal for this type of tumor. Analysis of tumor volume in each treatment regimen indicated that treatment of HT29 tumor bearing mice with the described therapy slowed tumor growth greater than gemcitabine alone, with either 200 mg/kg or 400 mg/kg Chk1 inhibitor plus gemcitabine again showing dose-dependent effects of the Chk1 inhibitor.

### Example 12

### An Assay to Determine Whether an Agent is a Chk1 Activator

To determine whether an agent is a Chk1 activator, the phosphorylation state of Chk1 can be measured using phospho-specific antibodies to specific phosphorylation sites on Chk1. Serines 317 and 345 have been shown to be phosphorylated after treatment of cells with ionizing radiation, ultraviolet radiation, hydroxyurea, N-methyl-N′-nitro-N-nitrosoguanidine (MNNG), temozolamide and gemcitabine. Liu, Q., et al., (2000) Genes Dev. 14, 1448-1459; Zhao, H., et al., (2001) Mol. Cell. Biol. 21, 4129-4139; Lopez-Girona, A., et al., (2001) Proc. Natl. Acad. Sci. U.S.A. 98, 11289-11294; Guo, Z., et al., (2000). Genes Dev. 14, 2745-2756; Gatei, M., et al., (2003) J. Biol. Chem. 278, 14806-14811; Ng C P, et al., J Biol Chem. 2004 Mar. 5; 279(10):8808-19; Wang Y, et al., Natl Acad Sci USA. 2003 Dec. 23; 100(26):15387-92; Stojic L, et al., Genes Dev. 2004 Jun. 1; 18(11):1331-44. These serine sites are phosphorylated by upstream checkpoint kinases, Atm and Atr. Liu, Q., et al., S. J. (2000) Genes Dev. 14, 1448-1459; Zhao, H., et al. (2001) Mol. Cell. Biol. 21, 4129-4139).

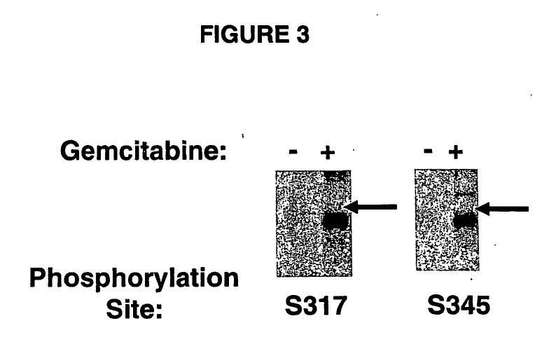

The phosphorylation of these sites in response to a candidate Chk1 activator can be monitored by Western blot or immunohistochemistry of tumor cells. For example, the following procedure was used to demonstrate that gemcitabine results in Chk1 activation at serine 345 and 317. HT29 cells were treated with 20 μM gemcitabine for two hours. The gemcitabine was washed out of the cell growth media and cells were incubated for 22 additional hours. Protein lysates were prepared and separated by an SDS-polyacrylamide gel electrophoresis. Proteins were transferred to PVDF membranes and probed with antisera (Cell Signalling) specific for either phosphorylated serine 317 or 345 (Cell. Signalling). FIG. 3 shows, by Western blot, that gemcitabine treatment of HT29-colon carcinoma cells results in the phosphorylation of both serines 317 and 345.

### Example 13

### An Assay to Monitor Chk1 Activity in Response to a Chk1 Inhibitor

Applicants have found that phosphorylation of Chk1 at serine 296 is stimulated by treatment of tumor cells with gemcitabine, and that phosphorylation at this site is inhibited by Chk1 inhibitors. Phosphorylation at this site is not inhibited by Wortinannin, which inhibits Atm and Atr. Therefore the phosphorylation of serine 296 is distinct from phosphorylation at serines 317 and 345 described in Example 12. In addition, Applicants have found that this site is phosphorylated in purified Chk1 preparations, suggesting that the purified enzyme is able to phosphorylate itself or other Chk1 molecules at serine 296. Taken together, these data suggest that phosphorylation at serine 296 is performed by Chk1 itself. Therefore, this approach may be used to monitor Chk1 activity in tumors in response to Chk1 activators. Further, this approach may be used to measure inhibition of Chk1 activation by Chk1 inhibitors.

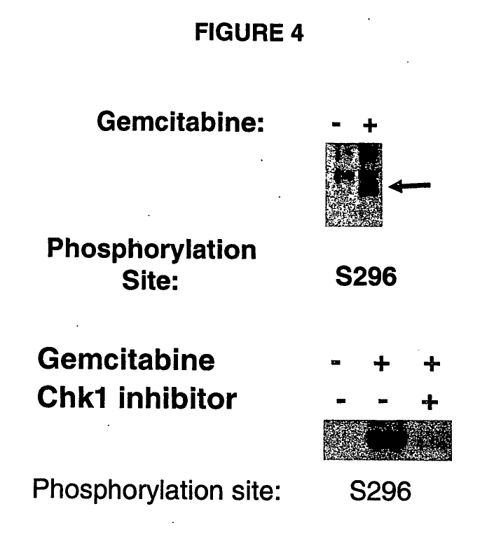

Thus, HT 29 cells were treated with 20 μM gemcitabine for two hours. The gemcitabine was washed out of the cell growth media and cells were incubated for 22 additional hours. Protein lysates were prepared and separated by an SDS-polyacrylamide gel electrophoresis. Proteins were transferred to polyvinylidene fluoride (PVDF) membranes and probed with antisera (Cell Signalling) specific for phosphorlyated serine 296 (Cell Signalling). FIG. 4 shows, by Western blot, that gemcitabine treatment of HT29 colon carcinoma cells results in the phosphorylation of serine 296. Further, HT29 cells treated with selective Chk1 inhibitors for 15 minutes show no serine 296 phosphorylation. These data suggest that serine 296 phosphorylation is performed by the Chk1 kinase.

The present invention is not to be limited in scope by the exemplified embodiments, which are intended as illustrations of single aspects of the invention, and compositions, and methods which are functionally equivalent are within the scope of the invention. Indeed, numerous modifications and variations in the practice of the invention are expected to occur to those skilled in the art upon consideration of the present preferred embodiments. Consequently, the only limitations that should be placed upon the scope of the invention are those that appear in the appended claims. All references cited within the body of the instant specification are hereby incorporated by reference in their entirety.

We claim:

1. A method for controlling aberrant cell proliferation comprising
a) contacting a cell population comprising aberrantly proliferating cells with at least one Chk1 activator in am amount sufficient to substantially synchronize cell cycle arrest among said aberrantly proliferating cells at a target phase, and
b) upon achieving said substantial synchronization of cell cycle arrest among said aberrantly proliferating cells, contacting said cell population with a selective Chk1 inhibitor in an amount sufficient to substantially abrogate said cell cycle arrest.

2. The method of claim 1, wherein said Chk1 inhibitor is a specific Chk1 inhibitor.

3. The method of claim 1, wherein said cell population is contacted with a Chk1 activator for from about 30 minutes to about 96 hours, and subsequently contacted with a selective Chk1 inhibitor for from up to about 1 hour to up to about 72 hours.

4. The method of claim 3, wherein said cell population is contacted with a Chk1 activator for from about 30 minutes to about 48 hours.

5. The method of claim 1, wherein said Chk1 activator induces substantial synchronization of cell cycle arrest cells at the target phase G1.

6. The method of claim 1, wherein said Chk1 activator induces substantial synchronization of cell cycle arrest at the target phase S.

7. The method of claim 1, wherein said Chk1 activator induces substantial synchronization of cell cycle arrest at the target phase G2.

8. The method of claim 1, wherein said cell population is ex vivo.

9. The method of claim 1, wherein said cell population is in vivo.

10. The method of claim 9, wherein said cell population is in a human.

11. The method of claim 1, wherein said Chk1 activator comprises a chemotherapeutic agent.

12. The method of claim 1, wherein said Chk1 activator is an alkylating agent.

13. The method of claim 12, wherein said alkylating agent is a nitrogen mustard.

14. The method of claim 13, wherein said nitrogen mustard is mechlorethamine, cyclophosphamide, ifosfamide, melphalan, or chlorambucil.

15. The method of claim 1, wherein said Chk1 activator is a nitrosourea.

16. The method of claim 15, wherein said nitrosourea is carmustine (BCNU), lomustine (CCNU), or semustine (methyl-CCNU).

17. The method of claim 1, wherein said Chk1 activator is an ethylenimine or a methyl-melamine.

18. The method of claim 17, wherein said ethylenimine or said methyl-melanine is triethylenemelamine (TEM), triethylene thiophosphoramide (thiotepa), or hexamethylmelamine (HMM, altretamine).

19. The method of claim 1, wherein said Chk1 activator is an alkyl sulfonate.

20. The method of claim 19, wherein said alkyl sulfonate is busulfan.

21. The method of claim 1, wherein said Chk1 activator is a triazine.

22. The method of claim 21, wherein said triazine is dacarbazine (DTIC).

23. The method of claim 1, wherein said Chk1 activator is an antimetabolite.

24. The method of claim 23, wherein said antimetabolite is a folic acid analog.

25. The method of claim 24, wherein said folic acid analog is methotrexate, trimetrexate, or pemetrexed (multi-targeted antifolate).

26. The method of claim 23, wherein said antimetabolite is a pyrimidine analog.

27. The method of claim 26, wherein said pyrimidine analog is 5-fluorouracil (5-FU), fluorodeoxyuridine, gemcitabine, cytosine arabinoside (AraC, cytarabine), 5-azacytidine, or 2,2′-difluorodeoxycytidine.

28. The method of claim 23, wherein said antimetabolite is a purine analog.

29. The method of claim 28, wherein said purine analog is 6-mercaptopurine, 6-thioguanine, azathioprine, 2′-deoxycoformycin (pentostatin), erythrohydroxynonyladenine (EHNA), a fludarabine salt, or 2-chlorodeoxyadenosine (cladribine, 2-CdA).

30. The method of claim 23, wherein said antimetabolite is a type I topoisomerase inhibitor.

31. The method of claim 30, wherein said type I topoisomerase inhibitor is camptothecin (CPT), topotecan, or irinotecan.

32. The method of claim 1, wherein said Chk1 activator is derived from a natural product.

33. The method of claim 32, wherein said natural product is a epipodophylotoxin.

34. The method of claim 33, wherein said epipodophylotoxin is etoposide or teniposide.

35. The method of claim 32, wherein said natural product is a vinca alkaloid.

36. The method of claim 35, wherein said vinca alkaloid is vinblastine, vincristine, or vinorelbine.

37. The method of claim 1, wherein said Chk1 activator is an antibiotic.

38. The method of claim 37, wherein said antibiotic is actinomycin D, doxorubicin, or bleomycin.

39. The method of claim 1, wherein said Chk1 activator is a radiosensitizer.

40. The method of claim 39, wherein said radiosensitizer is 5-bromodeozyuridine, 5-iododeoxyuridine, or bromodeoxycytidine.

41. The method of claim 1, wherein said Chk1 activator is a platinum coordination complex.

42. The method of claim 41, wherein said platinum coordination complex is a cisplatin, carboplatin, or oxaliplatin.

43. The method of claim 1, wherein said Chk1 activator is hydroxyurea.

44. The method of claim 1, wherein said Chk1 activator is a methylhydrazine derivative.

45. The method of claim 44, wherein said methylhydrazine derivative is 8 N-methylhydrazine (MIH) or procarbazine.

46. The method of claim 1, wherein said Chk1 activator comprises radiation.

47. The method of claim 46, wherein said radiation is x-ray radiation or ultraviolet radiation.

48. The method of claim 47, wherein said radiation is administered in conjunction with a radiosensitizer and/or a photosensitizer.

49. The method of claim 1, further comprising the administration of at least one chemotherapeutic agent or at least one radiotherapeutic agent that does not activate Chk1.

50. The method of claim 1, further comprising the administration of at least one side effect reducing agent.

51. The method of claim 1, wherein said cell population is contacted with a Chk1 inhibitor after a time sufficient to allow said Chk1 activator to induce a maximum degree of synchronization in said cell population of cell cycle arrest and a minimum number of cells in mitosis.

52. The method of claim 1, wherein the substantially synchronized cell cycle arrest achieved by contacting said cell population with said Chk1 activator comprises at least about a 50% increase in the number of aberrantly proliferating cells in the target phase of said Chk1 activator in comparison to the number of aberrantly proliferating cells in the target phase prior to contact with said Chk1 activator.

53. The method of claim 52, wherein said increase is at least about 100%.

54. The method of claim 53, wherein said increase is at least about 200%.

55. The method of claim 54, wherein said increase is at least about 300%.

56. The method of claim 55, wherein said increase is at least about 400%.

57. The method of claim 1, wherein said cell population is contacted with said Chk1 activator for at least one doubling period typical of aberrantly proliferating cells in said cell population.

58. The method of claim 1, wherein said cell population is contacted with said Chk1 activator for at least two doubling periods typical of aberrantly proliferating cells in said cell population.

59. The method of claim 1, further comprising determining the presence or absence of substantial synchronization of cell cycle arrest in a biological sample.

60. The method of claim 59 wherein the biological sample is a fluid sample or a tissue sample.

61. The method of claim 1, wherein said Chk1 inhibitor is administered over a plurality of doses.

62. The method of claim 25, wherein said doses comprise a frequency of (q4d×4), (q3d×4), (qd×5), (qwk3), or (5/2/5).

63. The method of claim 1, wherein said aberrantly proliferating cells are cancerous.

64. The method of claim 63, wherein said cancerous cells comprise cells from myxoid and round cell carcinomas, locally advanced tumors, metastatic cancer, Ewing's sarcoma, cancer metastases, lymphatic metastases, squamous cell carcinomas, esophageal squamous cell carcinomas, oral carcinomas, multiple myelomas, acute lymphocytic leukemias, acute non-lymphocytic leukemias, chronic lymphocytic leukemias, chronic myelocytic leukemias, hairy cell leukemias, effusion lymphomas (body cavity based lymphomas), thymic lymphoma lung cancers, small cell carcinomas of the lung, cutaneous T cell lymphomas, Hodgkin's lymphomas, non-Hodgkin's lymphomas, cancers of the adrenal cortex, ACTH-producing tumors, non-small cell lung cancers, breast cancers, small cell carcinomas, ductal carcinomas, stomach cancers, colon cancers, colorectal cancers, polyps associated with colorectal neoptasias, pancreatic cancers, liver cancers, bladder cancers, primary superficial bladder tumors, invasive transitional cell carcinomas of the bladder, muscle-invasive bladder cancers, prostate cancers, ovarian carcinomas, primary peritoneal epithelial neoplasms, cervical carcinomas, uterine endometrial cancers, vaginal cancers, cancers of the vulva, uterine cancers and solid tumors in the ovarian follicle, testicular cancers, penile cancers, renal cell carcinomas, intrinsic brain tumors, neuroblastomas, astrocytic brain tumors, gliomas, metastatic tumor cell invasions in the central nervous system, osteomas and osteosarcomas, malignant melanomas, tumor progressions of human skin keratinocytes, squamous cell cancers, thyroid cancers, retinoblastomas, neuroblastomas, peritoneal effusions, malignant pleural effusions, mesotheliomas, Wilms's tumors, gall bladder cancers, trophoblastic neoplasms, hemangiopericytomas, Kaposi's sarcomas or other cancers treatable with chemotherapy agents or inhibitors of cell cycle checkpoint proteins.

65. The method of claim 1, wherein said aberrantly proliferating cells are non-cancerous.

66. The method of claim 63, wherein said non-cancerous cells comprise cells originating from atherosclerosis, restenosis, vasculitis, nephritis, retinopathy, renal disease, proliferative skin disorders, psoriasis, keloid scarring, actinic keratosis, Stevens-Johnson Syndrome, rheumatoid arthritis, systemic-onset juvenile chronic arthritis, osteoporosis, systemic lupus erythematosus, hyperproliferative diseases of the eye including epithelial down growth, proliferative vitreoretinopathy (PVR), hemangio-proliferative diseases, ichthyosis, or papillomas.

67. Use of a composition comprising at least one Chk1 inhibitor in the manufacture of a medicament for the inhibition of aberrant cell proliferation.

68. An article of manufacture comprising a Chk1 inhibitor and a label indicating a method according to claim 1.
